(19) United States
(12) Patent Application Publication
(10) Pub. No.: US 2026/0285834 A1
(43) Pub. Date: Sep. 24, 2026

(54) SUBSTITUTED PYRIDINE DERIVATIVES AS SARM1 INHIBITORS

(71) Applicant: Nura Bio, Inc., South San Francisco, CA (US)

(72) Inventors: Christopher Michael TEGLEY, San Carlos, CA (US); Andrew Stewart TASKER, Simi Valley, CA (US); Firoz JAIPURI, Fremont, CA (US); Cheryl A. GRICE, Castle Rock, CO (US)

(21) Appl. No.: 18/998,073

(22) PCT Filed: Jul. 26, 2023
(86) PCT No.: PCT/US2023/071058

§ 371 Date: Jan. 23, 2025

Related U.S. Application Data
(60) Provisional application No. 63/392,818, filed on Jul. 27, 2022.
(60) Provisional application No. 63/387,460, filed on Dec. 14, 2022.

(51) Int. Cl. C07D 401/14; A61K 31/4439; A61K 45/06; C07D 401/06; C07D 413/06

(52) U.S. Cl. CPC C07D 401/14; A61K 31/4439; A61K 45/06; C07D 401/06; C07D 413/06

(57) ABSTRACT

This disclosure is drawn to pyridine derivatives, compositions thereof, and associated methods, useful for inhibition of SARM1 activity and/or for treating or preventing neurological disorders.

### CROSS-REFERENCE

This application claims benefit of U.S. Provisional Patent Application No. 63/392,818, filed on Jul. 27, 2022 and U.S. Provisional Patent Application No. 63/387,460, filed on Dec. 14, 2022, each of which is incorporated herein by reference in its entirety.

### FIELD OF THE INVENTION

Described herein are compounds and compositions, and associated methods, useful for inhibition of SARM1 activity and/or for treating or preventing a neurological disorder.

### BACKGROUND OF THE INVENTION

Aging constitutes the main risk factor for the development of neurodegenerative diseases. Axonal degeneration is an important pathological event in many neurodegenerative and neurological disorders, including peripheral neuropathy and traumatic brain injury (Gerdts, J. et al., Neuron, 2016, 89, 449-60). Axonal degeneration has also been implicated in, for example, Alzheimer's disease, Parkinson's disease, and Amyotrophic Lateral Sclerosis, where degeneration precedes symptom onset and widespread neuronal loss (Kurowska, Z. et al., J.

Parkinson's Dis., 2016, 6, 703-07). While these neurological conditions have unique underlying etiologies, inhibition of axonal degeneration in the conditions' early stages may slow or prevent their progression by preventing the loss of functional synapses and maintaining neuronal connectivity (Essuman, K. et al., Neuron, 2017 Mar. 22, 93(6), 1334-43).

Axonal degeneration after injury occurs both toward the proximal cell body (termed retrograde degeneration) and toward the distal axon terminal (termed Wallerian or orthograde degeneration) (Kanamori A. et al., Am. J. Pathol. 2012 July; 181(1):62-73). Wallerian degeneration, which occurs in that section of the axon that is distal to the site of injury, occurs after axonal injury in both the peripheral nervous system (PNS) and the central nervous system (CNS). Wallerian degeneration usually begins within 24-36 hours of a lesion. Prior to degeneration, the distal section of the axon tends to remain electrically excitable, while after injury, the axonal skeleton disintegrates and the axonal membrane breaks apart.

The processes of death of the cell body and degeneration of the axon are independent events. As alluded to above, evidence exists indicating that the degeneration of axons precedes clinical symptoms in neurodegenerative diseases and occurs before cell body loss. Thus, axonal degeneration constitutes an early event in pathological processes an d provides a potential therapeutic target to treat neurodegeneration prior to neuronal cell death (Salvadores, N. et al., Front. Neurosci., 2017, 11, 451).

In view of the above, new modalities are needed for the treatment of neurological disorders such as neurodegenerative disease by the prevention of axonal degeneration.

### SUMMARY OF THE INVENTION

In one aspect described herein is a compound of Formula (I′), or a pharmaceutically acceptable salt thereof:

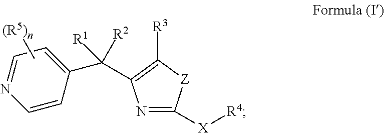

wherein:
Z is selected from O or N(R⁶);
X is a bond or —C(R⁷)(R⁸)—
R¹ is H or C₁₋₆alkyl;
R² is H or C₁₋₆alkyl;
R³ is selected from H, halogen, C₁₋₆alkyl, and C₁₋₆haloalkyl;
R⁴ is selected from C₃₋₆cycloalkyl and C₂₋₉heterocycloalkyl, wherein C₃₋₆cycloalkyl and C₂₋₉heterocycloalkyl are substituted with 1, 2, 3, 4, or 5 groups independently selected from halogen, —CN, C₁₋₆haloalkyl, and C₁₋₄haloalkoxy, and wherein C₃₋₆cycloalkyl and C₂₋₉heterocycloalkyl are optionally substituted with 1, 2, 3, 4, or 5 groups independently selected from —OH, —NH₂, —N(H)(C₁₋₄alkyl), —N(C₁₋₄alkyl)₂, C₁₋₄alkyl, and C₁₋₄alkoxy;
each R⁵ is independently selected from halogen, C₁₋₆alkyl, and C₁₋₆haloalkyl;
R⁶ is selected from H and C₁₋₆alkyl;
R⁷ and R⁸ are each independently selected from H, —OR⁹, C₁₋₄alkyl, and C₁₄haloalkyl;
each R⁹ is independently selected from H, C₁₋₆alkyl, and C₁₋₆haloalkyl, wherein said C₁₋₆alkyl is optionally substituted with 1, 2, or 3 substituents independently selected from —OH, —CN, —NH₂, —N(H)(C₁₋₄alkyl), —N(C₁₋₄alkyl)₂, halogen, C₁₋₄alkyl, C₁₋₄alkoxy, C₁₋₄haloalkyl, and C₁₋₄haloalkoxy; and
n is 0, 1, 2, 3, or 4.

In one aspect described herein is a compound of Formula (I), or a pharmaceutically acceptable salt thereof:

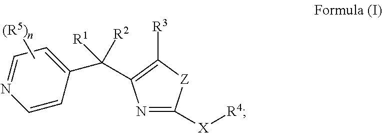

wherein:
Z is selected from O or N(R⁶);
X is a bond or —C(R⁷)(R⁸)—;
R¹ is H or C₁₋₆alkyl;
R² is H or C₁₋₆alkyl;
R³ is selected from H, halogen, C₁₋₆alkyl, and C₁₋₆haloalkyl;
R⁴ is selected from C₃₋₆cycloalkyl and C₂₋₉heterocycloalkyl, wherein C₃₋₆cycloalkyl and C₂₋₉heterocycloalkyl are substituted with 1, 2, 3, 4, or 5 groups independently selected from halogen and C₁₋₆haloalkyl and wherein C₃₋₆cycloalkyl and C₂₋₉heterocycloalkyl are optionally substituted with 1, 2, 3, 4, or 5 groups independently selected from —OH, —CN, —NH₂, —N(H)(C₁₋₄ alkyl), —N(C₁₋₄alkyl)₂, C₁₋₄alkyl, C₁₋₄alkoxy, and C₁₋₄haloalkoxy;
each R⁵ is independently selected from halogen, C₁₋₆alkyl, and C₁₋₆haloalkyl;
R⁶ is selected from H and C₁₋₆alkyl;
R⁷ and R⁸ are each independently selected from H, —OR⁹, C₁₋₄alkyl, and C₁₋₄haloalkyl; each R⁹ is independently selected from H, C₁₋₆alkyl, and C₁₋₆haloalkyl, wherein said C₁₋₆alkyl is optionally substituted with 1, 2, or 3 substituents independently selected from —OH, —CN, —NH₂, —N(H)(C₁₋₄alkyl), —N(C₁₋₄alkyl)₂, halogen, C₁₋₄alkyl, C₁₋₄alkoxy, C₁₋₄haloalkyl, and C₁₋₄haloalkoxy; and
n is 0, 1, 2, 3, or 4.

In some embodiments is a compound of Formula (I) or (I′), wherein X is a bond. In some embodiments is a compound of Formula (I) or (I′), wherein X is —C(R⁷)(R⁸)—. In some embodiments is a compound of Formula (I) or (I′), wherein X is —CH₂—. In some embodiments is a compound of Formula (I) or (I′), wherein Z is N(R⁶). In some embodiments is a compound of Formula (I) or (I′), wherein R⁶ is H. In some embodiments is a compound of Formula (I) or (I′), wherein R⁶ is C₁₋₆alkyl. In some embodiments is a compound of Formula (I), wherein Z is O. In some embodiments is a compound of Formula (I) or (I′), wherein R¹ is H. In some embodiments is a compound of Formula (I) or (I′), wherein R² is H. In some embodiments is a compound of Formula (I) or (I′), wherein R² is C₁₋₆alkyl. In some embodiments is a compound of Formula (I) or (I′), wherein R³ is H.

In some embodiments is a compound of Formula (I′), or a pharmaceutically acceptable salt thereof, having Formula (Ia′):

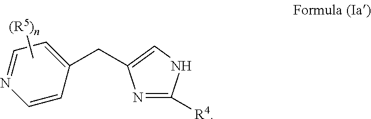

In some embodiments is a compound of Formula (I′), or a pharmaceutically acceptable salt thereof, having Formula (Ib′):

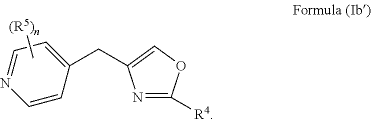

In some embodiments is a compound of Formula (I′), or a pharmaceutically acceptable salt thereof, having Formula (Ic′):

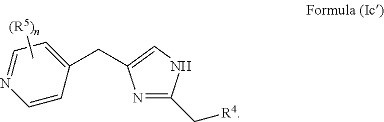

In some embodiments is a compound of Formula (I), or a pharmaceutically acceptable salt thereof, having Formula (Ia):

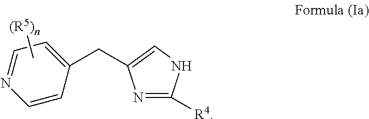

In some embodiments is a compound of Formula (I), or a pharmaceutically acceptable salt thereof, having Formula (Ib):

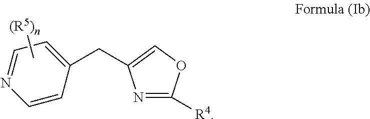

In some embodiments is a compound of Formula (I), or a pharmaceutically acceptable salt thereof, having Formula (Ic):

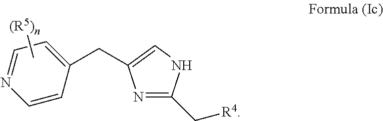

In some embodiments is a compound of Formula (I′), (Ia′), (Ib′), or (Ic′), wherein R⁴ is C₃₋₆cycloalkyl substituted with 1, 2, 3, 4, or 5 groups independently selected from halogen, CN, C₁₋₆haloalkyl, and C₁₋₄haloalkoxy, wherein said C₃₋₆cycloalkyl is optionally substituted with 1, 2, 3, 4, or 5 groups independently selected from —OH, —NH₂, —N(H)(C₁₋₄alkyl), —N(C₁₋₄alkyl)₂, C₁₋₄ alkyl, and C₁₋₄alkoxy. In some embodiments is a compound of Formula (I′), (Ia′), (Ib′), or (Ic′), wherein R⁴ is C₃₋₆cycloalkyl substituted with 1, 2, or 3 groups independently selected from halogen, —CN, C₁₋₆haloalkyl, and C₁₋₄haloalkoxy, wherein said C₃₋₆cycloalkyl is optionally substituted with 1 or 2 groups independently selected from —OH, C₁₋₄alkyl, and C₁₋₄alkoxy. In some embodiments is a compound of Formula (I), (Ia), (Ib), or (Ic), wherein R⁴ is C₃₋₆cycloalkyl substituted with 1, 2, 3, 4, or 5 groups independently selected from halogen and C₁₋₆haloalkyl, and wherein said C₃₋₆cycloalkyl is optionally substituted with 1, 2, 3, 4, or 5 groups independently selected from —OH, —CN, —NH₂, —N(H)(C₁₋₄alkyl), —N(C₁₋₄alkyl)₂, C₁₋₄alkyl, C₁₋₄alkoxy, and C₁₋₄haloalkoxy. In some embodiments is a compound of Formula (I), (Ia), (Ib), or (Ic), wherein R⁴ is C₃₋₆cycloalkyl substituted with 1, 2, 3, or 4 groups independently selected from halogen and C₁₋₆haloalkyl, and wherein said C₃₋₆cycloalkyl is optionally substituted with 1, 2, or 3 groups independently selected from —OH, —CN, C₁₋₄alkyl, C₁₋₄alkoxy, and C₁₋₄haloalkoxy. In some embodiments is a compound of Formula (I), (I′), (Ia), (Ia′), (Ib), (Ib′), (Ic), or (Ic′), wherein R⁴ is C₃₋₆cycloalkyl substituted only with 1, 2, or 3 groups independently selected from halogen and C₁₋₆haloalkyl. In some embodiments is a compound of Formula (I′), (Ia′), (Ib′), or (Ic′), wherein R⁴ is selected from:

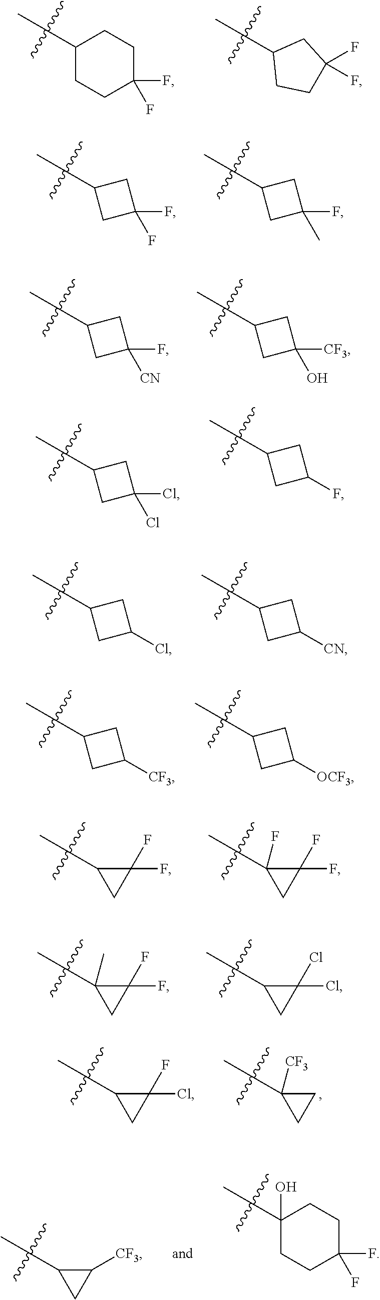

In some embodiments is a compound of Formula (I), (I′), (Ia), (Ia′), (Ib), (Ib′), (Ic), or (Ic′), wherein R⁴ is selected from:

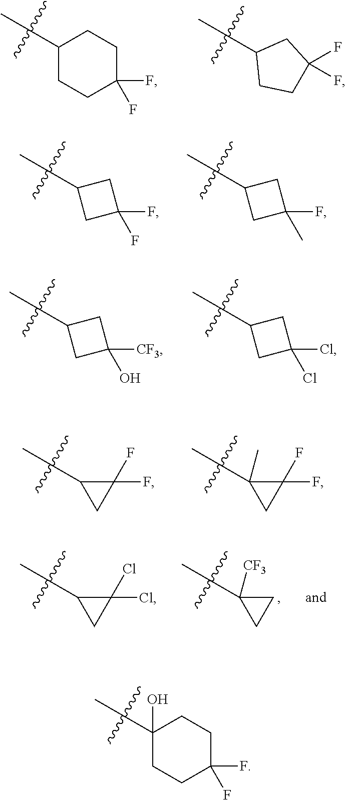

In some embodiments is a compound of Formula (I′), (Ia′), (Ib′), or (Ic′), wherein R⁴ is C₂₋₉heterocycloalkyl substituted with 1, 2, 3, 4, or 5 groups independently selected from halogen, —CN, C₁₋₆haloalkyl, and C₁₋₄haloalkoxy, wherein said C₂₋₉heterocycloalkyl is optionally substituted with 1, 2, 3, 4, or 5 groups independently selected from —OH, —NH₂, —N(H)(C₁₋₄alkyl), —N(C₁₋₄alkyl)₂, C₁₋₄alkyl, and C₁₋₄alkoxy. In some embodiments is a compound of Formula (I′), (Ia′), (Ib′), or (Ic′), wherein R⁴ is C₂₋₉heterocycloalkyl substituted with 1, 2, or 3 groups independently selected from halogen, —CN, C₁₋₆haloalkyl, and C₁₋₄haloalkoxy, wherein said C₂₋₉heterocycloalkyl is optionally substituted with 1 or 2 groups independently selected from —OH, C₁₋₄alkyl, and C₁₋₄alkoxy. In some embodiments is a compound of Formula (I), (Ia), (Ib), or (Ic), wherein R⁴ is C₂₋₉heterocycloalkyl substituted with 1, 2, 3, 4, or 5 groups independently selected from halogen and C₁₋₆haloalkyl, and wherein said C₂₋₉heterocycloalkyl is optionally substituted with 1, 2, 3, 4, or 5 groups independently selected from —OH, —CN, —NH₂, —N(H)(C₁₋₄alkyl), —N(C₁₋₄alkyl)₂, C₁₋₄alkyl, C₁₋₄alkoxy, and C₁₋₄haloalkoxy. In some embodiments is a compound of Formula (I), (I′), (Ia), (Ia′), (Ib), (Ib′), (Ic), or (Ic′), wherein R⁴ is C₂₋₉heterocycloalkyl substituted with 1, 2, 3, or 4 groups independently selected from halogen. In some embodiments is a compound of Formula (I), (I), (Ia), (Ia′), (Ib), (Ib′), (Ic), or (Ic′), wherein R⁴ is selected from:

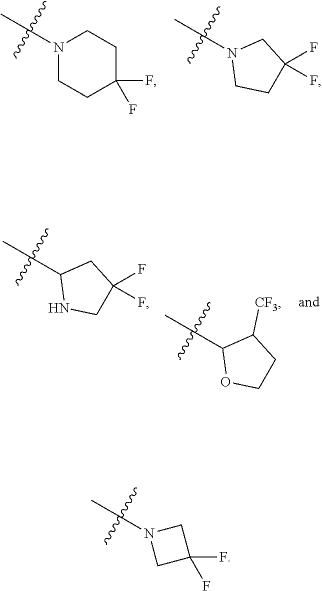 [SMILES: *N1CCC(F)(F)CC1 ; *N1CCC(F)(F)C1 ; *C1CC(F)(F)CN1 ; *C1OCCC1C(F)(F)F ; *N1CC(F)(F)C1]

In some embodiments is a compound of Formula (I), (I′), (Ia), (Ia′), (Ib), (Ib′), (Ic), or (Ic′), wherein R⁴ is selected from:

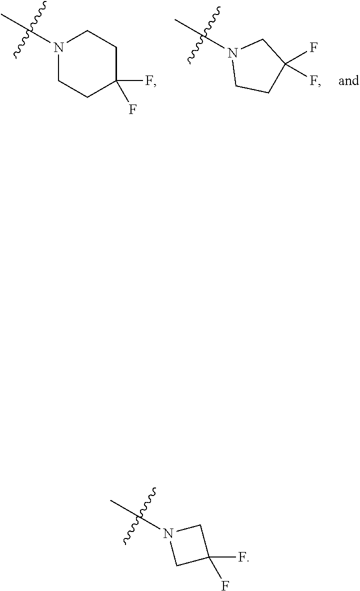 [SMILES: *N1CCC(F)(F)CC1 ; *N1CCC(F)(F)C1 ; *N1CC(F)(F)C1]

In some embodiments is a compound of Formula (I), (I′), (Ia), (Ia′), (Ib), (Ib′), (Ic), or (Ic′), wherein n is 0.

In another aspect is a pharmaceutical composition comprising at least one pharmaceutically acceptable carrier and a compound described herein, or a pharmaceutically acceptable salt thereof.

In another aspect is a method of inhibiting SARM1 comprising contacting the SARM1 with a compound described herein, or a pharmaceutically acceptable salt thereof. In some embodiments is a method of inhibiting SARM1 comprising contacting the SARM1 with a compound described herein, or a pharmaceutically acceptable salt thereof, wherein the contacting is carried out in vitro. In some embodiments is a method of inhibiting SARM1 comprising contacting the SARM1 with a compound described herein, or a pharmaceutically acceptable salt thereof, wherein the contacting is carried out in vivo.

In another aspect is a method of inhibiting axonal degeneration in a patient in need thereof comprising administering to the patient an inhibiting amount of a compound described herein, or a pharmaceutically acceptable salt thereof. In some embodiments is a method of inhibiting axonal degeneration in a patient in need thereof comprising administering to the patient an inhibiting amount of a compound described herein, or a pharmaceutically acceptable salt thereof, wherein the axonal degeneration is caused by abnormal reduction or depletion of NAD+ in the axons.

In another aspect is a method of treating or preventing a neurological disorder in a patient in need thereof comprising administering to the patient a therapeutically effective amount of a compound described herein, or a pharmaceutically acceptable salt thereof. In some embodiments is a method of treating or preventing a neurological disorder in a patient in need thereof comprising administering to the patient a therapeutically effective amount of a compound described herein, or a pharmaceutically acceptable salt thereof, wherein the neurological disorder is a neurodegenerative disease.

In some embodiments is a method of treating or preventing a neurological disorder in a patient in need thereof comprising administering to the patient a therapeutically effective amount of a compound described herein, or a pharmaceutically acceptable salt thereof, in combination with a further pharmaceutically active agent.

In some embodiments is a use of a compound described herein, or a pharmaceutically acceptable salt thereof, in treating or preventing a neurological disorder in a patient in need thereof.

In some embodiments is a compound described herein, or a pharmaceutically acceptable salt thereof, for use in the preparation of a medicament for treating or preventing a neurological disorder in a patient in need thereof.

### DETAILED DESCRIPTION

Similar to programmed cell death pathways (e.g., apoptosis), axonal degeneration in response to injury or disease stimulates a local signaling cascade that causes destruction of the injured axon segment (Summers D. W., et al., PNAS USA, 2016 Oct. 11, 113(41):E6271-E6280). Following injury, the axonal skeleton disintegrates, and the axonal membrane breaks apart. Subsequent to axonal degeneration, the myelin sheath degrades and infiltration by macrophages follows; the macrophages, along with Schwann cells, clear the cellular debris resulting from the degeneration (Coleman M. P., et al., PNAS USA, 1998 August, 95(17):9985-90).

SARM1 (sterile alpha and TIR motif-containing 1) protein (NP_055892) is a 724 amino acid protein involved in axon degeneration. It has also been implicated in infectious and inflammatory disorders. The SARM1 protein, also known as FLJ36296, KIAA0524, MyD88-5, SAM domain-containing protein 2, and SAMD2, comprises four domains, i) a mitochondrial localization signal, ii) an auto-inhibitory N-terminus region consisting of armadillo/HEAT motifs, iii) two sterile alpha motifs responsible for multimerization, and iv) a C-terminus Toll/Interleukin-1 receptor that possesses enzymatic activity (Essuman K., et al., Neuron 2017 March, 93(6):1334-43.e5).

SARM1 protein plays a critical role in the Wallerian degeneration pathway. Activation of SARM1 triggers a rapid collapse of NAD⁺ levels in the distal section of the injured axon, which then undergoes degeneration (Gerdts J. et al., Science 2015 April 348(6233):453-57). Promoting dimerization of the Toll/interleukin receptor (TIR) domain of SARM1 has been shown to be sufficient to promote NAD⁺ loss and axon degeneration.

SARM1's activity is responsible for, at least in part, the protective nature of the survival factor NMNAT2, as NMKNAT enzymes have been found to prevent SARM1-mediated depletion of NAD+. Other pro-degeneration signaling pathways, including the MAP kinase pathway, have been linked to SARM1 activation. MAPK signaling has been shown to promote the loss of NMNAT2, which promotes SARM1 activation (See, e.g., Yang J. et al., Cell 2015 January 160(1-2):161-76).

SARM1 is involved in the innate immune response. It promotes neuronal cell death in response to stress and other stimuli. SARM1 acts as a negative regulator of TICAM1/TRIF-dependent Toll-like receptor signaling by inhibiting induction of TLR3- and TLR4-dependent genes, which play a pivotal role in activating axonal degeneration following injury. In addition, SARM1 specifically blocks TICAM1/TRIF-dependent transcription factor activation and gene induction, without affecting the MYD88-dependent pathway or non-TLR signaling. It is also a negative regulator of NF-kappa-B and IRF activation. (See, e.g., Summers, D. W. et al., J Neurosci., 2014 Jul. 9, 34(28):9338-50).

In some embodiments described herein are inhibitors of SARM1. SARM1 activation can cause a rapid reduction in NAD⁺ levels in injured axons, which then undergo degeneration. In particular embodiments, the compounds inhibit axonal degeneration, including axonal degeneration that results from reduction or depletion of NAD+(e.g., inhibition of SARM1 NADase).

Further described herein are active-site SARM1 NAD hydrolase inhibitors. In some aspects, the inhibitors described herein actin the catalytic pocket but do not directly compete with substrate binding. In some aspects, the inhibitors are uncompetitive, pro-inhibitors that function by opportunistically intercepting the NAD hydrolysis reaction and undergoing covalent conjugation with the reaction product adenosine diphosphate ribose (ADPR). In some embodiments, the resulting small molecule-ADPR adducts confer knock-out like axon protection in vivo—reducing levels of the translatable biomarker neurofilament light and conferring functional protection. In some embodiments, described herein is a mode of pharmacologic inhibition that has implications not just for SARM1 but for a broader panel of related NAD hydrolases linked to age-related decline and disease.

### Compounds

Compounds described herein, including pharmaceutically acceptable salts, active metabolites and pharmaceutically acceptable solvates thereof, are inhibitors of SARM1.

In some embodiments is a compound of Formula (I′):

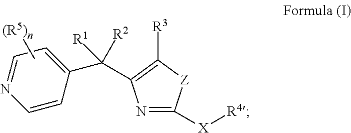

or a pharmaceutically acceptable salt thereof, wherein:
Z is selected from O or N(R⁶);
X is a bond or —C(R⁷)(R⁸)—;
R¹ is H or C₁₋₆alkyl;
R² is H or C₁₋₆alkyl;
R³ is selected from H, halogen, C₁₋₆alkyl, and C₁₋₆haloalkyl;
R⁴ is selected from C₃₋₆cycloalkyl and C₂₋₉heterocycloalkyl, wherein C₃₋₆cycloalkyl and C₂₋₉heterocycloalkyl are substituted with 1, 2, 3, 4, or 5 groups independently selected from halogen, —CN, C₁₋₆haloalkyl, and C₁₋₄haloalkoxy, and wherein C₃₋₆cycloalkyl and C₂₋₉heterocycloalkyl are optionally substituted with 1, 2, 3, 4, or 5 groups independently selected from —OH, —NH₂, —N(H)(C₁₋₄alkyl), —N(C₁₋₄alkyl)₂, C₁₋₄alkyl, and C₁₋₄alkoxy;
each R⁵ is independently selected from halogen, C₁₋₆alkyl, and C₁₋₆haloalkyl;
R⁶ is selected from H and C₁₋₆alkyl;
R⁷ and R⁸ are each independently selected from H, —OR⁹, C₁₋₄alkyl, and C₁₋₄haloalkyl;
each R⁹ is independently selected from H, C₁₋₆alkyl, and C₁₋₆haloalkyl, wherein said C₁₋₆alkyl is optionally substituted with 1, 2, or 3 substituents independently selected from —OH, —CN, —NH₂, —N(H)(C₁₋₄alkyl), —N(C₁₋₄alkyl)₂, halogen, C₁₋₄alkyl, C₁₋₄alkoxy, C₁₋₄haloalkyl, and C₁₋₄haloalkoxy; and
n is 0, 1, 2, 3, or 4.

In some embodiments is a compound of Formula (I′), wherein Z is N(R⁶). In some embodiments is a compound of Formula (I′), wherein Z is N(R⁶) and R⁶ is H. In some embodiments is a compound of Formula (I′), wherein Z is N(R⁶) and R⁶ is C₁₋₆alkyl. In some embodiments is a compound of Formula (I′), wherein Z is O.

In some embodiments is a compound of Formula (I′), wherein X is a bond. In some embodiments is a compound of Formula (I′), wherein X is —C(R⁷)(R⁸)—. In some embodiments is a compound of Formula (I′), wherein X is —C(R⁷)(R⁸)— and R⁷ and R⁸ are each independently selected from H and C₁₋₄alkyl. In some embodiments is a compound of Formula (I′), wherein X is —C(R⁷)(R⁸)—, and R⁷ and R⁸ are H.

In some embodiments is a compound of Formula (I′), wherein R¹ is H. In some embodiments is a compound of Formula (I′), wherein R¹ is C₁₋₆alkyl. In some embodiments is a compound of Formula (I′), wherein R² is H. In some embodiments is a compound of Formula (I′), wherein R²is C₁₋₆alkyl. In some embodiments is a compound of Formula (I′), wherein R¹ and R² are H. In some embodiments is a compound of Formula (I′), wherein R¹ is H and R²is C₁₋₆alkyl.

In some embodiments is a compound of Formula (I′), wherein R³ is H. In some embodiments is a compounds of Formula (I′), wherein R³ is halogen. In some embodiments is a compound of Formula (I′), wherein R³ is C₁₋₆alkyl. In some embodiments is a compound of Formula (I′), wherein R³ is C₁₋₆haloalkyl.

In some embodiments is a compound of Formula (I′), wherein R⁴ is C₃₋₆cycloalkyl substituted with 1, 2, 3, 4, or 5 groups independently selected from halogen, —CN, C₁₋₆haloalkyl, and C₁₋₄haloalkoxy, wherein said C₃₋₆cycloalkyl is optionally substituted with 1, 2, 3, 4, or 5 groups independently selected from —OH, —NH₂, —N(H)(C₁₋₄alkyl), —N(C₁₋₄alkyl)₂, C₁₋₄alkyl, and C₁₋₄alkoxy. In some embodiments is a compound of Formula (I′), wherein R⁴ is C₃₋₆cycloalkyl substituted with 1, 2, 3, or 4 groups independently selected from halogen, —CN, C₁₋₆haloalkyl, and C₁₋₄haloalkoxy, wherein said C₃₋₆cycloalkyl is optionally substituted with 1, 2, or 3 groups independently selected from —OH, C₁₋₄alkyl, and C₁₋₄alkoxy. In some embodiments is a compound of Formula (I′), wherein R⁴ is C₃₋₆cycloalkyl substituted with 1, 2, or 3 groups independently selected from halogen, —CN, C₁₋₆haloalkyl, and C₁₋₄haloalkoxy, wherein said C₃₋₆ cycloalkyl is optionally substituted with 1 or 2 groups independently selected from —OH, C₁₋₄ alkyl, and C₁₋₄alkoxy. In some embodiments is a compound of Formula (I′), wherein R⁴ is C₃₋₆ cycloalkyl substituted with 1, 2, or 3 groups independently selected from halogen, wherein said C₃₋₆cycloalkyl is optionally substituted with 1, 2, or 3 groups independently selected from —OH, C₁₋₄alkyl, and C₁₋₄alkoxy. In some embodiments is a compound of Formula (I′), wherein R⁴ is C₃₋₆cycloalkyl substituted with 1, 2, or 3 groups independently selected from C₁₋₆haloalkyl, wherein said C₃₋₆cycloalkyl is optionally substituted with 1, 2, or 3 groups independently selected from —OH, C₁₋₄alkyl, and C₁₋₄alkoxy. In some embodiments is a compound of Formula (I′), wherein R⁴ is C₃₋₆cycloalkyl substituted with 1, 2, or 3 groups independently selected from C₁₋₄haloalkoxy, wherein said C₃₋₆cycloalkyl is optionally substituted with 1, 2, or 3 groups independently selected from —OH, C₁₋₄alkyl, and C₁₋₄alkoxy. In some embodiments is a compound of Formula (I′), wherein R⁴ is C₃₋₆cycloalkyl substituted with 1 or 2 —CN, wherein said C₃₋₆cycloalkyl is optionally substituted with 1, 2, or 3 groups independently selected from —OH, C₁₋₄alkyl, and C₁₋₄alkoxy. In some embodiments is a compound of Formula (I′), wherein R⁴ is cyclopropyl substituted with 1, 2, 3, 4, or 5 groups independently selected from halogen, —CN, C₁₋₆haloalkyl, and C₁₋₄haloalkoxy, wherein said cyclopropyl is optionally substituted with 1, 2, 3, 4, or 5 groups independently selected from —OH, —NH₂, —N(H)(C₁₋₄alkyl), —N(C₁₋₄alkyl)₂, C₁₋₄ alkyl, and C₁₋₄alkoxy. In some embodiments is a compound of Formula (I′), wherein R⁴ is cyclopropyl substituted with 1, 2, 3, or 4 groups independently selected from halogen, —CN, C₁₋₆ haloalkyl, and C₁₋₄haloalkoxy, wherein said cyclopropyl is optionally substituted with 1, 2, or 3 groups independently selected from —OH, C₁₋₄alkyl, and C₁₋₄alkoxy. In some embodiments is a compound of Formula (I′), wherein R⁴ is cyclopropyl substituted with 1, 2, 3, or 4 groups independently selected from halogen, —CN, C₁₋₆haloalkyl, and C₁₋₄haloalkoxy, wherein said cyclopropyl is optionally substituted with 1, 2, or 3 groups independently selected from —OH, C₁₋₄ alkyl, and C₁₋₄alkoxy. In some embodiments is a compound of Formula (I′), wherein R⁴ is cyclopropyl substituted with 1 or 2 groups independently selected from halogen, —CN, C₁₋₆ haloalkyl, and C₁₋₄haloalkoxy, wherein said cyclopropyl is optionally substituted with 1, 2, or 3 groups independently selected from —OH, C₁₋₄alkyl, and C₁₋₄alkoxy. In some embodiments is a compound of Formula (I′), wherein R⁴ is cyclopropyl substituted with 1 or 2 groups independently selected from halogen and C₁₋₆haloalkyl. In some embodiments is a compound of Formula (I′), wherein R⁴ is cyclopropyl substituted with 1 or 2 groups independently selected from halogen. In some embodiments is a compound of Formula (I′), wherein R⁴ is cyclopropyl substituted with 1 or 2 groups independently selected from C₁₋₆haloalkyl. In some embodiments is a compound of Formula (I′), wherein R⁴ is cyclopropyl substituted with 1 or 2 groups independently selected from C₁₋₄haloalkoxy. In some embodiments is a compound of Formula (I′), wherein R⁴ is cyclopropyl substituted with 1 or 2 groups independently selected from —CN. In some embodiments is a compound of Formula (I′), wherein R⁴ is cyclobutyl substituted with 1, 2, 3, 4, or 5 groups independently selected from halogen, —CN, C₁₋₆haloalkyl, and C₁₋₄ haloalkoxy, wherein said cyclobutyl is optionally substituted with 1, 2, 3, 4, or 5 groups independently selected from —OH, —NH₂, —N(H)(C₁₋₄alkyl), —N(C₁₋₄alkyl)₂, C₁₋₄alkyl, and C₁₋₄alkoxy. In some embodiments is a compound of Formula (I′), wherein R⁴ is cyclobutyl substituted with 1, 2, 3, or 4 groups independently selected from halogen, —CN, C₁₋₆haloalkyl, and C₁₋₄haloalkoxy, wherein said cyclobutyl is optionally substituted with 1, 2, or 3 groups independently selected from —OH, C₁₋₄alkyl, and C₁₋₄alkoxy. In some embodiments is a compound of Formula (I′), wherein R⁴ is cyclobutyl substituted with 1, 2, 3, or 4 groups independently selected from halogen, —CN, C₁₋₆haloalkyl, and C₁₋₄haloalkoxy, wherein said cyclobutyl is optionally substituted with 1, 2, or 3 groups independently selected from —OH, C₁₋₄ alkyl, and C₁₋₄alkoxy. In some embodiments is a compound of Formula (I′), wherein R⁴ is cyclobutyl substituted with 1 or 2 groups independently selected from halogen, —CN, C₁₋₆ haloalkyl, and C₁₋₄haloalkoxy, wherein said cyclobutyl is optionally substituted with 1, 2, or 3 groups independently selected from —OH, C₁₋₄alkyl, and C₁₋₄alkoxy. In some embodiments is a compound of Formula (I′), wherein R⁴ is cyclobutyl substituted with 1 or 2 groups independently selected from halogen and C₁₋₆haloalkyl. In some embodiments is a compound of Formula (I′), wherein R⁴ is cyclobutyl substituted with 1 or 2 groups independently selected from halogen. In some embodiments is a compound of Formula (I′), wherein R⁴ is cyclobutyl substituted with 1 or 2 groups independently selected from C₁₋₆haloalkyl. In some embodiments is a compound of Formula (I′), wherein R⁴ is cyclobutyl substituted with 1 or 2 groups independently selected from C₁₋₄haloalkoxy. In some embodiments is a compound of Formula (I′), wherein R⁴ is cyclobutyl substituted with 1 or 2 groups independently selected from —CN. In some embodiments is a compound of Formula (I′), wherein R⁴ is cyclopentyl substituted with 1, 2, 3, 4, or 5 groups independently selected from halogen, —CN, C₁₋₆haloalkyl, and C₁₋₄ haloalkoxy, wherein said cyclopentyl is optionally substituted with 1, 2, 3, 4, or 5 groups independently selected from —OH, —NH₂, —N(H)(C₁₋₄alkyl), —N(C₁₋₄alkyl)₂, C₁₋₄alkyl, and C₁₋₄alkoxy. In some embodiments is a compound of Formula (I′), wherein R⁴ is cyclopentyl substituted with 1, 2, 3, or 4 groups independently selected from halogen, —CN, C₁₋₆haloalkyl, and C₁₋₄haloalkoxy, wherein said cyclopentyl is optionally substituted with 1, 2, or 3 groups independently selected from —OH, C₁₋₄alkyl, and C₁₋₄alkoxy. In some embodiments is a compound of Formula (I′), wherein R⁴ is cyclopentyl substituted with 1, 2, 3, or 4 groups independently selected from halogen, —CN, C₁₋₆haloalkyl, and C₁₋₄haloalkoxy, wherein said cyclopentyl is optionally substituted with 1, 2, or 3 groups independently selected from —OH, C₁₋₄ alkyl, and C₁₋₄alkoxy. In some embodiments is a compound of Formula (I′), wherein R⁴ is cyclopentyl substituted with 1 or 2 groups independently selected from halogen, —CN, C₁₋₆ haloalkyl, and C₁₋₄haloalkoxy, wherein said cyclopentyl is optionally substituted with 1, 2, or 3 groups independently selected from —OH, C₁₋₄alkyl, and C₁₋₄alkoxy. In some embodiments is a compound of Formula (I′), wherein R⁴ is cyclopentyl substituted with 1 or 2 groups independently selected from halogen and C₁₋₆haloalkyl. In some embodiments is a compound of Formula (I′), wherein R⁴ is cyclopentyl substituted with 1 or 2 groups independently selected from halogen. In some embodiments is a compound of Formula (I′), wherein R⁴ is cyclopentyl substituted with 1 or 2 groups independently selected from C₁₋₆haloalkyl. In some embodiments is a compound of Formula (I′), wherein R⁴ is cyclopentyl substituted with 1 or 2 groups independently selected from C₁₋₄haloalkoxy. In some embodiments is a compound of Formula (I′), wherein R⁴ is cyclopentyl substituted with 1 or 2 groups independently selected from —CN. In some embodiments is a compound of Formula (I′), wherein R⁴ is cyclohexyl substituted with 1, 2, 3, 4, or 5 groups independently selected from halogen, —CN, C₁₋₆haloalkyl, and C₁₋₄ haloalkoxy, wherein said cyclohexyl is optionally substituted with 1, 2, 3, 4, or 5 groups independently selected from —OH, —NH₂, —N(H)(C₁₋₄alkyl), —N(C₁₋₄alkyl)₂, C₁₋₄alkyl, and C₁₋₄alkoxy. In some embodiments is a compound of Formula (I′), wherein R⁴ is cyclohexyl substituted with 1, 2, 3, or 4 groups independently selected from halogen, —CN, C₁₋₆haloalkyl, and C₁₋₄haloalkoxy, wherein said cyclohexyl is optionally substituted with 1, 2, or 3 groups independently selected from —OH, C₁₋₄alkyl, and C₁₋₄alkoxy. In some embodiments is a compound of Formula (I′), wherein R⁴ is cyclohexyl substituted with 1, 2, 3, or 4 groups independently selected from halogen, —CN, C₁₋₆haloalkyl, and C₁₋₄haloalkoxy, wherein said cyclohexyl is optionally substituted with 1, 2, or 3 groups independently selected from —OH, C₁₋₄ alkyl, and C₁₋₄alkoxy. In some embodiments is a compound of Formula (I′), wherein R⁴ is cyclohexyl substituted with 1 or 2 groups independently selected from halogen, —CN, C₁₋₆ haloalkyl, and C₁₋₄haloalkoxy, wherein said cyclohexyl is optionally substituted with 1, 2, or 3 groups independently selected from —OH, C₁₋₄alkyl, and C₁₋₄alkoxy. In some embodiments is a compound of Formula (I′), wherein R⁴ is cyclohexyl substituted with 1 or 2 groups independently selected from halogen and C₁₋₆haloalkyl. In some embodiments is a compound of Formula (I′), wherein R⁴ is cyclohexyl substituted with 1 or 2 groups independently selected from halogen. In some embodiments is a compound of Formula (I′), wherein R⁴ is cyclohexyl substituted with 1 or 2 groups independently selected from C₁₋₆haloalkyl. In some embodiments is a compound of Formula (I′), wherein R⁴ is cyclohexyl substituted with 1 or 2 groups independently selected from C₁₋₄haloalkoxy. In some embodiments is a compound of Formula (I′), wherein R⁴ is cyclohexyl substituted with 1 or 2 groups independently selected from —CN.

In some embodiments is a compound of Formula (I′), wherein R⁴ is selected from:

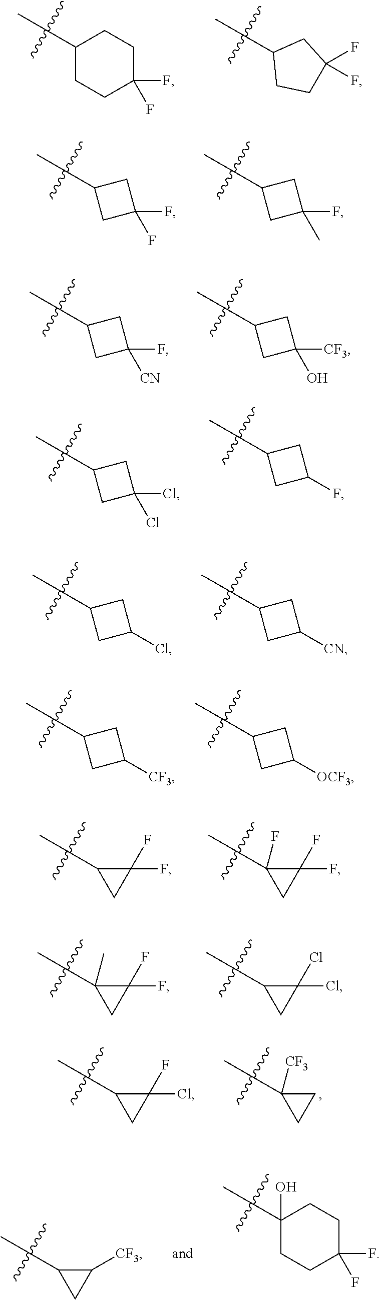

In some embodiments is a compound of Formula (I′), wherein R⁴ is selected from:

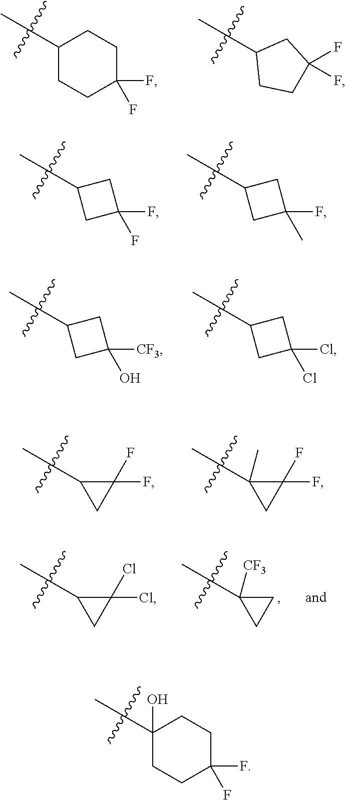

In some embodiments is a compound of Formula (I′), wherein R⁴ is C₂₋₉heterocycloalkyl substituted with 1, 2, 3, 4, or 5 groups independently selected from halogen, —CN, C₁₋₆haloalkyl, and C₁₋₄haloalkoxy, wherein said C₂₋₉heterocycloalkyl is optionally substituted with 1, 2, 3, 4, or 5 groups independently selected from —OH, —NH₂, —N(H)(C₁₋₄alkyl), —N(C₁₋₄alkyl)₂, C₁₋₄alkyl, and C₁₋₄alkoxy. In some embodiments is a compound of Formula (I′), wherein R⁴ is C₂₋₉heterocycloalkyl substituted with 1, 2, 3, 4, or 5 groups independently selected from halogen and C₁₋₆haloalkyl, and wherein said C₂₋₉heterocycloalkyl is optionally substituted with 1, 2, 3, 4, or 5 groups independently selected from —OH, —NH₂, —N(H)(C₁₋₄alkyl), —N(C₁₋₄alkyl)₂, C₁₋₄alkyl, and C₁₋₄alkoxy. In some embodiments is a compound of Formula (I′), wherein R⁴ is C₂₋₉heterocycloalkyl substituted with 1, 2, or 3 groups independently selected from halogen, —CN, C₁₋₆haloalkyl, and C₁₋₄haloalkoxy, wherein said C₂₋₉heterocycloalkyl is optionally substituted with 1 or 2 groups independently selected from —OH, C₁₋₄alkyl, and C₁₋₄alkoxy. In some embodiments is a compound of Formula (I′), wherein R⁴ is C₂₋₉heterocycloalkyl substituted with 1, 2, 3, or 4 groups independently selected from halogen. In some embodiments is a compound of Formula (I′), wherein R⁴ is azetidinyl substituted with 1, 2, 3, 4, or 5 groups independently selected from halogen and C₁₋₆haloalkyl and wherein said azetidinyl is optionally substituted with 1, 2, 3, 4, or 5 groups independently selected from —OH, —NH₂, —N(H)(C₁₋₄alkyl), —N(C₁₋₄ alkyl)₂, C₁₋₄alkyl, and C₁₋₄alkoxy. In some embodiments is a compound of Formula (I′), wherein R⁴ is azetidinyl substituted with 1, 2, or 3 groups independently selected from halogen, —CN, C₁₋₆haloalkyl, and C₁₋₄haloalkoxy, wherein said azetidinyl is optionally substituted with 1 or 2 groups independently selected from —OH, C₁₋₄alkyl, and C₁₋₄alkoxy. In some embodiments is a compound of Formula (I′), wherein R⁴ is azetidinyl substituted with 1, 2, 3, or 4 groups independently selected from halogen and C₁₋₆haloalkyl, and wherein said azetidinyl is optionally substituted with 1, 2, or 3 groups independently selected from —OH, C₁₋₄alkyl, and C₁₋₄alkoxy. In some embodiments is a compound of Formula (I′), wherein R⁴ is azetidinyl substituted with 1, 2, 3, or 4 groups independently selected from halogen, and wherein said azetidinyl is optionally substituted with 1, 2, or 3 groups independently selected from —OH, C₁₋₄alkyl, and C₁₋₄alkoxy. In some embodiments is a compound of Formula (I′), wherein R⁴ is azetidinyl substituted with 1 or 2 groups independently selected from halogen, and wherein said azetidinyl is optionally substituted with 1, 2, or 3 groups independently selected from —OH, C₁₋₄alkyl, and C₁₋₄alkoxy. In some embodiments is a compound of Formula (I′), wherein R⁴ is azetidinyl substituted with 1 or 2 groups independently selected from halogen. In some embodiments is a compound of Formula (I′), wherein R⁴ is pyrrolidinyl substituted with 1, 2, 3, 4, or 5 groups independently selected from halogen and C₁₋₆haloalkyl and wherein said pyrrolidinyl is optionally substituted with 1, 2, 3, 4, or 5 groups independently selected from —OH, —NH₂, —N(H)(C₁₋₄alkyl), —N(C₁₋₄ alkyl)₂, C₁₋₄alkyl, and C₁₋₄alkoxy. In some embodiments is a compound of Formula (I′), wherein R⁴ is pyrrolidinyl substituted with 1, 2, 3, or 4 groups independently selected from halogen and C₁₋₆haloalkyl, and wherein said pyrrolidinyl is optionally substituted with 1, 2, or 3 groups independently selected from —OH, C₁₋₄alkyl, and C₁₋₄alkoxy. In some embodiments is a compound of Formula (I′), wherein R⁴ is pyrrolidinyl substituted with 1, 2, or 3 groups independently selected from halogen, —CN, C₁₋₆haloalkyl, and C₁₋₄haloalkoxy, wherein said pyrrolidinyl is optionally substituted with 1 or 2 groups independently selected from —OH, C₁₋₄ alkyl, and C₁₋₄alkoxy. In some embodiments is a compound of Formula (I′), wherein R⁴ is pyrrolidinyl substituted with 1, 2, 3, or 4 groups independently selected from halogen, and wherein said pyrrolidinyl is optionally substituted with 1, 2, or 3 groups independently selected from —OH, C₁₋₄alkyl, and C₁₋₄alkoxy. In some embodiments is a compound of Formula (I′), wherein R⁴ is pyrrolidinyl substituted with 1, 2, or 3 groups independently selected from halogen, —CN, C₁₋₆haloalkyl, and C₁₋₄haloalkoxy, wherein said pyrrolidinyl is optionally substituted with 1 or 2 groups independently selected from —OH, C₁₋₄alkyl, and C₁₋₄alkoxy. In some embodiments is a compound of Formula (I′), wherein R⁴ is pyrrolidinyl substituted with 1 or 2 groups independently selected from halogen, and wherein said pyrrolidinyl is optionally substituted with 1, 2, or 3 groups independently selected from —OH, C₁₋₄alkyl, and C₁₋₄alkoxy. In some embodiments is a compound of Formula (I′), wherein R⁴ is pyrrolidinyl substituted with 1 or 2 groups independently selected from halogen. In some embodiments is a compound of Formula (I′), wherein R⁴ is piperidinyl substituted with 1, 2, 3, 4, or 5 groups independently selected from halogen and C₁₋₆haloalkyl and wherein said piperidinyl is optionally substituted with 1, 2, 3, 4, or 5 groups independently selected from —OH, —NH₂, —N(H)(C₁₋₄alkyl), —N(C₁₋₄ alkyl)₂, C₁₋₄alkyl, and C₁₋₄alkoxy. In some embodiments is a compound of Formula (I′), wherein R⁴ is piperidinyl substituted with 1, 2, 3, or 4 groups independently selected from halogen and C₁₋₆haloalkyl, and wherein said piperidinyl is optionally substituted with 1, 2, or 3 groups independently selected from —OH, C₁₋₄alkyl, and C₁₋₄alkoxy. In some embodiments is a compound of Formula (I′), wherein R⁴ is piperidinyl substituted with 1, 2, or 3 groups independently selected from halogen, —CN, C₁₋₆haloalkyl, and C₁₋₄haloalkoxy, wherein said piperidinyl is optionally substituted with 1 or 2 groups independently selected from —OH, C₁₋₄ alkyl, and C₁₋₄alkoxy. In some embodiments is a compound of Formula (I′), wherein R⁴ is piperidinyl substituted with 1, 2, 3, or 4 groups independently selected from halogen, and wherein said piperidinyl is optionally substituted with 1, 2, or 3 groups independently selected from —OH, C₁₋₄alkyl, and C₁₋₄alkoxy. In some embodiments is a compound of Formula (I′), wherein R⁴ is piperidinyl substituted with 1 or 2 groups independently selected from halogen, and wherein said piperidinyl is optionally substituted with 1, 2, or 3 groups independently selected from —OH, C₁₋₄alkyl, and C₁₋₄alkoxy. In some embodiments is a compound of Formula (I′), wherein R⁴ is piperidinyl substituted with 1 or 2 groups independently selected from halogen.

In some embodiments is a compound of Formula (I′), wherein R⁴ is selected from:

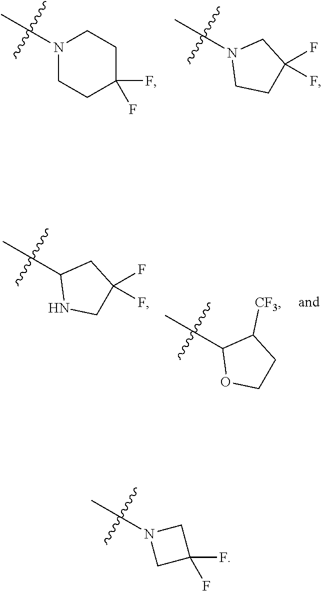 [SMILES: *N1CCC(F)(F)CC1 ; *N1CCC(F)(F)C1 ; *C1CC(F)(F)CN1 ; *C1OCCC1C(F)(F)F ; *N1CC(F)(F)C1]

In some embodiments is a compound of Formula (I′), wherein R⁴ is selected from:

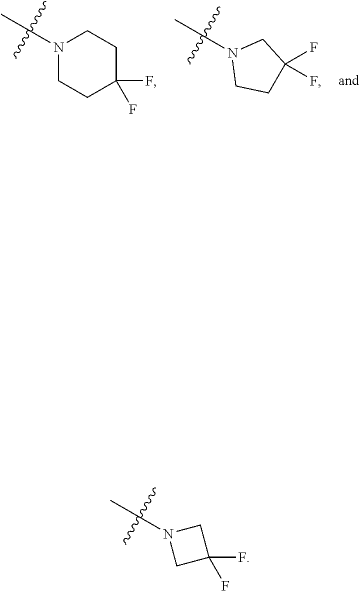 [SMILES: *N1CCC(F)(F)CC1 ; *N1CCC(F)(F)C1 ; *N1CC(F)(F)C1]

In some embodiments is a compound of Formula (I′), wherein each R⁵ is independently selected from halogen and C₁₋₆alkyl. In some embodiments is a compound of Formula (I′), wherein each R⁵ is independently selected from halogen and C₁₋₆haloalkyl. In some embodiments is a compound of Formula (I′), wherein each R⁵ is independently selected from C₁₋₆alkyl and C₁₋₆haloalkyl. In some embodiments is a compound of Formula (I′), wherein each R⁵ is independently selected from halogen. In some embodiments is a compound of Formula (I′), wherein each R⁵ is independently selected from C₁₋₆alkyl. In some embodiments is a compound of Formula (I′), wherein each R⁵ is independently selected from C₁₋₆haloalkyl. In some embodiments is a compound of Formula (I′), wherein n is 0, 1, 2, or 3. In some embodiments is a compound of Formula (I′), wherein n is 1, 2, or 3. In some embodiments is a compound of Formula (I′), wherein n is 1. In some embodiments is a compound of Formula (I′), wherein n is 2. In some embodiments is a compound of Formula (I′), wherein n is 3.

In some embodiments is a compound of Formula (I′), wherein n is 0.

In some embodiments is a compound of Formula (I):

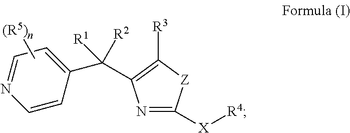

or a pharmaceutically acceptable salt thereof, wherein:
Z is selected from O or N(R⁶);
X is a bond or —C(R⁷)(R⁸)—
R¹ is H or C₁₋₆alkyl;
R² is H or C₁₋₆alkyl;
R³ is selected from H, halogen, C₁₋₆alkyl, and C₁₋₆haloalkyl;
R⁴ is selected from C₃₋₆cycloalkyl and C₂₋₉heterocycloalkyl, wherein C₃₋₆cycloalkyl and C₂₋₉heterocycloalkyl are substituted with 1, 2, 3, 4, or 5 groups independently selected from halogen and C₁₋₆haloalkyl and wherein C₃₋₆cycloalkyl and C₂₋₉heterocycloalkyl are optionally substituted with 1, 2, 3, 4, or 5 groups independently selected from —OH, —CN, —NH₂, —N(H)(C₁₋₄ alkyl), —N(C₁₋₄alkyl)₂, C₁₋₄alkyl, C₁₋₄alkoxy, and C₁₋₄haloalkoxy;
each R⁵ is independently selected from halogen, C₁₋₆alkyl, and C₁₋₆haloalkyl;
R⁶ is selected from H and C₁₋₆alkyl;
R⁷ and R⁸ are each independently selected from H, —OR⁹, C₁₋₄alkyl, and C₁₋₄haloalkyl;
each R⁹ is independently selected from H, C₁₋₆alkyl, and C₁₋₆haloalkyl, wherein said C₁₋₆alkyl is optionally substituted with 1, 2, or 3 substituents independently selected from —OH, —CN, —NH₂, —N(H)(C₁₋₄alkyl), —N(C₁₋₄alkyl)₂, halogen, C₁₋₄alkyl, C₁₋₄alkoxy, C₁₋₄haloalkyl, and C₁₋₄haloalkoxy; and
n is 0, 1, 2, 3, or 4.

In some embodiments is a compound of Formula (I), wherein Z is N(R⁶). In some embodiments is a compound of Formula (I), wherein Z is N(R⁶) and R⁶ is H. In some embodiments is a compound of Formula (I), wherein Z is N(R⁶) and R⁶ is C₁₋₆alkyl. In some embodiments is a compound of Formula (I), wherein Z is O.

In some embodiments is a compound of Formula (I), wherein X is a bond. In some embodiments is a compound of Formula (I), wherein X is —C(R⁷)(R⁸)—. In some embodiments is a compound of Formula (I), wherein X is —C(R⁷)(R⁸)— and R⁷ and R⁸ are each independently selected from H and C₁₋₄alkyl. In some embodiments is a compound of Formula (I), wherein X is —C(R⁷)(R⁸)—, and R⁷ and R⁸ are H.

In some embodiments is a compound of Formula (I), wherein R¹ is H. In some embodiments is a compound of Formula (I), wherein R¹ is C₁₋₆alkyl. In some embodiments is a compound of Formula (I), wherein R² is H. In some embodiments is a compound of Formula (I), wherein R²is C₁₋₆alkyl. In some embodiments is a compound of Formula (I), wherein R¹ and R² are H. In some embodiments is a compound of Formula (I), wherein R¹ is H and R²is C₁₋₆alkyl.

In some embodiments is a compound of Formula (I), wherein R³ is H. In some embodiments is a compounds of Formula (I), wherein R³ is halogen. In some embodiments is a compound of Formula (I), wherein R³ is C₁₋₆alkyl. In some embodiments is a compound of Formula (I), wherein R³ is C₁₋₆haloalkyl.

In some embodiments is a compound of Formula (I), wherein R⁴ is C₃₋₆cycloalkyl substituted with 1, 2, 3, 4, or 5 groups independently selected from halogen and C₁₋₆haloalkyl and wherein said C₃₋₆cycloalkyl is optionally substituted with 1, 2, 3, 4, or 5 groups independently selected from —OH, —CN, —NH₂, —N(H)(C₁₋₄alkyl), —N(C₁₋₄alkyl)₂, C₁₋₄alkyl, C₁₋₄alkoxy, and C₁₋₄haloalkoxy. In some embodiments is a compound of Formula (I), wherein R⁴ is C₃₋₆cycloalkyl substituted with 1, 2, 3, or 4 groups independently selected from halogen and C₁₋₆haloalkyl, and wherein said C₃₋₆cycloalkyl is optionally substituted with 1, 2, or 3 groups independently selected from —OH, —CN, C₁₋₄alkyl, C₁₋₄alkoxy, and C₁₋₄haloalkoxy. In some embodiments is a compound of Formula (I), wherein R⁴ is C₃₋₆cycloalkyl substituted with 1, 2, 3, or 4 groups independently selected from halogen, and wherein said C₃₋₆cycloalkyl is optionally substituted with 1, 2, or 3 groups independently selected from —OH, —CN, C₁₋₄alkyl, C₁₋₄alkoxy, and C₁₋₄haloalkoxy. In some embodiments is a compound of Formula (I), wherein R⁴ is cyclopropyl substituted with 1, 2, 3, 4, or 5 groups independently selected from halogen and C₁₋₆haloalkyl and wherein said cyclopropyl is optionally substituted with 1, 2, 3, 4, or 5 groups independently selected from —OH, —CN, —NH₂, —N(H)(C₁₋₄alkyl), —N(C₁₋₄alkyl)₂, C₁₋₄alkyl, C₁₋₄alkoxy, and C₁₋₄haloalkoxy. In some embodiments is a compound of Formula (I), wherein R⁴ is cyclopropyl substituted with 1, 2, 3, or 4 groups independently selected from halogen and C₁₋₆haloalkyl, and wherein said cyclopropyl is optionally substituted with 1, 2, or 3 groups independently selected from —OH, —CN, C₁₋₄alkyl, C₁₋₄alkoxy, and C₁₋₄haloalkoxy. In some embodiments is a compound of Formula (I), wherein R⁴ is cyclopropyl substituted with 1, 2, 3, or 4 groups independently selected from halogen, and wherein said cyclopropyl is optionally substituted with 1, 2, or 3 groups independently selected from —OH, —CN, C₁₋₄alkyl, C₁₋₄alkoxy, and C₁₋₄haloalkoxy. In some embodiments is a compound of Formula (I), wherein R⁴ is cyclopropyl substituted with 1 or 2 groups independently selected from halogen, and wherein said cyclopropyl is optionally substituted with 1, 2, or 3 groups independently selected from —OH, —CN, C₁₋₄alkyl, C₁₋₄alkoxy, and C₁₋₄haloalkoxy. In some embodiments is a compound of Formula (I), wherein R⁴ is cyclopropyl substituted with 1 or 2 groups independently selected from halogen. In some embodiments is a compound of Formula (I), wherein R⁴ is cyclobutyl substituted with 1, 2, 3, 4, or 5 groups independently selected from halogen and C₁₋₆haloalkyl and wherein said cyclobutyl is optionally substituted with 1, 2, 3, 4, or 5 groups independently selected from —OH, —CN, —NH₂, —N(H)(C₁₋₄alkyl), —N(C₁₋₄alkyl)₂, C₁₋₄alkyl, C₁₋₄alkoxy, and C₁₋₄ haloalkoxy. In some embodiments is a compound of Formula (I), wherein R⁴ is cyclobutyl substituted with 1, 2, 3, or 4 groups independently selected from halogen and C₁₋₆haloalkyl, and wherein said cyclobutyl is optionally substituted with 1, 2, or 3 groups independently selected from —OH, —CN, C₁₋₄alkyl, C₁₋₄alkoxy, and C₁₋₄haloalkoxy. In some embodiments is a compound of Formula (I), wherein R⁴ is cyclobutyl substituted with 1, 2, 3, or 4 groups independently selected from halogen, and wherein said cyclobutyl is optionally substituted with 1, 2, or 3 groups independently selected from —OH, —CN, C₁₋₄alkyl, C₁₋₄alkoxy, and C₁₋₄haloalkoxy. In some embodiments is a compound of Formula (I), wherein R⁴ is cyclobutyl substituted with 1 or 2 groups independently selected from halogen, and wherein said cyclobutyl is optionally substituted with 1, 2, or 3 groups independently selected from —OH, —CN, C₁₋₄alkyl, C₁₋₄alkoxy, and C₁₋₄haloalkoxy. In some embodiments is a compound of Formula (I), wherein R⁴ is cyclobutyl substituted with 1 or 2 groups independently selected from halogen. In some embodiments is a compound of Formula (I), wherein R⁴ is cyclopentyl substituted with 1, 2, 3, 4, or 5 groups independently selected from halogen and C₁₋₆haloalkyl and wherein said cyclopentyl is optionally substituted with 1, 2, 3, 4, or 5 groups independently selected from —OH, —CN, —NH₂, —N(H)(C₁₋₄alkyl), —N(C₁₋₄alkyl)₂, C₁₋₄alkyl, C₁₋₄alkoxy, and C₁₋₄haloalkoxy. In some embodiments is a compound of Formula (I), wherein R⁴ is cyclopentyl substituted with 1, 2, 3, or 4 groups independently selected from halogen and C₁₋₆haloalkyl, and wherein said cyclopentyl is optionally substituted with 1, 2, or 3 groups independently selected from —OH, —CN, C₁₋₄alkyl, C₁₋₄alkoxy, and C₁₋₄haloalkoxy. In some embodiments is a compound of Formula (I), wherein R⁴ is cyclopentyl substituted with 1, 2, 3, or 4 groups independently selected from halogen, and wherein said cyclopentyl is optionally substituted with 1, 2, or 3 groups independently selected from —OH, —CN, C₁₋₄alkyl, C₁₋₄alkoxy, and C₁₋₄haloalkoxy. In some embodiments is a compound of Formula (I), wherein R⁴ is cyclopentyl substituted with 1 or 2 groups independently selected from halogen, and wherein said cyclopentyl is optionally substituted with 1, 2, or 3 groups independently selected from —OH, —CN, C₁₋₄alkyl, C₁₋₄alkoxy, and C₁₋₄haloalkoxy. In some embodiments is a compound of Formula (I), wherein R⁴ is cyclopentyl substituted with 1 or 2 groups independently selected from halogen. In some embodiments is a compound of Formula (I), wherein R⁴ is cyclohexyl substituted with 1, 2, 3, 4, or 5 groups independently selected from halogen and C₁₋₆haloalkyl and wherein said cyclohexyl is optionally substituted with 1, 2, 3, 4, or 5 groups independently selected from —OH, —CN, —NH₂, —N(H)(C₁₋₄alkyl), —N(C₁₋₄alkyl)₂, C₁₋₄alkyl, C₁₋₄alkoxy, and C₁₋₄haloalkoxy. In some embodiments is a compound of Formula (I), wherein R⁴ is cyclohexyl substituted with 1, 2, 3, or 4 groups independently selected from halogen and C₁₋₆haloalkyl, and wherein said cyclohexyl is optionally substituted with 1, 2, or 3 groups independently selected from —OH, —CN, C₁₋₄alkyl, C₁₋₄alkoxy, and C₁₋₄haloalkoxy. In some embodiments is a compound of Formula (I), wherein R⁴ is cyclohexyl substituted with 1, 2, 3, or 4 groups independently selected from halogen, and wherein said cyclohexyl is optionally substituted with 1, 2, or 3 groups independently selected from —OH, —CN, C₁₋₄alkyl, C₁₋₄alkoxy, and C₁₋₄haloalkoxy. In some embodiments is a compound of Formula (I), wherein R⁴ is cyclohexyl substituted with 1 or 2 groups independently selected from halogen, and wherein said cyclohexyl is optionally substituted with 1, 2, or 3 groups independently selected from —OH, —CN, C₁₋₄alkyl, C₁₋₄alkoxy, and C₁₋₄haloalkoxy. In some embodiments is a compound of Formula (I), wherein R⁴ is cyclohexyl substituted with 1 or 2 groups independently selected from halogen.

In some embodiments is a compound of Formula (I), wherein R⁴ is selected from:

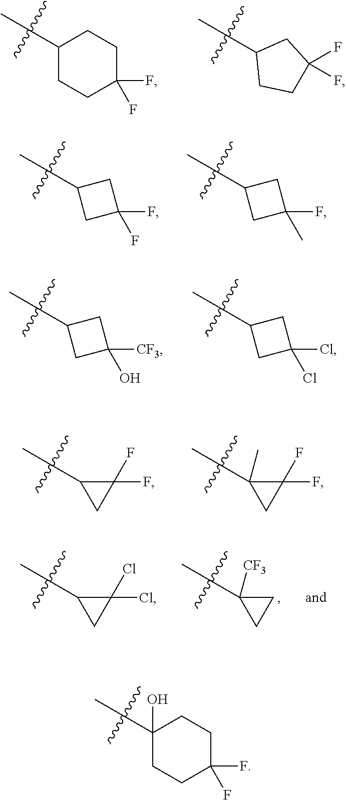

In some embodiments is a compound of Formula (I), wherein R⁴ is C₂₋₉heterocycloalkyl substituted with 1, 2, 3, 4, or 5 groups independently selected from halogen and C₁₋₆haloalkyl, and wherein said C₂₋₉heterocycloalkyl is optionally substituted with 1, 2, 3, 4, or 5 groups independently selected from —OH, —CN, —NH₂, —N(H)(C₁₋₄alkyl), —N(C₁₋₄alkyl)₂, C₁₋₄alkyl, C₁₋₄alkoxy, and C₁₋₄haloalkoxy. In some embodiments is a compound of Formula (I), wherein R⁴ is C₂₋₉heterocycloalkyl substituted with 1, 2, 3, or 4 groups independently selected from halogen. In some embodiments is a compound of Formula (I), wherein R⁴ is azetidinyl substituted with 1, 2, 3, 4, or 5 groups independently selected from halogen and C₁₋₆haloalkyl and wherein said azetidinyl is optionally substituted with 1, 2, 3, 4, or 5 groups independently selected from —OH, —CN, —NH₂, —N(H)(C₁₋₄alkyl), —N(C₁₋₄alkyl)₂, C₁₋₄alkyl, C₁₋₄alkoxy, and C₁₋₄haloalkoxy. In some embodiments is a compound of Formula (I), wherein R⁴ is azetidinyl substituted with 1, 2, 3, or 4 groups independently selected from halogen and C₁₋₆haloalkyl, and wherein said azetidinyl is optionally substituted with 1, 2, or 3 groups independently selected from —OH, —CN, C₁₋₄alkyl, C₁₋₄alkoxy, and C₁₋₄haloalkoxy. In some embodiments is a compound of Formula (I), wherein R⁴ is azetidinyl substituted with 1, 2, 3, or 4 groups independently selected from halogen, and wherein said azetidinyl is optionally substituted with 1, 2, or 3 groups independently selected from —OH, —CN, C₁₋₄alkyl, C₁₋₄alkoxy, and C₁₋₄haloalkoxy. In some embodiments is a compound of Formula (I), wherein R⁴ is azetidinyl substituted with 1 or 2 groups independently selected from halogen, and wherein said azetidinyl is optionally substituted with 1, 2, or 3 groups independently selected from —OH, —CN, C₁₋₄alkyl, C₁₋₄alkoxy, and C₁₋₄haloalkoxy. In some embodiments is a compound of Formula (I), wherein R⁴ is azetidinyl substituted with 1 or 2 groups independently selected from halogen. In some embodiments is a compound of Formula (I), wherein R⁴ is pyrrolidinyl substituted with 1, 2, 3, 4, or 5 groups independently selected from halogen and C₁₋₆haloalkyl and wherein said pyrrolidinyl is optionally substituted with 1, 2, 3, 4, or 5 groups independently selected from —OH, —CN, —NH₂, —N(H)(C₁₋₄alkyl), —N(C₁₋₄alkyl)₂, C₁₋₄alkyl, C₁₋₄alkoxy, and C₁₋₄haloalkoxy. In some embodiments is a compound of Formula (I), wherein R⁴ is pyrrolidinyl substituted with 1, 2, 3, or 4 groups independently selected from halogen and C₁₋₆haloalkyl, and wherein said pyrrolidinyl is optionally substituted with 1, 2, or 3 groups independently selected from —OH, —CN, C₁₋₄alkyl, C₁₋₄alkoxy, and C₁₋₄haloalkoxy. In some embodiments is a compound of Formula (I), wherein R⁴ is pyrrolidinyl substituted with 1, 2, 3, or 4 groups independently selected from halogen, and wherein said pyrrolidinyl is optionally substituted with 1, 2, or 3 groups independently selected from —OH, —CN, C₁₋₄alkyl, C₁₋₄alkoxy, and C₁₋₄haloalkoxy. In some embodiments is a compound of Formula (I), wherein R⁴ is pyrrolidinyl substituted with 1 or 2 groups independently selected from halogen, and wherein said pyrrolidinyl is optionally substituted with 1, 2, or 3 groups independently selected from —OH, —CN, C₁₋₄alkyl, C₁₋₄alkoxy, and C₁₋₄haloalkoxy. In some embodiments is a compound of Formula (I), wherein R⁴ is pyrrolidinyl substituted with 1 or 2 groups independently selected from halogen. In some embodiments is a compound of Formula (I), wherein R⁴ is piperidinyl substituted with 1, 2, 3, 4, or 5 groups independently selected from halogen and C₁₋₆haloalkyl and wherein said piperidinyl is optionally substituted with 1, 2, 3, 4, or 5 groups independently selected from —OH, —CN, —NH₂, —N(H)(C₁₋₄alkyl), —N(C₁₋₄alkyl)₂, C₁₋₄ alkyl, C₁₋₄alkoxy, and C₁₋₄haloalkoxy. In some embodiments is a compound of Formula (I), wherein R⁴ is piperidinyl substituted with 1, 2, 3, or 4 groups independently selected from halogen and C₁₋₆haloalkyl, and wherein said piperidinyl is optionally substituted with 1, 2, or 3 groups independently selected from —OH, —CN, C₁₋₄alkyl, C₁₋₄alkoxy, and C₁₋₄haloalkoxy. In some embodiments is a compound of Formula (I), wherein R⁴ is piperidinyl substituted with 1, 2, 3, or 4 groups independently selected from halogen, and wherein said piperidinyl is optionally substituted with 1, 2, or 3 groups independently selected from —OH, —CN, C₁₋₄alkyl, C₁₋₄alkoxy, and C₁₋₄haloalkoxy. In some embodiments is a compound of Formula (I), wherein R⁴ is piperidinyl substituted with 1 or 2 groups independently selected from halogen, and wherein said piperidinyl is optionally substituted with 1, 2, or 3 groups independently selected from —OH, —CN, C₁₋₄alkyl, C₁₋₄alkoxy, and C₁₋₄haloalkoxy. In some embodiments is a compound of Formula (I), wherein R⁴ is piperidinyl substituted with 1 or 2 groups independently selected from halogen.

In some embodiments is a compound of Formula (I), wherein R⁴ is selected from:

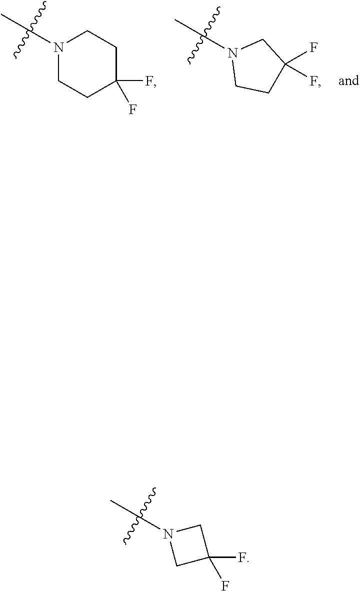 [SMILES: *N1CCC(F)(F)CC1 ; *N1CCC(F)(F)C1 ; *N1CC(F)(F)C1]

In some embodiments is a compound of Formula (I), wherein each R⁵ is independently selected from halogen and C₁₋₆alkyl. In some embodiments is a compound of Formula (I), wherein each R⁵ is independently selected from halogen and C₁₋₆haloalkyl. In some embodiments is a compound of Formula (I), wherein each R⁵ is independently selected from C₁₋₆alkyl and C₁₋₆haloalkyl. In some embodiments is a compound of Formula (I), wherein each R⁵ is independently selected from halogen. In some embodiments is a compound of Formula (I), wherein each R⁵ is independently selected from C₁₋₆alkyl. In some embodiments is a compound of Formula (I), wherein each R⁵ is independently selected from C₁₋₆haloalkyl. In some embodiments is a compound of Formula (I), wherein n is 0, 1, 2, or 3. In some embodiments is a compound of Formula (I), wherein n is 1, 2, or 3. In some embodiments is a compound of Formula (I), wherein n is 1. In some embodiments is a compound of Formula (I), wherein n is 2. In some embodiments is a compound of Formula (I), wherein n is 3.

In some embodiments is a compound of Formula (I), wherein n is 0.

In some embodiments is a compound of Formula (Ia):

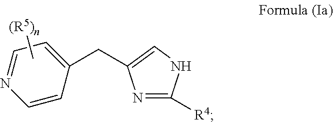

or a pharmaceutically acceptable salt thereof, wherein:
R⁴ is selected from C₃₋₆cycloalkyl and C₂₋₉heterocycloalkyl, wherein C₃₋₆cycloalkyl and C₂₋₉heterocycloalkyl are substituted with 1, 2, 3, 4, or 5 groups independently selected from halogen and C₁₋₆haloalkyl and wherein C₃₋₆cycloalkyl and C₂₋₉heterocycloalkyl are optionally substituted with 1, 2, 3, 4, or 5 groups independently selected from —OH, —CN, —NH₂, —N(H)(C₁₋₄ alkyl), —N(C₁₋₄alkyl)₂, C₁₋₄alkyl, C₁₋₄alkoxy, and C₁₋₄haloalkoxy;
each R⁵ is independently selected from halogen, C₁₋₆alkyl, and C₁₋₆haloalkyl; and
n is 0, 1, 2, 3, or 4.

In some embodiments is a compound of Formula (Ia), wherein R⁴ is C₃₋₆cycloalkyl substituted with 1, 2, 3, 4, or 5 groups independently selected from halogen and C₁₋₆haloalkyl and wherein said C₃₋₆cycloalkyl is optionally substituted with 1, 2, 3, 4, or 5 groups independently selected from —OH, —CN, —NH₂, —N(H)(C₁₋₄alkyl), —N(C₁₋₄alkyl)₂, C₁₋₄alkyl, C₁₋₄alkoxy, and C₁₋₄haloalkoxy. In some embodiments is a compound of Formula (Ia), wherein R⁴ is C₃₋₆cycloalkyl substituted with 1, 2, 3, or 4 groups independently selected from halogen and C₁₋₆haloalkyl, and wherein said C₃₋₆cycloalkyl is optionally substituted with 1, 2, or 3 groups independently selected from —OH, —CN, C₁₋₄alkyl, C₁₋₄alkoxy, and C₁₋₄haloalkoxy. In some embodiments is a compound of Formula (Ia), wherein R⁴ is C₃₋₆cycloalkyl substituted with 1, 2, 3, or 4 groups independently selected from halogen, and wherein said C₃₋₆cycloalkyl is optionally substituted with 1, 2, or 3 groups independently selected from —OH, —CN, C₁₋₄alkyl, C₁₋₄alkoxy, and C₁₋₄haloalkoxy. In some embodiments is a compound of Formula (Ia), wherein R⁴ is cyclopropyl substituted with 1, 2, 3, 4, or 5 groups independently selected from halogen and C₁₋₆haloalkyl and wherein said cyclopropyl is optionally substituted with 1, 2, 3, 4, or 5 groups independently selected from —OH, —CN, —NH₂, —N(H)(C₁₋₄alkyl), —N(C₁₋₄alkyl)₂, C₁₋₄alkyl, C₁₋₄alkoxy, and C₁₋₄haloalkoxy. In some embodiments is a compound of Formula (Ia), wherein R⁴ is cyclopropyl substituted with 1, 2, 3, or 4 groups independently selected from halogen and C₁₋₆haloalkyl, and wherein said cyclopropyl is optionally substituted with 1, 2, or 3 groups independently selected from —OH, —CN, C₁₋₄alkyl, C₁₋₄alkoxy, and C₁₋₄haloalkoxy. In some embodiments is a compound of Formula (Ia), wherein R⁴ is cyclopropyl substituted with 1, 2, 3, or 4 groups independently selected from halogen, and wherein said cyclopropyl is optionally substituted with 1, 2, or 3 groups independently selected from —OH, —CN, C₁₋₄alkyl, C₁₋₄alkoxy, and C₁₋₄haloalkoxy. In some embodiments is a compound of Formula (Ia), wherein R⁴ is cyclopropyl substituted with 1 or 2 groups independently selected from halogen, and wherein said cyclopropyl is optionally substituted with 1, 2, or 3 groups independently selected from —OH, —CN, C₁₋₄alkyl, C₁₋₄alkoxy, and C₁₋₄haloalkoxy. In some embodiments is a compound of Formula (Ia), wherein R⁴ is cyclopropyl substituted with 1 or 2 groups independently selected from halogen. In some embodiments is a compound of Formula (Ia), wherein R⁴ is cyclobutyl substituted with 1, 2, 3, 4, or 5 groups independently selected from halogen and C₁₋₆haloalkyl and wherein said cyclobutyl is optionally substituted with 1, 2, 3, 4, or 5 groups independently selected from —OH, —CN, —NH₂, —N(H)(C₁₋₄alkyl), —N(C₁₋₄alkyl)₂, C₁₋₄alkyl, C₁₋₄alkoxy, and C₁₋₄ haloalkoxy. In some embodiments is a compound of Formula (Ia), wherein R⁴ is cyclobutyl substituted with 1, 2, 3, or 4 groups independently selected from halogen and C₁₋₆haloalkyl, and wherein said cyclobutyl is optionally substituted with 1, 2, or 3 groups independently selected from —OH, —CN, C₁₋₄alkyl, C₁₋₄alkoxy, and C₁₋₄haloalkoxy. In some embodiments is a compound of Formula (Ia), wherein R⁴ is cyclobutyl substituted with 1, 2, 3, or 4 groups independently selected from halogen, and wherein said cyclobutyl is optionally substituted with 1, 2, or 3 groups independently selected from —OH, —CN, C₁₋₄alkyl, C₁₋₄alkoxy, and C₁₋₄haloalkoxy. In some embodiments is a compound of Formula (Ia), wherein R⁴ is cyclobutyl substituted with 1 or 2 groups independently selected from halogen, and wherein said cyclobutyl is optionally substituted with 1, 2, or 3 groups independently selected from —OH, —CN, C₁₋₄alkyl, C₁₋₄alkoxy, and C₁₋₄haloalkoxy. In some embodiments is a compound of Formula (Ia), wherein R⁴ is cyclobutyl substituted with 1 or 2 groups independently selected from halogen. In some embodiments is a compound of Formula (Ia), wherein R⁴ is cyclopentyl substituted with 1, 2, 3, 4, or 5 groups independently selected from halogen and C₁₋₆haloalkyl and wherein said cyclopentyl is optionally substituted with 1, 2, 3, 4, or 5 groups independently selected from —OH, —CN, —NH₂, —N(H)(C₁₋₄alkyl), —N(C₁₋₄alkyl)₂, C₁₋₄alkyl, C₁₋₄alkoxy, and C₁₋₄haloalkoxy. In some embodiments is a compound of Formula (Ia), wherein R⁴ is cyclopentyl substituted with 1, 2, 3, or 4 groups independently selected from halogen and C₁₋₆haloalkyl, and wherein said cyclopentyl is optionally substituted with 1, 2, or 3 groups independently selected from —OH, —CN, C₁₋₄alkyl, C₁₋₄alkoxy, and C₁₋₄haloalkoxy. In some embodiments is a compound of Formula (Ia), wherein R⁴ is cyclopentyl substituted with 1, 2, 3, or 4 groups independently selected from halogen, and wherein said cyclopentyl is optionally substituted with 1, 2, or 3 groups independently selected from —OH, —CN, C₁₋₄alkyl, C₁₋₄alkoxy, and C₁₋₄haloalkoxy. In some embodiments is a compound of Formula (Ia), wherein R⁴ is cyclopentyl substituted with 1 or 2 groups independently selected from halogen, and wherein said cyclopentyl is optionally substituted with 1, 2, or 3 groups independently selected from —OH, —CN, C₁₋₄alkyl, C₁₋₄alkoxy, and C₁₋₄haloalkoxy. In some embodiments is a compound of Formula (Ia), wherein R⁴ is cyclopentyl substituted with 1 or 2 groups independently selected from halogen. In some embodiments is a compound of Formula (Ia), wherein R⁴ is cyclohexyl substituted with 1, 2, 3, 4, or 5 groups independently selected from halogen and C₁₋₆haloalkyl and wherein said cyclohexyl is optionally substituted with 1, 2, 3, 4, or 5 groups independently selected from —OH, —CN, —NH₂, —N(H)(C₁₋₄alkyl), —N(C₁₋₄alkyl)₂, C₁₋₄alkyl, C₁₋₄alkoxy, and C₁₋₄haloalkoxy. In some embodiments is a compound of Formula (Ia), wherein R⁴ is cyclohexyl substituted with 1, 2, 3, or 4 groups independently selected from halogen and C₁₋₆haloalkyl, and wherein said cyclohexyl is optionally substituted with 1, 2, or 3 groups independently selected from —OH, —CN, C₁₋₄alkyl, C₁₋₄alkoxy, and C₁₋₄haloalkoxy. In some embodiments is a compound of Formula (Ia), wherein R⁴ is cyclohexyl substituted with 1, 2, 3, or 4 groups independently selected from halogen, and wherein said cyclohexyl is optionally substituted with 1, 2, or 3 groups independently selected from —OH, —CN, C₁₋₄alkyl, C₁₋₄alkoxy, and C₁₋₄haloalkoxy. In some embodiments is a compound of Formula (Ia), wherein R⁴ is cyclohexyl substituted with 1 or 2 groups independently selected from halogen, and wherein said cyclohexyl is optionally substituted with 1, 2, or 3 groups independently selected from —OH, —CN, C₁₋₄alkyl, C₁₋₄alkoxy, and C₁₋₄haloalkoxy. In some embodiments is a compound of Formula (Ia), wherein R⁴ is cyclohexyl substituted with 1 or 2 groups independently selected from halogen.

In some embodiments is a compound of Formula (Ia), wherein R⁴ is selected from:

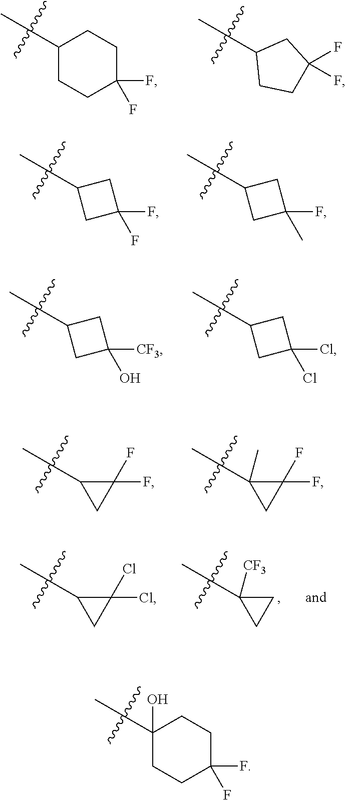

In some embodiments is a compound of Formula (Ia), wherein R⁴ is C₂₋₉heterocycloalkyl substituted with 1, 2, 3, 4, or 5 groups independently selected from halogen and C₁₋₆haloalkyl, and wherein said C₂₋₉heterocycloalkyl is optionally substituted with 1, 2, 3, 4, or 5 groups independently selected from —OH, —CN, —NH₂, —N(H)(C₁₋₄alkyl), —N(C₁₋₄alkyl)₂, C₁₋₄alkyl, C₁₋₄alkoxy, and C₁₋₄haloalkoxy. In some embodiments is a compound of Formula (Ia), wherein R⁴ is C₂₋₉heterocycloalkyl substituted with 1, 2, 3, or 4 groups independently selected from halogen. In some embodiments is a compound of Formula (Ia), wherein R⁴ is azetidinyl substituted with 1, 2, 3, 4, or 5 groups independently selected from halogen and C₁₋₆haloalkyl and wherein said azetidinyl is optionally substituted with 1, 2, 3, 4, or 5 groups independently selected from —OH, —CN, —NH₂, —N(H)(C₁₋₄alkyl), —N(C₁₋₄alkyl)₂, C₁₋₄alkyl, C₁₋₄alkoxy, and C₁₋₄haloalkoxy. In some embodiments is a compound of Formula (Ia), wherein R⁴ is azetidinyl substituted with 1, 2, 3, or 4 groups independently selected from halogen and C₁₋₆haloalkyl, and wherein said azetidinyl is optionally substituted with 1, 2, or 3 groups independently selected from —OH, —CN, C₁₋₄alkyl, C₁₋₄alkoxy, and C₁₋₄haloalkoxy. In some embodiments is a compound of Formula (Ia), wherein R⁴ is azetidinyl substituted with 1, 2, 3, or 4 groups independently selected from halogen, and wherein said azetidinyl is optionally substituted with 1, 2, or 3 groups in dependently selected from —OH, —CN, C₁₋₄alkyl, C₁₋₄alkoxy, and C₁₋₄haloalkoxy. In some embodiments is a compound of Formula (Ia), wherein R⁴ is azetidinyl substituted with 1 or 2 groups independently selected from halogen, and wherein said azetidinyl is optionally substituted with 1, 2, or 3 groups independently selected from —OH, —CN, C₁₋₄alkyl, C₁₋₄alkoxy, and C₁₋₄haloalkoxy. In some embodiments is a compound of Formula (Ia), wherein R⁴ is azetidinyl substituted with 1 or 2 groups independently selected from halogen. In some embodiments is a compound of Formula (Ia), wherein R⁴ is pyrrolidinyl substituted with 1, 2, 3, 4, or 5 groups independently selected from halogen and C₁₋₆haloalkyl and wherein said pyrrolidinyl is optionally substituted with 1, 2, 3, 4, or 5 groups independently selected from —OH, —CN, —NH₂, —N(H)(C₁₋₄alkyl), —N(C₁₋₄alkyl)₂, C₁₋₄alkyl, C₁₋₄alkoxy, and C₁₋₄haloalkoxy. In some embodiments is a compound of Formula (Ia), wherein R⁴ is pyrrolidinyl substituted with 1, 2, 3, or 4 groups independently selected from halogen and C₁₋₆haloalkyl, and wherein said pyrrolidinyl is optionally substituted with 1, 2, or 3 groups independently selected from —OH, —CN, C₁₋₄alkyl, C₁₋₄alkoxy, and C₁₋₄haloalkoxy. In some embodiments is a compound of Formula (Ia), wherein R⁴ is pyrrolidinyl substituted with 1, 2, 3, or 4 groups independently selected from halogen, and wherein said pyrrolidinyl is optionally substituted with 1, 2, or 3 groups independently selected from —OH, —CN, C₁₋₄alkyl, C₁₋₄alkoxy, and C₁₋₄haloalkoxy. In some embodiments is a compound of Formula (Ia), wherein R⁴ is pyrrolidinyl substituted with 1 or 2 groups independently selected from halogen, and wherein said pyrrolidinyl is optionally substituted with 1, 2, or 3 groups independently selected from —OH, —CN, C₁₋₄alkyl, C₁₋₄alkoxy, and C₁₋₄haloalkoxy. In some embodiments is a compound of Formula (Ia), wherein R⁴ is cyclopentyl substituted with 1 or 2 groups independently selected from halogen. In some embodiments is a compound of Formula (Ia), wherein R⁴ is piperidinyl substituted with 1, 2, 3, 4, or 5 groups independently selected from halogen and C₁₋₆haloalkyl and wherein said piperidinyl is optionally substituted with 1, 2, 3, 4, or 5 groups independently selected from —OH, —CN, —NH₂, —N(H)(C₁₋₄alkyl), —N(C₁₋₄alkyl)₂, C₁₋₄ alkyl, C₁₋₄alkoxy, and C₁₋₄haloalkoxy. In some embodiments is a compound of Formula (Ia), wherein R⁴ is piperidinyl substituted with 1, 2, 3, or 4 groups independently selected from halogen and C₁₋₆haloalkyl, and wherein said piperidinyl is optionally substituted with 1, 2, or 3 groups independently selected from —OH, —CN, C₁₋₄alkyl, C₁₋₄alkoxy, and C₁₋₄haloalkoxy. In some embodiments is a compound of Formula (Ia), wherein R⁴ is piperidinyl substituted with 1, 2, 3, or 4 groups independently selected from halogen, and wherein said piperidinyl is optionally substituted with 1, 2, or 3 groups independently selected from —OH, —CN, C₁₋₄alkyl, C₁₋₄alkoxy, and C₁₋₄haloalkoxy. In some embodiments is a compound of Formula (Ia), wherein R⁴ is piperidinyl substituted with 1 or 2 groups independently selected from halogen, and wherein said piperidinyl is optionally substituted with 1, 2, or 3 groups independently selected from —OH, —CN, C₁₋₄alkyl, C₁₋₄alkoxy, and C₁₋₄haloalkoxy. In some embodiments is a compound of Formula (Ia), wherein R⁴ is piperidinyl substituted with 1 or 2 groups independently selected from halogen.

In some embodiments is a compound of Formula (Ia), wherein R⁴ is selected from:

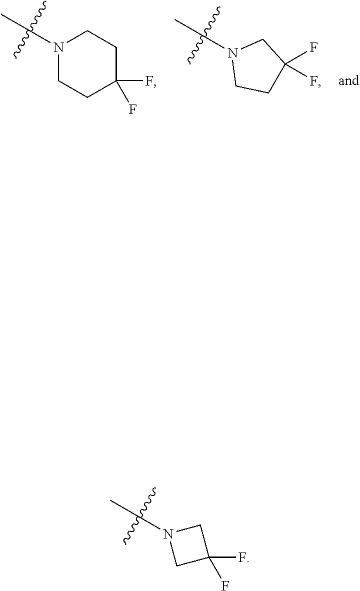 [SMILES: *N1CCC(F)(F)CC1 ; *N1CCC(F)(F)C1 ; *N1CC(F)(F)C1]

In some embodiments is a compound of Formula (Ia), wherein each R⁵ is independently selected from halogen and C₁₋₆alkyl. In some embodiments is a compound of Formula (Ia), wherein each R⁵ is independently selected from halogen and C₁₋₆haloalkyl. In some embodiments is a compound of Formula (Ia), wherein each R⁵ is independently selected from C₁₋₆alkyl and C₁₋₆haloalkyl. In some embodiments is a compound of Formula (Ia), wherein each R⁵ is independently selected from halogen. In some embodiments is a compound of Formula (Ia), wherein each R⁵ is independently selected from C₁₋₆alkyl. In some embodiments is a compound of Formula (Ia), wherein each R⁵ is independently selected from C₁₋₆haloalkyl. In some embodiments is a compound of Formula (Ia), wherein n is 0, 1, 2, or 3. In some embodiments is a compound of Formula (Ia), wherein n is 1, 2, or 3. In some embodiments is a compound of Formula (Ia), wherein n is 1. In some embodiments is a compound of Formula (Ia), wherein n is 2. In some embodiments is a compound of Formula (Ia), wherein n is 3.

In some embodiments is a compound of Formula (Ia), wherein n is 0.

In some embodiments is a compound of Formula (Ia′):

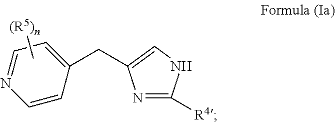

or a pharmaceutically acceptable salt thereof, wherein:
R⁴ is selected from C₃₋₆cycloalkyl and C₂₋₉heterocycloalkyl, wherein C₃₋₆cycloalkyl and C₂₋₉heterocycloalkyl are substituted with 1, 2, 3, 4, or 5 groups independently selected from halogen, —CN, C₁₋₆haloalkyl, and C₁₋₄haloalkoxy, and wherein C₃₋₆cycloalkyl and C₂₋₉heterocycloalkyl are optionally substituted with 1, 2, 3, 4, or 5 groups independently selected from —OH, —NH₂, —N(H)(C₁₋₄alkyl), —N(C₁₋₄alkyl)₂, C₁₋₄alkyl, and C₁₋₄alkoxy;
each R⁵ is independently selected from halogen, C₁₋₆alkyl, and C₁₋₆haloalkyl; and
n is 0, 1, 2, 3, or 4.

In some embodiments is a compound of Formula (Ia′), wherein R⁴ is C₃₋₆cycloalkyl substituted with 1, 2, 3, 4, or 5 groups independently selected from halogen, —CN, C₁₋₆haloalkyl, and C₁₋₄haloalkoxy, wherein said C₃₋₆cycloalkyl is optionally substituted with 1, 2, 3, 4, or 5 groups independently selected from —OH, —NH₂, —N(H)(C₁₋₄alkyl), —N(C₁₋₄alkyl)₂, C₁₋₄alkyl, and C₁₋₄alkoxy. In some embodiments is a compound of Formula (Ia′), wherein R⁴ is C₃₋₆cycloalkyl substituted with 1, 2, 3, or 4 groups independently selected from halogen, —CN, C₁₋₆haloalkyl, and C₁₋₄haloalkoxy, wherein said C₃₋₆cycloalkyl is optionally substituted with 1, 2, or 3 groups independently selected from —OH, C₁₋₄alkyl, and C₁₋₄alkoxy. In some embodiments is a compound of Formula (Ia′), wherein R⁴ is C₃₋₆cycloalkyl substituted with 1, 2, or 3 groups independently selected from halogen, —CN, C₁₋₆haloalkyl, and C₁₋₄haloalkoxy, wherein said C₃₋₆ cycloalkyl is optionally substituted with 1 or 2 groups independently selected from —OH, C₁₋₄ alkyl, and C₁₋₄alkoxy. In some embodiments is a compound of Formula (Ia′), wherein R⁴ is C₃₋₆ cycloalkyl substituted with 1, 2, or 3 groups independently selected from halogen, wherein said C₃₋₆cycloalkyl is optionally substituted with 1, 2, or 3 groups independently selected from —OH, C₁₋₄alkyl, and C₁₋₄alkoxy. In some embodiments is a compound of Formula (Ia′), wherein R⁴ is C₃₋₆cycloalkyl substituted with 1, 2, or 3 groups independently selected from C₁₋₆haloalkyl, wherein said C₃₋₆cycloalkyl is optionally substituted with 1, 2, or 3 groups independently selected from —OH, C₁₋₄alkyl, and C₁₋₄alkoxy. In some embodiments is a compound of Formula (Ia′), wherein R⁴ is C₃₋₆cycloalkyl substituted with 1, 2, or 3 groups independently selected from C₁₋₄haloalkoxy, wherein said C₃₋₆cycloalkyl is optionally substituted with 1, 2, or 3 groups independently selected from —OH, C₁₋₄alkyl, and C₁₋₄alkoxy. In some embodiments is a compound of Formula (Ia′), wherein R⁴ is C₃₋₆cycloalkyl substituted with 1 or 2 —CN, wherein said C₃₋₆cycloalkyl is optionally substituted with 1, 2, or 3 groups independently selected from —OH, C₁₋₄alkyl, and C₁₋₄alkoxy. In some embodiments is a compound of Formula (Ia′), wherein R⁴ is cyclopropyl substituted with 1, 2, 3, 4, or 5 groups independently selected from halogen, —CN, C₁₋₆haloalkyl, and C₁₋₄haloalkoxy, wherein said cyclopropyl is optionally substituted with 1, 2, 3, 4, or 5 groups independently selected from —OH, —NH₂, —N(H)(C₁₋₄alkyl), —N(C₁₋₄alkyl)₂, C₁₋₄ alkyl, and C₁₋₄alkoxy. In some embodiments is a compound of Formula (Ia′), wherein R⁴ is cyclopropyl substituted with 1, 2, 3, or 4 groups independently selected from halogen, —CN, C₁₋₆ haloalkyl, and C₁₋₄haloalkoxy, wherein said cyclopropyl is optionally substituted with 1, 2, or 3 groups independently selected from —OH, C₁₋₄alkyl, and C₁₋₄alkoxy. In some embodiments is a compound of Formula (Ia′), wherein R⁴ is cyclopropyl substituted with 1, 2, 3, or 4 groups independently selected from halogen, —CN, C₁₋₆haloalkyl, and C₁₋₄haloalkoxy, wherein said cyclopropyl is optionally substituted with 1, 2, or 3 groups independently selected from —OH, C₁₋₄ alkyl, and C₁₋₄alkoxy. In some embodiments is a compound of Formula (Ia′), wherein R⁴ is cyclopropyl substituted with 1 or 2 groups independently selected from halogen, —CN, C₁₋₆ haloalkyl, and C₁₋₄haloalkoxy, wherein said cyclopropyl is optionally substituted with 1, 2, or 3 groups independently selected from —OH, C₁₋₄alkyl, and C₁₋₄alkoxy. In some embodiments is a compound of Formula (Ia′), wherein R⁴ is cyclopropyl substituted with 1 or 2 groups independently selected from halogen and C₁₋₆haloalkyl. In some embodiments is a compound of Formula (Ia′), wherein R⁴ is cyclopropyl substituted with 1 or 2 groups independently selected from halogen. In some embodiments is a compound of Formula (Ia′), wherein R⁴ is cyclopropyl substituted with 1 or 2 groups independently selected from C₁₋₆haloalkyl. In some embodiments is a compound of Formula (Ia′), wherein R⁴ is cyclopropyl substituted with 1 or 2 groups independently selected from C₁₋₄haloalkoxy. In some embodiments is a compound of Formula (Ia′), wherein R⁴ is cyclopropyl substituted with 1 or 2 groups independently selected from —CN. In some embodiments is a compound of Formula (Ia′), wherein R⁴ is cyclobutyl substituted with 1, 2, 3, 4, or 5 groups independently selected from halogen, —CN, C₁₋₆haloalkyl, and C₁₋₄ haloalkoxy, wherein said cyclobutyl is optionally substituted with 1, 2, 3, 4, or 5 groups independently selected from —OH, —NH₂, —N(H)(C₁₋₄alkyl), —N(C₁₋₄alkyl)₂, C₁₋₄alkyl, and C₁₋₄alkoxy. In some embodiments is a compound of Formula (Ia′), wherein R⁴ is cyclobutyl substituted with 1, 2, 3, or 4 groups independently selected from halogen, —CN, C₁₋₆haloalkyl, and C₁₋₄haloalkoxy, wherein said cyclobutyl is optionally substituted with 1, 2, or 3 groups independently selected from —OH, C₁₋₄alkyl, and C₁₋₄alkoxy. In some embodiments is a compound of Formula (Ia′), wherein R⁴ is cyclobutyl substituted with 1, 2, 3, or 4 groups independently selected from halogen, —CN, C₁₋₆haloalkyl, and C₁₋₄haloalkoxy, wherein said cyclobutyl is optionally substituted with 1, 2, or 3 groups independently selected from —OH, C₁₋₄ alkyl, and C₁₋₄alkoxy. In some embodiments is a compound of Formula (Ia′), wherein R⁴ is cyclobutyl substituted with 1 or 2 groups independently selected from halogen, —CN, C₁₋₆ haloalkyl, and C₁₋₄haloalkoxy, wherein said cyclobutyl is optionally substituted with 1, 2, or 3 groups independently selected from —OH, C₁₋₄alkyl, and C₁₋₄alkoxy. In some embodiments is a compound of Formula (Ia′), wherein R⁴ is cyclobutyl substituted with 1 or 2 groups independently selected from halogen and C₁₋₆haloalkyl. In some embodiments is a compound of Formula (Ia′), wherein R⁴ is cyclobutyl substituted with 1 or 2 groups independently selected from halogen. In some embodiments is a compound of Formula (Ia′), wherein R⁴ is cyclobutyl substituted with 1 or 2 groups independently selected from C₁₋₆haloalkyl. In some embodiments is a compound of Formula (Ia′), wherein R⁴ is cyclobutyl substituted with 1 or 2 groups independently selected from C₁₋₄haloalkoxy. In some embodiments is a compound of Formula (Ia′), wherein R⁴ is cyclobutyl substituted with 1 or 2 groups independently selected from —CN. In some embodiments is a compound of Formula (Ia′), wherein R⁴ is cyclopentyl substituted with 1, 2, 3, 4, or 5 groups independently selected from halogen, —CN, C₁₋₆haloalkyl, and C₁₋₄ haloalkoxy, wherein said cyclopentyl is optionally substituted with 1, 2, 3, 4, or 5 groups independently selected from —OH, —NH₂, —N(H)(C₁₋₄alkyl), —N(C₁₋₄alkyl)₂, C₁₋₄alkyl, and C₁₋₄alkoxy. In some embodiments is a compound of Formula (Ia′), wherein R⁴ is cyclopentyl substituted with 1, 2, 3, or 4 groups independently selected from halogen, —CN, C₁₋₆haloalkyl, and C₁₋₄haloalkoxy, wherein said cyclopentyl is optionally substituted with 1, 2, or 3 groups independently selected from —OH, C₁₋₄alkyl, and C₁₋₄alkoxy. In some embodiments is a compound of Formula (Ia′), wherein R⁴ is cyclopentyl substituted with 1, 2, 3, or 4 groups independently selected from halogen, —CN, C₁₋₆haloalkyl, and C₁₋₄haloalkoxy, wherein said cyclopentyl is optionally substituted with 1, 2, or 3 groups independently selected from —OH, C₁₋₄ alkyl, and C₁₋₄alkoxy. In some embodiments is a compound of Formula (Ia′), wherein R⁴ is cyclopentyl substituted with 1 or 2 groups independently selected from halogen, —CN, C₁₋₆ haloalkyl, and C₁₋₄haloalkoxy, wherein said cyclopentyl is optionally substituted with 1, 2, or 3 groups independently selected from —OH, C₁₋₄alkyl, and C₁₋₄alkoxy. In some embodiments is a compound of Formula (Ia′), wherein R⁴ is cyclopentyl substituted with 1 or 2 groups independently selected from halogen and C₁₋₆haloalkyl. In some embodiments is a compound of Formula (Ia′), wherein R⁴ is cyclopentyl substituted with 1 or 2 groups independently selected from halogen. In some embodiments is a compound of Formula (Ia′), wherein R⁴ is cyclopentyl substituted with 1 or 2 groups independently selected from C₁₋₆haloalkyl. In some embodiments is a compound of Formula (Ia′), wherein R⁴ is cyclopentyl substituted with 1 or 2 groups independently selected from C₁₋₄haloalkoxy. In some embodiments is a compound of Formula (Ia′), wherein R⁴ is cyclopentyl substituted with 1 or 2 groups independently selected from —CN. In some embodiments is a compound of Formula (Ia′), wherein R⁴ is cyclohexyl substituted with 1, 2, 3, 4, or 5 groups independently selected from halogen, —CN, C₁₋₆haloalkyl, and C₁₋₄ haloalkoxy, wherein said cyclohexyl is optionally substituted with 1, 2, 3, 4, or 5 groups independently selected from —OH, —NH₂, —N(H)(C₁₋₄alkyl), —N(C₁₋₄alkyl)₂, C₁₋₄alkyl, and C₁₋₄alkoxy. In some embodiments is a compound of Formula (Ia′), wherein R⁴ is cyclohexyl substituted with 1, 2, 3, or 4 groups independently selected from halogen, —CN, C₁₋₆haloalkyl, and C₁₋₄haloalkoxy, wherein said cyclohexyl is optionally substituted with 1, 2, or 3 groups independently selected from —OH, C₁₋₄alkyl, and C₁₋₄alkoxy. In some embodiments is a compound of Formula (Ia′), wherein R⁴ is cyclohexyl substituted with 1, 2, 3, or 4 groups independently selected from halogen, —CN, C₁₋₆haloalkyl, and C₁₋₄haloalkoxy, wherein said cyclohexyl is optionally substituted with 1, 2, or 3 groups independently selected from —OH, C₁₋₄ alkyl, and C₁₋₄alkoxy. In some embodiments is a compound of Formula (Ia′), wherein R⁴ is cyclohexyl substituted with 1 or 2 groups independently selected from halogen, —CN, C₁₋₆ haloalkyl, and C₁₋₄haloalkoxy, wherein said cyclohexyl is optionally substituted with 1, 2, or 3 groups independently selected from —OH, C₁₋₄alkyl, and C₁₋₄alkoxy. In some embodiments is a compound of Formula (Ia′), wherein R⁴ is cyclohexyl substituted with 1 or 2 groups independently selected from halogen and C₁₋₆haloalkyl. In some embodiments is a compound of Formula (Ia′), wherein R⁴ is cyclohexyl substituted with 1 or 2 groups independently selected from halogen. In some embodiments is a compound of Formula (Ia′), wherein R⁴ is cyclohexyl substituted with 1 or 2 groups independently selected from C₁₋₆haloalkyl. In some embodiments is a compound of Formula (Ia′), wherein R⁴ is cyclohexyl substituted with 1 or 2 groups independently selected from C₁₋₄haloalkoxy. In some embodiments is a compound of Formula (Ia′), wherein R⁴ is cyclohexyl substituted with 1 or 2 groups independently selected from —CN.

In some embodiments is a compound of Formula (Ia′), wherein R⁴ is selected from:

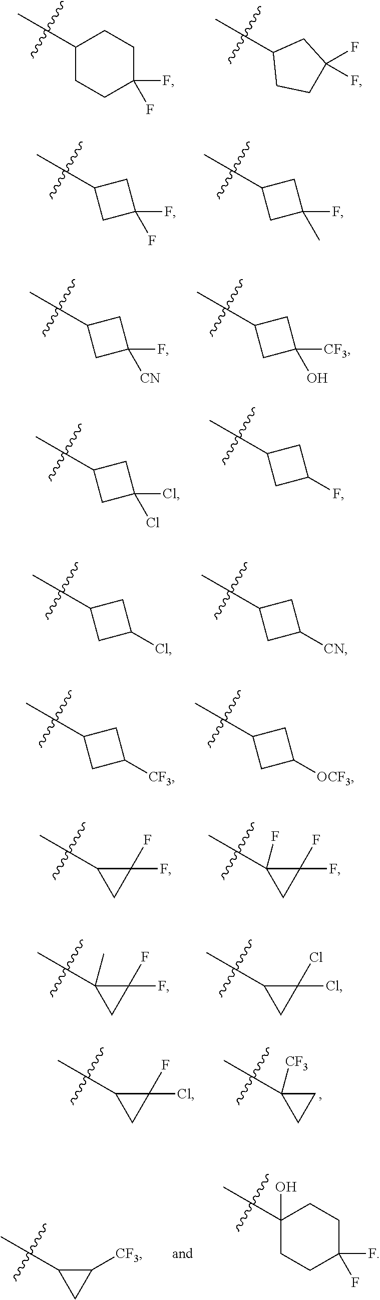

In some embodiments is a compound of Formula (Ia′), wherein R⁴ is selected from:

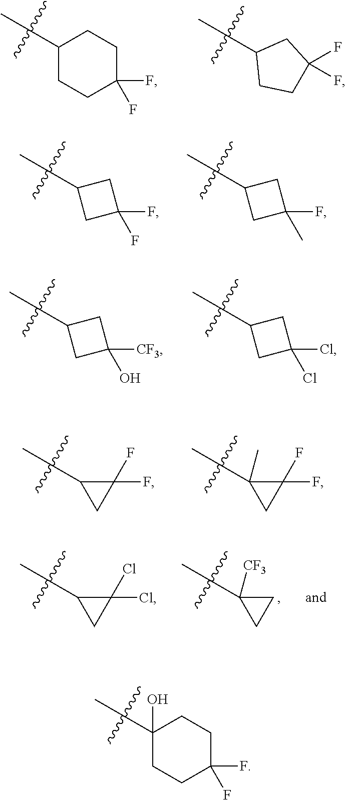

In some embodiments is a compound of Formula (Ia′), wherein R⁴ is C₂₋₉heterocycloalkyl substituted with 1, 2, 3, 4, or 5 groups independently selected from halogen, —CN, C₁₋₆haloalkyl, and C₁₋₄haloalkoxy, wherein said C₂₋₉heterocycloalkyl is optionally substituted with 1, 2, 3, 4, or 5 groups independently selected from —OH, —NH₂, —N(H)(C₁₋₄alkyl), —N(C₁₋₄alkyl)₂, C₁₋₄alkyl, and C₁₋₄alkoxy. In some embodiments is a compound of Formula (Ia′), wherein R⁴ is C₂₋₉heterocycloalkyl substituted with 1, 2, 3, 4, or 5 groups independently selected from halogen and C₁₋₆haloalkyl, and wherein said C₂₋₉heterocycloalkyl is optionally substituted with 1, 2, 3, 4, or 5 groups independently selected from —OH, —NH₂, —N(H)(C₁₋₄alkyl), —N(C₁₋₄ alkyl)₂, C₁₋₄alkyl, and C₁₋₄alkoxy. In some embodiments is a compound of Formula (Ia′), wherein R⁴ is C₂₋₉heterocycloalkyl substituted with 1, 2, or 3 groups independently selected from halogen, —CN, C₁₋₆haloalkyl, and C₁₋₄haloalkoxy, wherein said C₂₋₉heterocycloalkyl is optionally substituted with 1 or 2 groups independently selected from —OH, C₁₋₄alkyl, and C₁₋₄alkoxy. In some embodiments is a compound of Formula (Ia′), wherein R⁴ is C₂₋₉heterocycloalkyl substituted with 1, 2, 3, or 4 groups independently selected from halogen. In some embodiments is a compound of Formula (Ia′), wherein R⁴ is azetidinyl substituted with 1, 2, 3, 4, or 5 groups independently selected from halogen and C₁₋₆haloalkyl and wherein said azetidinyl is optionally substituted with 1, 2, 3, 4, or 5 groups independently selected from —OH, —NH₂, —N(H)(C₁₋₄alkyl), —N(C₁₋₄alkyl)₂, C₁₋₄alkyl, and C₁₋₄alkoxy. In some embodiments is a compound of Formula (Ia′), wherein R⁴ is azetidinyl substituted with 1, 2, or 3 groups independently selected from halogen, —CN, C₁₋₆haloalkyl, and C₁₋₄haloalkoxy, wherein said azetidinyl is optionally substituted with 1 or 2 groups independently selected from —OH, C₁₋₄ alkyl, and C₁₋₄alkoxy. In some embodiments is a compound of Formula (Ia′), wherein R⁴ is azetidinyl substituted with 1, 2, 3, or 4 groups independently selected from halogen and C₁₋₆ haloalkyl, and wherein said azetidinyl is optionally substituted with 1, 2, or 3 groups independently selected from —OH, C₁₋₄alkyl, and C₁₋₄alkoxy. In some embodiments is a compound of Formula (Ia′), wherein R⁴ is azetidinyl substituted with 1, 2, 3, or 4 groups independently selected from halogen, and wherein said azetidinyl is optionally substituted with 1, 2, or 3 groups independently selected from —OH, C₁₋₄alkyl, and C₁₋₄alkoxy. In some embodiments is a compound of Formula (Ia′), wherein R⁴ is azetidinyl substituted with 1 or 2 groups independently selected from halogen, and wherein said azetidinyl is optionally substituted with 1, 2, or 3 groups independently selected from —OH, C₁₋₄alkyl, and C₁₋₄alkoxy. In some embodiments is a compound of Formula (Ia′), wherein R⁴ is azetidinyl substituted with 1 or 2 groups independently selected from halogen. In some embodiments is a compound of Formula (Ia′), wherein R⁴ is pyrrolidinyl substituted with 1, 2, 3, 4, or 5 groups independently selected from halogen and C₁₋₆haloalkyl and wherein said pyrrolidinyl is optionally substituted with 1, 2, 3, 4, or 5 groups independently selected from —OH, —NH₂, —N(H)(C₁₋₄alkyl), —N(C₁₋₄ alkyl)₂, C₁₋₄alkyl, and C₁₋₄alkoxy. In some embodiments is a compound of Formula (Ia′), wherein R⁴ is pyrrolidinyl substituted with 1, 2, 3, or 4 groups independently selected from halogen and C₁₋₆haloalkyl, and wherein said pyrrolidinyl is optionally substituted with 1, 2, or 3 groups independently selected from —OH, C₁₋₄alkyl, and C₁₋₄alkoxy. In some embodiments is a compound of Formula (Ia′), wherein R⁴ is pyrrolidinyl substituted with 1, 2, or 3 groups independently selected from halogen, —CN, C₁₋₆haloalkyl, and C₁₋₄haloalkoxy, wherein said pyrrolidinyl is optionally substituted with 1 or 2 groups independently selected from —OH, C₁₋₄ alkyl, and C₁₋₄alkoxy. In some embodiments is a compound of Formula (Ia′), wherein R⁴ is pyrrolidinyl substituted with 1, 2, 3, or 4 groups independently selected from halogen, and wherein said pyrrolidinyl is optionally substituted with 1, 2, or 3 groups independently selected from —OH, C₁₋₄alkyl, and C₁₋₄alkoxy. In some embodiments is a compound of Formula (Ia′), wherein R⁴ is pyrrolidinyl substituted with 1, 2, or 3 groups independently selected from halogen, —CN, C₁₋₆haloalkyl, and C₁₋₄haloalkoxy, wherein said pyrrolidinyl is optionally substituted with 1 or 2 groups independently selected from —OH, C₁₋₄alkyl, and C₁₋₄alkoxy. In some embodiments is a compound of Formula (Ia′), wherein R⁴ is pyrrolidinyl substituted with 1 or 2 groups independently selected from halogen, and wherein said pyrrolidinyl is optionally substituted with 1, 2, or 3 groups independently selected from —OH, C₁₋₄alkyl, and C₁₋₄alkoxy. In some embodiments is a compound of Formula (Ia′), wherein R⁴ is pyrrolidinyl substituted with 1 or 2 groups independently selected from halogen. In some embodiments is a compound of Formula (Ia′), wherein R⁴ is piperidinyl substituted with 1, 2, 3, 4, or 5 groups independently selected from halogen and C₁₋₆haloalkyl and wherein said piperidinyl is optionally substituted with 1, 2, 3, 4, or 5 groups independently selected from —OH, —NH₂, —N(H)(C₁₋₄alkyl), —N(C₁₋₄ alkyl)₂, C₁₋₄alkyl, and C₁₋₄alkoxy. In some embodiments is a compound of Formula (Ia′), wherein R⁴ is piperidinyl substituted with 1, 2, 3, or 4 groups independently selected from halogen and C₁₋₆haloalkyl, and wherein said piperidinyl is optionally substituted with 1, 2, or 3 groups independently selected from —OH, C₁₋₄alkyl, and C₁₋₄alkoxy. In some embodiments is a compound of Formula (Ia′), wherein R⁴ is piperidinyl substituted with 1, 2, or 3 groups independently selected from halogen, —CN, C₁₋₆haloalkyl, and C₁₋₄haloalkoxy, wherein said piperidinyl is optionally substituted with 1 or 2 groups independently selected from —OH, C₁₋₄ alkyl, and C₁₋₄alkoxy. In some embodiments is a compound of Formula (Ia′), wherein R⁴ is piperidinyl substituted with 1, 2, 3, or 4 groups independently selected from halogen, and wherein said piperidinyl is optionally substituted with 1, 2, or 3 groups independently selected from —OH, C₁₋₄alkyl, and C₁₋₄alkoxy. In some embodiments is a compound of Formula (Ia′), wherein R⁴ is piperidinyl substituted with 1 or 2 groups independently selected from halogen, and wherein said piperidinyl is optionally substituted with 1, 2, or 3 groups independently selected from —OH, C₁₋₄alkyl, and C₁₋₄alkoxy. In some embodiments is a compound of Formula (Ia′), wherein R⁴ is piperidinyl substituted with 1 or 2 groups independently selected from halogen.

In some embodiments is a compound of Formula (Ia′), wherein R⁴ is selected from:

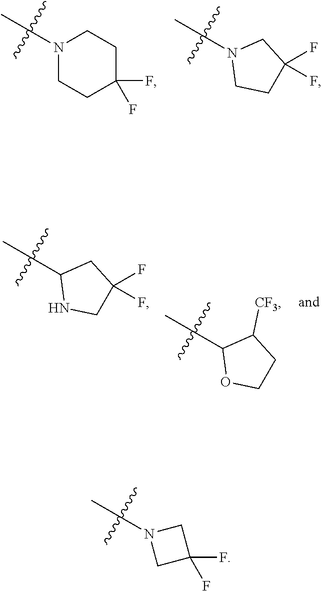 [SMILES: *N1CCC(F)(F)CC1 ; *N1CCC(F)(F)C1 ; *C1CC(F)(F)CN1 ; *C1OCCC1C(F)(F)F ; *N1CC(F)(F)C1]

In some embodiments is a compound of Formula (Ia′), wherein R⁴ is selected from:

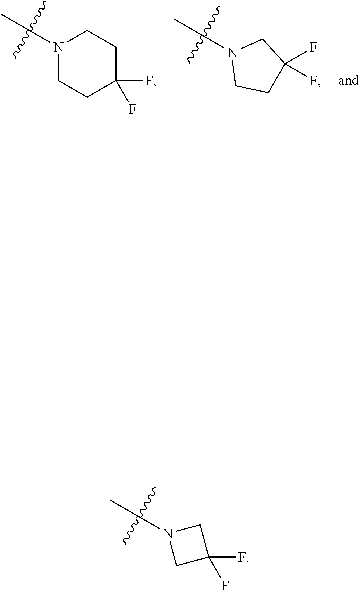 [SMILES: *N1CCC(F)(F)CC1 ; *N1CCC(F)(F)C1 ; *N1CC(F)(F)C1]

In some embodiments is a compound of Formula (Ia′), wherein each R⁵ is independently selected from halogen and C₁₋₆alkyl. In some embodiments is a compound of Formula (Ia′), wherein each R⁵ is independently selected from halogen and C₁₋₆haloalkyl. In some embodiments is a compound of Formula (Ia′), wherein each R⁵ is independently selected from C₁₋₆alkyl and C₁₋₆haloalkyl. In some embodiments is a compound of Formula (Ia′), wherein each R⁵ is independently selected from halogen. In some embodiments is a compound of Formula (Ia′), wherein each R⁵ is independently selected from C₁₋₆alkyl. In some embodiments is a compound of Formula (Ia′), wherein each R⁵ is independently selected from C₁₋₆haloalkyl. In some embodiments is a compound of Formula (Ia′), wherein n is 0, 1, 2, or 3. In some embodiments is a compound of Formula (Ia′), wherein n is 1, 2, or 3. In some embodiments is a compound of Formula (Ia′), wherein n is 1. In some embodiments is a compound of Formula (Ia′), wherein n is 2. In some embodiments is a compound of Formula (Ia′), wherein n is 3.

In some embodiments is a compound of Formula (Ia′), wherein n is 0.

In some embodiments is a compound of Formula (Ib):

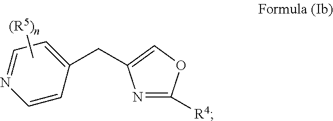

or a pharmaceutically acceptable salt thereof, wherein:
R⁴ is selected from C₃₋₆cycloalkyl and C₂₋₉heterocycloalkyl, wherein C₃₋₆cycloalkyl and C₂₋₉heterocycloalkyl are substituted with 1, 2, 3, 4, or 5 groups independently selected from halogen and C₁₋₆haloalkyl and wherein C₃₋₆cycloalkyl and C₂₋₉heterocycloalkyl are optionally substituted with 1, 2, 3, 4, or 5 groups independently selected from —OH, —CN, —NH₂, —N(H)(C₁₋₄ alkyl), —N(C₁₋₄alkyl)₂, C₁₋₄alkyl, C₁₋₄alkoxy, and C₁₋₄haloalkoxy;
each R⁵ is independently selected from halogen, C₁₋₆alkyl, and C₁₋₆haloalkyl; and
n is 0, 1, 2, 3, or 4.

In some embodiments is a compound of Formula (Ib), wherein R⁴ is C₃₋₆cycloalkyl substituted with 1, 2, 3, 4, or 5 groups independently selected from halogen and C₁₋₆haloalkyl and wherein said C₃₋₆cycloalkyl is optionally substituted with 1, 2, 3, 4, or 5 groups independently selected from —OH, —CN, —NH₂, —N(H)(C₁₋₄alkyl), —N(C₁₋₄alkyl)₂, C₁₋₄alkyl, C₁₋₄alkoxy, and C₁₋₄haloalkoxy. In some embodiments is a compound of Formula (Ib), wherein R⁴ is C₃₋₆cycloalkyl substituted with 1, 2, 3, or 4 groups independently selected from halogen and C₁₋₆haloalkyl, and wherein said C₃₋₆cycloalkyl is optionally substituted with 1, 2, or 3 groups independently selected from —OH, —CN, C₁₋₄alkyl, C₁₋₄alkoxy, and C₁₋₄haloalkoxy. In some embodiments is a compound of Formula (Ib), wherein R⁴ is C₃₋₆cycloalkyl substituted with 1, 2, 3, or 4 groups independently selected from halogen, and wherein said C₃₋₆cycloalkyl is optionally substituted with 1, 2, or 3 groups independently selected from —OH, —CN, C₁₋₄alkyl, C₁₋₄alkoxy, and C₁₋₄haloalkoxy. In some embodiments is a compound of Formula (Ib), wherein R⁴ is cyclopropyl substituted with 1, 2, 3, 4, or 5 groups independently selected from halogen and C₁₋₆haloalkyl and wherein said cyclopropyl is optionally substituted with 1, 2, 3, 4, or 5 groups independently selected from —OH, —CN, —NH₂, —N(H)(C₁₋₄alkyl), —N(C₁₋₄alkyl)₂, C₁₋₄alkyl, C₁₋₄alkoxy, and C₁₋₄haloalkoxy. In some embodiments is a compound of Formula (Ib), wherein R⁴ is cyclopropyl substituted with 1, 2, 3, or 4 groups independently selected from halogen and C₁₋₆haloalkyl, and wherein said cyclopropyl is optionally substituted with 1, 2, or 3 groups independently selected from —OH, —CN, C₁₋₄alkyl, C₁₋₄alkoxy, and C₁₋₄haloalkoxy. In some embodiments is a compound of Formula (Ib), wherein R⁴ is cyclopropyl substituted with 1, 2, 3, or 4 groups independently selected from halogen, and wherein said cyclopropyl is optionally substituted with 1, 2, or 3 groups independently selected from —OH, —CN, C₁₋₄alkyl, C₁₋₄alkoxy, and C₁₋₄haloalkoxy. In some embodiments is a compound of Formula (Ib), wherein R⁴ is cyclopropyl substituted with 1 or 2 groups independently selected from halogen, and wherein said cyclopropyl is optionally substituted with 1, 2, or 3 groups independently selected from —OH, —CN, C₁₋₄alkyl, C₁₋₄alkoxy, and C₁₋₄haloalkoxy. In some embodiments is a compound of Formula (Ib), wherein R⁴ is cyclopropyl substituted with 1 or 2 groups independently selected from halogen. In some embodiments is a compound of Formula (Ib), wherein R⁴ is cyclobutyl substituted with 1, 2, 3, 4, or 5 groups independently selected from halogen and C₁₋₆haloalkyl and wherein said cyclobutyl is optionally substituted with 1, 2, 3, 4, or 5 groups independently selected from —OH, —CN, —NH₂, —N(H)(C₁₋₄alkyl), —N(C₁₋₄alkyl)₂, C₁₋₄alkyl, C₁₋₄alkoxy, and C₁₋₄ haloalkoxy. In some embodiments is a compound of Formula (Ib), wherein R⁴ is cyclobutyl substituted with 1, 2, 3, or 4 groups independently selected from halogen and C₁₋₆haloalkyl, and wherein said cyclobutyl is optionally substituted with 1, 2, or 3 groups independently selected from —OH, —CN, C₁₋₄alkyl, C₁₋₄alkoxy, and C₁₋₄haloalkoxy. In some embodiments is a compound of Formula (Ib), wherein R⁴ is cyclobutyl substituted with 1, 2, 3, or 4 groups independently selected from halogen, and wherein said cyclobutyl is optionally substituted with 1, 2, or 3 groups independently selected from —OH, —CN, C₁₋₄alkyl, C₁₋₄alkoxy, and C₁₋₄haloalkoxy. In some embodiments is a compound of Formula (Ib), wherein R⁴ is cyclobutyl substituted with 1 or 2 groups independently selected from halogen, and wherein said cyclobutyl is optionally substituted with 1, 2, or 3 groups independently selected from —OH, —CN, C₁₋₄alkyl, C₁₋₄alkoxy, and C₁₋₄haloalkoxy. In some embodiments is a compound of Formula (Ib), wherein R⁴ is cyclobutyl substituted with 1 or 2 groups independently selected from halogen. In some embodiments is a compound of Formula (Ib), wherein R⁴ is cyclopentyl substituted with 1, 2, 3, 4, or 5 groups independently selected from halogen and C₁₋₆haloalkyl and wherein said cyclopentyl is optionally substituted with 1, 2, 3, 4, or 5 groups independently selected from —OH, —CN, —NH₂, —N(H)(C₁₋₄alkyl), —N(C₁₋₄alkyl)₂, C₁₋₄alkyl, C₁₋₄alkoxy, and C₁₋₄haloalkoxy. In some embodiments is a compound of Formula (Ib), wherein R⁴ is cyclopentyl substituted with 1, 2, 3, or 4 groups independently selected from halogen and C₁₋₆haloalkyl, and wherein said cyclopentyl is optionally substituted with 1, 2, or 3 groups independently selected from —OH, —CN, C₁₋₄alkyl, C₁₋₄alkoxy, and C₁₋₄haloalkoxy. In some embodiments is a compound of Formula (Ib), wherein R⁴ is cyclopentyl substituted with 1, 2, 3, or 4 groups independently selected from halogen, and wherein said cyclopentyl is optionally substituted with 1, 2, or 3 groups independently selected from —OH, —CN, C₁₋₄alkyl, C₁₋₄alkoxy, and C₁₋₄haloalkoxy. In some embodiments is a compound of Formula (Ib), wherein R⁴ is cyclopentyl substituted with 1 or 2 groups independently selected from halogen, and wherein said cyclopentyl is optionally substituted with 1, 2, or 3 groups independently selected from —OH, —CN, C₁₋₄alkyl, C₁₋₄alkoxy, and C₁₋₄haloalkoxy. In some embodiments is a compound of Formula (Ib), wherein R⁴ is cyclopentyl substituted with 1 or 2 groups independently selected from halogen. In some embodiments is a compound of Formula (Ib), wherein R⁴ is cyclohexyl substituted with 1, 2, 3, 4, or 5 groups independently selected from halogen and C₁₋₆haloalkyl and wherein said cyclohexyl is optionally substituted with 1, 2, 3, 4, or 5 groups independently selected from —OH, —CN, —NH₂, —N(H)(C₁₋₄alkyl), —N(C₁₋₄alkyl)₂, C₁₋₄alkyl, C₁₋₄alkoxy, and C₁₋₄haloalkoxy. In some embodiments is a compound of Formula (Ib), wherein R⁴ is cyclohexyl substituted with 1, 2, 3, or 4 groups independently selected from halogen and C₁₋₆haloalkyl, and wherein said cyclohexyl is optionally substituted with 1, 2, or 3 groups independently selected from —OH, —CN, C₁₋₄alkyl, C₁₋₄alkoxy, and C₁₋₄haloalkoxy. In some embodiments is a compound of Formula (Ib), wherein R⁴ is cyclohexyl substituted with 1, 2, 3, or 4 groups independently selected from halogen, and wherein said cyclohexyl is optionally substituted with 1, 2, or 3 groups independently selected from —OH, —CN, C₁₋₄alkyl, C₁₋₄alkoxy, and C₁₋₄haloalkoxy. In some embodiments is a compound of Formula (Ib), wherein R⁴ is cyclohexyl substituted with 1 or 2 groups independently selected from halogen, and wherein said cyclohexyl is optionally substituted with 1, 2, or 3 groups independently selected from —OH, —CN, C₁₋₄alkyl, C₁₋₄alkoxy, and C₁₋₄haloalkoxy. In some embodiments is a compound of Formula (Ib), wherein R⁴ is cyclohexyl substituted with 1 or 2 groups independently selected from halogen.

In some embodiments is a compound of Formula (Ib), wherein R⁴ is selected from:

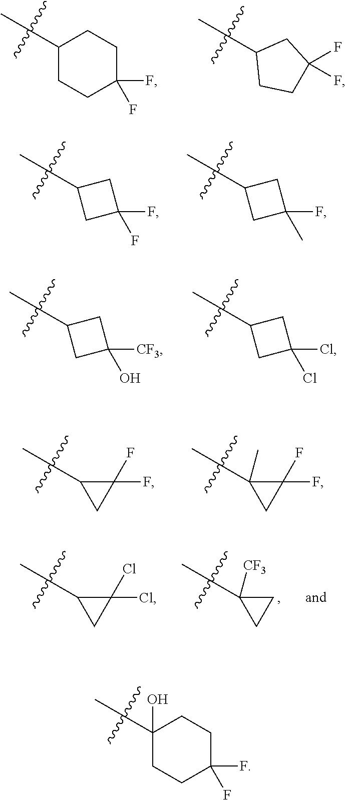

In some embodiments is a compound of Formula (Ib), wherein R⁴ is C₂₋₉heterocycloalkyl substituted with 1, 2, 3, 4, or 5 groups independently selected from halogen and C₁₋₆haloalkyl, and wherein said C₂₋₉heterocycloalkyl is optionally substituted with 1, 2, 3, 4, or 5 groups independently selected from —OH, —CN, —NH₂, —N(H)(C₁₋₄alkyl), —N(C₁₋₄alkyl)₂, C₁₋₄alkyl, C₁₋₄alkoxy, and C₁₋₄haloalkoxy. In some embodiments is a compound of Formula (Ib), wherein R⁴ is C₂₋₉heterocycloalkyl substituted with 1, 2, 3, or 4 groups independently selected from halogen. In some embodiments is a compound of Formula (Ib), wherein R⁴ is azetidinyl substituted with 1, 2, 3, 4, or 5 groups independently selected from halogen and C₁₋₆haloalkyl and wherein said azetidinyl is optionally substituted with 1, 2, 3, 4, or 5 groups independently selected from —OH, —CN, —NH₂, —N(H)(C₁₋₄alkyl), —N(C₁₋₄alkyl)₂, C₁₋₄alkyl, C₁₋₄alkoxy, and C₁₋₄haloalkoxy. In some embodiments is a compound of Formula (Ib), wherein R⁴ is azetidinyl substituted with 1, 2, 3, or 4 groups independently selected from halogen and C₁₋₆haloalkyl, and wherein said azetidinyl is optionally substituted with 1, 2, or 3 groups independently selected from —OH, —CN, C₁₋₄alkyl, C₁₋₄alkoxy, and C₁₋₄haloalkoxy. In some embodiments is a compound of Formula (Ib), wherein R⁴ is azetidinyl substituted with 1, 2, 3, or 4 groups independently selected from halogen, and wherein said azetidinyl is optionally substituted with 1, 2, or 3 groups in dependently selected from —OH, —CN, C₁₋₄alkyl, C₁₋₄alkoxy, and C₁₋₄haloalkoxy. In some embodiments is a compound of Formula (Ib), wherein R⁴ is azetidinyl substituted with 1 or 2 groups independently selected from halogen, and wherein said azetidinyl is optionally substituted with 1, 2, or 3 groups independently selected from —OH, —CN, C₁₋₄alkyl, C₁₋₄alkoxy, and C₁₋₄haloalkoxy. In some embodiments is a compound of Formula (Ib), wherein R⁴ is azetidinyl substituted with 1 or 2 groups independently selected from halogen. In some embodiments is a compound of Formula (Ib), wherein R⁴ is pyrrolidinyl substituted with 1, 2, 3, 4, or 5 groups independently selected from halogen and C₁₋₆haloalkyl and wherein said pyrrolidinyl is optionally substituted with 1, 2, 3, 4, or 5 groups independently selected from —OH, —CN, —NH₂, —N(H)(C₁₋₄alkyl), —N(C₁₋₄alkyl)₂, C₁₋₄alkyl, C₁₋₄alkoxy, and C₁₋₄haloalkoxy. In some embodiments is a compound of Formula (Ib), wherein R⁴ is pyrrolidinyl substituted with 1, 2, 3, or 4 groups independently selected from halogen and C₁₋₆haloalkyl, and wherein said pyrrolidinyl is optionally substituted with 1, 2, or 3 groups independently selected from —OH, —CN, C₁₋₄alkyl, C₁₋₄alkoxy, and C₁₋₄haloalkoxy. In some embodiments is a compound of Formula (Ib), wherein R⁴ is pyrrolidinyl substituted with 1, 2, 3, or 4 groups independently selected from halogen, and wherein said pyrrolidinyl is optionally substituted with 1, 2, or 3 groups independently selected from —OH, —CN, C₁₋₄alkyl, C₁₋₄alkoxy, and C₁₋₄haloalkoxy. In some embodiments is a compound of Formula (Ib), wherein R⁴ is pyrrolidinyl substituted with 1 or 2 groups independently selected from halogen, and wherein said pyrrolidinyl is optionally substituted with 1, 2, or 3 groups independently selected from —OH, —CN, C₁₋₄alkyl, C₁₋₄alkoxy, and C₁₋₄haloalkoxy. In some embodiments is a compound of Formula (Ib), wherein R⁴ is cyclopentyl substituted with 1 or 2 groups independently selected from halogen. In some embodiments is a compound of Formula (Ib), wherein R⁴ is piperidinyl substituted with 1, 2, 3, 4, or 5 groups independently selected from halogen and C₁₋₆haloalkyl and wherein said piperidinyl is optionally substituted with 1, 2, 3, 4, or 5 groups independently selected from —OH, —CN, —NH₂, —N(H)(C₁₋₄alkyl), —N(C₁₋₄alkyl)₂, C₁₋₄ alkyl, C₁₋₄alkoxy, and C₁₋₄haloalkoxy. In some embodiments is a compound of Formula (Ib), wherein R⁴ is piperidinyl substituted with 1, 2, 3, or 4 groups independently selected from halogen and C₁₋₆haloalkyl, and wherein said piperidinyl is optionally substituted with 1, 2, or 3 groups independently selected from —OH, —CN, C₁₋₄alkyl, C₁₋₄alkoxy, and C₁₋₄haloalkoxy. In some embodiments is a compound of Formula (Ib), wherein R⁴ is piperidinyl substituted with 1, 2, 3, or 4 groups independently selected from halogen, and wherein said piperidinyl is optionally substituted with 1, 2, or 3 groups independently selected from —OH, —CN, C₁₋₄alkyl, C₁₋₄alkoxy, and C₁₋₄haloalkoxy. In some embodiments is a compound of Formula (Ib), wherein R⁴ is piperidinyl substituted with 1 or 2 groups independently selected from halogen, and wherein said piperidinyl is optionally substituted with 1, 2, or 3 groups independently selected from —OH, —CN, C₁₋₄alkyl, C₁₋₄alkoxy, and C₁₋₄haloalkoxy. In some embodiments is a compound of Formula (Ib), wherein R⁴ is piperidinyl substituted with 1 or 2 groups independently selected from halogen.

In some embodiments is a compound of Formula (Ib), wherein R⁴ is selected from:

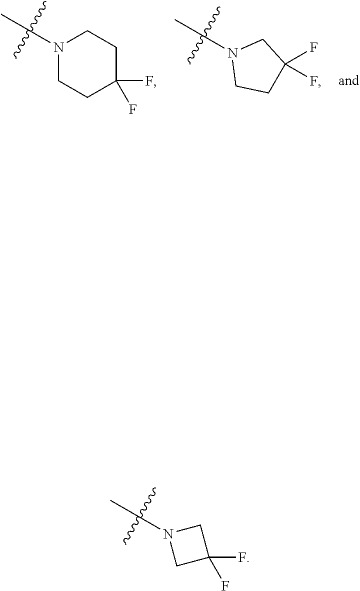 [SMILES: *N1CCC(F)(F)CC1 ; *N1CCC(F)(F)C1 ; *N1CC(F)(F)C1]

In some embodiments is a compound of Formula (Ib), wherein each R⁵ is independently selected from halogen and C₁₋₆alkyl. In some embodiments is a compound of Formula (Ib), wherein each R⁵ is independently selected from halogen and C₁₋₆haloalkyl. In some embodiments is a compound of Formula (Ib), wherein each R⁵ is independently selected from C₁₋₆alkyl and C₁₋₆haloalkyl. In some embodiments is a compound of Formula (Ib), wherein each R⁵ is independently selected from halogen. In some embodiments is a compound of Formula (Ib), wherein each R⁵ is independently selected from C₁₋₆alkyl. In some embodiments is a compound of Formula (Ib), wherein each R⁵ is independently selected from C₁₋₆haloalkyl. In some embodiments is a compound of Formula (Ib), wherein n is 0, 1, 2, or 3. In some embodiments is a compound of Formula (Ib), wherein n is 1, 2, or 3. In some embodiments is a compound of Formula (Ib), wherein n is 1. In some embodiments is a compound of Formula (Ib), wherein n is 2. In some embodiments is a compound of Formula (Ib), wherein n is 3.

In some embodiments is a compound of Formula (Ib), wherein n is 0.

In some embodiments is a compound of Formula (Ib′):

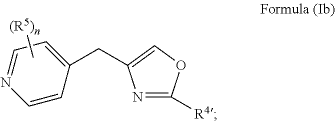

or a pharmaceutically acceptable salt thereof, wherein:
R⁴ is selected from C₃₋₆cycloalkyl and C₂₋₉heterocycloalkyl, wherein C₃₋₆cycloalkyl and C₂₋₉heterocycloalkyl are substituted with 1, 2, 3, 4, or 5 groups independently selected from halogen, —CN, C₁₋₆haloalkyl, and C₁₋₄haloalkoxy, and wherein C₃₋₆cycloalkyl and C₂₋₉heterocycloalkyl are optionally substituted with 1, 2, 3, 4, or 5 groups independently selected from —OH, —NH₂, —N(H)(C₁₋₄alkyl), —N(C₁₋₄alkyl)₂, C₁₋₄alkyl, and C₁₋₄alkoxy;
each R⁵ is independently selected from halogen, C₁₋₆alkyl, and C₁₋₆haloalkyl; and
n is 0, 1, 2, 3, or 4.

In some embodiments is a compound of Formula (Ib′), wherein R⁴ is C₃₋₆cycloalkyl substituted with 1, 2, 3, 4, or 5 groups independently selected from halogen, —CN, C₁₋₆haloalkyl, and C₁₋₄haloalkoxy, wherein said C₃₋₆cycloalkyl is optionally substituted with 1, 2, 3, 4, or 5 groups independently selected from —OH, —NH₂, —N(H)(C₁₋₄alkyl), —N(C₁₋₄alkyl)₂, C₁₋₄alkyl, and C₁₋₄alkoxy. In some embodiments is a compound of Formula (Ib′), wherein R⁴ is C₃₋₆cycloalkyl substituted with 1, 2, 3, or 4 groups independently selected from halogen, —CN, C₁₋₆haloalkyl, and C₁₋₄haloalkoxy, wherein said C₃₋₆cycloalkyl is optionally substituted with 1, 2, or 3 groups independently selected from —OH, C₁₋₄alkyl, and C₁₋₄alkoxy. In some embodiments is a compound of Formula (Ib′), wherein R⁴ is C₃₋₆cycloalkyl substituted with 1, 2, or 3 groups independently selected from halogen, —CN, C₁₋₆haloalkyl, and C₁₋₄haloalkoxy, wherein said C₃₋₆ cycloalkyl is optionally substituted with 1 or 2 groups independently selected from —OH, C₁₋₄ alkyl, and C₁₋₄alkoxy. In some embodiments is a compound of Formula (Ib′), wherein R⁴ is C₃₋₆ cycloalkyl substituted with 1, 2, or 3 groups independently selected from halogen, wherein said C₃₋₆cycloalkyl is optionally substituted with 1, 2, or 3 groups independently selected from —OH, C₁₋₄alkyl, and C₁₋₄alkoxy. In some embodiments is a compound of Formula (Ib′), wherein R⁴ is C₃₋₆cycloalkyl substituted with 1, 2, or 3 groups independently selected from C₁₋₆haloalkyl, wherein said C₃₋₆cycloalkyl is optionally substituted with 1, 2, or 3 groups independently selected from —OH, C₁₋₄alkyl, and C₁₋₄alkoxy. In some embodiments is a compound of Formula (Ib′), wherein R⁴ is C₃₋₆cycloalkyl substituted with 1, 2, or 3 groups independently selected from C₁₋₄haloalkoxy, wherein said C₃₋₆cycloalkyl is optionally substituted with 1, 2, or 3 groups independently selected from —OH, C₁₋₄alkyl, and C₁₋₄alkoxy. In some embodiments is a compound of Formula (Ib′), wherein R⁴ is C₃₋₆cycloalkyl substituted with 1 or 2 —CN, wherein said C₃₋₆cycloalkyl is optionally substituted with 1, 2, or 3 groups independently selected from —OH, C₁₋₄alkyl, and C₁₋₄alkoxy. In some embodiments is a compound of Formula (Ib′), wherein R⁴ is cyclopropyl substituted with 1, 2, 3, 4, or 5 groups independently selected from halogen, —CN, C₁₋₆haloalkyl, and C₁₋₄haloalkoxy, wherein said cyclopropyl is optionally substituted with 1, 2, 3, 4, or 5 groups independently selected from —OH, —NH₂, —N(H)(C₁₋₄alkyl), —N(C₁₋₄alkyl)₂, C₁₋₄ alkyl, and C₁₋₄alkoxy. In some embodiments is a compound of Formula (Ib′), wherein R⁴ is cyclopropyl substituted with 1, 2, 3, or 4 groups independently selected from halogen, —CN, C₁₋₆ haloalkyl, and C₁₋₄haloalkoxy, wherein said cyclopropyl is optionally substituted with 1, 2, or 3 groups independently selected from —OH, C₁₋₄alkyl, and C₁₋₄alkoxy. In some embodiments is a compound of Formula (Ib′), wherein R⁴ is cyclopropyl substituted with 1, 2, 3, or 4 groups independently selected from halogen, —CN, C₁₋₆haloalkyl, and C₁₋₄haloalkoxy, wherein said cyclopropyl is optionally substituted with 1, 2, or 3 groups independently selected from —OH, C₁₋₄ alkyl, and C₁₋₄alkoxy. In some embodiments is a compound of Formula (Ib′), wherein R⁴ is cyclopropyl substituted with 1 or 2 groups independently selected from halogen, —CN, C₁₋₆ haloalkyl, and C₁₋₄haloalkoxy, wherein said cyclopropyl is optionally substituted with 1, 2, or 3 groups independently selected from —OH, C₁₋₄alkyl, and C₁₋₄alkoxy. In some embodiments is a compound of Formula (Ib′), wherein R⁴ is cyclopropyl substituted with 1 or 2 groups independently selected from halogen and C₁₋₆haloalkyl. In some embodiments is a compound of Formula (Ib′), wherein R⁴ is cyclopropyl substituted with 1 or 2 groups independently selected from halogen. In some embodiments is a compound of Formula (Ib′), wherein R⁴ is cyclopropyl substituted with 1 or 2 groups independently selected from C₁₋₆haloalkyl. In some embodiments is a compound of Formula (Ib′), wherein R⁴ is cyclopropyl substituted with 1 or 2 groups independently selected from C₁₋₄haloalkoxy. In some embodiments is a compound of Formula (Ib′), wherein R⁴ is cyclopropyl substituted with 1 or 2 groups independently selected from —CN. In some embodiments is a compound of Formula (Ib′), wherein R⁴ is cyclobutyl substituted with 1, 2, 3, 4, or 5 groups independently selected from halogen, —CN, C₁₋₆haloalkyl, and C₁₋₄ haloalkoxy, wherein said cyclobutyl is optionally substituted with 1, 2, 3, 4, or 5 groups independently selected from —OH, —NH₂, —N(H)(C₁₋₄alkyl), —N(C₁₋₄alkyl)₂, C₁₋₄alkyl, and C₁₋₄alkoxy. In some embodiments is a compound of Formula (Ib′), wherein R⁴ is cyclobutyl substituted with 1, 2, 3, or 4 groups independently selected from halogen, —CN, C₁₋₆haloalkyl, and C₁₋₄haloalkoxy, wherein said cyclobutyl is optionally substituted with 1, 2, or 3 groups independently selected from —OH, C₁₋₄alkyl, and C₁₋₄alkoxy. In some embodiments is a compound of Formula (Ib′), wherein R⁴ is cyclobutyl substituted with 1, 2, 3, or 4 groups independently selected from halogen, —CN, C₁₋₆haloalkyl, and C₁₋₄haloalkoxy, wherein said cyclobutyl is optionally substituted with 1, 2, or 3 groups independently selected from —OH, C₁₋₄ alkyl, and C₁₋₄alkoxy. In some embodiments is a compound of Formula (Ib′), wherein R⁴ is cyclobutyl substituted with 1 or 2 groups independently selected from halogen, —CN, C₁₋₆ haloalkyl, and C₁₋₄haloalkoxy, wherein said cyclobutyl is optionally substituted with 1, 2, or 3 groups independently selected from —OH, C₁₋₄alkyl, and C₁₋₄alkoxy. In some embodiments is a compound of Formula (Ib′), wherein R⁴ is cyclobutyl substituted with 1 or 2 groups independently selected from halogen and C₁₋₆haloalkyl. In some embodiments is a compound of Formula (Ib′), wherein R⁴ is cyclobutyl substituted with 1 or 2 groups independently selected from halogen. In some embodiments is a compound of Formula (Ib′), wherein R⁴ is cyclobutyl substituted with 1 or 2 groups independently selected from C₁₋₆haloalkyl. In some embodiments is a compound of Formula (Ib′), wherein R⁴ is cyclobutyl substituted with 1 or 2 groups independently selected from C₁₋₄haloalkoxy. In some embodiments is a compound of Formula (Ib′), wherein R⁴ is cyclobutyl substituted with 1 or 2 groups independently selected from —CN. In some embodiments is a compound of Formula (Ib′), wherein R⁴ is cyclopentyl substituted with 1, 2, 3, 4, or 5 groups independently selected from halogen, —CN, C₁₋₆haloalkyl, and C₁₋₄ haloalkoxy, wherein said cyclopentyl is optionally substituted with 1, 2, 3, 4, or 5 groups independently selected from —OH, —NH₂, —N(H)(C₁₋₄alkyl), —N(C₁₋₄alkyl)₂, C₁₋₄alkyl, and C₁₋₄alkoxy. In some embodiments is a compound of Formula (Ib′), wherein R⁴ is cyclopentyl substituted with 1, 2, 3, or 4 groups independently selected from halogen, —CN, C₁₋₆haloalkyl, and C₁₋₄haloalkoxy, wherein said cyclopentyl is optionally substituted with 1, 2, or 3 groups independently selected from —OH, C₁₋₄alkyl, and C₁₋₄alkoxy. In some embodiments is a compound of Formula (Ib′), wherein R⁴ is cyclopentyl substituted with 1, 2, 3, or 4 groups independently selected from halogen, —CN, C₁₋₆haloalkyl, and C₁₋₄haloalkoxy, wherein said cyclopentyl is optionally substituted with 1, 2, or 3 groups independently selected from —OH, C₁₋₄ alkyl, and C₁₋₄alkoxy. In some embodiments is a compound of Formula (Ib′), wherein R⁴ is cyclopentyl substituted with 1 or 2 groups independently selected from halogen, —CN, C₁₋₆ haloalkyl, and C₁₋₄haloalkoxy, wherein said cyclopentyl is optionally substituted with 1, 2, or 3 groups independently selected from —OH, C₁₋₄alkyl, and C₁₋₄alkoxy. In some embodiments is a compound of Formula (Ib′), wherein R⁴ is cyclopentyl substituted with 1 or 2 groups independently selected from halogen and C₁₋₆haloalkyl. In some embodiments is a compound of Formula (Ib′), wherein R⁴ is cyclopentyl substituted with 1 or 2 groups independently selected from halogen. In some embodiments is a compound of Formula (Ib′), wherein R⁴ is cyclopentyl substituted with 1 or 2 groups independently selected from C₁₋₆haloalkyl. In some embodiments is a compound of Formula (Ib′), wherein R⁴ is cyclopentyl substituted with 1 or 2 groups independently selected from C₁₋₄haloalkoxy. In some embodiments is a compound of Formula (Ib′), wherein R⁴ is cyclopentyl substituted with 1 or 2 groups independently selected from —CN. In some embodiments is a compound of Formula (Ib′), wherein R⁴ is cyclohexyl substituted with 1, 2, 3, 4, or 5 groups independently selected from halogen, —CN, C₁₋₆haloalkyl, and C₁₋₄ haloalkoxy, wherein said cyclohexyl is optionally substituted with 1, 2, 3, 4, or 5 groups independently selected from —OH, —NH₂, —N(H)(C₁₋₄alkyl), —N(C₁₋₄alkyl)₂, C₁₋₄alkyl, and C₁₋₄alkoxy. In some embodiments is a compound of Formula (Ib′), wherein R⁴ is cyclohexyl substituted with 1, 2, 3, or 4 groups independently selected from halogen, —CN, C₁₋₆haloalkyl, and C₁₋₄haloalkoxy, wherein said cyclohexyl is optionally substituted with 1, 2, or 3 groups independently selected from —OH, C₁₋₄alkyl, and C₁₋₄alkoxy. In some embodiments is a compound of Formula (Ib′), wherein R⁴ is cyclohexyl substituted with 1, 2, 3, or 4 groups independently selected from halogen, —CN, C₁₋₆haloalkyl, and C₁₋₄haloalkoxy, wherein said cyclohexyl is optionally substituted with 1, 2, or 3 groups independently selected from —OH, C₁₋₄ alkyl, and C₁₋₄alkoxy. In some embodiments is a compound of Formula (Ib′), wherein R⁴ is cyclohexyl substituted with 1 or 2 groups independently selected from halogen, —CN, C₁₋₆ haloalkyl, and C₁₋₄haloalkoxy, wherein said cyclohexyl is optionally substituted with 1, 2, or 3 groups independently selected from —OH, C₁₋₄alkyl, and C₁₋₄alkoxy. In some embodiments is a compound of Formula (Ib′), wherein R⁴ is cyclohexyl substituted with 1 or 2 groups independently selected from halogen and C₁₋₆haloalkyl. In some embodiments is a compound of Formula (Ib′), wherein R⁴ is cyclohexyl substituted with 1 or 2 groups independently selected from halogen. In some embodiments is a compound of Formula (Ib′), wherein R⁴ is cyclohexyl substituted with 1 or 2 groups independently selected from C₁₋₆haloalkyl. In some embodiments is a compound of Formula (Ib′), wherein R⁴ is cyclohexyl substituted with 1 or 2 groups independently selected from C₁₋₄haloalkoxy. In some embodiments is a compound of Formula (Ib′), wherein R⁴ is cyclohexyl substituted with 1 or 2 groups independently selected from —CN.

In some embodiments is a compound of Formula (Ib′), wherein R⁴ is selected from:

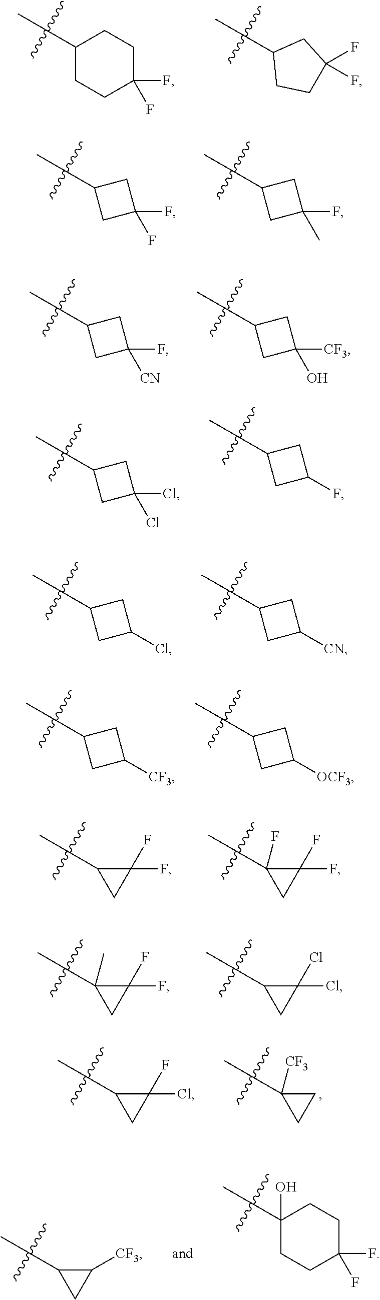

In some embodiments is a compound of Formula (Ib′), wherein R⁴ is selected from:

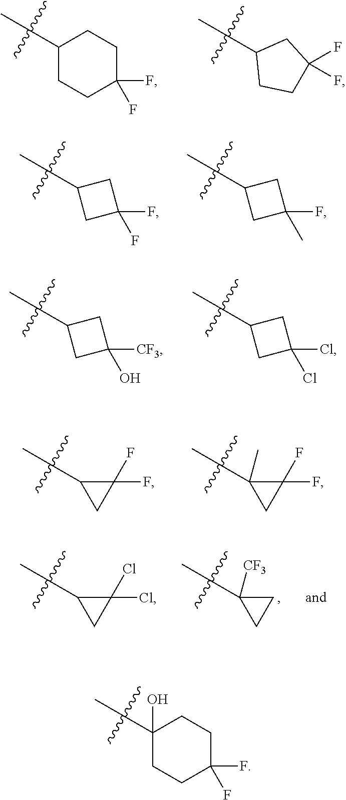

In some embodiments is a compound of Formula (Ib′), wherein R⁴ is C₂₋₉heterocycloalkyl substituted with 1, 2, 3, 4, or 5 groups independently selected from halogen, —CN, C₁₋₆haloalkyl, and C₁₋₄haloalkoxy, wherein said C₂₋₉heterocycloalkyl is optionally substituted with 1, 2, 3, 4, or 5 groups independently selected from —OH, —NH₂, —N(H)(C₁₋₄alkyl), —N(C₁₋₄alkyl)₂, C₁₋₄alkyl, and C₁₋₄alkoxy. In some embodiments is a compound of Formula (Ib′), wherein R⁴ is C₂₋₉heterocycloalkyl substituted with 1, 2, 3, 4, or 5 groups independently selected from halogen and C₁₋₆haloalkyl, and wherein said C₂₋₉heterocycloalkyl is optionally substituted with 1, 2, 3, 4, or 5 groups independently selected from —OH, —NH₂, —N(H)(C₁₋₄alkyl), —N(C₁₋₄ alkyl)₂, C₁₋₄alkyl, and C₁₋₄alkoxy. In some embodiments is a compound of Formula (Ib′), wherein R⁴ is C₂₋₉heterocycloalkyl substituted with 1, 2, or 3 groups independently selected from halogen, —CN, C₁₋₆haloalkyl, and C₁₋₄haloalkoxy, wherein said C₂₋₉heterocycloalkyl is optionally substituted with 1 or 2 groups independently selected from —OH, C₁₋₄alkyl, and C₁₋₄alkoxy. In some embodiments is a compound of Formula (Ib′), wherein R⁴ is C₂₋₉heterocycloalkyl substituted with 1, 2, 3, or 4 groups independently selected from halogen. In some embodiments is a compound of Formula (Ib′), wherein R⁴ is azetidinyl substituted with 1, 2, 3, 4, or 5 groups independently selected from halogen and C₁₋₆haloalkyl and wherein said azetidinyl is optionally substituted with 1, 2, 3, 4, or 5 groups independently selected from —OH, —NH₂, —N(H)(C₁₋₄alkyl), —N(C₁₋₄alkyl)₂, C₁₋₄alkyl, and C₁₋₄alkoxy. In some embodiments is a compound of Formula (Ib′), wherein R⁴ is azetidinyl substituted with 1, 2, or 3 groups independently selected from halogen, —CN, C₁₋₆haloalkyl, and C₁₋₄haloalkoxy, wherein said azetidinyl is optionally substituted with 1 or 2 groups independently selected from —OH, C₁₋₄ alkyl, and C₁₋₄alkoxy. In some embodiments is a compound of Formula (Ib′), wherein R⁴ is azetidinyl substituted with 1, 2, 3, or 4 groups independently selected from halogen and C₁₋₆haloalkyl, and wherein said azetidinyl is optionally substituted with 1, 2, or 3 groups independently selected from —OH, C₁₋₄alkyl, and C₁₋₄alkoxy. In some embodiments is a compound of Formula (Ib′), wherein R⁴ is azetidinyl substituted with 1, 2, 3, or 4 groups independently selected from halogen, and wherein said azetidinyl is optionally substituted with 1, 2, or 3 groups independently selected from —OH, C₁₋₄alkyl, and C₁₋₄alkoxy. In some embodiments is a compound of Formula (Ib′), wherein R⁴ is azetidinyl substituted with 1 or 2 groups independently selected from halogen, and wherein said azetidinyl is optionally substituted with 1, 2, or 3 groups independently selected from —OH, C₁₋₄alkyl, and C₁₋₄alkoxy. In some embodiments is a compound of Formula (Ib′), wherein R⁴ is azetidinyl substituted with 1 or 2 groups independently selected from halogen. In some embodiments is a compound of Formula (Ib′), wherein R⁴ is pyrrolidinyl substituted with 1, 2, 3, 4, or 5 groups independently selected from halogen and C₁₋₆haloalkyl and wherein said pyrrolidinyl is optionally substituted with 1, 2, 3, 4, or 5 groups independently selected from —OH, —NH₂, —N(H)(C₁₋₄alkyl), —N(C₁₋₄ alkyl)₂, C₁₋₄alkyl, and C₁₋₄alkoxy. In some embodiments is a compound of Formula (Ib′), wherein R⁴ is pyrrolidinyl substituted with 1, 2, 3, or 4 groups independently selected from halogen and C₁₋₆haloalkyl, and wherein said pyrrolidinyl is optionally substituted with 1, 2, or 3 groups independently selected from —OH, C₁₋₄alkyl, and C₁₋₄alkoxy. In some embodiments is a compound of Formula (Ib′), wherein R⁴ is pyrrolidinyl substituted with 1, 2, or 3 groups independently selected from halogen, —CN, C₁₋₆haloalkyl, and C₁₋₄haloalkoxy, wherein said pyrrolidinyl is optionally substituted with 1 or 2 groups independently selected from —OH, C₁₋₄ alkyl, and C₁₋₄alkoxy. In some embodiments is a compound of Formula (Ib′), wherein R⁴ is pyrrolidinyl substituted with 1, 2, 3, or 4 groups independently selected from halogen, and wherein said pyrrolidinyl is optionally substituted with 1, 2, or 3 groups independently selected from —OH, C₁₋₄alkyl, and C₁₋₄alkoxy. In some embodiments is a compound of Formula (Ib′), wherein R⁴ is pyrrolidinyl substituted with 1, 2, or 3 groups independently selected from halogen, —CN, C₁₋₆haloalkyl, and C₁₋₄haloalkoxy, wherein said pyrrolidinyl is optionally substituted with 1 or 2 groups independently selected from —OH, C₁₋₄alkyl, and C₁₋₄alkoxy. In some embodiments is a compound of Formula (Ib′), wherein R⁴ is pyrrolidinyl substituted with 1 or 2 groups independently selected from halogen, and wherein said pyrrolidinyl is optionally substituted with 1, 2, or 3 groups independently selected from —OH, C₁₋₄alkyl, and C₁₋₄alkoxy. In some embodiments is a compound of Formula (Ib′), wherein R⁴ is pyrrolidinyl substituted with 1 or 2 groups independently selected from halogen. In some embodiments is a compound of Formula (Ib′), wherein R⁴ is piperidinyl substituted with 1, 2, 3, 4, or 5 groups independently selected from halogen and C₁₋₆haloalkyl and wherein said piperidinyl is optionally substituted with 1, 2, 3, 4, or 5 groups independently selected from —OH, —NH₂, —N(H)(C₁₋₄alkyl), —N(C₁₋₄ alkyl)₂, C₁₋₄alkyl, and C₁₋₄alkoxy. In some embodiments is a compound of Formula (Ib′), wherein R⁴ is piperidinyl substituted with 1, 2, 3, or 4 groups independently selected from halogen and C₁₋₆haloalkyl, and wherein said piperidinyl is optionally substituted with 1, 2, or 3 groups independently selected from —OH, C₁₋₄alkyl, and C₁₋₄alkoxy. In some embodiments is a compound of Formula (Ib′), wherein R⁴ is piperidinyl substituted with 1, 2, or 3 groups independently selected from halogen, —CN, C₁₋₆haloalkyl, and C₁₋₄haloalkoxy, wherein said piperidinyl is optionally substituted with 1 or 2 groups independently selected from —OH, C₁₋₄ alkyl, and C₁₋₄alkoxy. In some embodiments is a compound of Formula (Ib′), wherein R⁴ is piperidinyl substituted with 1, 2, 3, or 4 groups independently selected from halogen, and wherein said piperidinyl is optionally substituted with 1, 2, or 3 groups independently selected from —OH, C₁₋₄alkyl, and C₁₋₄alkoxy. In some embodiments is a compound of Formula (Ib′), wherein R⁴ is piperidinyl substituted with 1 or 2 groups independently selected from halogen, and wherein said piperidinyl is optionally substituted with 1, 2, or 3 groups independently selected from —OH, C₁₋₄alkyl, and C₁₋₄alkoxy. In some embodiments is a compound of Formula (Ib′), wherein R⁴ is piperidinyl substituted with 1 or 2 groups independently selected from halogen.

In some embodiments is a compound of Formula (Ib′), wherein R⁴ is selected from:

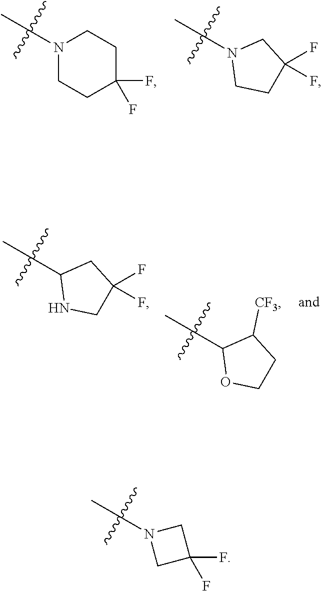 [SMILES: *N1CCC(F)(F)CC1 ; *N1CCC(F)(F)C1 ; *C1CC(F)(F)CN1 ; *C1OCCC1C(F)(F)F ; *N1CC(F)(F)C1]

In some embodiments is a compound of Formula (Ib′), wherein R⁴ is selected from:

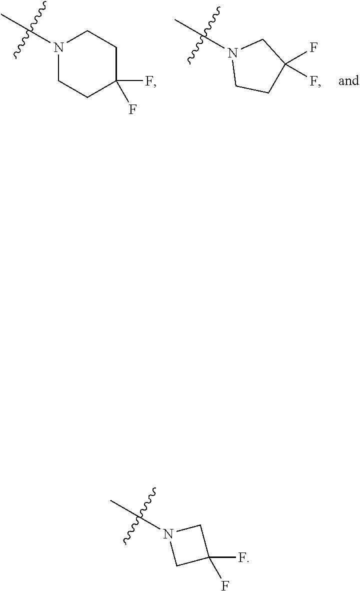 [SMILES: *N1CCC(F)(F)CC1 ; *N1CCC(F)(F)C1 ; *N1CC(F)(F)C1]

In some embodiments is a compound of Formula (Ib′), wherein each R⁵ is independently selected from halogen and C₁₋₆alkyl. In some embodiments is a compound of Formula (Ib′), wherein each R⁵ is independently selected from halogen and C₁₋₆haloalkyl. In some embodiments is a compound of Formula (Ib′), wherein each R⁵ is independently selected from C₁₋₆alkyl and C₁₋₆haloalkyl. In some embodiments is a compound of Formula (Ib′), wherein each R⁵ is independently selected from halogen. In some embodiments is a compound of Formula (Ib′), wherein each R⁵ is independently selected from C₁₋₆alkyl. In some embodiments is a compound of Formula (Ib′), wherein each R⁵ is independently selected from C₁₋₆haloalkyl. In some embodiments is a compound of Formula (Ib′), wherein n is 0, 1, 2, or 3. In some embodiments is a compound of Formula (Ib′), wherein n is 1, 2, or 3. In some embodiments is a compound of Formula (Ib′), wherein n is 1. In some embodiments is a compound of Formula (Ib′), wherein n is 2. In some embodiments is a compound of Formula (Ib′), wherein n is 3.

In some embodiments is a compound of Formula (Ib′), wherein n is 0.

In some embodiments is a compound of Formula (Ic):

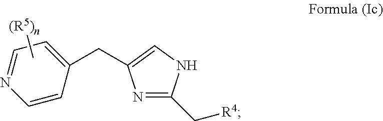

or a pharmaceutically acceptable salt thereof, wherein:
R⁴ is selected from C₃₋₆cycloalkyl and C₂₋₉heterocycloalkyl, wherein C₃₋₆cycloalkyl and C₂₋₉heterocycloalkyl are substituted with 1, 2, 3, 4, or 5 groups independently selected from halogen and C₁₋₆haloalkyl and wherein C₃₋₆cycloalkyl and C₂₋₉heterocycloalkyl are optionally substituted with 1, 2, 3, 4, or 5 groups independently selected from —OH, —CN, —NH₂, —N(H)(C₁₋₄ alkyl), —N(C₁₋₄alkyl)₂, C₁₋₄alkyl, C₁₋₄alkoxy, and C₁₋₄haloalkoxy;
each R⁵ is independently selected from halogen, C₁₋₆alkyl, and C₁₋₆haloalkyl; and
n is 0, 1, 2, 3, or 4.

In some embodiments is a compound of Formula (Ic), wherein R⁴ is C₃₋₆cycloalkyl substituted with 1, 2, 3, 4, or 5 groups independently selected from halogen and C₁₋₆haloalkyl and wherein said C₃₋₆cycloalkyl is optionally substituted with 1, 2, 3, 4, or 5 groups independently selected from —OH, —CN, —NH₂, —N(H)(C₁₋₄alkyl), —N(C₁₋₄alkyl)₂, C₁₋₄alkyl, C₁₋₄alkoxy, and C₁₋₄haloalkoxy. In some embodiments is a compound of Formula (Ic), wherein R⁴ is C₃₋₆cycloalkyl substituted with 1, 2, 3, or 4 groups independently selected from halogen and C₁₋₆haloalkyl, and wherein said C₃₋₆cycloalkyl is optionally substituted with 1, 2, or 3 groups independently selected from —OH, —CN, C₁₋₄alkyl, C₁₋₄alkoxy, and C₁₋₄haloalkoxy. In some embodiments is a compound of Formula (Ic), wherein R⁴ is C₃₋₆cycloalkyl substituted with 1, 2, 3, or 4 groups independently selected from halogen, and wherein said C₃₋₆cycloalkyl is optionally substituted with 1, 2, or 3 groups independently selected from —OH, —CN, C₁₋₄alkyl, C₁₋₄alkoxy, and C₁₋₄haloalkoxy. In some embodiments is a compound of Formula (Ic), wherein R⁴ is cyclopropyl substituted with 1, 2, 3, 4, or 5 groups independently selected from halogen and C₁₋₆haloalkyl and wherein said cyclopropyl is optionally substituted with 1, 2, 3, 4, or 5 groups independently selected from —OH, —CN, —NH₂, —N(H)(C₁₋₄alkyl), —N(C₁₋₄alkyl)₂, C₁₋₄alkyl, C₁₋₄alkoxy, and C₁₋₄haloalkoxy. In some embodiments is a compound of Formula (Ic), wherein R⁴ is cyclopropyl substituted with 1, 2, 3, or 4 groups independently selected from halogen and C₁₋₆haloalkyl, and wherein said cyclopropyl is optionally substituted with 1, 2, or 3 groups independently selected from —OH, —CN, C₁₋₄alkyl, C₁₋₄alkoxy, and C₁₋₄haloalkoxy. In some embodiments is a compound of Formula (Ic), wherein R⁴ is cyclopropyl substituted with 1, 2, 3, or 4 groups independently selected from halogen, and wherein said cyclopropyl is optionally substituted with 1, 2, or 3 groups independently selected from —OH, —CN, C₁₋₄alkyl, C₁₋₄alkoxy, and C₁₋₄haloalkoxy. In some embodiments is a compound of Formula (Ic), wherein R⁴ is cyclopropyl substituted with 1 or 2 groups independently selected from halogen, and wherein said cyclopropyl is optionally substituted with 1, 2, or 3 groups independently selected from —OH, —CN, C₁₋₄alkyl, C₁₋₄alkoxy, and C₁₋₄haloalkoxy. In some embodiments is a compound of Formula (Ic), wherein R⁴ is cyclopropyl substituted with 1 or 2 groups independently selected from halogen. In some embodiments is a compound of Formula (Ic), wherein R⁴ is cyclobutyl substituted with 1, 2, 3, 4, or 5 groups independently selected from halogen and C₁₋₆haloalkyl and wherein said cyclobutyl is optionally substituted with 1, 2, 3, 4, or 5 groups independently selected from —OH, —CN, —NH₂, —N(H)(C₁₋₄alkyl), —N(C₁₋₄alkyl)₂, C₁₋₄alkyl, C₁₋₄alkoxy, and C₁₋₄ haloalkoxy. In some embodiments is a compound of Formula (Ic), wherein R⁴ is cyclobutyl substituted with 1, 2, 3, or 4 groups independently selected from halogen and C₁₋₆haloalkyl, and wherein said cyclobutyl is optionally substituted with 1, 2, or 3 groups independently selected from —OH, —CN, C₁₋₄alkyl, C₁₋₄alkoxy, and C₁₋₄haloalkoxy. In some embodiments is a compound of Formula (Ic), wherein R⁴ is cyclobutyl substituted with 1, 2, 3, or 4 groups independently selected from halogen, and wherein said cyclobutyl is optionally substituted with 1, 2, or 3 groups independently selected from —OH, —CN, C₁₋₄alkyl, C₁₋₄alkoxy, and C₁₋₄haloalkoxy. In some embodiments is a compound of Formula (Ic), wherein R⁴ is cyclobutyl substituted with 1 or 2 groups independently selected from halogen, and wherein said cyclobutyl is optionally substituted with 1, 2, or 3 groups independently selected from —OH, —CN, C₁₋₄alkyl, C₁₋₄alkoxy, and C₁₋₄haloalkoxy. In some embodiments is a compound of Formula (Ic), wherein R⁴ is cyclobutyl substituted with 1 or 2 groups independently selected from halogen. In some embodiments is a compound of Formula (Ic), wherein R⁴ is cyclopentyl substituted with 1, 2, 3, 4, or 5 groups independently selected from halogen and C₁₋₆haloalkyl and wherein said cyclopentyl is optionally substituted with 1, 2, 3, 4, or 5 groups independently selected from —OH, —CN, —NH₂, —N(H)(C₁₋₄alkyl), —N(C₁₋₄alkyl)₂, C₁₋₄alkyl, C₁₋₄alkoxy, and C₁₋₄haloalkoxy. In some embodiments is a compound of Formula (Ic), wherein R⁴ is cyclopentyl substituted with 1, 2, 3, or 4 groups independently selected from halogen and C₁₋₆haloalkyl, and wherein said cyclopentyl is optionally substituted with 1, 2, or 3 groups independently selected from —OH, —CN, C₁₋₄alkyl, C₁₋₄alkoxy, and C₁₋₄haloalkoxy. In some embodiments is a compound of Formula (Ic), wherein R⁴ is cyclopentyl substituted with 1, 2, 3, or 4 groups independently selected from halogen, and wherein said cyclopentyl is optionally substituted with 1, 2, or 3 groups independently selected from —OH, —CN, C₁₋₄alkyl, C₁₋₄alkoxy, and C₁₋₄haloalkoxy. In some embodiments is a compound of Formula (Ic), wherein R⁴ is cyclopentyl substituted with 1 or 2 groups independently selected from halogen, and wherein said cyclopentyl is optionally substituted with 1, 2, or 3 groups independently selected from —OH, —CN, C₁₋₄alkyl, C₁₋₄alkoxy, and C₁₋₄haloalkoxy. In some embodiments is a compound of Formula (Ic), wherein R⁴ is cyclopentyl substituted with 1 or 2 groups independently selected from halogen. In some embodiments is a compound of Formula (Ic), wherein R⁴ is cyclohexyl substituted with 1, 2, 3, 4, or 5 groups independently selected from halogen and C₁₋₆haloalkyl and wherein said cyclohexyl is optionally substituted with 1, 2, 3, 4, or 5 groups independently selected from —OH, —CN, —NH₂, —N(H)(C₁₋₄alkyl), —N(C₁₋₄alkyl)₂, C₁₋₄alkyl, C₁₋₄alkoxy, and C₁₋₄haloalkoxy. In some embodiments is a compound of Formula (Ic), wherein R⁴ is cyclohexyl substituted with 1, 2, 3, or 4 groups independently selected from halogen and C₁₋₆haloalkyl, and wherein said cyclohexyl is optionally substituted with 1, 2, or 3 groups independently selected from —OH, —CN, C₁₋₄alkyl, C₁₋₄alkoxy, and C₁₋₄haloalkoxy. In some embodiments is a compound of Formula (Ic), wherein R⁴ is cyclohexyl substituted with 1, 2, 3, or 4 groups independently selected from halogen, and wherein said cyclohexyl is optionally substituted with 1, 2, or 3 groups independently selected from —OH, —CN, C₁₋₄alkyl, C₁₋₄alkoxy, and C₁₋₄haloalkoxy. In some embodiments is a compound of Formula (Ic), wherein R⁴ is cyclohexyl substituted with 1 or 2 groups independently selected from halogen, and wherein said cyclohexyl is optionally substituted with 1, 2, or 3 groups independently selected from —OH, —CN, C₁₋₄alkyl, C₁₋₄alkoxy, and C₁₋₄haloalkoxy. In some embodiments is a compound of Formula (Ic), wherein R⁴ is cyclohexyl substituted with 1 or 2 groups independently selected from halogen.

In some embodiments is a compound of Formula (Ic), wherein R⁴ is selected from:

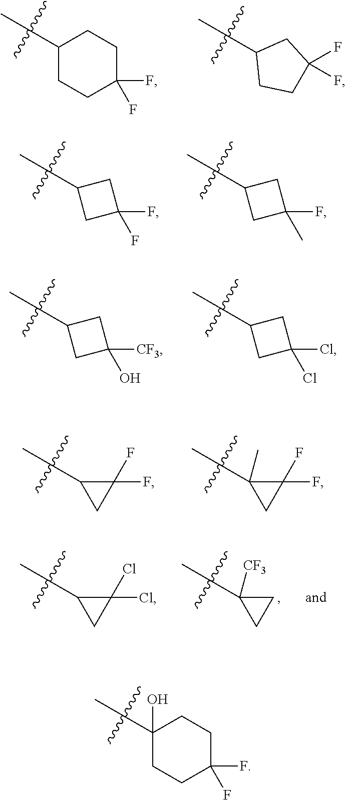

In some embodiments is a compound of Formula (Ic), wherein R⁴ is C₂₋₉heterocycloalkyl substituted with 1, 2, 3, 4, or 5 groups independently selected from halogen and C₁₋₆haloalkyl, and wherein said C₂₋₉heterocycloalkyl is optionally substituted with 1, 2, 3, 4, or 5 groups independently selected from —OH, —CN, —NH₂, —N(H)(C₁₋₄alkyl), —N(C₁₋₄alkyl)₂, C₁₋₄alkyl, C₁₋₄alkoxy, and C₁₋₄haloalkoxy. In some embodiments is a compound of Formula (Ic), wherein R⁴ is C₂₋₉heterocycloalkyl substituted with 1, 2, 3, or 4 groups independently selected from halogen. In some embodiments is a compound of Formula (Ic), wherein R⁴ is azetidinyl substituted with 1, 2, 3, 4, or 5 groups independently selected from halogen and C₁₋₆haloalkyl and wherein said azetidinyl is optionally substituted with 1, 2, 3, 4, or 5 groups independently selected from —OH, —CN, —NH₂, —N(H)(C₁₋₄alkyl), —N(C₁₋₄alkyl)₂, C₁₋₄alkyl, C₁₋₄alkoxy, and C₁₋₄haloalkoxy. In some embodiments is a compound of Formula (Ic), wherein R⁴ is azetidinyl substituted with 1, 2, 3, or 4 groups independently selected from halogen and C₁₋₆haloalkyl, and wherein said azetidinyl is optionally substituted with 1, 2, or 3 groups independently selected from —OH, —CN, C₁₋₄alkyl, C₁₋₄alkoxy, and C₁₋₄haloalkoxy. In some embodiments is a compound of Formula (Ic), wherein R⁴ is azetidinyl substituted with 1, 2, 3, or 4 groups independently selected from halogen, and wherein said azetidinyl is optionally substituted with 1, 2, or 3 groups independently selected from —OH, —CN, C₁₋₄alkyl, C₁₋₄alkoxy, and C₁₋₄haloalkoxy. In some embodiments is a compound of Formula (Ic), wherein R⁴ is azetidinyl substituted with 1 or 2 groups independently selected from halogen, and wherein said azetidinyl is optionally substituted with 1, 2, or 3 groups independently selected from —OH, —CN, C₁₋₄alkyl, C₁₋₄alkoxy, and C₁₋₄haloalkoxy. In some embodiments is a compound of Formula (Ic), wherein R⁴ is azetidinyl substituted with 1 or 2 groups independently selected from halogen. In some embodiments is a compound of Formula (Ic), wherein R⁴ is pyrrolidinyl substituted with 1, 2, 3, 4, or 5 groups independently selected from halogen and C₁₋₆haloalkyl and wherein said pyrrolidinyl is optionally substituted with 1, 2, 3, 4, or 5 groups independently selected from —OH, —CN, —NH₂, —N(H)(C₁₋₄alkyl), —N(C₁₋₄alkyl)₂, C₁₋₄alkyl, C₁₋₄alkoxy, and C₁₋₄haloalkoxy. In some embodiments is a compound of Formula (Ic), wherein R⁴ is pyrrolidinyl substituted with 1, 2, 3, or 4 groups independently selected from halogen and C₁₋₆haloalkyl, and wherein said pyrrolidinyl is optionally substituted with 1, 2, or 3 groups independently selected from —OH, —CN, C₁₋₄alkyl, C₁₋₄alkoxy, and C₁₋₄haloalkoxy. In some embodiments is a compound of Formula (Ic), wherein R⁴ is pyrrolidinyl substituted with 1, 2, 3, or 4 groups independently selected from halogen, and wherein said pyrrolidinyl is optionally substituted with 1, 2, or 3 groups independently selected from —OH, —CN, C₁₋₄alkyl, C₁₋₄alkoxy, and C₁₋₄haloalkoxy. In some embodiments is a compound of Formula (Ic), wherein R⁴ is pyrrolidinyl substituted with 1 or 2 groups independently selected from halogen, and wherein said pyrrolidinyl is optionally substituted with 1, 2, or 3 groups independently selected from —OH, —CN, C₁₋₄alkyl, C₁₋₄alkoxy, and C₁₋₄haloalkoxy. In some embodiments is a compound of Formula (Ic), wherein R⁴ is cyclopentyl substituted with 1 or 2 groups independently selected from halogen. In some embodiments is a compound of Formula (Ic), wherein R⁴ is piperidinyl substituted with 1, 2, 3, 4, or 5 groups independently selected from halogen and C₁₋₆haloalkyl and wherein said piperidinyl is optionally substituted with 1, 2, 3, 4, or 5 groups independently selected from —OH, —CN, —NH₂, —N(H)(C₁₋₄alkyl), —N(C₁₋₄alkyl)₂, C₁₋₄ alkyl, C₁₋₄alkoxy, and C₁₋₄haloalkoxy. In some embodiments is a compound of Formula (Ic), wherein R⁴ is piperidinyl substituted with 1, 2, 3, or 4 groups independently selected from halogen and C₁₋₆haloalkyl, and wherein said piperidinyl is optionally substituted with 1, 2, or 3 groups independently selected from —OH, —CN, C₁₋₄alkyl, C₁₋₄alkoxy, and C₁₋₄haloalkoxy. In some embodiments is a compound of Formula (Ic), wherein R⁴ is piperidinyl substituted with 1, 2, 3, or 4 groups independently selected from halogen, and wherein said piperidinyl is optionally substituted with 1, 2, or 3 groups independently selected from —OH, —CN, C₁₋₄alkyl, C₁₋₄alkoxy, and C₁₋₄haloalkoxy. In some embodiments is a compound of Formula (Ic), wherein R⁴ is piperidinyl substituted with 1 or 2 groups independently selected from halogen, and wherein said piperidinyl is optionally substituted with 1, 2, or 3 groups independently selected from —OH, —CN, C₁₋₄alkyl, C₁₋₄alkoxy, and C₁₋₄haloalkoxy. In some embodiments is a compound of Formula (Ic), wherein R⁴ is piperidinyl substituted with 1 or 2 groups independently selected from halogen.

In some embodiments is a compound of Formula (Ic), wherein R⁴ is selected from:

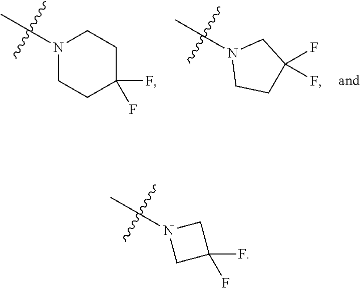 [SMILES: *N1CCC(F)(F)CC1 ; *N1CCC(F)(F)C1 ; *N1CC(F)(F)C1]

In some embodiments is a compound of Formula (Ic), wherein each R⁵ is independently selected from halogen and C₁₋₆alkyl. In some embodiments is a compound of Formula (Ic), wherein each R⁵ is independently selected from halogen and C₁₋₆haloalkyl. In some embodiments is a compound of Formula (Ic), wherein each R⁵ is independently selected from C₁₋₆alkyl and C₁₋₆haloalkyl. In some embodiments is a compound of Formula (Ic), wherein each R⁵ is independently selected from halogen. In some embodiments is a compound of Formula (Ic), wherein each R⁵ is independently selected from C₁₋₆alkyl. In some embodiments is a compound of Formula (Ic), wherein each R⁵ is independently selected from C₁₋₆haloalkyl. In some embodiments is a compound of Formula (Ic), wherein n is 0, 1, 2, or 3. In some embodiments is a compound of Formula (Ic), wherein n is 1, 2, or 3. In some embodiments is a compound of Formula (Ic), wherein n is 1. In some embodiments is a compound of Formula (Ic), wherein n is 2. In some embodiments is a compound of Formula (Ic), wherein n is 3.

In some embodiments is a compound of Formula (Ic), wherein n is 0.

In some embodiments is a compound of Formula (Ic′):

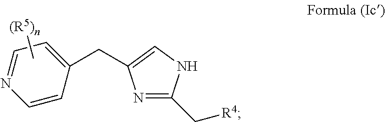

or a pharmaceutically acceptable salt thereof, wherein:
R⁴ is selected from C₃₋₆cycloalkyl and C₂₋₉heterocycloalkyl, wherein C₃₋₆cycloalkyl and C₂₋₉heterocycloalkyl are substituted with 1, 2, 3, 4, or 5 groups independently selected from halogen, —CN, C₁₋₆haloalkyl, and C₁₋₄haloalkoxy, and wherein C₃₋₆cycloalkyl and C₂₋₉heterocycloalkyl are optionally substituted with 1, 2, 3, 4, or 5 groups independently selected from —OH, —NH₂, —N(H)(C₁₋₄alkyl), —N(C₁₋₄alkyl)₂, C₁₋₄alkyl, and C₁₋₄alkoxy;
each R⁵ is independently selected from halogen, C₁₋₆alkyl, and C₁₋₆haloalkyl; and
n is 0, 1, 2, 3, or 4.

In some embodiments is a compound of Formula (Ic′), wherein R⁴ is C₃₋₆cycloalkyl substituted with 1, 2, 3, 4, or 5 groups independently selected from halogen, —CN, C₁₋₆haloalkyl, and C₁₋₄haloalkoxy, wherein said C₃₋₆cycloalkyl is optionally substituted with 1, 2, 3, 4, or 5 groups independently selected from —OH, —NH₂, —N(H)(C₁₋₄alkyl), —N(C₁₋₄alkyl)₂, C₁₋₄alkyl, and C₁₋₄alkoxy. In some embodiments is a compound of Formula (Ic′), wherein R⁴ is C₃₋₆cycloalkyl substituted with 1, 2, 3, or 4 groups independently selected from halogen, —CN, C₁₋₆haloalkyl, and C₁₋₄haloalkoxy, wherein said C₃₋₆cycloalkyl is optionally substituted with 1, 2, or 3 groups independently selected from —OH, C₁₋₄alkyl, and C₁₋₄alkoxy. In some embodiments is a compound of Formula (Ic′), wherein R⁴ is C₃₋₆cycloalkyl substituted with 1, 2, or 3 groups independently selected from halogen, —CN, C₁₋₆haloalkyl, and C₁₋₄haloalkoxy, wherein said C₃₋₆ cycloalkyl is optionally substituted with 1 or 2 groups independently selected from —OH, C₁₋₄ alkyl, and C₁₋₄alkoxy. In some embodiments is a compound of Formula (Ic′), wherein R⁴ is C₃₋₆ cycloalkyl substituted with 1, 2, or 3 groups independently selected from halogen, wherein said C₃₋₆cycloalkyl is optionally substituted with 1, 2, or 3 groups independently selected from —OH, C₁₋₄alkyl, and C₁₋₄alkoxy. In some embodiments is a compound of Formula (Ic′), wherein R⁴ is C₃₋₆cycloalkyl substituted with 1, 2, or 3 groups independently selected from C₁₋₆haloalkyl, wherein said C₃₋₆cycloalkyl is optionally substituted with 1, 2, or 3 groups independently selected from —OH, C₁₋₄alkyl, and C₁₋₄alkoxy. In some embodiments is a compound of Formula (Ic′), wherein R⁴ is C₃₋₆cycloalkyl substituted with 1, 2, or 3 groups independently selected from C₁₋₄haloalkoxy, wherein said C₃₋₆cycloalkyl is optionally substituted with 1, 2, or 3 groups independently selected from —OH, C₁₋₄alkyl, and C₁₋₄alkoxy. In some embodiments is a compound of Formula (Ic′), wherein R⁴ is C₃₋₆cycloalkyl substituted with 1 or 2 —CN, wherein said C₃₋₆cycloalkyl is optionally substituted with 1, 2, or 3 groups independently selected from —OH, C₁₋₄alkyl, and C₁₋₄alkoxy. In some embodiments is a compound of Formula (Ic′), wherein R⁴ is cyclopropyl substituted with 1, 2, 3, 4, or 5 groups independently selected from halogen, —CN, C₁₋₆haloalkyl, and C₁₋₄haloalkoxy, wherein said cyclopropyl is optionally substituted with 1, 2, 3, 4, or 5 groups independently selected from —OH, —NH₂, —N(H)(C₁₋₄alkyl), —N(C₁₋₄alkyl)₂, C₁₋₄ alkyl, and C₁₋₄alkoxy. In some embodiments is a compound of Formula (Ic′), wherein R⁴ is cyclopropyl substituted with 1, 2, 3, or 4 groups independently selected from halogen, —CN, C₁₋₆ haloalkyl, and C₁₋₄haloalkoxy, wherein said cyclopropyl is optionally substituted with 1, 2, or 3 groups independently selected from —OH, C₁₋₄alkyl, and C₁₋₄alkoxy. In some embodiments is a compound of Formula (Ic′), wherein R⁴ is cyclopropyl substituted with 1, 2, 3, or 4 groups independently selected from halogen, —CN, C₁₋₆haloalkyl, and C₁₋₄haloalkoxy, wherein said cyclopropyl is optionally substituted with 1, 2, or 3 groups independently selected from —OH, C₁₋₄ alkyl, and C₁₋₄alkoxy. In some embodiments is a compound of Formula (Ic′), wherein R⁴ is cyclopropyl substituted with 1 or 2 groups independently selected from halogen, —CN, C₁₋₆ haloalkyl, and C₁₋₄haloalkoxy, wherein said cyclopropyl is optionally substituted with 1, 2, or 3 groups independently selected from —OH, C₁₋₄alkyl, and C₁₋₄alkoxy. In some embodiments is a compound of Formula (Ic′), wherein R⁴ is cyclopropyl substituted with 1 or 2 groups independently selected from halogen and C₁₋₆haloalkyl. In some embodiments is a compound of Formula (Ic′), wherein R⁴ is cyclopropyl substituted with 1 or 2 groups independently selected from halogen. In some embodiments is a compound of Formula (Ic′), wherein R⁴ is cyclopropyl substituted with 1 or 2 groups independently selected from C₁₋₆haloalkyl. In some embodiments is a compound of Formula (Ic′), wherein R⁴ is cyclopropyl substituted with 1 or 2 groups independently selected from C₁₋₄haloalkoxy. In some embodiments is a compound of Formula (Ic′), wherein R⁴ is cyclopropyl substituted with 1 or 2 groups independently selected from —CN. In some embodiments is a compound of Formula (Ic′), wherein R⁴ is cyclobutyl substituted with 1, 2, 3, 4, or 5 groups independently selected from halogen, —CN, C₁₋₆haloalkyl, and C₁₋₄ haloalkoxy, wherein said cyclobutyl is optionally substituted with 1, 2, 3, 4, or 5 groups independently selected from —OH, —NH₂, —N(H)(C₁₋₄alkyl), —N(C₁₋₄alkyl)₂, C₁₋₄alkyl, and C₁₋₄alkoxy. In some embodiments is a compound of Formula (Ic′), wherein R⁴ is cyclobutyl substituted with 1, 2, 3, or 4 groups independently selected from halogen, —CN, C₁₋₆haloalkyl, and C₁₋₄haloalkoxy, wherein said cyclobutyl is optionally substituted with 1, 2, or 3 groups independently selected from —OH, C₁₋₄alkyl, and C₁₋₄alkoxy. In some embodiments is a compound of Formula (Ic′), wherein R⁴ is cyclobutyl substituted with 1, 2, 3, or 4 groups independently selected from halogen, —CN, C₁₋₆haloalkyl, and C₁₋₄haloalkoxy, wherein said cyclobutyl is optionally substituted with 1, 2, or 3 groups independently selected from —OH, C₁₋₄ alkyl, and C₁₋₄alkoxy. In some embodiments is a compound of Formula (Ic′), wherein R⁴ is cyclobutyl substituted with 1 or 2 groups independently selected from halogen, —CN, C₁₋₆ haloalkyl, and C₁₋₄haloalkoxy, wherein said cyclobutyl is optionally substituted with 1, 2, or 3 groups independently selected from —OH, C₁₋₄alkyl, and C₁₋₄alkoxy. In some embodiments is a compound of Formula (Ic′), wherein R⁴ is cyclobutyl substituted with 1 or 2 groups independently selected from halogen and C₁₋₆haloalkyl. In some embodiments is a compound of Formula (Ic′), wherein R⁴ is cyclobutyl substituted with 1 or 2 groups independently selected from halogen. In some embodiments is a compound of Formula (Ic′), wherein R⁴ is cyclobutyl substituted with 1 or 2 groups independently selected from C₁₋₆haloalkyl. In some embodiments is a compound of Formula (Ic′), wherein R⁴ is cyclobutyl substituted with 1 or 2 groups independently selected from C₁₋₄haloalkoxy. In some embodiments is a compound of Formula (Ic′), wherein R⁴ is cyclobutyl substituted with 1 or 2 groups independently selected from —CN. In some embodiments is a compound of Formula (Ic′), wherein R⁴ is cyclopentyl substituted with 1, 2, 3, 4, or 5 groups independently selected from halogen, —CN, C₁₋₆haloalkyl, and C₁₋₄ haloalkoxy, wherein said cyclopentyl is optionally substituted with 1, 2, 3, 4, or 5 groups independently selected from —OH, —NH₂, —N(H)(C₁₋₄alkyl), —N(C₁₋₄alkyl)₂, C₁₋₄alkyl, and C₁₋₄alkoxy. In some embodiments is a compound of Formula (Ic′), wherein R⁴ is cyclopentyl substituted with 1, 2, 3, or 4 groups independently selected from halogen, —CN, C₁₋₆haloalkyl, and C₁₋₄haloalkoxy, wherein said cyclopentyl is optionally substituted with 1, 2, or 3 groups independently selected from —OH, C₁₋₄alkyl, and C₁₋₄alkoxy. In some embodiments is a compound of Formula (Ic′), wherein R⁴ is cyclopentyl substituted with 1, 2, 3, or 4 groups independently selected from halogen, —CN, C₁₋₆haloalkyl, and C₁₋₄haloalkoxy, wherein said cyclopentyl is optionally substituted with 1, 2, or 3 groups independently selected from —OH, C₁₋₄ alkyl, and C₁₋₄alkoxy. In some embodiments is a compound of Formula (Ic′), wherein R⁴ is cyclopentyl substituted with 1 or 2 groups independently selected from halogen, —CN, C₁₋₆ haloalkyl, and C₁₋₄haloalkoxy, wherein said cyclopentyl is optionally substituted with 1, 2, or 3 groups independently selected from —OH, C₁₋₄alkyl, and C₁₋₄alkoxy. In some embodiments is a compound of Formula (Ic′), wherein R⁴ is cyclopentyl substituted with 1 or 2 groups independently selected from halogen and C₁₋₆haloalkyl. In some embodiments is a compound of Formula (Ic′), wherein R⁴ is cyclopentyl substituted with 1 or 2 groups independently selected from halogen. In some embodiments is a compound of Formula (Ic′), wherein R⁴ is cyclopentyl substituted with 1 or 2 groups independently selected from C₁₋₆haloalkyl. In some embodiments is a compound of Formula (Ic′), wherein R⁴ is cyclopentyl substituted with 1 or 2 groups independently selected from C₁₋₄haloalkoxy. In some embodiments is a compound of Formula (Ic′), wherein R⁴ is cyclopentyl substituted with 1 or 2 groups independently selected from —CN. In some embodiments is a compound of Formula (Ic′), wherein R⁴ is cyclohexyl substituted with 1, 2, 3, 4, or 5 groups independently selected from halogen, —CN, C₁₋₆haloalkyl, and C₁₋₄ haloalkoxy, wherein said cyclohexyl is optionally substituted with 1, 2, 3, 4, or 5 groups independently selected from —OH, —NH₂, —N(H)(C₁₋₄alkyl), —N(C₁₋₄alkyl)₂, C₁₋₄alkyl, and C₁₋₄alkoxy. In some embodiments is a compound of Formula (Ic′), wherein R⁴ is cyclohexyl substituted with 1, 2, 3, or 4 groups independently selected from halogen, —CN, C₁₋₆haloalkyl, and C₁₋₄haloalkoxy, wherein said cyclohexyl is optionally substituted with 1, 2, or 3 groups independently selected from —OH, C₁₋₄alkyl, and C₁₋₄alkoxy. In some embodiments is a compound of Formula (Ic′), wherein R⁴ is cyclohexyl substituted with 1, 2, 3, or 4 groups independently selected from halogen, —CN, C₁₋₆haloalkyl, and C₁₋₄haloalkoxy, wherein said cyclohexyl is optionally substituted with 1, 2, or 3 groups independently selected from —OH, C₁₋₄ alkyl, and C₁₋₄alkoxy. In some embodiments is a compound of Formula (Ic′), wherein R⁴ is cyclohexyl substituted with 1 or 2 groups independently selected from halogen, —CN, C₁₋₆ haloalkyl, and C₁₋₄haloalkoxy, wherein said cyclohexyl is optionally substituted with 1, 2, or 3 groups independently selected from —OH, C₁₋₄alkyl, and C₁₋₄alkoxy. In some embodiments is a compound of Formula (Ic′), wherein R⁴ is cyclohexyl substituted with 1 or 2 groups independently selected from halogen and C₁₋₆haloalkyl. In some embodiments is a compound of Formula (Ic′), wherein R⁴ is cyclohexyl substituted with 1 or 2 groups independently selected from halogen. In some embodiments is a compound of Formula (Ic′), wherein R⁴ is cyclohexyl substituted with 1 or 2 groups independently selected from C₁₋₆haloalkyl. In some embodiments is a compound of Formula (Ic′), wherein R⁴ is cyclohexyl substituted with 1 or 2 groups independently selected from C₁₋₄haloalkoxy. In some embodiments is a compound of Formula (Ic′), wherein R⁴ is cyclohexyl substituted with 1 or 2 groups independently selected from —CN.

In some embodiments is a compound of Formula (Ic′), wherein R⁴ is selected from:

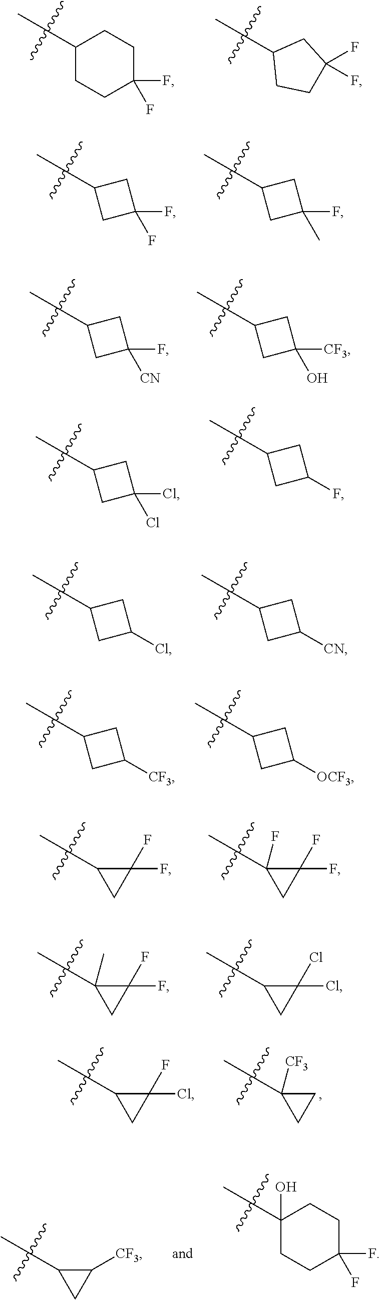

In some embodiments is a compound of Formula (Ic′), wherein R⁴ is selected from:

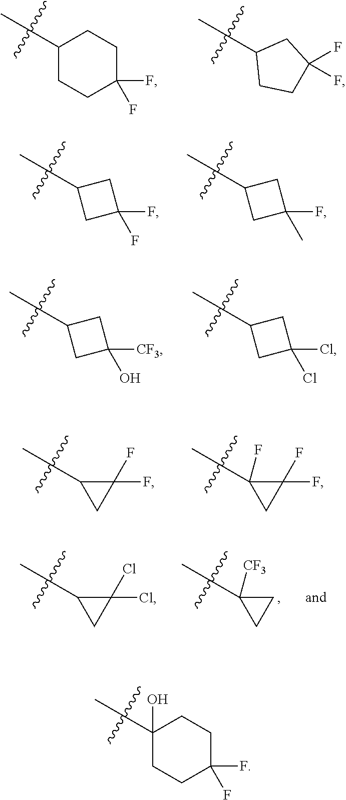

In some embodiments is a compound of Formula (Ic′), wherein R⁴ is C₂₋₉heterocycloalkyl substituted with 1, 2, 3, 4, or 5 groups independently selected from halogen, —CN, C₁₋₆haloalkyl, and C₁₋₄haloalkoxy, wherein said C₂₋₉heterocycloalkyl is optionally substituted with 1, 2, 3, 4, or 5 groups independently selected from —OH, —NH₂, —N(H)(C₁₋₄alkyl), —N(C₁₋₄alkyl)₂, C₁₋₄alkyl, and C₁₋₄alkoxy. In some embodiments is a compound of Formula (Ic′), wherein R⁴ is C₂₋₉heterocycloalkyl substituted with 1, 2, 3, 4, or 5 groups independently selected from halogen and C₁₋₆haloalkyl, and wherein said C₂₋₉heterocycloalkyl is optionally substituted with 1, 2, 3, 4, or 5 groups independently selected from —OH, —NH₂, —N(H)(C₁₋₄alkyl), —N(C₁₋₄ alkyl)₂, C₁₋₄alkyl, and C₁₋₄alkoxy. In some embodiments is a compound of Formula (Ic′), wherein R⁴ is C₂₋₉heterocycloalkyl substituted with 1, 2, or 3 groups independently selected from halogen, —CN, C₁₋₆haloalkyl, and C₁₋₄haloalkoxy, wherein said C₂₋₉heterocycloalkyl is optionally substituted with 1 or 2 groups independently selected from —OH, C₁₋₄alkyl, and C₁₋₄alkoxy. In some embodiments is a compound of Formula (Ic′), wherein R⁴ is C₂₋₉heterocycloalkyl substituted with 1, 2, 3, or 4 groups independently selected from halogen. In some embodiments is a compound of Formula (Ic′), wherein R⁴ is azetidinyl substituted with 1, 2, 3, 4, or 5 groups independently selected from halogen and C₁₋₆haloalkyl and wherein said azetidinyl is optionally substituted with 1, 2, 3, 4, or 5 groups independently selected from —OH, —NH₂, —N(H)(C₁₋₄alkyl), —N(C₁₋₄alkyl)₂, C₁₋₄alkyl, and C₁₋₄alkoxy. In some embodiments is a compound of Formula (Ic′), wherein R⁴ is azetidinyl substituted with 1, 2, or 3 groups independently selected from halogen, —CN, C₁₋₆haloalkyl, and C₁₋₄haloalkoxy, wherein said azetidinyl is optionally substituted with 1 or 2 groups independently selected from —OH, C₁₋₄ alkyl, and C₁₋₄alkoxy. In some embodiments is a compound of Formula (Ic′), wherein R⁴ is azetidinyl substituted with 1, 2, 3, or 4 groups independently selected from halogen and C₁₋₆ haloalkyl, and wherein said azetidinyl is optionally substituted with 1, 2, or 3 groups independently selected from —OH, C₁₋₄alkyl, and C₁₋₄alkoxy. In some embodiments is a compound of Formula (Ic′), wherein R⁴ is azetidinyl substituted with 1, 2, 3, or 4 groups independently selected from halogen, and wherein said azetidinyl is optionally substituted with 1, 2, or 3 groups independently selected from —OH, C₁₋₄alkyl, and C₁₋₄alkoxy. In some embodiments is a compound of Formula (Ic′), wherein R⁴ is azetidinyl substituted with 1 or 2 groups independently selected from halogen, and wherein said azetidinyl is optionally substituted with 1, 2, or 3 groups independently selected from —OH, C₁₋₄alkyl, and C₁₋₄alkoxy. In some embodiments is a compound of Formula (Ic′), wherein R⁴ is azetidinyl substituted with 1 or 2 groups independently selected from halogen. In some embodiments is a compound of Formula (Ic′), wherein R⁴ is pyrrolidinyl substituted with 1, 2, 3, 4, or 5 groups independently selected from halogen and C₁₋₆haloalkyl and wherein said pyrrolidinyl is optionally substituted with 1, 2, 3, 4, or 5 groups independently selected from —OH, —NH₂, —N(H)(C₁₋₄alkyl), —N(C₁₋₄ alkyl)₂, C₁₋₄alkyl, and C₁₋₄alkoxy. In some embodiments is a compound of Formula (Ic′), wherein R⁴ is pyrrolidinyl substituted with 1, 2, 3, or 4 groups independently selected from halogen and C₁₋₆haloalkyl, and wherein said pyrrolidinyl is optionally substituted with 1, 2, or 3 groups independently selected from —OH, C₁₋₄alkyl, and C₁₋₄alkoxy. In some embodiments is a compound of Formula (Ic′), wherein R⁴ is pyrrolidinyl substituted with 1, 2, or 3 groups independently selected from halogen, —CN, C₁₋₆haloalkyl, and C₁₋₄haloalkoxy, wherein said pyrrolidinyl is optionally substituted with 1 or 2 groups independently selected from —OH, C₁₋₄alkyl, and C₁₋₄alkoxy. In some embodiments is a compound of Formula (Ic′), wherein R⁴ is pyrrolidinyl substituted with 1, 2, 3, or 4 groups independently selected from halogen, and wherein said pyrrolidinyl is optionally substituted with 1, 2, or 3 groups independently selected from —OH, C₁₋₄alkyl, and C₁₋₄alkoxy. In some embodiments is a compound of Formula (Ic′), wherein R⁴ is pyrrolidinyl substituted with 1, 2, or 3 groups independently selected from halogen, —CN, C₁₋₆haloalkyl, and C₁₋₄haloalkoxy, wherein said pyrrolidinyl is optionally substituted with 1 or 2 groups independently selected from —OH, C₁₋₄alkyl, and C₁₋₄alkoxy. In some embodiments is a compound of Formula (Ic′), wherein R⁴ is pyrrolidinyl substituted with 1 or 2 groups independently selected from halogen, and wherein said pyrrolidinyl is optionally substituted with 1, 2, or 3 groups independently selected from —OH, C₁₋₄alkyl, and C₁₋₄alkoxy. In some embodiments is a compound of Formula (Ic′), wherein R⁴ is pyrrolidinyl substituted with 1 or 2 groups independently selected from halogen. In some embodiments is a compound of Formula (Ic′), wherein R⁴ is piperidinyl substituted with 1, 2, 3, 4, or 5 groups independently selected from halogen and C₁₋₆haloalkyl and wherein said piperidinyl is optionally substituted with 1, 2, 3, 4, or 5 groups independently selected from —OH, —NH₂, —N(H)(C₁₋₄alkyl), —N(C₁₋₄ alkyl)₂, C₁₋₄alkyl, and C₁₋₄alkoxy. In some embodiments is a compound of Formula (Ic′), wherein R⁴ is piperidinyl substituted with 1, 2, 3, or 4 groups independently selected from halogen and C₁₋₆haloalkyl, and wherein said piperidinyl is optionally substituted with 1, 2, or 3 groups independently selected from —OH, C₁₋₄alkyl, and C₁₋₄alkoxy. In some embodiments is a compound of Formula (Ic′), wherein R⁴ is piperidinyl substituted with 1, 2, or 3 groups independently selected from halogen, —CN, C₁₋₆haloalkyl, and C₁₋₄haloalkoxy, wherein said piperidinyl is optionally substituted with 1 or 2 groups independently selected from —OH, C₁₋₄ alkyl, and C₁₋₄alkoxy. In some embodiments is a compound of Formula (Ic′), wherein R⁴ is piperidinyl substituted with 1, 2, 3, or 4 groups independently selected from halogen, and wherein said piperidinyl is optionally substituted with 1, 2, or 3 groups independently selected from —OH, C₁₋₄alkyl, and C₁₋₄alkoxy. In some embodiments is a compound of Formula (Ic′), wherein R⁴ is piperidinyl substituted with 1 or 2 groups independently selected from halogen, and wherein said piperidinyl is optionally substituted with 1, 2, or 3 groups independently selected from —OH, C₁₋₄alkyl, and C₁₋₄alkoxy. In some embodiments is a compound of Formula (Ic′), wherein R⁴ is piperidinyl substituted with 1 or 2 groups independently selected from halogen.

In some embodiments is a compound of Formula (Ic′), wherein R⁴ is selected from:

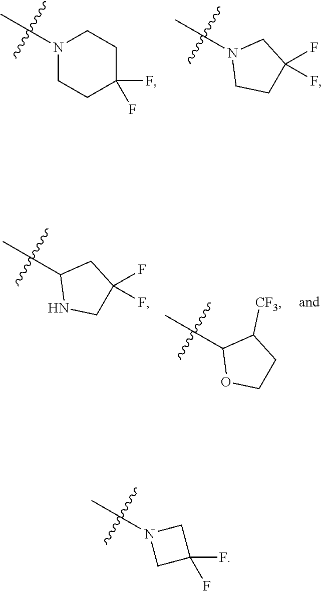 [SMILES: *N1CCC(F)(F)CC1 ; *N1CCC(F)(F)C1 ; *C1CC(F)(F)CN1 ; *C1OCCC1C(F)(F)F ; *N1CC(F)(F)C1]

In some embodiments is a compound of Formula (Ic′), wherein R⁴ is selected from:

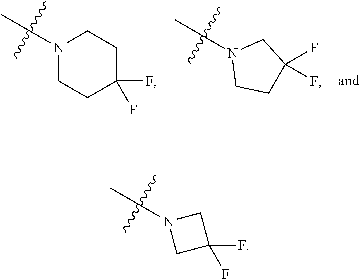 [SMILES: *N1CCC(F)(F)CC1 ; *N1CCC(F)(F)C1 ; *N1CC(F)(F)C1]

In some embodiments is a compound of Formula (Ic′), wherein each R⁵ is independently selected from halogen and C₁₋₆alkyl. In some embodiments is a compound of Formula (Ic′), wherein each R⁵ is independently selected from halogen and C₁₋₆haloalkyl. In some embodiments is a compound of Formula (Ic′), wherein each R⁵ is independently selected from C₁₋₆alkyl and C₁₋₆haloalkyl. In some embodiments is a compound of Formula (Ic′), wherein each R⁵ is independently selected from halogen. In some embodiments is a compound of Formula (Ic′), wherein each R⁵ is independently selected from C₁₋₆alkyl. In some embodiments is a compound of Formula (Ic′), wherein each R⁵ is independently selected from C₁₋₆haloalkyl. In some embodiments is a compound of Formula (Ic′), wherein n is 0, 1, 2, or 3. In some embodiments is a compound of Formula (Ic′), wherein n is 1, 2, or 3. In some embodiments is a compound of Formula (Ic′), wherein n is 1. In some embodiments is a compound of Formula (Ic′), wherein n is 2. In some embodiments is a compound of Formula (Ic′), wherein n is 3.

In some embodiments is a compound of Formula (Ic′), wherein n is 0.

In some embodiments is a compound selected from:

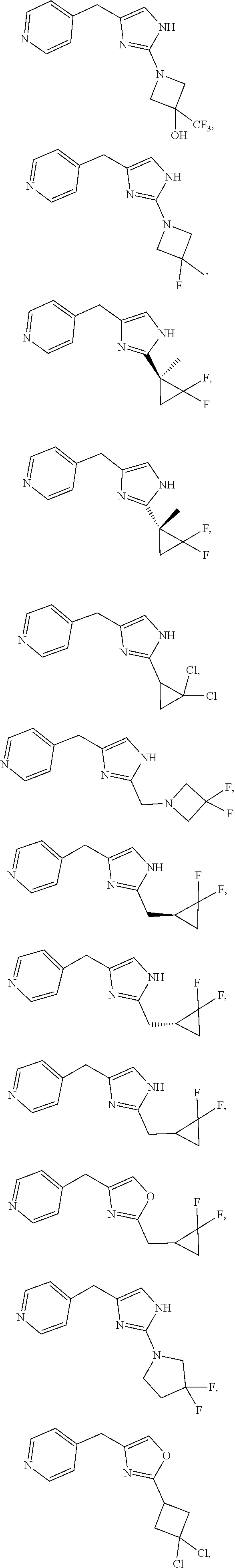

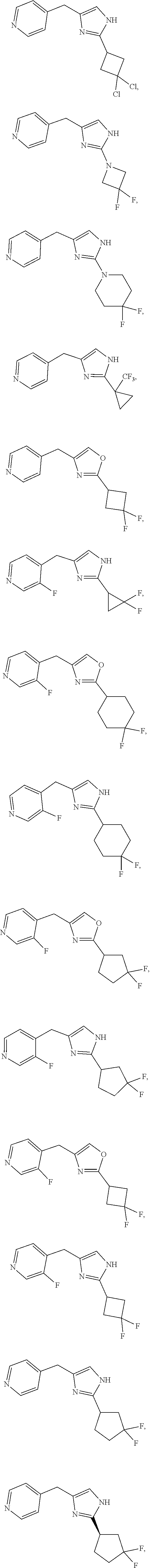

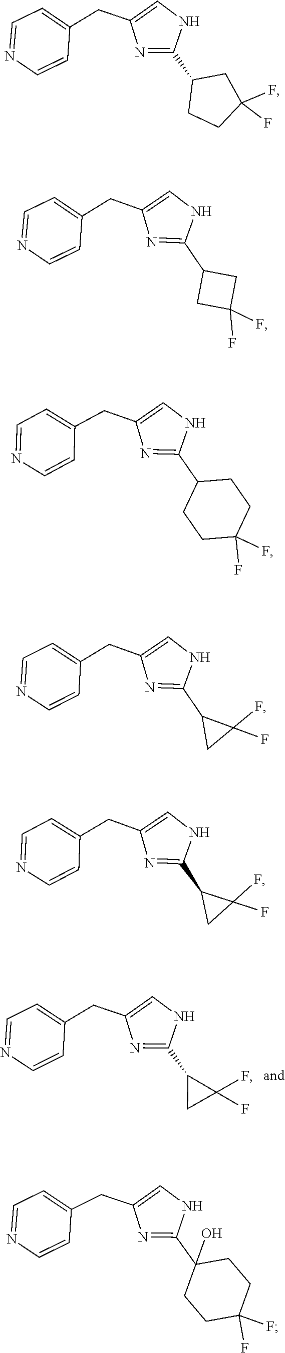

or a pharmaceutically acceptable salt thereof.

In some embodiments is a compound selected from:

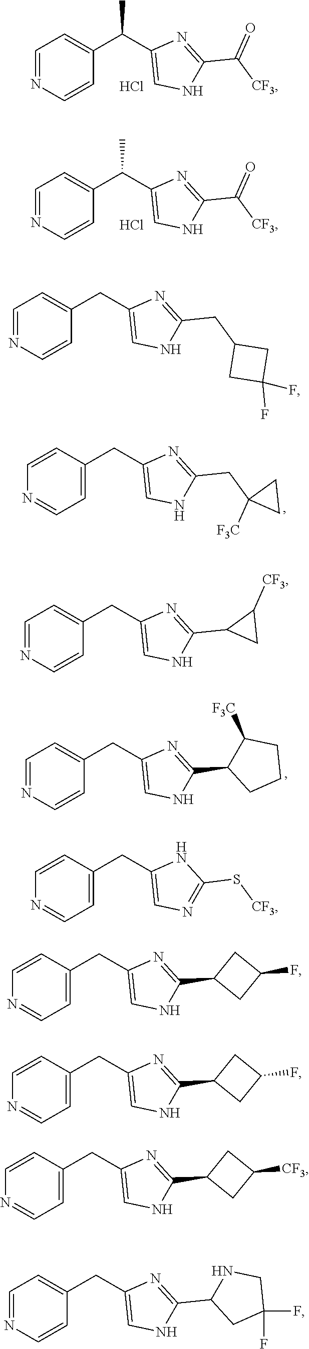

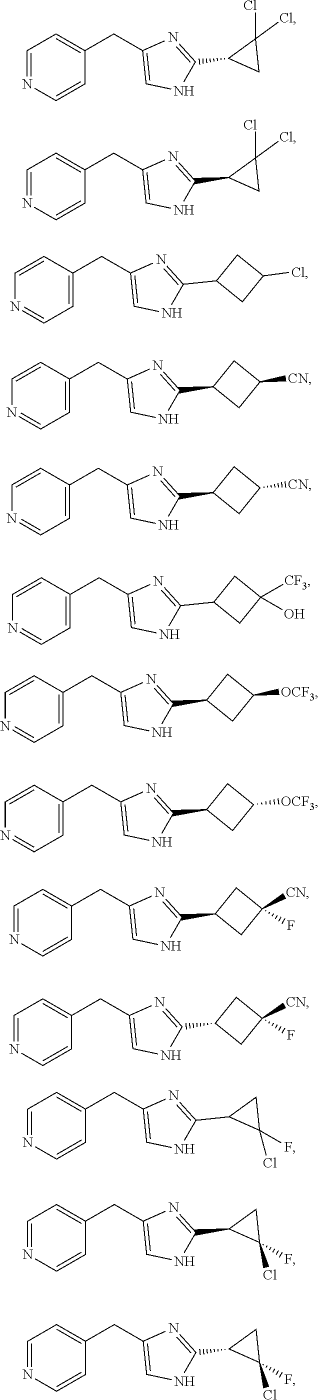

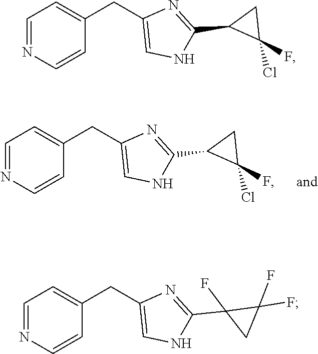 [SMILES: F[C@]1(Cl)C[C@@H]1c1nc(Cc2ccncc2)c[nH]1 ; F[C@]1(Cl)C[C@H]1c1nc(Cc2ccncc2)c[nH]1 ; "and" ; FC1(F)CC1(F)c1nc(Cc2ccncc2)c[nH]1]

or a pharmaceutically acceptable salt thereof. It is further appreciated that certain features of the disclosure described herein, which are, for clarity, described in the context of separate embodiments, can also be provided in combination in a single embodiment.

Conversely, various features of the disclosure described herein which are, for brevity, described in the context of a single embodiment, can also be provided separately or in any suitable subcombination.

At various places in the present specification, substituents of compounds described herein are disclosed in groups or in ranges. It is specifically intended that the groups or ranges include each and every individual subcombination of the members of such groups and ranges.

For example, the term “C₁₋₆ alkyl” is specifically intended to individually disclose methyl, ethyl, C₃ alkyl, C₄ alkyl, C₅ alkyl, and C₆ alkyl.

At various places in the present specification various aryl, heteroaryl, cycloalkyl, and heterocycloalkyl rings are described. Unless otherwise specified, these rings can be attached to the rest of the molecule at any ring member as permitted by valency. For example, the term “pyridinyl,” “pyridyl,” or “a pyridine ring” may refer to a pyridin-2-yl, pyridin-3-yl, or pyridin-4-yl ring.

At various places in the present specification a di-valent or linking group may be present. Each linking group is understood as linking in either direction. That is, if a linking group is described as -A-B-, then it is understood, unless otherwise specified, that the linking group is not directionally limited and can also be -B-A-. For example, when a linking group is written as

—C(═O)—O—, it also means —O—C(═O)—.

The term “n-membered,” where “n” is an integer, typically describes the number of ring-forming atoms in a moiety where the number of ring-forming atoms is “n”. For example, piperidinyl is an example of a 6-membered heterocycloalkyl ring, pyrazolyl is an example of a 5-membered heteroaryl ring, pyridyl is an example of a 6-membered heteroaryl ring, and 1,2,3,4-tetrahydro-naphthalene is an example of a 10-membered cycloalkyl group.

For compounds described herein in which a variable appears more than once, each variable can be a different moiety independently selected from the group defining the variable. For example, where a structure is described having two R groups that are simultaneously present on the same compound, the two R groups can represent different moieties independently selected from the group defined for R.

As used herein, the phrase “optionally substituted” means unsubstituted or substituted.

As used herein, the term “substituted” means that a hydrogen atom is replaced by a non-hydrogen group. Itis to be understood that substitution at a given atom is limited by valency. In some embodiments, an atom substituted by oxo (═O) has two hydrogen atoms replaced by the oxo substituent.

As used herein, the term “Cᵢ₋ⱼ,” where i and j are integers, employed in combination with a chemical group, designates a range of the number of carbon atoms in the chemical group with i-j defining the range. For example, C₁0.6 alkyl refers to an alkyl group having 1, 2, 3, 4, 5, or 6 carbon atoms.

As used herein, the term “alkyl,” employed alone or in combination with other terms, refers to a saturated hydrocarbon group that may be straight-chain or branched. In some embodiments, the alkyl group contains 1 to 7, 1 to 6, 1 to 4, or 1 to 3 carbon atoms. Examples of alkyl moieties include, but are not limited to, chemical groups such as methyl, ethyl, n-propyl, isopropyl, n-butyl, isobutyl, sec-butyl, tert-butyl, n-pentyl, 2-methyl-1-butyl, 3-pentyl, n-hexyl, 1,2,2-trimethylpropyl, n-heptyl, and the like. In some embodiments, the alkyl group is methyl, ethyl, or propyl. In some embodiments, the alkyl group is methyl.

As used herein, “halo” or “halogen”, employed alone or in combination with other terms, includes fluoro, chloro, bromo, and iodo. In some embodiments, halo is F or Cl.

As used herein, the term “haloalkyl,” employed alone or in combination with other terms, refers to an alkyl group having up to the full valency of halogen atom substituents, which may either be the same or different. In some embodiments, the halogen atoms are fluoro atoms.

In some embodiments, the alkyl group has 1 to 6 or 1 to 4 carbon atoms. Example haloalkyl groups include CF₃, C₂F₅, CHF₂, CCl₃, CHCl₂, C₂Cl₅, and the like.

As used herein, the term “alkoxy,” employed alone or in combination with other terms, refers to a group of formula —O-alkyl. Example alkoxy groups include methoxy, ethoxy, propoxy (e.g., n-propoxy and isopropoxy), t-butoxy, and the like. In some embodiments, the alkyl group has 1 to 6 or 1 to 4 carbon atoms.

As used herein, “haloalkoxy,” employed alone or in combination with other terms, refers to a group of formula —O-(haloalkyl). In some embodiments, the alkyl group has 1 to 6 or 1 to 4 carbon atoms. An example haloalkoxy group is —OCF₃.

As used herein, “amino,” employed alone or in combination with other terms, refers to NH₂.

As used herein, the term “cycloalkyl,” employed alone or in combination with other terms, refers to a non-aromatic cyclic hydrocarbon including cyclized alkyl and alkenyl groups.

Cycloalkyl groups can include mono- or polycyclic (e.g., having 2, 3, or 4 fused, bridged, or spiro rings) ring systems. Also included in the definition of cycloalkyl are moieties that have one or more aromatic rings (e.g., aryl or heteroaryl rings) fused (i.e., having a bond in common with) to the cycloalkyl ring, for example, benzo derivatives of cyclopentane, cyclohexene, cyclohexane, and the like, or pyrido derivatives of cyclopentane or cyclohexane. Ring-forming carbon atoms of a cycloalkyl group can be optionally substituted by oxo. Cycloalkyl groups also include cycloalkylidenes. The term “cycloalkyl” also includes bridgehead cycloalkyl groups (e.g., non-aromatic cyclic hydrocarbon moieties containing at least one bridgehead carbon, such as admantan-1-yl) and spirocycloalkyl groups (e.g., non-aromatic hydrocarbon moieties containing at least two rings fused at a single carbon atom, such as spiro[2.5]octane and the like). In some embodiments, the cycloalkyl group has 3 to 10 ring members, or 3 to 7 ring members. In some embodiments, the cycloalkyl group is monocyclic or bicyclic. In some embodiments, the cycloalkyl group is monocyclic. In some embodiments, the cycloalkyl group is a C₃₋₇ monocyclic cycloalkyl group. Example cycloalkyl groups include cyclopropyl, cyclobutyl, cyclopentyl, cyclohexyl, cycloheptyl, cyclopentenyl, cyclohexenyl, cyclohexadienyl, cycloheptatrienyl, norbornyl, norpinyl, norcarnyl, tetrahydronaphthalenyl, octahydronaphthalenyl, indanyl, and the like. In some embodiments, the cycloalkyl group is cyclopropyl, cyclobutyl, cyclopentyl, or cyclohexyl.

As used herein, the term “heterocycloalkyl,” employed alone or in combination with other terms, refers to a non-aromatic ring or ring system, which may optionally contain one or more alkenylene or alkynylene groups as part of the ring structure, which has at least one heteroatom ring member independently selected from nitrogen, sulfur, oxygen, and phosphorus. Heterocycloalkyl groups can include mono- or polycyclic (e.g., having 2, 3 or 4 fused, bridged, or spiro rings) ring systems. In some embodiments, the heterocycloalkyl group is a mono cyclic or bicyclic group having 1, 2, 3, or 4 heteroatoms independently selected from nitrogen, sulfur and oxygen. Also included in the definition of heterocycloalkyl are moieties that have one or more aromatic rings (e.g., aryl or heteroaryl rings) fused (i.e., having a bond in common with) to the non-aromatic heterocycloalkyl ring, for example, 1,2,3,4-tetrahydro-quinoline and the like. Heterocycloalkyl groups can also include bridgehead heterocycloalkyl groups (e.g., a heterocycloalkyl moiety containing at least one bridgehead atom, such as azaadmantan-1-yl and the like) and spiroheterocycloalkyl groups (e.g., a heterocycloalkyl moiety containing at least two rings fused at a single atom, such as [1,4-dioxa-8-aza-spiro[4.5]decan-N-yl] and the like). In some embodiments, the heterocycloalkyl group has 3 to 10 ring-forming atoms, 4 to 10 ring-forming atoms, or about 3 to 8 ring forming atoms. In some embodiments, the heterocycloalkyl group has 2 to 20 carbon atoms, 2 to 15 carbon atoms, 2 to 10 carbon atoms, or about 2 to 8 carbon atoms. In some embodiments, the heterocycloalkyl group has 1 to 5 heteroatoms, 1 to 4 heteroatoms, 1 to 3 heteroatoms, or 1 to 2 heteroatoms. The carbon atoms or heteroatoms in the ring(s) of the heterocycloalkyl group can be oxidized to form a carbonyl, an N-oxide, or a sulfonyl group (or other oxidized linkage) or a nitrogen atom can be quaternized. In some embodiments, the heterocycloalkyl portion is a C₂₇monocyclic heterocycloalkyl group. In some embodiments, the heterocycloalkyl group is a morpholine ring, pyrrolidine ring, piperazine ring, piperidine ring, tetrahydropyran ring, tetrahydropyridine, azetidine ring, or tetrahydrofuran ring.

As used herein, the term “aryl,” employed alone or in combination with other terms, refers to a monocyclic or polycyclic (e.g., a fused ring system) aromatic hydrocarbon moiety, such as, but not limited to, phenyl, 1-naphthyl, 2-naphthyl, and the like. In some embodiments, aryl groups have from 6 to 10 carbon atoms or 6 carbon atoms. In some embodiments, the aryl group is a monocyclic or bicyclic group. In some embodiments, the aryl group is phenyl or naphthyl. In some embodiments, the aryl group is phenyl.

As used herein, the term “heteroaryl,” employed alone or in combination with other terms, refers to a monocyclic or polycyclic (e.g., a fused ring system) aromatic hydrocarbon moiety, having one or more heteroatom ring members independently selected from nitrogen, sulfur and oxygen. In some embodiments, the heteroaryl group is a monocyclic or a bicyclic group having 1, 2, 3, or 4 heteroatoms independently selected from nitrogen, sulfur and oxygen.

In some embodiments, the heteroaryl group is a 5-14 membered heteroaryl group. In some embodiments, the heteroaryl group is a 5-10 membered heteroaryl group. In some embodiments, the heteroaryl group is a 5-6 membered heteroaryl group. Example heteroaryl groups include, but are not limited to, pyridyl, pyrimidinyl, pyrazinyl, pyridazinyl, triazinyl, furyl, thienyl, imidazolyl, thiazolyl, indolyl, pyrryl, oxazolyl, benzofuryl, benzothienyl, benzthiazolyl, isoxazolyl, pyrazolyl, triazolyl, tetrazolyl, indazolyl, 1,2,4-thiadiazolyl, isothiazolyl, purinyl, carbazolyl, benzimidazolyl, indolinyl, pyrrolyl, azolyl, quinolinyl, isoquinolinyl, benzisoxazolyl, imidazo[1,2-b]thiazolyl or the like. The carbon atoms or heteroatoms in the ring(s) of the heteroaryl group can be oxidized to form a carbonyl, an N-oxide, or a sulfonyl group (or other oxidized linkage) or a nitrogen atom can be quaternized, provided the aromatic nature of the ring is preserved. In some embodiments, the heteroaryl group has from 3 to 10 carbon atoms, from 3 to 8 carbon atoms, from 3 to 5 carbon atoms, from 1 to 5 carbon atoms, or from 5 to 10 carbon atoms. In some embodiments, the heteroaryl group contains 3 to 14, 4 to 12, 4 to 8, 9 to 10, or 5 to 6 ring-forming atoms. In some embodiments, the heteroaryl group has 1 to 4, 1 to 3, or 1 to 2 heteroatoms.

The compounds described herein can be asymmetric (e.g., having one or more stereocenters). All stereoisomers, such as enantiomers and diastereomers, are intended unless otherwise indicated. Compounds described herein that contain asymmetrically substituted carbon atoms can be isolated in optically active or racemic forms. Methods on how to prepare optically active forms from optically inactive starting materials are known in the art, such as by resolution of racemic mixtures or by stereoselective synthesis. Geometric isomers of olefins, C═N double bonds, and the like can also be present in the compounds described herein, and all such stable isomers are contemplated herein. Cis and trans geometric isomers of the compounds described herein may be isolated as a mixture of isomers or as separated isomeric forms.

Compounds described herein also include tautomeric forms. Tautomeric forms result from the swapping of a single bond with an adjacent double bond together with the concomitant migration of a proton. Tautomeric forms include prototropic tautomers which are isomeric protonation states having the same empirical formula and total charge. Example prototropic tautomers include ketone-enol pairs, amide-imidic acid pairs, lactam-lactim pairs, enamine imine pairs, and annular forms where a proton can occupy two or more positions of a heterocyclic system, for example, 1H- and 3H-imidazole, 1H-, 2H- and 4H-1,2,4-triazole, 1H- and 2H-isoindole, and 1H- and 2H-pyrazole.

Compounds described herein also include all isotopes of atoms occurring in the intermediates or final compounds. Isotopes include those atoms having the same atomic number but different mass numbers. For example, isotopes of hydrogen include tritium and deuterium. In some embodiments, the compounds described herein include at least one deuterium atom.

The term, “compound,” as used herein is meant to include all stereoisomers, geometric isomers, tautomers, and isotopes of the structures depicted, unless otherwise specified. The term “compound” is also not limited by the way in which it was made. Thus, a compound described herein includes molecules that were made by a synthetic process or by a biological process (such as through enzyme conversion or metabolism), or combinations thereof.

All compounds, and pharmaceutically acceptable salts thereof, can be found together with other substances such as water and solvents (e.g., in the form of hydrates and solvates) or can be isolated.

In some embodiments, the compounds described herein, or salts thereof, are substantially isolated. By “substantially isolated” is meant that the compound is at least partially or substantially separated from the environment in which it was formed or detected. Partial separation can include, for example, a composition enriched in the compounds described herein. Substantial separation can include compositions containing at least about 50%, at least about 60%, at least about 70%, at least about 80%, at least about 90%, at least about 95%, at least about 97%, or at least about 99% by weight of a compound described herein, or salt thereof. Methods for isolating compounds and their salts are routine in the art.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, “pharmaceutically acceptable salts” refers to derivatives of the disclosed compounds wherein the parent compound is modified by converting an existing acid or base moiety to its salt form. Examples of pharmaceutically acceptable salts include, but are not limited to, mineral or organic acid salts of basic residues such as amines; alkali or organic salts of acidic residues such as carboxylic acids; and the like. The pharmaceutically acceptable salts of the compounds described herein include the non-toxic salts of the parent compound formed, for example, from non-toxic inorganic or organic acids. The pharmaceutically acceptable salts of the compounds described herein can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two. Lists of suitable salts are found in Remington's Pharmaceutical Sciences, 17th ed., Mack Publishing Company, Easton, Pa., 1985, p. 1418 and Journal of Pharmaceutical Science, 66, 2 (1977), each of which is incorporated herein by reference in its entirety.

### Methods of Use

Compounds described herein can inhibit the activity of SARM1. For example, the compounds described herein can be used to inhibit activity or a function of SARM1 in a cell or in an individual or patient in need of inhibition of the enzyme by administering an inhibiting amount of a compound described herein to the cell, individual, or patient. As used herein, the term “in a cell” includes both inside the cell membrane and on the surface of the cell membrane.

Compounds described herein, as SARM1 inhibitors, can increase levels of NAD+ in a cell. Accordingly, the present disclosure is further directed to a method of increasing the level of NAD+ in a sample or in a patient, comprising contacting the sample or administering to the patient a compound described herein, or a pharmaceutically acceptable salt thereof, wherein the increased level of NAD+ is relative to the level of NAD+ prior to the contacting or administering.

Compounds described herein, as SARM1 inhibitors, can inhibit axonal degeneration. Accordingly, the present disclosure is further directed to a method of inhibiting axonal degeneration in a sample or in a patient, comprising contacting the sample or administering to the patient an inhibiting amount of a compound described herein, or a pharmaceutically acceptable salt thereof.

The compounds described herein are useful in the treatment and prevention of various diseases associated with abnormal expression or activity of SARM1. For example, the compounds described herein are useful in the treatment and prevention of neurological disorders. In some embodiments, the compounds described herein are useful in the treatment of neurological disorders. In some embodiments, the compounds described herein are useful in the prevention of neurological disorders. The term “neurological disorder” generally refers to a disorder affecting the nervous system, including the central nervous system or the peripheral nervous system. The term “neurological disorder” also includes ocular indications having a nexus to the nervous system.

In some embodiments, the neurological disorder treatable or preventable by administration of a compound described herein includes neurodegenerative diseases. In some embodiments, the compounds described herein are useful in the treatment of neurodegenerative diseases. In some embodiments, the compounds described herein are useful in the prevention of neurodegenerative diseases. Neurodegenerative diseases are characterized by damage to the central nervous system and can be identified by progressive dysfunction, degeneration and death of specific populations of neurons which are often synaptically interconnected. Examples of neurodegenerative diseases include Parkinson's disease (PD), Alzheimer's disease (AD), Huntington's disease (HD), prion disease, motor neuron diseases (MND), spinocerebellar ataxia (SCA), spinal muscular atrophy (SMA), amyotrophic lateral sclerosis (ALS), and epilepsy.

Examples of neurological disorders treatable or preventable according to the methods described herein include spinal muscular atrophy (SMA), Chemotherapy Induced Peripheral Neuropathy (representative chemotherapeutic agents include vinca-alkaloids, taxols and platins), multiple sclerosis (MS), traumatic brain injury (TBI), spinal cord injury, stroke, Parkinson' disease, glaucoma, Huntington's disease, Alzheimer's disease, Charcot-Marie-Tooth disease (CMT), retinitis pigmentosa (RP), age-related macular degeneration (AMD), small fiber neuropathies, peripheral neuropathy (e.g., viral neuropathy), spinocerebellar ataxias, cystic fibrosis, familial amyloidotic polyneuropathy, spongiform encephalopathies, spinal and bulbar muscular atrophy, hereditary dentatorubral-pallidoluysian atrophy, adrenoleukody strophy, adrenomyeloneuropathy, Alexander's disease, amyotrophic lateral sclerosis (ALS), Bassen-Kornzweig syndrome, Bell's palsy, progressive supra nuclear palsy (PSP), central pontine myelolysis, cluster headache, congenital hypomyelination, corticobasal degeneration, Creutzfeldt-Jakob disease, epilepsy, dementia (e.g., frontotemporal dementia and Lewy body dementia), demyelination disorders (e.g., ischemic demyelination), encephalomyelitis, Friedrich's ataxia, Gaucher's disease, hereditary sensory and autonomic neuropathy (HSAN), Hurler syndrome, Krabbe's disease, metachromatic leukodystrophy, migraine and tension headaches, mild cognitive impairment, motor spinoneuron disease, neuromyelitis optica, Niemann-Pick disease, optic neuritis, Pelizaeus Merzbacher disease, peripheral neuropathy, periventricular leukomalacia, post-herpetic neuralgia, prion disease, progressive supranuclear palsy, progressive multifocal leukoencephalopathy, Tay-Sacks disease, thoracic disc herniation, traverse myelitis, trigeminal neuralgia, Wallerian degeneration, cerebellar degeneration, chiari malformation, dystonia, encephalitis (e.g., pediatric viral encephalitis and La Crosse virus encephalitis), hyperekplexia, multifocal motor neuropathy, muscular dystrophy, myasthenia gravis, myopathy, neurofibromatosis, neuronal ceroid lipofuscinosis, neuropathies (e.g., peripheral neuropathy), pseudobulbar affect, restless legs syndrome, spina bifida, syringomyelia, thoracic outlet syndrome, and transverse myelitis.

In other embodiments, the neurological disorder treatable or preventable by administration of a compound described herein is a neuropathy. As used herein, the term “neuropathy” refers broadly to diseased conditions of the nervous system, including polyneuropathy; neuropathy, ataxia, and retinosa pigmentosa (NARP); familial amyloid neuropathies; diabetic neuropathy (peripheral neuropathy due to diabetes mellitus); peripheral neuropathy (e.g., chemotherapy-induced peripheral neuropathy (CIPN), including CIPN caused by vinca alkaloids, bortezomib, lxabepilone, thalidomide and its analogs, taxanes, and platinum-based agents); and cranial neuropathy (e.g., auditory neuropathy and optic neuropathy). The term also includes other neuropathies associated with genetic disorders (e.g., NMNAT2 genetic mutation disorders).

In still other embodiments, the neurological disorder treatable or preventable by administration of a compound described herein is an ocular neuropathy (e.g., optic neuropathy). The term “optic neuropathy” refers to damage to the optic nerve from a number of causes. Types of optic neuropathy include ischemic optic neuropathy (e.g., anterior and posterior ischemic optic neuropathy); optic neuritis (e.g., chronic relapsing inflammatory optic neuropathy (CRION), single isolated optic neuritis (SION), and relapsing isolated optic neuritis); compressive optic neuropathy; infiltrative optic neuropathy; traumatic optic neuropathy; mitochondrial optic neuropathies; and hereditary optic neuropathies (e.g., Leber's hereditary optic neuropathy (LHON), hereditary neuropathy with liability to pressure palsy (HNPP), and dominant optic atrophy).

In still other embodiments, the neurological disorder treatable or preventable by administration of a compound described herein is multiple sclerosis (MS), chemotherapy-induced peripheral neuropathy (CIPN), amyotrophic lateral sclerosis (ALS), glaucoma, traumatic brain injury (TBI), or stroke.

As used herein, the term “cell” is meant to refer to a cell that is in vitro, ex vivo or in vivo. In some embodiments, an ex vivo cell can be part of a tissue sample excised from an organism such as a mammal. In some embodiments, an in vitro cell can be a cell in a cell culture. In some embodiments, an in vivo cell is a cell living in an organism such as a mammal.

As used herein, the term “contacting” refers to the bringing together of indicated moieties in an in vitro system or an in vivo system. For example, “contacting” SARM1 or “contacting” a cell with a compound described herein includes the administration of a compound described herein to an individual or patient, such as a human, having SARM1, as well as, for example, introducing a compound described herein into a sample containing a cellular or purified preparation containing SARM1.

As used herein, the term “individual” or “patient,” used interchangeably, refers to mammals, and particularly humans. The individual or patient can be in need of treatment.

As used herein, the phrase “therapeutically effective amount” refers to the amount of active compound or pharmaceutical agent that elicits the biological or medicinal response in a tissue, system, animal, individual or human that is being sought by a researcher, veterinarian, medical doctor or other clinician.

As used herein, the phrase “inhibiting amount” refers to the amount of active compound or pharmaceutical agent that elicits a measurable SARM1 inhibition or axonal degeneration in a tissue, system, animal, individual or human.

As used herein the term “treating” or “treatment” refers to 1) inhibiting the disease in an individual who is experiencing or displaying the pathology or symptomatology of the disease (i.e., arresting further development of the pathology and/or symptomatology), or 2) ameliorating the disease in an individual who is experiencing or displaying the pathology or symptomatology of the disease (i.e., reversing the pathology and/or symptomatology).

As used herein the term “preventing” or “prevention” refers to preventing the disease in an individual who may be predisposed to the disease but does not yet experience or display the pathology or symptomatology of the disease. In some embodiments is a method of preventing a disease in a patient, by administering to the patient a therapeutically effective amount of a compound described herein, or a pharmaceutically acceptable salt thereof.

### Combination Therapy

One or more additional pharmaceutically active agents or treatment methods can be used in combination with the compounds described herein. The agents can be combined with the present compounds in a single dosage form, or the agents can be administered simultaneously or sequentially as separate dosage forms. Examples of additional agents include acamprosate, agomelatine, almotriptan, amantadine, amisulpride, amitriptyline, apomorphine, aripiprazole, asenapine, atomoxetine, baclofen, botulinum toxin type A, bromocriptine, buccal midazolam, buprenorphine, buspirone, cabergoline, carbamazepine, chlordiazepoxide, chlorpromazine, citalopram, clobazam, clomethiazole, clomipramine, clonazepam, clozapine, denzapine, co-beneldopa, co-careldopa, dantrolene, dexamfetamine, diazepam, divalproex sodium, donepezil, doxepin, duloxetine, eletriptan, entacapone, epinephrine, escitalopram, eslicarbazepine, ethosuximide, fingolimod, fluoxetine, flupentixol, flupentixol, fluphenazine long-acting injection (modecate), fluvoxamine (Faverin), frovatriptan, gabapentin, galantamine, haloperidol, imipramine, lacosamide, lamotrigine, levetiracetam, levomepromazine, lisdexamfetamine, lithium, lofepramine, loprazolam, lorazepam, lormetazepam, lurasidone, melatonin, memantine, methylphenidate, mianserin, mirtazapine, moclobemide, modafinil, naratriptan, neostigmine, nitrazepam, nortriptyline, olanzapine, orlistat, orphenadrine, oxazepam, oxcarbazepine, paliperidone, paliperidone, paroxetine, perampanel, pergolide, pericyazine, phenobarbital, phenytoin, piracetam, pizotifen, pramipexole, pregabalin, primidone, prochlorperazine, procyclidine, pyridostigmine, quetiapine, rasagiline, reboxetine, risperidone, rivastigmine, rizatriptan, ropinirole, rotigotine, rufinamide, selegiline, sertraline, sodium oxybate, sodium valproate, sulpiride, sumatriptan, temazepam, tetrabenazine, tiagabine, tizanidine, tolcapone, topiramate, trazodone, trihexyphenidyl, trimipramine, valproate semisodium, venlafaxine, vigabatrin, vortioxetine, zolmitriptan, zolpidem, zonisamide, zopiclone, and zuclopenthixol.

In some embodiments, the one or more additional pharmaceutically active agent can include a neuroprotective agent. In some embodiments, the neuroprotective agent is a dual leucine-zipper kinase (DLK) inhibitor. In some embodiments, the neuroprotective agent is a nicotinamide phosphoribosyltransferase (NAMPT) inhibitor.

In some embodiments, the one or more additional pharmaceutically active agent can be NAD+ or an NAD+ precursor. NAD+ precursors include, for example, nicotinamide riboside (NR), nicotinic acid (NA), nicotinic acid riboside (NaR), nicotinamide (NAM), nicotinamide mononucleotide (NMN), nicotinic acid mononucleotide (NaMN), tryptophan, vitamin B3, and nicotinic acid adenine dinucleotide (NAAD).

### Pharmaceutical Formulations and Dosage Forms

When employed as pharmaceuticals, the compounds described herein can be administered in the form of pharmaceutical compositions. A pharmaceutical composition refers to a combination of a compound described herein, or its pharmaceutically acceptable salt, and at least one pharmaceutically acceptable carrier.

These compositions can be prepared in a manner well known in the pharmaceutical art, and can be administered by a variety of routes, depending upon whether local or systemic treatment is desired and upon the area to be treated. Pharmaceutical compositions may be formulated in a conventional manner using one or more physiologically acceptable carriers including excipients and auxiliaries which facilitate processing of the active compounds into preparations which can be used pharmaceutically. Proper formulation is dependent upon the route of administration chosen. Additional details about suitable excipients for pharmaceutical compositions described herein may be found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pennsylvania 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999), herein incorporated by reference for such disclosure.

Administration may be oral, topical (including ophthalmic and to mucous membranes including intranasal, vaginal and rectal delivery), pulmonary (e.g., by inhalation or insufflation of powders or aerosols, including by nebulizer; intratracheal, intranasal, epidermal and transdermal), ocular (e.g., eye drops or intravitreal, subconjunctival, subtenon, or retrobulbar injection), or parenteral.

Also described herein are pharmaceutical compositions which contain, as the active ingredient, one or more of the compounds described herein above in combination with one or more pharmaceutically acceptable carriers. In making the compositions described herein, the active ingredient is typically mixed with an excipient, diluted by an excipient or enclosed within such a carrier in the form of, for example, a capsule, sachet, paper, or other container. When the excipient serves as a diluent, it can be a solid, semi-solid, or liquid material, which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments, soft and hard gelatin capsules, suppositories, sterile injectable solutions, and sterile packaged powders.

The compositions can be formulated in a unit dosage form. The term “unit dosage form” refers to a physically discrete unit suitable as unitary dosages for human subjects and other mammals, each unit containing a predetermined quantity of active material calculated to produce the desired therapeutic effect, in association with a suitable pharmaceutic al excipient.

The active compound can be effective over a wide dosage range and is generally administered in a pharmaceutically effective amount. It will be understood, however, that the amount of the compound actually administered will usually be determined by a physician, according to the relevant circumstances, including the condition to be treated, the chosen route of administration, the actual compound administered, the age, weight, and response of the individual patient, the severity of the patient's symptoms, and the like.

For preparing solid compositions such as tablets, the principal active ingredient is mixed with a pharmaceutical excipientto form a solid pre-formulation composition containing a homogeneous mixture of a compound described herein. When referring to these pre-formulation compositions as homogeneous, the active ingredient is typically dispersed evenly throughout the composition so that the composition can be readily subdivided into equally effective unit dosage forms such as tablets, pills and capsules. This solid pre-formulation is then subdivided into unit dosage forms of the type described above.

The tablets or pills described herein can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer which serves to resist disintegration in the stomach and permit the inner component to pass intact into the duodenum or to be delayed in release. A variety of materials can be used for such enteric layers or coatings, such materials including a number of polymeric acids and mixtures of polymeric acids with such materials as shellac, cetyl alcohol, and cellulose acetate.

The liquid forms in which the compounds and compositions described herein can be incorporated for administration orally or by injection include aqueous solutions, suitably flavored syrups, aqueous or oil suspensions, and flavored emulsions with edible oils such as cottonseed oil, sesame oil, coconut oil, or peanut oil, as well as elixirs and similar pharmaceutical vehicles.

Compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as described supra. In some embodiments, the compositions are administered by the oral or nasal respiratory route for local or systemic effect. Compositions can be nebulized by use of inert gases. Nebulized solutions may be breathed directly from the nebulizing device or the nebulizing device can be attached to a face masks tent, or intermittent positive pressure breathing machine. Solution, suspension, or powder compositions can be administered orally or nasally from devices which deliver the formulation in an appropriate manner.

The amount of compound or composition administered to a patient will vary depending upon what is being administered, the purpose of the administration, such as prophylaxis or therapy, the state of the patient, the manner of administration, and the like. In therapeutic applications, compositions can be administered to a patient already suffering from a disease in an amount sufficient to cure or at least partially arrest the symptoms of the disease and its complications. Effective doses will depend on the disease condition being treated as well as by the judgment of the attending clinician depending upon factors such as the severity of the disease, the age, weight and general condition of the patient, and the like.

The compositions administered to a patient can be in the form of pharmaceutical compositions described above. These compositions can be sterilized by conventional sterilization techniques, or may be sterile filtered. Aqueous solutions can be packaged for use as is, or lyophilized, the lyophilized preparation being combined with a sterile aqueous carrier prior to administration.

The therapeutic dosage of the compounds described herein can vary according to, for example, the particular use for which the treatment is made, the manner of administration of the compound, the health and condition of the patient, and the judgment of the prescribing physician. The proportion or concentration of a compound described herein in a pharmaceutical composition can vary depending upon a number of factors including do sage, chemical characteristics (e.g., hydrophobicity), and the route of administration. The dosage is likely to depend on such variables as the type and extent of progression of the disease or disorder, the overall health status of the particular patient, the relative biological efficacy of the compound selected, formulation of the excipient, and its route of administration. Effective doses can be extrapolated from dose-response curves derived from in vitro or animal model test systems.

### EXAMPLES

### General Experimental

The following examples are offered for purposes of illustration and are not intended to limit the scope of the claims provided herein. Those of skill in the art will readily recognize a variety of non-critical parameters which can be changed or modified to yield essentially the same results. The compounds of the Examples were found to be inhibitors of SARM1 according to one or more of the assays provided herein.

All reactions sensitive to air or moisture were carried out in flame-dried glassware under an atmosphere of nitrogen. All commercially available reagents were purchased from suppliers such as Sigma-Aldrich (MilliporeSigma), Combi-Blocks, Enamine, Sinopharm Chemical Reagent Co. (SCRC), and Alfa Aesar and were used without purification unless otherwise noted. Proton nuclear magnetic resonance (¹H NMR) spectra were recorded on Bruker AVIII 400 or Bruker AVIII 500 spectrometers. Samples were dissolved in deuterated chloroform (CDCl₃), dimethyl sulfoxide (DMSO-d₆), acetonitrile (CD₃CN) or methanol (CD₃OD).

Chemical shifts are recorded in parts per million (ppm) and are referenced to the centerline of deuterochloroform (δ 7.26 ppm), of DMSO-d₆ (δ 2.50 ppm), of CD₃CN (δ 1.94 ppm) or of CD₃OD (δ 3.31 ppm). Data were recorded as follows: chemical shift, multiplicity (s=singlet, d=doublet, t=triplet, q=quartet, qt=quintet, m=multiplet, br=broad). Coupling constants (J values) are given in Hertz (Hz). Low resolution ESI mass spectra were recorded on a either an Agilent 1200 HPLC/6100 SQ system or an Agilent 1260 Infinity II HPLC/6125 SQ system. LCMS measurements were recorded on Agilent 1200 HPLC/6100 SQ System.

### LIST OF ABBREVIATIONS

aq aqueous
Boc tert-butyloxycarbonyl
Boc₂O Boc-anhydride or di-tert-butyl dicarbonate
CDI 1,1′-carbonyl-diimidazole
d day(s)
D ²H (deuterium)
DABCO 1,4-diazabicyclo[2.2.2]octane
dba dibenzylideneacetone
DCM dichloromethane
DMP Dess-Martin periodinane or 3-Oxo-1λ⁵,2-benziodoxole-1,1,1(3H)-triyl
triacetate
DEA diethylamine
DHP 3,4-dihydropyran
DIAD diisopropyl azodicarboxylate
DIPEA N,N-diisopropylethylamine
DMAP 4-(dimethylamino)pyridine
DMF dimethylformamide
DMSO dimethylsulfoxide
DPPA diphenyl phosphoryl azide
EDC HCl N-ethyl-N′-(3-dimethylaminopropyl)carbodiimide hydrochloride
ESI-MS electrospray ionization-mass spectrometry
Et ethyl
EtOAc ethyl acetate
EtOH ethanol
equiv equivalent(s)
FA formic acid
(g) gaseous
h hour(s)
HATU 1-[bis(dimethylamino)methylene]-1H-1,2,3-triazolo[4,5-b]pyridinium 3-oxide hexafluorophosphate
HOBt 1-hydroxybenzotriazole
LAH lithium aluminum hydride
LCMS liquid chromatography mass spectrometry
LiHMDS lithium bis(trimethylsilyl)amide
MS mass spectrometry
MeCN acetonitrile
MeOH methanol
MHz megahertz
min minute(s)
mg milligram(s)
mL milliliter(s)
mmol millimole(s)
M molar
MeCN acetonitrile
mol mole(s)
Ms methanesulfonyl
MW microwave
N normal
NBS N-bromosuccinimide
NCS N-chlorosuccinimide
OTf trifluoromethanesulfonate
Pd/C palladium on carbon
PE petroleum ether
Ph phenyl
PTSA p-toluenesulfonic acid
¹H NMR proton nuclear magnetic resonance
RP-HPLC reverse-phase high performance liquid chromatography
RT retention time
rt room temperature
sat saturated
SEM 2-(trimethylsilyl)ethoxymethyl
SFC supercritical fluid chromatography
T3P propylphosphonic anhydride
TBAF tetrabutylammonium fluoride
TBPH tert-butyl hydroperoxide solution (Luperox®, TBH70X)
t-BuOK potassium tert-butoxide
TEA triethylamine
TFA trifluoroacetic acid
THF tetrahydrofuran
THP tetrahydropyran
TLC thin layer chromatography
Tol toluene
TosMIC p-toluenesulfonylmethyl isocyanide
wt % weight percent
v/v % volume by volume percent
w/v % weight by volume percent
XPhos 2-dicyclohexylphosphino-2′,4′,6′-triisopropylbiphenyl

### Example 1: 4-((2-(4,4-Difluoropiperidin-1-yl)-1H-imidazol-4-yl)pyridine

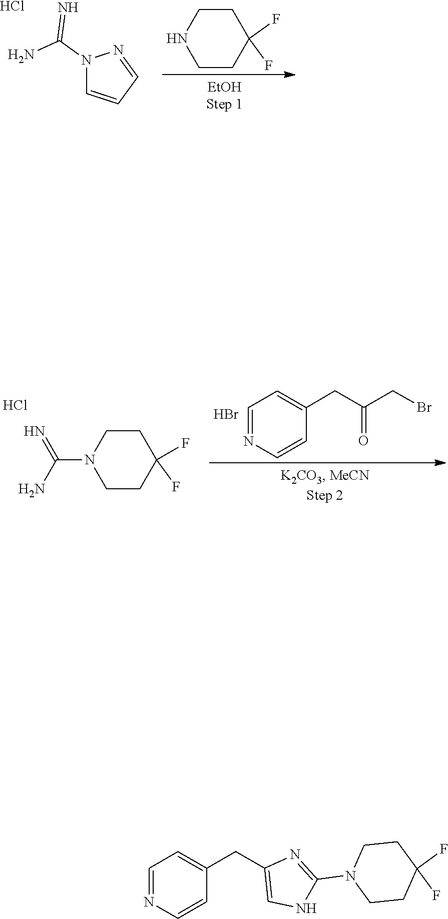

Step 1: 4,4-Difluoropiperidine-1-carboximidamide. To a stirred solution of pyrazole-1-carboximidamide (3 g, 21 mmol) was added 4,4-difluoropiperidine (4.9 g, 41 mmol) in portions at rt under a nitrogen atmosphere. The resulting mixture was stirred for 5 h at 80° C. under a nitrogen atmosphere. The resulting mixture was cooled to rt and concentrated under reduced pressure. The crude product was slurried with EtOAc (10 mL) and stirred for 10 min at rt. The resulting mixture was filtered, and the filter cake was washed with EtOAc (2×5 mL) to afford 4,4-difluoropiperidine-1-carboximidamide (3.1 g, 97%) as a white solid. LCMS ESI-MS m/z:=164 [M+H]⁺.

Step 2: 4-((2-(4,4-Difluoropiperidin-1-yl)-1H-imidazol-4-yl)pyridine. To a stirred solution of 4,4-difluoropiperidine-1-carboximidamide (2.5 g, 12.5 mmol) in MeCN (25 mL) was added K₂CO₃ (5.1 g, 38 mmol) and 1-bromo-3-(pyridine-4-yl)propan-2-one hydrobromide (3.6 g, 12.5 mmol) in portions at rt under a nitrogen atmosphere. The resulting mixture was stirred overnight at 50° C. under a nitrogen atmosphere. The resulting mixture was cooled to rt then filtered, and the filter cake was washed with MeCN (2×10 mL). The combined filtrate was concentrated under reduced pressure. The crude product was purified by prep-HPLC to afford 4-((2-(4,4-difluoropiperidin-1-yl)-1H-imidazol-4-yl)pyridine (17 mg, 0.3%) as a brown solid. ¹H NMR: (300 MHz, CDCl₃, ppm): δ 8.45 (dd, J=1.8 Hz, 4.5 Hz, 2H), 7.19 (d, J=6.1 Hz, 2H), 6.34 (s, 1H), 3.83 (s, 2H), 3.48-3.44 (m, 4H), 2.07-2.03 (m, 4H). LCMS ESI-MS m/z:=279 [M+H]⁺.

### Example 2: 4-((2-(3,3-Difluoroazetidin-1-yl)-1H-imidazol-4-yl)methyl)pyridine

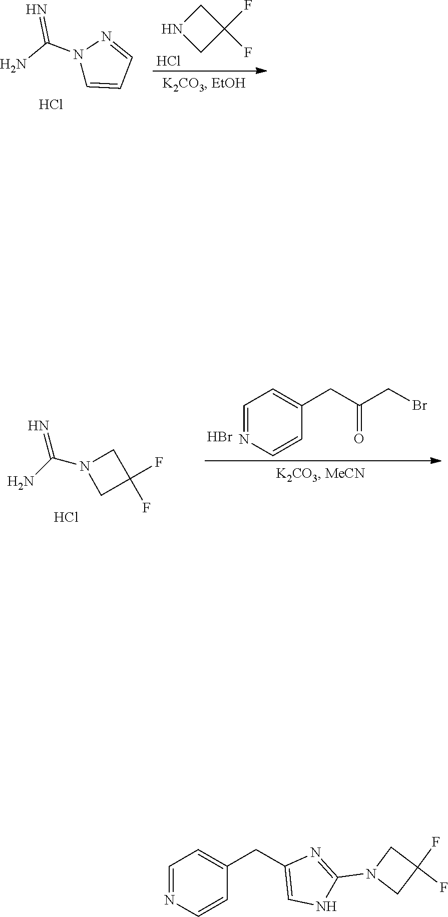

Step 1: 3,3-Difluoroazetidine-1-carboximidamide hydrochloride. To a stirred mixture of 3,3-difluoroazetidine hydrochloride (884 mg, 6.8 mmol) in EtOH (5 mL) was added K₂CO₃ (943 mg, 6.8 mmol) in portions at rt under a nitrogen atmosphere. The resulting mixture was stirred for 1 h at rt, filtered, and the filter cake was rinsed with EtOH (1×5 mL). To the above filtrate was added pyrazole-1-carboximidamide hydrochloride (500 mg, 3.4 mmol) in portions at rt. The resulting mixture was stirred for an additional 5 h under reflux. The resulting mixture was cooled to rt and concentrated under reduced pressure. The crude product was dissolved in EtOAc (20 mL) and slurried for 2 h. The resulting mixture was filtered, and the filter cake was rinsed with EtOAc (2×5 mL). The filter cake was dried under reduced pressure to afford 3,3-difluoroazetidine-1-carboximidamide hydrochloride (249 mg) as a white solid. LCMS ESI-MS m/z:=136 [M+H-HCl]⁺.

Step 2: 4-((2-(3,3-Difluoroazetidin-1-yl)-1H-imidazol-4-yl)methyl)pyridine. To a stirred mixture of 3,3-difluoroazetidine-1-carboximidamide hydrochloride (249 mg, 1.4 mmol) in MeCN (2.5 mL) was added K₂CO₃ (550 mg, 4.1 mmol) and 1-bromo-3-(pyridin-4-yl) propan-2-one hydrobromide (398 mg, 1.4 mmol) in portions at rt under a nitrogen atmosphere. The resulting mixture was stirred overnight at 50° C. under a nitrogen atmosphere. The resulting mixture was cooled to rt, filtered, and the filter cake was rinsed with MeCN (2×10 mL). The combined filtrate was concentrated under reduced pressure. The crude product was purified by Prep-HPLC to afford 4-((2-(3,3-difluoroazetidin-1-yl)-1H-imidazol-4-yl)methyl)pyridine (58 mg, 7%) as a brown solid. ¹H NMR: (300 MHz, CDCl₃, ppm): δ 8.45 (dd, J=1.5 Hz, 4.5 Hz, 2H), 7.17 (d, J=6.0 Hz, 2H), 6.37 (s, 1H), 4.32 (t, J=12.0 Hz, 4H), 3.83 (s, 2H). LCMS ESI-MS m/z:=251 [M+H]⁺.

### Example 3: 4-((2-(3,3-Difluoropyrrolidin-1yl)-1H-imidazol-4-yl)methylpyridine

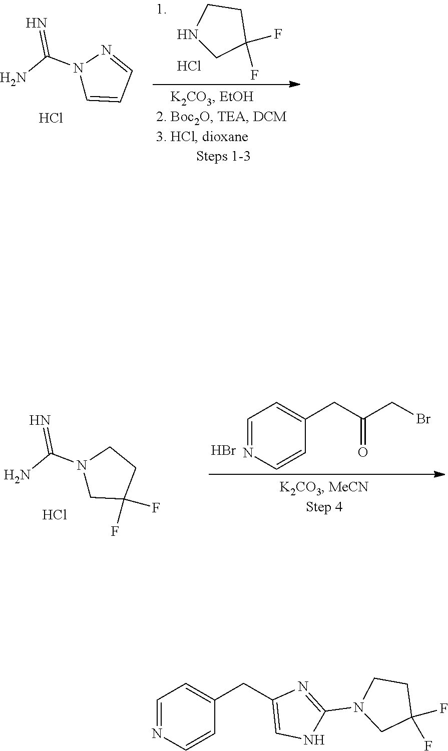

Step 1: 3,3-Difluoropyrrolidine-1-carboximidamide hydrochloride. To a stirred mixture of 3,3-difluoropyrrolidine hydrochloride (979 mg, 6.8 mmol) in EtOH (5 mL) was added K₂CO₃ (943 mg, 6.8 mmol) in portions at rt under a nitrogen atmosphere. The resulting mixture was stirred for 1 h at rt, filtered, and the filter cake was rinsed with EtOH (1×5 mL). To the above filtrate was added pyrazole-1-carboximidamide hydrochloride (500 mg, 3.4 mmol) in portions at rt and stirred for an additional 5 h under reflux. The resulting mixture was cooled to rt and concentrated under reduced pressure. The crude product was dissolved in EtOAc (10 mL) and purified by trituration for 2 h. The resulting mixture was filtered, and the filter cake was rinsed with EtOAc (2×5 mL). The filter cake was dried under reduced pressure to afford 3,3-difluoropyrrolidine-1-carboximidamide hydrochloride (1.2 g) as a yellow solid. LCMS ESI-MS m/z:=150 [M+H-HCl]⁺.

Step 2: tert-Butyl-N-(3,3-difluoropyrrolidine-1-carboximidoyl)carbamate. To a stirred mixture of 3,3-difluoropyrrolidine-1-carboximidamide hydrochloride (1 g, 2.7 mmol) in DCM (10 mL) was added Boc₂O (900 mg, 4.1 mmol) and TEA (556 mg, 5.5 mmol) dropwise at rt under a nitrogen atmosphere. The resulting mixture was stirred for 5 h at 40° C. under a nitrogen atmosphere. The resulting mixture was cooled to rt and diluted with water (20 mL). The resulting mixture was extracted with DCM (3×20 mL). The combined organic layers were dried over anhydrous Na₂SO₄, filtered, and the filtrate was concentrated under reduced pressure. The crude product was purified by reverse phase flash chromatography to afford tert-butyl-N-(3,3-difluoropyrrolidine-1-carboximidoyl)carbamate (325 mg, 38% yield for two steps) as a white solid. LCMS ESI-MS m/z:=250 [M+H]⁺.

Step 3: 3,3-Difluoropyrrolidine-1-carboximidamide hydrochloride. To a stirred solution of tert-butyl-N-(3,3-difluoropyrrolidine-1-carboximidoyl)carbamate (325 mg, 1.2 mmol) in 1,4-dioxane (3.2 mL) was added HCl (g) in 1,4-dioxane (1.6 mL) dropwise at rt under a nitrogen atmosphere. The resulting mixture was stirred overnight at 70° C. under a nitrogen atmosphere. The resulting mixture was concentrated under reduced pressure to afford 3,3-difluoropyrrolidine-1-carboximidamide hydrochloride (147 mg) as a white solid. LCMS ESI-MS m/z:=150 [M+H-HCl]⁺.

Step 4: 4-((2-(3,3-Difluoropyrrolidin-1yl)-1H-imidazol-4-yl)methylpyridine. To a stirred mixture of 3,3-difluoropyrrolidine-1-carboximidamide hydrochloride (101 mg, 0.54 mmol) in MeCN (1 mL) was added Cs₂CO₃ (1 g, 3.3 mmol) and 1-bromo-3-(pyridin-4-yl)propan-2-one hydrobromide (482 mg, 1.6 mmol) in portions at rt under a nitrogen atmosphere. The resulting mixture was stirred overnight at 50° C. under a nitrogen atmosphere. The resulting mixture was cooled to rt, filtered, and the filter cake was rinsed with MeCN (2×10 mL). The combined filtrate was concentrated under reduced pressure. The crude product was purified by Prep-HPLC to afford 4-((2-(3,3-difluoropyrrolidin-1yl)-1H-imidazol-4-yl)methylpyridine (19 mg, 5.7% for two steps) as a brown solid. ¹H NMR: (300 MHz, CDCl₃, ppm): δ 8.45 (dd, J=1.5 Hz, 4.5 Hz, 2H), 7.17 (d, J=6.0 Hz, 2H), 6.33 (s, 1H), 3.82 (s, 2H), 3.74 (t, J=12.9 Hz, 4H), 3.59 (t, J=7.2 Hz, 2H), 2.47-2.38 (m, 2H). LCMS ESI-MS m/z: =265 [M+H]⁺.

### Example 4 and 4A: 4-((2-(3,3-Difluorocyclobutyl)-1H-imidazol-4-yl)methyl)-3-fluoropyridine and 2-(3,3-Difluorocyclobutyl)-4-((3-fluoropyridin-4-yl)methyl)oxazole

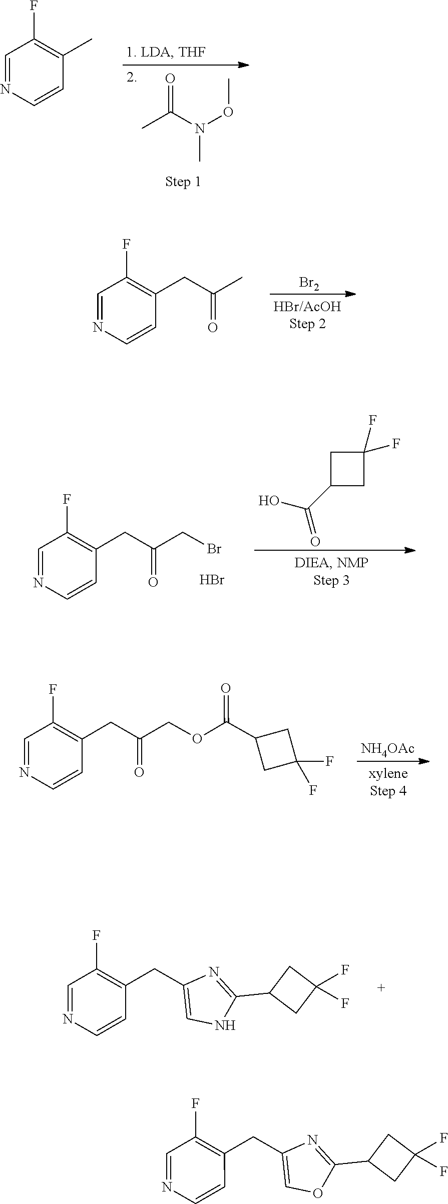

Step 1: 1-(3-Fluoropyridin-4-yl)propan-2-one. To a stirred solution of 3-fluoro-4-methylpyridine (20 g, 180 mmol) in THF (400 mL) was added LDA (108 mL, 216 mmol, 2.0 M) dropwise at −30° C. under a nitrogen atmosphere. The resulting mixture was stirred for 30 min at −30° C. under a nitrogen atmosphere. N-methoxy-N-methylacetamide (27.8 g, 270 mmol) was added dropwise at −30° C. and the resulting mixture was stirred for 2 h at −30° C. under a nitrogen atmosphere. The reaction was quenched with aqueous saturated NH₄Cl (500 mL) at −30° C. and extracted with EtOAc (3×100 mL). The combined organic layers were washed with brine (200 mL), dried over anhydrous Na₂SO₄, filtered, and the filtrate was concentrated under reduced pressure to afford crude product. The crude product was purified by reverse phase flash chromatography to afford 1-(3-fluoropyridin-4-yl)propan-2-one (12.5 g, 45%) as a white solid.

LCMS ESI-MS m/z: 154 [M+H]⁺.

Step 2: 1-Bromo-3-(3-fluoropyridin-4-yl)propan-2-one hydrobromide. To a stirred solution of 1-(3-fluoropyridin-4-yl)propan-2-one (12.5 g, 89.4 mmol) in HBr/AcOH (125 mL) was added Br₂ (13.1 g, 89.4 mmol) dropwise at 0° C. under a nitrogen atmosphere. The resulting mixture was stirred for 3 h at rt under a nitrogen atmosphere. To the mixture was added Et₂O (3 L) at rt. The mixture was filtered, and the filter cake was rinsed with Et₂O (3×200 mL). The filter cake was dried under reduced pressure to afford 1-bromo-3-(3-fluoropyridin-4-yl)propan-2-one hydrobromide (13 g, 51%) as a white solid. LCMS ESI-MS m/z: 312 [M+H]⁺.

Step 3: 2-Oxo-3-(pyridin-4-yl)propyl 2,2-difluorocyclopropane-1-carboxylate. To a stirred mixture of 3,3-difluorocyclobutane-1-carboxylic acid (1 g, 7.6 mmol) and DIEA (1.6 g, 12.7 mmol) in NMP (20 mL) was added 1-bromo-3-(3-fluoropyridin-4-yl)propan-2-one hydrobromide (2 g, 6.3 mmol) at room temperature under a nitrogen atmosphere. The resulting mixture was stirred for 3 h at rt under a nitrogen atmosphere. The reaction was quenched with water (60 mL) at rt and extracted with EtOAc (2×20 mL). The combined organic phase was washed with brine (5×50 mL), dried over anhydrous Na₂SO₄, filtered, and the filtrate was concentrated under reduced pressure. The crude product was purified by silica gel column chromatography to afford 2-oxo-3-(pyridin-4-yl)propyl-2,2-difluorocyclopropane-1-carboxylate (1.2 g, 65%) as brown oil. LCMS ESI-MS m/z: 288 [M+H]⁺.

Step 4: 4-((2-(3,3-Difluorocyclobutyl)-1H-imidazol-4-yl)methyl)-3-fluoropyridine and 2-(3,3-Difluorocyclobutyl)-4-((3-fluoropyridin-4-yl)methyl)oxazole. A mixture of 3-(3-fluoropyridin-4-yl)-2-oxopropyl 3,3-difluorocyclobutane-1-carboxylate (1.2 g, 4.1 mmol) and NH₄OAc (3.2 g, 41 mmol) in xylene (24 mL) was stirred and irradiated for 10 min at 140° C. in a microwave. The resulting mixture was quenched with water (30 mL) and extracted with DCM (3×10 mL). The combined organic layers were washed with brine (30 mL). dried over anhydrous Na₂SO₄, filtered, and the filtrate was concentrated under reduced pressure. The crude product was purified by reverse phase flash chromatography to afford 4-((2-(3,3-difluorocyclobutyl)-1H-imidazol-4-yl)methyl)-3-fluoropyridine and 2-(3,3-difluorocyclobutyl)-4-((3-fluoropyridin-4-yl)methyl)oxazole. 4-((2-(3,3-Difluorocyclobutyl)-1H-imidazol-4-yl)methyl)-3-fluoropyridine was further purified by prep-HPLC to afford 4-((2-(3,3-difluorocyclobutyl)-1H-imidazol-4-yl)methyl)-3-fluoropyridine (61 mg, 5%) as a white solid. ¹H NMR: (400 MHz, Chloroform-d, ppm): δ 8.42-8.32 (m, 2H), 7.24 (dd, J=6.4, 4.9 Hz, 1H), 6.73 (s, 1H), 4.00 (s, 2H), 3.47-3.38 (m, 1H), 3.03-2.88 (m, 4H). LCMS ESI-MS mi z:=268 [M+H]⁺. 2-(3,3-Difluorocyclobutyl)-4-((3-fluoropyridin-4-yl)methyl)oxazole was further purified by prep-HPLC to afford 2-(3,3-difluorocyclobutyl)-4-((3-fluoropyridin-4-yl)methyl)oxazole (1.3 mg, 0.1%) as a yellow oil. ¹H NMR: (400 MHz, DMSO-d₆, ppm): δ 8.55-8.45 (m, 1H), 8.36 (dd, J=4.8, 1.2 Hz, 1H), 7.89 (d, J=1.2 Hz, 1H), 7.36 (dd, J=6.6, 4.8 Hz, 1H), 3.91 (s, 2H), 3.55 (s, 1H), 3.08-3.00 (m, 2H), 2.91-2.82 (m, 2H). LCMS ESI-MS m/z:=269 [M+H]⁺.

### Example 5A and 5B: (S)-4-((2-(2,2-Difluorocyclopropyl)-1H-imidazol-4-yl)methyl)pyridine and (R)-4-((2-(2,2-Difluorocyclopropyl)-1H-imidazol-4-yl)methyl)pyridine

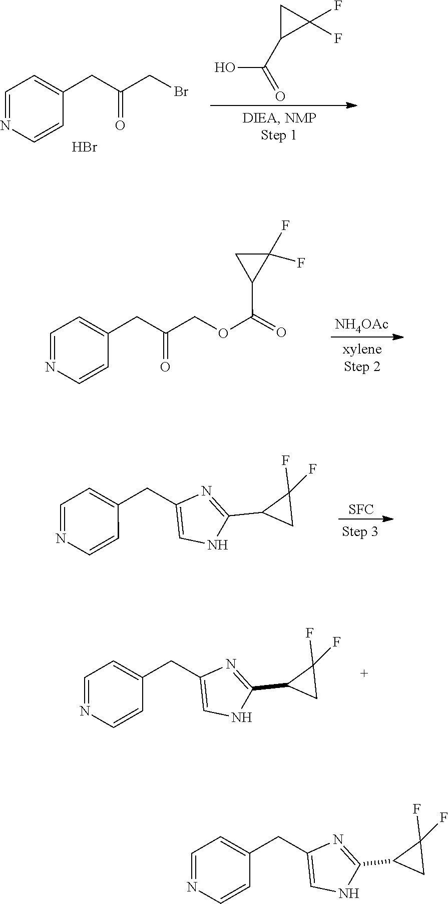

Step 1: 2-Oxo-3-(pyridin-4-yl)propyl-2,2-difluorocyclopropane-1-carboxylate. To a stirred mixture of 2,2-difluorocyclopropane-1-carboxylic acid (6.2 g, 11.8 mmol) and DIEA (13.2 g, 23.6 mmol) in NMP (150 mL) was added 1-bromo-3-(pyridin-4-yl)propan-2-one hydrobromide (15 g, 11.8 mmol) at room temperature under a nitrogen atmosphere. The resulting mixture was stirred for 2 h at rt under a nitrogen atmosphere. The reaction was quenched with water (500 mL) at rt. The resulting mixture was extracted with EtOAc (2×200 mL), and the combined organic phase was washed with brine (5×500 mL), dried over anhydrous Na₂SO₄, filtered, and the filtrate was concentrated under reduced pressure. The crude product was purified by silica gel column chromatography to afford 2-oxo-3-(pyridin-4-yl)propyl-2,2-difluorocyclopropane-1-carboxylate (7.9 g, 60%) as a yellow oil. LCMS ESI-MS m/z: 256 [M+H]⁺.

Step 2: 4-((2-(2,2-Difluorocyclopropyl)-1H-imidazol-4-yl)methyl)pyridine. A mixture of 2-oxo-3-(pyridin-4-yl)propyl-2,2-difluorocyclopropane-1-carboxylate (7.9 g, 3.9 mmol) and NH₄OAc (11.9 g, 19.5 mmol) in xylene (160 mL) was stirred and irradiated for 10 min at 140° C. in a microwave. The reaction was quenched with water (160 mL) at rt. The resulting mixture was extracted with EtOAc (3×50 mL). The combined organic phase was washed with brine (2×50 mL), dried over anhydrous Na₂SO₄, filtered, and the filtrate was concentrated under reduced pressure. The crude product was purified by reverse phase flash chromatography to afford 4-((2-(2,2-difluorocyclopropyl)-1H-imidazol-4-yl)methyl)pyridine. 4-((2-(2,2-difluorocyclopropyl)-1H-imidazol-4-yl)methyl)pyridine was further purified by prep-HPLC to afford 4-((2-(2,2-difluorocyclopropyl)-1H-imidazol-4-yl)methyl)pyridine (193 mg, 2.6%) as a white solid. LCMS ESI-MS m/z: 236 [M+H]⁺.

Step 3: (S)-4-((2-(2,2-Difluorocyclopropyl)-1H-imidazol-4-yl)methyl)pyridine and (R)-4-((2-(2,2-Difluorocyclopropyl)-1H-imidazol-4-yl)methyl)pyridine. 4-((2-(2,2-Difluorocyclopropyl)-1H-imidazol-4-yl)methyl)pyridine was separated by prep-SFC to afford Peak 1 (Example 5A, 52.7 mg) as a white solid. ¹H NMR:(400 MHz, DMSO-d6, ppm): δ 11.92 (d, J=42.8 Hz, 1H), 8.46 (ddd, J=19.8, 4.4, 1.6 Hz, 2H), 7.22 (td, J=4.4, 1.6 Hz, 2H), 6.86-6.60 (m, 1H), 3.90 (s, 2H), 2.93 (tt, J=11.6, 8.4 Hz, 1H), 2.10-1.92 (m, 2H); LCMS ESI-MS m/z:=236[M+H]⁺. Peak 2 (Example 5B, 63.8 mg) was isolated as a white solid. ¹H NMR:(400 MHz, DMSO-d6, ppm):δ 11.92 (d, J=42.8 Hz, 1H), 8.46 (ddd, J=19.8, 4.4, 1.6 Hz, 2H), 7.22 (td, J=4.4, 1.6 Hz, 2H), 6.86-6.60 (m, 1H), 3.90 (s, 2H), 2.93 (tt, J=11.6, 8.4 Hz, 1H), 2.10-1.92 (m, 2H). LCMS ESI-MS m/z: 236[M+H]⁺. The absolute stereochemistry was not determined for either isomer.

### Example 6: 4-((2-(2,2-Dichlorocyclopropyl)-1H-imidazol-4-yl)methyl)pyridine

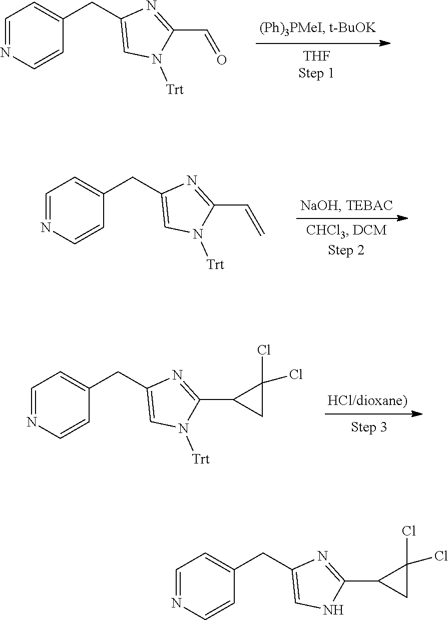

Step 1: 4-((2-Ethenyl-1-(triphenylmethyl)imidazol-4-yl)methyl)pyridine. To a stirred solution of (Ph)₃PMeI (9.9 g, 24.5 mmol) in THF (140 mL) was added t-BuOK (3.7 g, 32.6 mmol) in portions at 0° C. under a nitrogen atmosphere. The resulting mixture was stirred for 1 h at 0° C. under a nitrogen atmosphere. 4-(Pyridin-4-ylmethyl)-1-(triphenylmethyl)imidazole-2-carbaldehyde (7.0 g, 16.3 mmol) was added in portions at 0° C. under a nitrogen atmosphere. The resulting mixture was stirred for 3 h at 0° C. under a nitrogen atmosphere. The resulting mixture was quenched with water (200 mL) and extracted with EtOAc (3×200 mL). The combined organic layers were washed with brine (200 mL), dried over anhydrous Na₂SO₄, filtered, and the filtrate was concentrated under reduced pressure. The crude product was purified by reverse phase flash chromatography to afford 4-((2-ethenyl-1-(triphenylmethyl)imidazol-4-yl)methyl)pyridine (6.5 g) as a yellow solid. LCMS ESI-MS m/z: =428 [M+H]⁺.

Step 2: 4-((2-(2,2-Dichlorocyclopropyl)-1-(triphenylmethyl) imidazol-4-yl) methyl)pyridine. To a stirred solution of 4-((2-ethenyl-1-(triphenylmethyl)imidazol-4-yl)methyl)pyridine (3 g, 7 mmol) and CHCl₃ (4.2 g, 35.1 mmol) in DCM (30 mL) was added TEBAC (0.32 g, 1.4 mmol) and NaOH (1.4 g, 35 mmol) in portions at rt under a nitrogen atmosphere. The resulting mixture was stirred overnight at 40° C. under a nitrogen atmosphere. The resulting mixture was cooled to rt, then quenched with water (50 mL), extracted with DCM (3×50 mL), dried over anhydrous Na₂SO₄, filtered, and the filtrate was concentrated under reduced pressure. The crude product was purified by reverse phase flash chromatography to afford 4-((2-(2,2-dichlorocyclopropyl)-1-(triphenylmethyl)imidazol-4-yl)methyl)pyridine (79 mg) as a yellow solid. LCMS ESI-MS m/z: 510 [M+H]⁺.

Step 3: 4-((2-(2,2-Dichlorocyclopropyl)-1H-imidazol-4-yl)methyl)pyridine. A solution of 4-((2-(2,2-dichlorocyclopropyl)-1-(triphenylmethyl)imidazol-4-yl)methyl)pyridine (79 mg, 0.09 mmol) in dioxane (0.8 mL) was treated with 4 M HCl/dioxane (0.8 mL) overnight at 30° C. under a nitrogen atmosphere. The resulting mixture was concentrated under reduced pressure. The residue was dissolved in EtOAc (5 mL) and H₂O (5 mL). The mixture was basified to pH 9-10 with aq. sat. Na₂CO₃. The resulting mixture was extracted with EtOAc (3×5 mL). The organic layers were combined and dried over anhydrous Na₂SO₄, filtered, and the filter cake was rinsed with EtOAc (2×5 mL). The resulting organic solution was concentrated under reduced pressure. The crude product was purified by prep-HPLC to afford 4-((2-(2,2-dichlorocyclopropyl)-1H-imidazol-4-yl)methyl)pyridine (5.3 mg) as a light yellow solid. ¹H NMR: (400 MHz, DMSO-d₆, ppm): δ 12.06 (d, J=42.8 Hz, 1H), 8.53-8.37 (m, 2H), 7.21 (dt, J=4.6, 2.2 Hz, 2H), 6.97-6.60 (m, 1H), 3.86 (d, J=48.4 Hz, 2H), 2.99 (dt, J=11.1, 8.4 Hz, 1H), 2.31-2.16 (m, 1H), 2.09 (dd, J=10.9, 7.4 Hz, 1H). LCMS ESI-MS m/z: 268 [M+H]⁺.

### Example 7: 4-((2-((3,3-Difluoroazetidin-1yl)methyl)-1H-imidazol-5-yl)methylpyridine

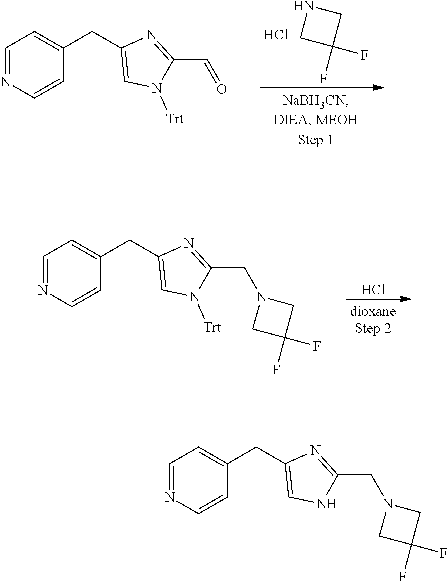

Step 1: 4-((2-((3,3-difluoroazetidin-1-yl)methyl)-trityl-1H-imidazol-5-yl)methyl)pyridine. To a stirred mixture of 3,3-difluoroazetidinehydrochloride (226 mg, 1.7 mmol) and DIEA (301 mg, 2.3 mmol) in MeOH (5 mL) was added 5-(pyridin-4-ylmethyl)-1-(triphenylmethyl)imidazole-2-carbaldehyde (500 mg, 1.2 mmol) in portions at 0° C. under a nitrogen atmosphere. The resulting mixture was stirred for 30 min at 0° C. under a nitrogen atmosphere. NaBH₃CN (146 mg, 2.3 mmol) was added in portions at 0° C. and the resulting mixture was stirred for another 1 h at 0° C. under a nitrogen atmosphere. The reaction was quenched with water (20 mL) at 0° C. and extracted with EtOAc (3×10 mL). The combined organic layers were washed with brine (20 mL), dried over anhydrous Na₂SO₄, filtered, and the filtrate was concentrated under reduced pressure. The crude product was purified by reverse phase flash chromatography to afford 4-((2-((3,3-difluoroazetidin-1-yl)methyl)-trityl-1H-imidazol-5-yl)methyl)pyridine (150 mg) as a yellow oil. LCMS ESI-MS m/z: 508 [M+H]⁺.

Step 2: 4-((2-((3,3-Difluoroazetidin-1yl)methyl)-1H-imidazol-5-yl)methylpyridine. To a stirred solution of 4-((2-((3,3-difluoroazetidin-1-yl)methyl)-trityl-1H-imidazol-5-yl)methyl)pyridine (150 mg, 0.28 mmol) in dioxane (1.5 mL) was added HCl (g) in dioxane (4 M, 0.7 mL) dropwise at rt under a nitrogen atmosphere. The resulting mixture was stirred for an additional 3 h at rt. The resulting mixture was concentrated under reduced pressure. The residue was dissolved in DCM (8 mL) and H₂O (8 mL) and basified to pH 9-10 with aq. sat. Na₂CO₃. The aqueous layer was extracted with DCM (3×5 mL). The combined organic layers were dried over anhydrous Na₂SO₄, filtered, and the filtrate was concentrated under reduced pressure. The crude product was purified by reverse phase flash chromatography to afford 4-((2-((3,3-difluoroazetidin-1yl)methyl)-1H-imidazol-5-yl)methylpyridine (28 mg) as a yellow oil. ¹H NMR: (300 MHz, DMSO-d₆, ppm): δ 11.85 (s, 1H), 8.62-8.24 (m, 2H), 7.38-7.11 (m, 2H), 6.81 (s, 1H), 3.81 (s, 2H), 3.67 (d, J=1.4 Hz, 2H), 3.61 (s, 4H). LCMS ESI-MS m/z: 265 [M+H]⁺.

### Example 8: 3-(4-(Pyridin-4-ylmethyl)-1H-imidazol-2-yl)-1-(trifluoromethyl)cyclobutan-1-ol

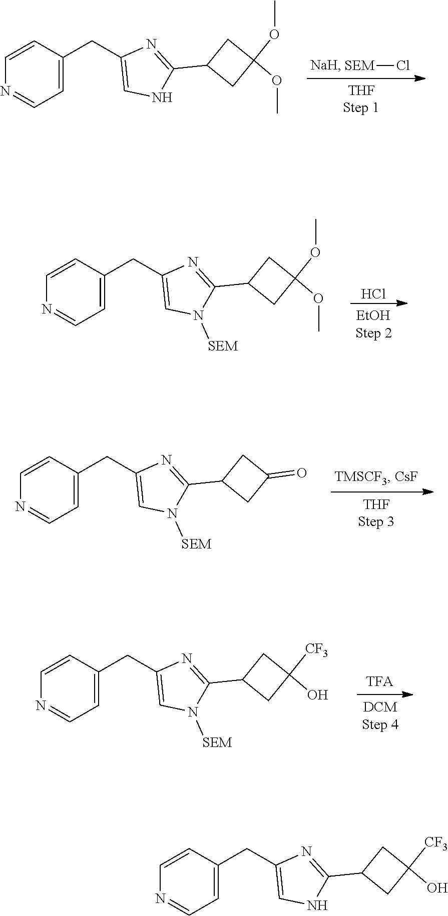

Step 1: 4-((2-(3,3-Dimethoxycyclobutyl)-1-((2-(trimethylsilyl)ethoxy)methyl)-1H-imidazol-4-yl)methyl)pyridine. To a stirred solution of 4-((2-(3,3-dimethoxycyclobutyl)-1H-imidazol-4-yl)methyl)pyridine (300 mg, 1.1 mmol) in THE (6 mL) was added NaH (48.3 mg, 1.2 mmol, 60%) in portions at 0° C. and stirred at 0° C. for 0.5 h under a nitrogen atmosphere. SEM-Cl (221 mg, 1.3 mmol) was added dropwise at 0° C. and stirred for 1.5 h at rt under a nitrogen atmosphere. The resulting mixture was poured into ice water (10 mL) slowly at 0° C. and extracted with EtOAc (2×10 mL). The combined organic phase was washed with brine (15 mL), dried over anhydrous Na₂SO₄, filtered, and the filtrate was concentrated under reduced pressure. The crude product was purified by reverse phase flash chromatography to afford 4-((2-(3,3-dimethoxycyclobutyl)-1-((2-(trimethylsilyl)ethoxy)methyl)-1H-imidazol-4-yl)methyl)pyridine (420 mg, 68%) as a brown oil. LCMS ESI-MS m/z:=404 [M+H]⁺.

Step 2: 3-(4-Pyridin-4-ylmethyl)-1-((2-(trimethylsilyl)ethoxy)methyl)-1H-imidazol-2-yl)cyclobutan-1-one. A mixture of 4-((2-(3,3-dimethoxycyclobutyl)-1-((2-(trimethylsilyl)ethoxy)methyl)-1H-imidazol-4-yl)methyl)pyridine (420 mg, 0.75 mmol) in EtOH (4.2 mL) was added HCl aq. (0.4 mL, 2.3 mmol, 6 M) dropwise at 0° C. under a nitrogen atmosphere. The resulting mixture was stirred for 4 h at 0° C. under a nitrogen atmosphere. The resulting mixture was basified to pH 9-10 with aq. sat. Na₂CO₃. The resulting mixture was extracted with EtOAc (3×10 mL). The organic layers were combined, dried over anhydrous Na₂SO₄, filtered, and the filtrate was concentrated under reduced pressure. The crude product was purified by reverse phase flash chromatography to afford 3-(4-pyridin-4-ylmethyl)-1-((2-(trimethylsilyl)ethoxy)methyl)-1H-imidazol-2-yl)cyclobutan-1-one (217 mg, 81%) as a brown oil. LCMS ESI-MS m/z:=358 [M+H]⁺.

Step 3: 3-(4-(Pyridin-4-ylmethyl)-1-((2-(trimethylsilyl)ethoxy)methyl)-1H-imidazol-2-yl)-1-(trifluoromethyl)cyclobutan-1-ol. A solution of 3-(4-pyridin-4-ylmethyl)-1-((2-(trimethylsilyl)ethoxy)methyl)-1H-imidazol-2-yl)cyclobutan-1-one (217 mg, 0.61 mmol) in THF (2.2 mL) was treated with TMSCF₃ (173 mg, 1.2 mmol) for 5 min at rt under a nitrogen atmosphere. CsF (182 mg, 1.2 mmol) was added in portions at rt under a nitrogen atmosphere.

The resulting mixture was stirred for 4 h at rt under a nitrogen atmosphere. The resulting mixture was quenched with water (10 mL) and extracted with EtOAc (3×10 mL). The combined organic layers were washed with brine (10 mL), dried over anhydrous Na₂SO₄, filtered, and the filtrate was concentrated under reduced pressure. The crude product was purified by reverse phase flash chromatography to afford 3-(4-(pyridin-4-ylmethyl)-1-((2-(trimethylsilyl)ethoxy)methyl)-1H-imidazol-2-yl)-1-(trifluoromethyl)cyclobutan-1-ol (130 mg, 50%) as a yellow solid. LCMS ESI-MS m/z:=428 [M+H]⁺.

Step 4: 3-(4-Pyridin-4-ylmethyl)-1H-imidazol-2-yl)-1-(trifluoromethyl)cylcobutan-1-ol. To a solution of 3-(4-(pyridin-4-ylmethyl)-1-((2-(trimethylsilyl)ethoxy)methyl)-1H-imidazol-2-yl)-1-(trifluoromethyl)cyclobutan-1-ol (70 mg, 0.16 mmol) in DCM (0.7 mL) was added TFA (0.7 mL) and the reaction mixture was stirred 4 h at rt under a nitrogen atmosphere. The resulting mixture was concentrated under reduced pressure. The residue was dissolved in DCM (5 mL) and H₂O (5 mL). The mixture was basified to pH 9-10 with aq. sat. Na₂CO₃ and extracted with DCM (3×5 mL). The combined organic layers were concentrated under reduced pressure. The crude product was purified by prep-HPLC to afford 3-(4-(pyridin-4-ylmethyl)-1H-imidazol-2-yl)-1-(trifluoromethyl)cyclobutan-1-ol (12 mg, 25%) as a white solid. ¹H NMR: (400 MHz, DMSO-d₆, ppm): δ 11.66 (d, J=30.9 Hz, 1H), 8.43 (d, J=5.3 Hz, 2H), 7.24 (d, J=4.7 Hz, 2H), 6.67 (t, J=24.6 Hz, 2H), 3.84 (d, J=22.8 Hz, 2H), 3.15 (t, J=8.9 Hz, 1H), 2.70 (t, J=10.3 Hz, 2H), 2.44 (d, J=11.5 Hz, 2H). LCMS ESI-MS m/z: 298 [M+H]⁺.

### Example 9: 4-((2-(3-Fluoro-3-methylcyclobutyl)-1H-imidazol-4-yl)methyl)pyridine

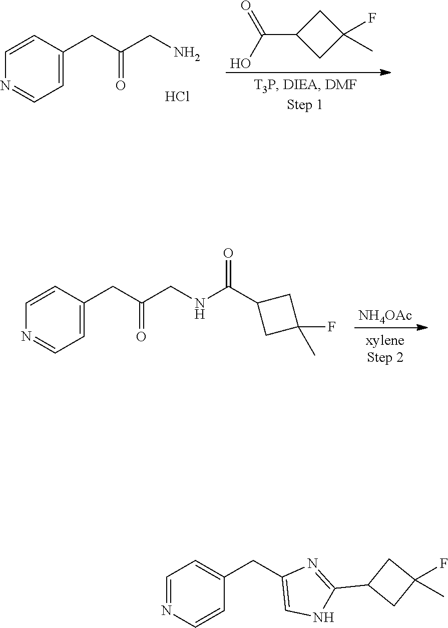

Step 1: 3-Fluoro-3-methyl-N-(2-oxo-3-(pyridine-4-yl)propyl)cyclobutene-1-carboxamide. To a stirred solution of 3-fluoro-3-methylcyclobutane-1-carboxylic acid (467 mg 3.5 mmol) and DIEA (1.14 g, 8.84 mmol) in DMF (5.5 mL) was added T₃P (2.3 mg, 3.5 mmol, 50% wt.) at 0° C. under a nitrogen atmosphere. The resulting mixture was added 1-amino-3-(pyridin-4-yl)propan-2-one hydrochloride (550 mg, 2.95 mmol) in portions at 0° C. and stirred for 2 h at rt under a nitrogen atmosphere. The reaction was quenched with water (20 mL) at 0° C. and extracted with EtOAc (3×10 mL). The combined organic layers were washed with brine (1×20 mL), dried over anhydrous Na₂SO₄, filtered, and the filtrate was concentrated under reduced pressure. The crude product was purified by reverse phase flash chromatography to afford 3-fluoro-3-methyl-N-(2-oxo-3-(pyridine-4-yl)propyl)cyclobutene-1-carboxamide (230 mg, 30%) as a yellow oil. LCMS ESI-MS m/z:=265 [M+H]⁺.

Step 2: 4-((2-(3-Fluoro-3-methylcyclobutyl)-1H-imidazol-4-yl)methyl)pyridine. To a stirred solution of 3-fluoro-3-methyl-N-(2-oxo-3-(pyridine-4-yl)propyl)cyclobutene-1-carboxamide (190 mg, 0.72 mmol) and NH₄OAc (1.1 g, 14.4 mmol) in xylene (3.8 mL) was stirred and irradiated for 1 h at 140° C. in a microwave. The resulting mixture was cooled to rt and quenched with water (10 mL) at rt. The resulting mixture was extracted with EtOAc (4×5 mL). The combined organic phase was washed with brine (1×10 mL), dried over anhydrous Na₂SO₄, filtered, and the filtrate was concentrated under reduced pressure. The crude product was purified by reverse phase flash chromatography and again by prep-achiral-SFC to afford 4-((2-(3-fluoro-3-methylcyclobutyl)-1H-imidazol-4-yl)methyl)pyridine (8.7 mg, 5%) as a yellow solid. ¹H NMR: (400 MHz, DMSO-d₆, ppm): δ 11.80-11.46 (m, 1H), 8.57-8.36 (m, 2H), 7.34-7.16 (m, 2H), 6.89-6.46 (m, 1H), 3.98-3.70 (m, 2H), 3.59-3.45 (m, 1H), 2.65-2.53 (m, 2H), 2.48-2.31 (m, 2H), 1.54-1.36 (m, 3H). LCMS EI-MS m/z:=246 [M+H]⁺.

### Example 10A and 10B: (R)-4-((2-(2,2-Difluoro-1-methylcyclopropyl)-1H-imidazol-4-yl)methyl)pyridine and (S)-4-((2-(2,2-Difluoro-1-methylcyclopropyl)-1H-imidazol-4-yl)methyl)pyridine

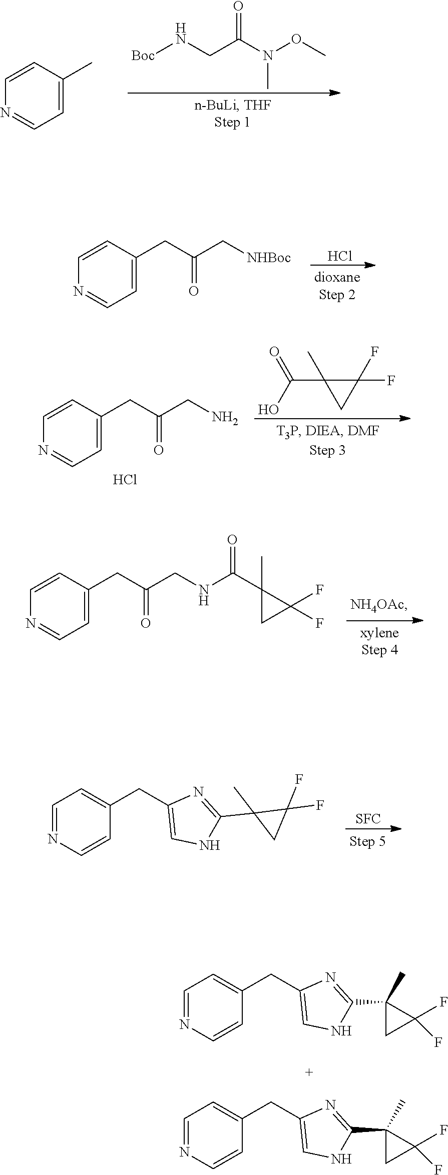

Step 1: tert-Butyl-(2-oxo-3-(pyridine-4-yl)propyl)carbamate. To a stirred solution of 4-methylpyridine (10 g, 107 mmol) in THE (100 mL) was added n-BuLi (129 mL, 323 mmol, 2.5 M) dropwise at −70° C. under a nitrogen atmosphere. The resulting mixture was stirred for 30 min at −70° C. under a nitrogen atmosphere. tert-Butyl-(2-(methoxy(methyl)amino)-2-oxoethyl)carbamate (30.4 g, 140 mmol) in THF (200 mL) was added dropwise at −70° C. and the resulting mixture was stirred for another 1.5 h at −70° C. under a nitrogen atmosphere. The resulting mixture was quenched with aq. sat. NH₄Cl (500 mL) at −70° C. and was extracted with EtOAc (3×300 mL). The combined organic layers were washed with brine (300 mL), dried over anhydrous Na₂SO₄, filtered, and the filtrate was concentrated under reduced pressure. The crude product was purified by silica gel column chromatography to afford tert-butyl-(2-oxo-3-(pyridine-4-yl)propyl)carbamate (12.3 g) as a light yellow solid. LCMS ESI-MS m/z: 251 [M+H]⁺.

Step 2: 1-Amino-3-(pyridine-4-yl)propan-2-one hydrochloride. To a stirred solution of tert-butyl-(2-oxo-3-(pyridine-4-yl)propyl)carbamate (12.3 g, 49 mmol) in dioxane (120 mL) was added HCl (g) in dioxane (120 mL, 4 M) dropwise at rt under a nitrogen atmosphere. The resulting mixture was stirred for 2 h at rt under a nitrogen atmosphere. The resulting mixture was filtered, and the filter cake was rinsed in the funnel with EtOAc (3×200 mL). The filter cake was dried under reduced pressure to afford 1-amino-3-(pyridine-4-yl)propan-2-one hydrochloride (5.2 g, 21%, after 2 steps) as a yellow solid. LCMS ESI-MS m/z: 151 [M+H−HCl]⁺.

Step 3: 2,2-Difluoro-1-methyl-N-(2-oxo-3-(pyridine-4-yl)propyl)cyclopropane-1-carboxamide. To a stirred mixture of 2,2-difluoro-1-methylcyclopropane-1-carboxylic acid (175 mg, 1.2 mmol) and DIEA (415 mg, 3.2 mmol) in DMF (2 mL) was added T₃P (821 mg, 1.2 mmol, 50 wt %) at rt under a nitrogen atmosphere. 1-Amino-3-(pyridin-4-yl)propan-2-one hydrochloride (200 mg, 1.0 mmol) was added at rt and the resulting mixture was stirred for 2 h at rt under a nitrogen atmosphere. The reaction was quenched by the addition of water (20 mL) at rt. The resulting mixture was extracted with EtOAc (3×10 mL). The combined organic phase was washed with brine (1×20 mL), dried over anhydrous Na₂SO₄, filtered, and the filtrate was concentrated under reduced pressure. The crude product was purified by reverse phase flash chromatography to afford 2,2-difluoro-1-methyl-N-(2-oxo-3-(pyridine-4-yl)propyl)cyclopropane-1-carboxamide (138 mg, 38%) as a brown solid. LCMS ESI-MS m/z: 269 [M+H]⁺.

Step 4: 4-((2-(2,2-Difluoro-1-methylcyclopropyl)-1H-imidazol-4-yl)methyl)pyridine. A mixture of 2,2-difluoro-1-methyl-N-(2-oxo-3-(pyridine-4-yl)propyl)cyclopropane-1-carboxamide (88 mg, 0.3 mmol) and NH₄OAc (507 mg, 6.5 mmol) in xylene (2 mL) was stirred and irradiated for 1 h at 140° C. in a microwave. The resulting mixture was cooled to rt and was then quenched by the addition of water (10 mL) at rt. The resulting mixture was extracted with EtOAc (3×10 mL). The combined organic phase was washed with brine (1×10 mL) and dried over anhydrous Na₂SO₄, filtered, and the filtrate was concentrated under reduced pressure. The crude product was purified by reverse phase flash chromatography to afford 4-((2-(2,2-difluoro-1-methylcyclopropyl)-1H-imidazol-4-yl)methyl)pyridine (63 mg, 50%) as a yellow solid. LCMS ESI-MS m/z:=250 [M+H]⁺.

Step 5: (R)-4-((2-(2,2-Difluoro-1-methylcyclopropyl)-1H-imidazol-4-yl)methyl)pyridine and (S)-4-((2-(2,2-Difluoro-1-methylcyclopropyl)-1H-imidazol-4-yl)methyl)pyridine. 4-((2-(2,2-Difluoro-1-methylcyclopropyl)-1H-imidazol-4-yl)methyl)pyridine (63 mg) was separated by prep-SFC to afford Peak 1 (Example 10A, 1.3 mg) as a yellow oil. ¹H NMR: (300 MHz, DMSO-d₆, ppm): δ 12.01-11.74 (m, 1H), 8.50-8.40 (m, 2H), 7.27-7.18 (m, 2H), 6.86-6.56 (m, 1H), 4.01-3.69 (m, 2H), 2.31-2.18 (m, 1H), 1.70-1.56 (m, 1H), 1.53-1.46 (m, 3H). LCMS ESI-MS m/z:=250 [M+H]⁺. Peak 2 (Example 10B, 1.2 mg) was isolated as a yellow oil. ¹H NMR: (300 MHz, DMSO-d₆, ppm): δ 12.04-11.65 (m, 1H), 8.53-8.37 (m, 2H), 7.29-7.19 (m, 2H), 6.93-6.59 (m, 1H), 4.05-3.72 (m, 2H), 2.36-2.11 (m, 1H), 1.74-1.51 (m, 1H), 1.59-1.41 (m, 3H). LCMS ESI-MS m/z:=250 [M+H]⁺.

The absolute stereochemistry was not determined for either isomer.

### Example 11A and 11B: (S)-4-((2-(3,3-Difluorocyclopentyl)-1H-imidazol-4-yl)methyl)pyridine and (R)-4-((2-(3,3-Difluorocyclopentyl)-1H-imidazol-4-yl)methyl)pyridine

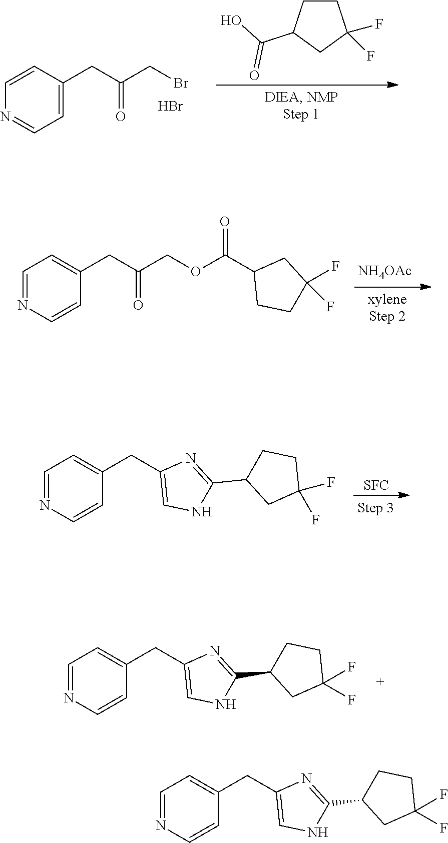

Step 1: 2-Oxo-3-(pyridin-4yl)propyl-3,3-difluorocyclopentane-1-carboxylate. To a stirred solution of 3,3-difluorocyclopentane-1-carboxylic acid (5.0 g, 33.9 mmol) in NMP (50 mL) were added DIEA (4.38 g, 33.9 mmol) and 1-bromo-3-(pyridin-4-yl)propan-2-one hydrobromide (5.0 g, 16.9 mmol) in portions at 0° C. under a nitrogen atmosphere. The resulting mixture was stirred for 3 h at 0° C. under a nitrogen atmosphere. The resulting mixture was quenched with water (100 mL). The resulting mixture was extracted with EtOAc (3×50 mL). The combined organic layers were washed with brine (5×50 mL), dried over anhydrous Na₂SO₄, filtered, and the filtrate was concentrated under reduced pressure to afford 2-oxo-3-(pyridin-4yl)propyl-3,3-difluorocyclopentane-1-carboxylate (3.9 g, 81%) as a brown solid. LCMS ESI-MS m/z:=284 [M+H]⁺.

Step 2: 4-((2-(3,3-Difluorocyclopentyl)-1H-imidazol-4-yl)methyl)pyridine. To a stirred solution of 2-oxo-3-(pyridin-4yl)propyl-3,3-difluorocyclopentane-1-carboxylate (3.8 g, 13.4 mmol) in xylene (76 mL) was added NH₄OAc (5.2 g, 67.1 mmol) in portions at rt under a nitrogen atmosphere. The resulting mixture was stirred for 2 h at 140° C. under a nitrogen atmosphere. The mixture was cooled to rt. The resulting mixture was quenched with the addition of water (100 mL). The resulting mixture was extracted with DCM (3×30 mL). The combined organic layers were washed with brine (1×100 mL), dried over anhydrous Na₂SO₄, filtered, and the filtrate was concentrated under reduced pressure. The crude product was purified by reverse phase flash chromatography and again by prep-HPLC to afford 4-((2-(3,3-difluorocyclopentyl)-1H-imidazol-4-yl)methyl)pyridine (63 mg, 1.5%) as a brown solid. LCMS ESI-MS m/z:=264 [M+H]⁺.

Step 3: (S)-4-((2-(3,3-Difluorocyclopentyl)-1H-imidazol-4-yl)methyl)pyridine and (R)-4-((2-(3,3-Difluorocyclopentyl)-1H-imidazol-4-yl)methyl)pyridine. 4-((2-(3,3-Difluorocyclopentyl)-1H-imidazol-4-yl)methyl)pyridine (63 mg) was separated by prep-SFC to afford Peak 1 (Example 11A, 1.2 mg) as a white solid. ¹H NMR: (400 MHz, DMSO-d₆, ppm): δ 11.67 (s, 1H), 8.44 (d, J=5.0 Hz, 2H), 7.23 (d, J=5.0 Hz, 2H), 6.70 (s, 1H), 3.81 (s, 2H), 2.50-2.31 (m, 3H), 2.27-2.05 (m, 3H), 1.92 (pt, J=8.1, 4.8 Hz, 1H). LCMS ESI-MS m/z:=264 [M+H]⁺. Peak 2 (Example 11B, 3.1 mg) was isolated as an off-white solid. ¹H NMR: (400 MHz, DMSO-d₆, ppm): δ 11.68 (s, 1H), 8.45 (d, J=5.4 Hz, 2H), 7.24 (d, J=5.2 Hz, 2H), 6.71 (s, 1H), 3.81 (s, 2H), 2.51-2.34 (m, 3H), 2.27-2.04 (m, 3H), 1.93 (tdd, J=12.3, 7.7, 3.9 Hz, 1H). LCMS ESI-MS m/z:=264 [M+H]⁺.

### Example 12: 4,4-Difluoro-1-(4-pyridin-4-ylmethyl)-1H-imidazol-2-yl)cyclohexan-1-ol

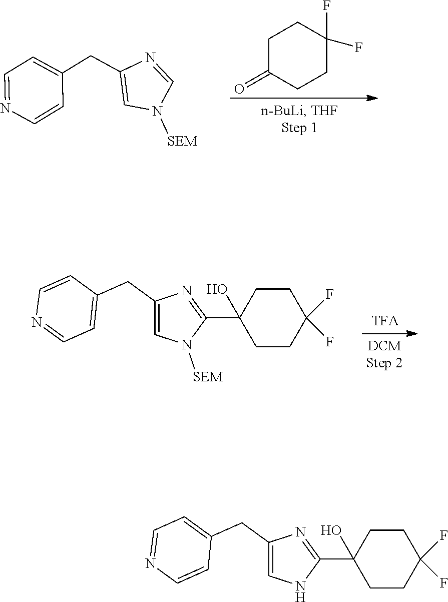 [SMILES: C[Si](C)(C)CCOCn1cnc(Cc2ccncc2)c1 ; O=C1CCC(F)(F)CC1 ; C[Si](C)(C)CCOCn1cc(Cc2ccncc2)nc1C1(O)CCC(F)(F)CC1 ; OC1(c2nc(Cc3ccncc3)c[nH]2)CCC(F)(F)CC1]

Step 1: 4,4-Difluoro-1-(4-pyridin-4-ylmethyl)-1-((2-(trimethylsilyl)ethoxy)methyl-1H-imidazol-2-yl)cyclohexen-1-ol. To a stirred solution of 4-((1-((2-(trimethylsilyl)ethoxy)methyl)-1H-imidazol-4-yl)methyl)pyridine (2.0 g, 6.9 mmol) in THF (30 mL) was added n-BuLi (8.3 mL, 20.7 mmol, 2.5 M) dropwise at −60° C. under a nitrogen atmosphere. The resulting mixture was stirred for 1 h at −60° C. under a nitrogen atmosphere. Then 4,4-difluorocyclohexan-1-one (2.3 mg, 17.3 mmol) was added dropwise at −60° C. and the resulting mixture was stirred for another 1 h at −60° C. under a nitrogen atmosphere. The reaction was quenched with aqueous saturated NH₄Cl (50 mL) at −60° C. The resulting mixture was extracted with EtOAc (3×20 mL). The combined organic layers were washed with brine (1×20 mL), dried over anhydrous Mg₂SO₄, filtered, and the filtrate was concentrated under reduced pressure. The crude product was purified by reverse phase flash chromatography and again by prep-HPLC to afford 4,4-difluoro-1-(4-pyridin-4-ylmethyl)-1-((2-(trimethylsilyl)ethoxy)methyl-1H-imidazol-2-yl)cyclohexen-1-ol (900 mg, 28%) as a white solid. LCMS ESI-MS m/z:=424 [M+H]⁺.

Step 2: 4,4-Difluoro-1-(4-pyridin-4-ylmethyl)-1H-imidazol-2-yl)cyclohexan-1-ol. To a stirred solution of 4,4-difluoro-1-(4-pyridin-4-ylmethyl)-1-((2-(trimethylsilyl)ethoxy)methyl-1H-imidazol-2-yl)cyclohexen-1-ol (300 mg, 0.65 mmol) in DCM (1.5 mL) was added TFA (1.5 mL) dropwise at 0° C. under a nitrogen atmosphere. The resulting mixture was stirred overnight at 30° C. The resulting mixture was concentrated under reduced pressure. The residue was dissolved in DCM (10 mL) and H₂O (10 mL), and the mixture was basified to pH 9-10 with aq. sat. Na₂CO₃. The resulting mixture was extracted with DCM (5×20 mL). The organic layers were combined and dried over anhydrous MgSO₄. The solids were removed by filtration and the filter cake was rinsed in the funnel with DCM (2×20 mL). The resulting organic solution was concentrated under reduced pressure. The crude product was purified by prep-HPLC to afford 4,4-difluoro-1-(4-pyridin-4-ylmethyl)-1H-imidazol-2-yl)cyclohexan-1-ol (75.3 mg, 40%) as a white solid. ¹H NMR: (400 MHz, DMSO-d₆ ppm): δ 8.50-8.34 (m, 2H), 7.33-7.15 (m, 2H), 6.68 (s, 1H), 5.98 (s, 1H), 3.82 (s, 2H), 2.20-1.77 (m, 8H). LCMS ESI-MS m/z:=294 [M+H]⁺.

### Example 13: 4-((2-(4,4-Difluorocyclohexyl)-1H-imidazol-4-yl)methyl)pyridine

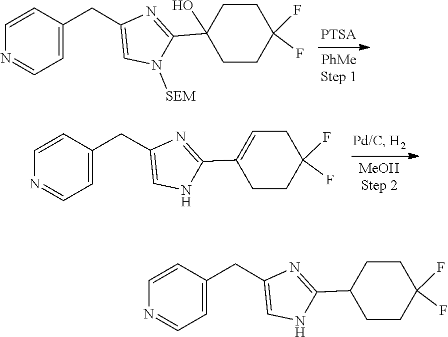

Step 1: 4-((2-(4,4-Difluorocyclohex-1-en-1-yl)-1H-imidazol-4-yl)methyl)pyridine. To a stirred solution of 4,4-difluoro-1-(4-(pyridin-4-ylmethyl)-1-((2-(trimethylsilyl)ethoxy)methyl)-1H-imidazol-2-yl)cyclohexan-1-ol (230 mg, 0.54 mmol) in toluene (2.3 mL) was added PTSA (93.5 mg, 0.54 mmol) in portions at rt under a nitrogen atmosphere. The resulting mixture was stirred additional overnight at 120° C. The resulting mixture was cooled to rt and diluted with water (5.0 mL). The mixture was basified to pH 8 with aqueous saturated Na₂CO₃. The resulting mixture was extracted with DCM (3×10 mL). The organic layers were combined and dried over anhydrous Na₂SO₄. The solids were filtered, and the filter cake was rinsed in the funnel with DCM (2×10 mL). The resulting organic solution was concentrated under reduced pressure. The crude was purified by reverse phase flash chromatography to afford 4-((2-(4,4-difluorocyclohex-1-en-1-yl)-1H-imidazol-4-yl)methyl)pyridine (94 mg, 61%) as a white solid. LCMS ESI-MS m/z:=276 [M+H]⁺.

Step 2: 4-((2-(4,4-Difluorocyclohexyl)-1H-imidazol-4-yl)methyl)pyridine. A mixture of 4-((2-(4,4-difluorocyclohex-1-en-1-yl)-1H-imidazol-4-yl)methyl)pyridine (94 mg, 0.33 mmol) and Pd/C (9.4 mg, 10 wt %) in MeOH (1 mL) was stirred overnight at rt under a hydrogen atmosphere. The resulting mixture was filtered, and the filter cake was washed with EtOAc (2×10 mL). The filtrate was collected and concentrated under reduced pressure. The crude product was purified by prep-HPLC to afford 4-((2-(4,4-difluorocyclohexyl)-1H-imidazol-4-yl)methyl)pyridine (23.6 mg, 25%) as a white solid. ¹H NMR: (400 MHz, DMSO-d₆ ppm): δ 11.54 (s, 1H), 8.43 (d, J=5.0 Hz, 2H), 7.23 (d, J=5.1 Hz, 2H), 6.72 (s, 1H), 3.78 (s, 2H), 2.76-2.81 (m, 1H), 2.09-1.86 (m, 6H), 1.83-1.47 (m, 2H). LCMS ESI-MS m/z:=278 [M+H]⁺.

### Example 14: 4-((2-(2,2-Difluorocyclopropyl)-1H-imidazol-4-yl)methyl)-3-fluoropyridine

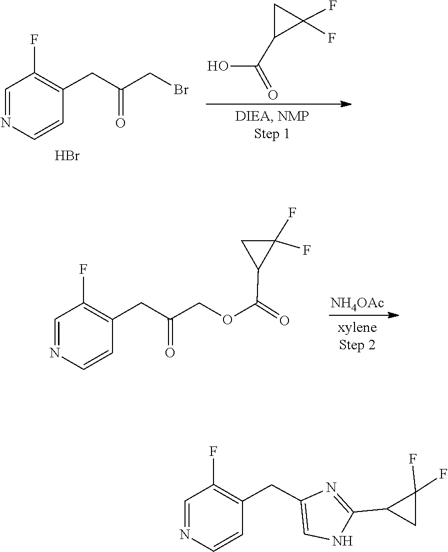 [SMILES: O=C(CBr)Cc1ccncc1F ; O=C(O)C1CC1(F)F ; Br ; O=C(COC(=O)C1CC1(F)F)Cc1ccncc1F ; Fc1cnccc1Cc1c[nH]c(C2CC2(F)F)n1 ; "NH4OAc"]

Step 1: 3-(3-Fluoropyridin-4-yl)-2-oxopropyl-2,2-difluorocyclopropane-1-carboxylate. To a stirred solution of 2,2-difluorocyclopropane-1-carboxylic acid (1.9 g, 15.3 mmol) in NMP (40 mL) was added DIEA (3.3 g, 26 mmol) and 1-bromo-3-(3-fluoropyridin-4-yl)propan-2-one hydrobromide (4 g, 13 mmol) in portions at rt under a nitrogen atmosphere. The resulting mixture was stirred for 3 h at rt under a nitrogen atmosphere. The reaction was quenched with water (120 mL) and extracted with EtOAc (3×50 mL). The combined organic layers were washed with brine (3×100 mL), dried over anhydrous Na₂SO₄, filtered, and the filtrate was concentrated under reduced pressure to afford 3-(3-fluoropyridin-4-yl)-2-oxopropyl-2,2-difluorocyclopropane-1-carboxylate (1.3 g) as a brown oil. The product was used directly in the next step without further purification. LCMS ESI-MS m/z:=274 [M+H]⁺.

Step 2: 4-((2-(2,2-Difluorocyclopropyl)-1H-imidizol-4-yl)methyl)-3-fluoropyridine. A mixture of 3-(3-fluoropyridin-4-yl)-2-oxopropyl-2,2-difluorocyclopropane-1-carboxylate (1.3 g, 3 mmol, 630% purity) and NH₄OAc (1.2 g, 15 mmol) in xylene (26 mL) was stirred and irradiated for 10 min at 140° C. in a microwave. The resulting mixture was quenched with water (40 mL), extracted with DCM (3×15 mL). The combined organic layers were washed with brine (40 mL), dried over anhydrous Na₂SO₄, filtered, and the filtrate was concentrated under reduced pressure. The crude product was purified by reverse phase flash chromatography and further purified by prep-HPLC to afford 4-((2-(2,2-difluorocyclopropyl)-1H-imidizol-4-yl)methyl)-3-fluoropyridine (25 mg, 0.8%) a light yellow oil. ¹H NMR: (400 MHz, DMSO-d₆, ppm): 68.42 (s, 1H), 8.34 (d, J=4.9 Hz, 1H), 7.25 (d, J=5.7 Hz, 1H), 6.80 (s, 1H), 4.03 (s, 2H), 2.93 (td, J=11.4, 7.8 Hz, 1H), 2.15-1.89 (m, 2H). LCMS ESI-MS m/z:=254 [M+H]⁺.

### Example 15A and 15B: 4-((2-(3,3-Difluorocyclobutyl)-1H-imidazol-4-yl)methyl)pyridine and 2-(3,3-Difluorocyclobutyl)-4-(pyridin-4-ylmethyl)oxazole

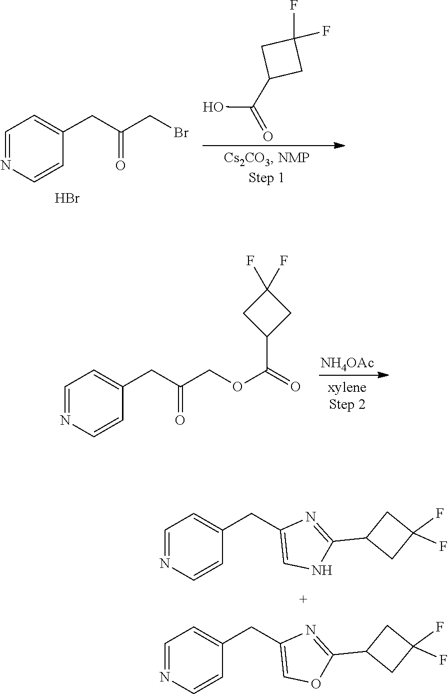

Step 1: 2-Oxo-3-(pyridin-4-yl)propyl-3,3-difluorocyclobutane-1-carboxylate. To a stirred solution of 3,3-difluorocyclobutane-1-carboxylic acid (13.4 g, 102 mmol) in NMP (300 mL) was added Cs₂CO₃ (99.4 g, 305 mmol) and 1-bromo-3-(pyridin-4-yl)propan-2-one hydrobromide (30 g, 102 mmol) in portions at 0° C. under a nitrogen atmosphere. The resulting mixture was stirred for 3 h at 0° C. under a nitrogen atmosphere. The reaction was quenched with water (900 mL) and extracted with EtOAc (3×50 mL). The combined organic layers were washed with brine (3×100 mL), dried over anhydrous Na₂SO₄, filtered, and the filtrate was concentrated under reduced pressure. The crude product was purified by silica gel flash chromatography to afford 2-oxo-3-(pyridine-4-yl)propyl-3,3-difluorocyclobutane-1-carboxylate (8.3 g, 28%) as a yellow oil. LCMS ESI-MS m/z:=270 [M+H]⁺.

Step 2: 4-((2-(3,3-Difluorocyclobutyl)-1H-imidazol-4-yl)methyl)pyridine and 2-(3,3-Difluorocyclobutyl)-4-(pyridin-4-ylmethyl)oxazole. To a stirred solution of 2-oxo-3-(pyridine-4-yl)propyl-3,3-difluorocyclobutane-1-carboxylate (8 g, 30 mmol) in xylene (160 mL) was added NH₄OAc (11.4 g, 149 mmol) in portions at rt under a nitrogen atmosphere. The resulting mixture was stirred for 2 h at 140° C. under a nitrogen atmosphere. The reaction mixture was cooled to rt, quenched with water (150 mL) and extracted with DCM (3×100 mL). The combined organic layers were washed with brine (100 mL), dried over anhydrous Na₂SO₄, filtered, and the filtrate was concentrated under reduced pressure. The crude product was purified by reverse phase flash chromatography to afford 4-((2-(3,3-difluorocyclobutyl)-1H-imidazol-4-yl)methyl)pyridine and 2-(3,3-difluorocyclobutyl)-4-(pyridin-4-ylmethyl)oxazole. 4-((2-(3,3-Difluorocyclobutyl)-1H-imidazol-4-yl)methyl)pyridine was further purified by achiral SFC to afford 4-((2-(3,3-difluorocyclobutyl)-1H-imidazol-4-yl)methyl)pyridine (802 mg, 12%) as a light yellow solid. ¹H NMR: (300 MHz, DMSO-d₆, ppm): δ 11.78 (d, J=27.0 Hz, 1H), 8.48-8.42 (m, 2H), 7.25-7.10 (m, 2H), 6.69 (d, J=58.5 Hz, 1H), 4.12-3.79 (m, 2H), 3.36-3.27 (m, 1H), 2.97-2.72 (m, 4H). LCMS ESI-MS m/z:=250 [M+H]⁺.

2-(3,3-Difluorocyclobutyl)-4-(pyridin-4-ylmethyl)oxazole was further purified by prep-HPLC to afford 2-(3,3-difluorocyclobutyl)-4-(pyridin-4-ylmethyl)oxazole (92 mg, 1%) as a yellow oil. ¹H NMR: (300 MHz, DMSO-d₆, ppm): δ 8.48 (d, J=5.9 Hz, 2H), 7.87 (s, 1H), 7.27 (d, J=5.1 Hz, 2H), 3.84 (s, 2H), 3.60-3.48 (m, 1H), 3.16-2.74 (m, 4H). LCMS ESI-MS m/z: =251 [M+H]⁺.

### Example 16: 4-((2-(1-Trifluoromethyl)cyclopropyl)-1H-imidazol-4-yl)methyl)pyridine

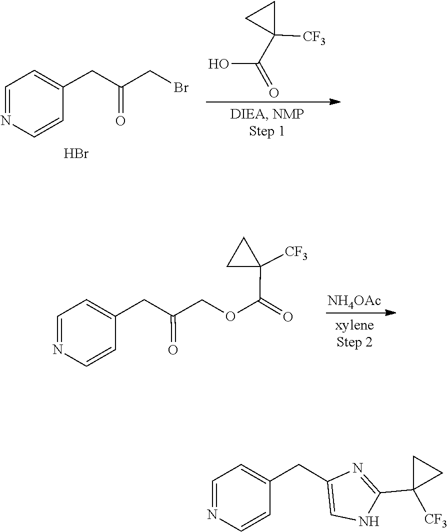 [SMILES: O=C(CBr)Cc1ccncc1 ; O=C(O)C1(C(F)(F)F)CC1 ; Br ; O=C(COC(=O)C1(C(F)(F)F)CC1)Cc1ccncc1 ; FC(F)(F)C1(c2nc(Cc3ccncc3)c[nH]2)CC1 ; "NH4OAc"]

Step 1: 2-Oxo-3-(pyridin-4-yl)propyl-1-(trifluoromethyl)cyclopropane-1-carboxylate. To a stirred solution of 1-(trifluoromethyl)cyclopropane-1-carboxylic acid (1.7 g, 11 mmol) in NMP (30 mL) was added DIEA (3.3 g, 25 mmol) and 1-bromo-3-(pyridin-4-yl)propan-2-one hydrobromide (3 g, 10 mmol) in portions at rt under a nitrogen atmosphere. The resulting mixture was stirred for 3 h at rt under a nitrogen atmosphere. The reaction was quenched with water (120 mL) and extracted with EtOAc (3×50 mL). The combined organic layers were washed with brine (100 mL), dried over anhydrous Na₂SO₄, filtered, and the filtrate was concentrated under reduced pressure to afford 2-oxo-3-(pyridin-4-yl)propyl-1-(trifluoromethyl)cyclopropane-1-carboxylate. The crude product was used directly in the next step without purification. LCMS ESI-MS m/z:=288 [M+H]⁺.

Step 2: 4-((2-(1-Trifluoromethyl)cyclopropyl)-1H-imidazol-4-yl)methyl)pyridine. A mixture of 2-oxo-3-(pyridin-4-yl)propyl-1-(trifluoromethyl)cyclopropane-1-carboxylate (1.0 g, 3.7 mmol, 81% purity) and NH₄OAc (5.6 g, 73 mmol) in xylene (26 mL) was stirred and irradiated for 10 min at 140° C. in a microwave. The reaction mixture was cooled to rt, quenched with water (40 mL), and extracted with DCM (3×15 mL). The combined organic layers were washed with brine (40 mL), dried over Na₂SO₄, filtered, and the filtrate was concentrated under reduced pressure. The crude product was purified by reverse phase flash chromatography and further purified by prep-HPLC to afford 4-((2-(1-trifluoromethyl)cyclopropyl)-1H-imidazol-4-yl)methyl)pyridine (18 mg, 0.8%) a light brown oil. ¹H NMR: (300 MHz, CDCl₃, ppm): δ 8.51 (d, J=6 Hz, 2H), 7.19 (d, J=6 Hz, 2H), 6.68 (s, 1H), 3.90 (s, 2H), 1.46-1.38 (m, 4H). LCMS ESI-MS m/z:=268 [M+H]⁺.

### Example 17A and 17B: 4-((2-(4,4-Difluorocyclohexyl)-1H-imidazol-4-yl)methyl)-3-fluoropyridine and 2-(4,4-Difluorocyclohexyl)-4-((3-fluoropyridin-4-yl)methyl)oxazole

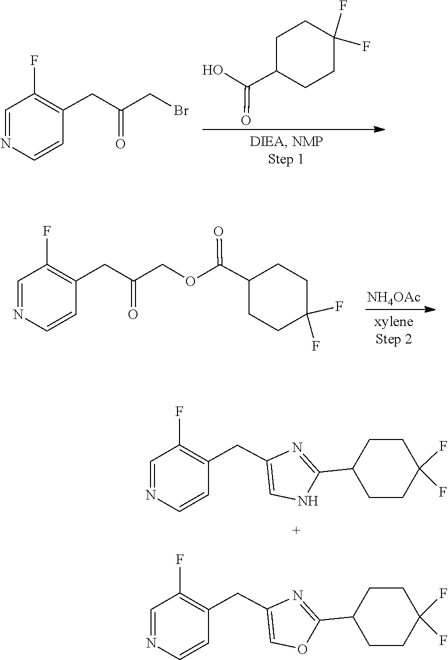 [SMILES: O=C(CBr)Cc1ccncc1F ; O=C(O)C1CCC(F)(F)CC1 ; O=C(COC(=O)C1CCC(F)(F)CC1)Cc1ccncc1F ; Fc1cnccc1Cc1c[nH]c(C2CCC(F)(F)CC2)n1 ; Fc1cnccc1Cc1coc(C2CCC(F)(F)CC2)n1 ; "NH4OAc"]

Step 1: 3-(3-Fluoropyridin-4-yl)-2-oxopropyl 4,4-difluorocyclohexane-1-carboxylate. To a stirred solution of 4,4-difluorocyclohexane-1-carboxylic acid (1.3 g, 7.7 mmol) in NMP (20 mL) was added DIEA (1.7 g, 13 mmol) and 1-bromo-3-(3-fluoropyridin-4-yl)propan-2-one hydrobromide (2 g, 6.4 mmol) in portions at rt under a nitrogen atmosphere. The resulting mixture was stirred for 3 h at rt under a nitrogen atmosphere. The resulting mixture was quenched with water (100 mL) and extracted with EtOAc (3×30 mL). The combined organic layers were washed with brine (3×100 mL), dried over anhydrous Na₂SO₄, filtered, and the filtrate was concentrated under reduced pressure to afford 3-(3-fluoropyridin-4-yl)-2-oxopropyl-4,4-difluorocyclohexane-1-carboxylate (662 mg, 33%) as brown oil. The product was used directly in the next step without further purification. LCMS ESI-MS m/z: =316 [M+H]⁺.

Step 2: 4-((2-(4,4-difluorocyclohexyl)-1H-imidazol-4-yl)methyl)-3-fluoropyridine and 2-(4,4-Difluorocyclohexyl)-4-((3-fluoropyridin-4-yl)methyl)oxazole. A mixture of 3-(3-fluoropyridin-4-yl)-2-oxopropyl-4,4-difluorocyclohexane-1-carboxylate (662 mg, 2.1 mmol) and NH₄OAc (1.6 g, 21 mmol) in xylene (13.2 mL) was stirred and irradiated for 10 min at 140° C. in a microwave. The resulting mixture was quenched with water (30 mL), extracted with DCM (3×10 mL). The combined organic layers were washed with brine (1×30 mL), dried over anhydrous Na₂SO₄, filtered, and the filtrate was concentrated under reduced pressure. The crude product was purified by reverse phase flash chromatography to afford 4-((2-(4,4-difluorocyclohexyl)-1H-imidazol-4-yl)methyl)-3-fluoropyridine and 2-(4,4-difluorocyclohexyl)-4-((3-fluoropyridin-4-yl)methyl)oxazole. 4-((2-(4,4-Difluorocyclohexyl)-1H-imidazol-4-yl)methyl)-3-fluoropyridine was further purified by prep-HPLC to afford 4-((2-(4,4-difluorocyclohexyl)-1H-imidazol-4-yl)methyl)-3-fluoropyridine (71 mg, 11%) as a white solid. ¹H NMR: (400 MHz, DMSO-d₆, ppm): δ 11.65 (d, J=37.4 Hz, 1H), 8.49 (dd, J=17.2, 1.7 Hz, 1H), 8.40-8.25 (m, 1H), 7.38-7.18 (m, 1H), 6.80-6.40 (m, 1H), 3.89 (d, J=44.3 Hz, 2H), 2.88-2.72 (m, 1H), 2.21-1.82 (m, 6H), 1.74 (dd, J=14.0, 10.4 Hz, 2H). LCMS ESI-MS m/z: =296 [M+H]⁺. 2-(4,4-Difluorocyclohexyl)-4-((3-fluoropyridin-4-yl)methyl)oxazole was further purified by prep-HPLC to afford 2-(4,4-difluorocyclohexyl)-4-((3-fluoropyridin-4-yl)methyl)oxazole (16 mg, 2.6%) as an off-white solid. ¹H NMR: (400 MHz, DMSO-d₆, ppm): δ 8.52 (d, J=1.7 Hz, 1H), 8.36 (dd, J=4.8, 1.2 Hz, 1H), 7.83 (d, J=1.1 Hz, 1H), 7.35 (dd, J=6.6, 4.9 Hz, 1H), 3.89 (s, 2H), 3.10-2.91 (m, 1H), 2.18-1.83 (m, 6H), 1.79-1.60 (m, 2H). LCMS ESI-MS m/z:=297 [M+H]⁺.

### Example 18A and 18B: 4-((2-(3,3-Difluorocyclopentyl)-1H-imidazol-4-yl)methyl)-3-fluoropyridine and 2-(3,3-Difluorocyclopentyl)-4-((3-fluoropyridin-4-yl)methyl)oxazole

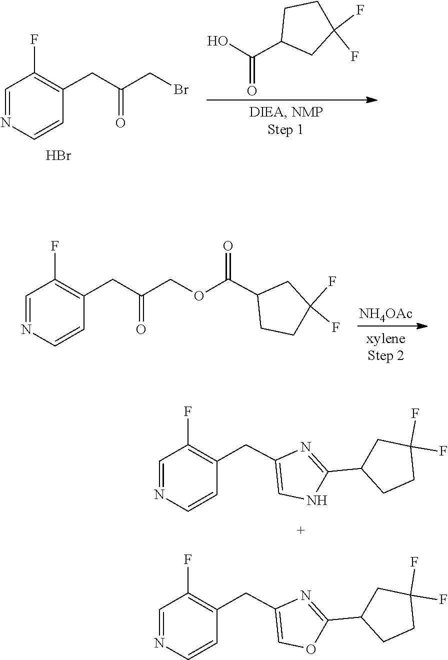

Step 1: 3-(3-Fluoropyridin-4-yl)-2-oxopropyl-3,3-difluorocyclopentane-1-carboxylate. To a stirred solution of 3,3-difluorocyclopentane-1-carboxylic acid (1.7 g, 11.5 mmol) in NMP (30 mL) was added DIEA (2.5 g, 19.2 mmol) and 1-bromo-3-(3-fluoropyridin-4-yl)propan-2-one hydrobromide (3 g, 9.6 mmol) in portions at rt under a nitrogen atmosphere. The resulting mixture was stirred for 3 h at rt under a nitrogen atmosphere. The resulting mixture was quenched with water (100 mL) and extracted with EtOAc (3×30 mL). The combined organic layers were washed with brine (3×100 mL), dried over anhydrous Na₂SO₄, filtered, and the filtrate was concentrated under reduced pressure to afford 3-(3-fluoropyridin-4-yl)-2-oxopropyl-3,3-difluorocyclopentane-1-carboxylate (771 mg, 27%) as a brown oil. The product was used directly in the next step without further purification. LCMS ESI-MS m/z: =302 [M+H]⁺.

Step 2: 4-((2-(3,3-Difluorocyclopentyl)-1H-imidazol-4-yl)methyl)-3-fluoropyridine and 2-(3,3-Difluorocyclopentyl)-4-((3-fluoropyridin-4-yl)methyl)oxazole. A mixture of 3-(3-fluoropyridin-4-yl)-2-oxopropyl-3,3-difluorocyclopentane-1-carboxylate (771 mg, 2.6 mmol) and NH₄OAc (4 g, 52 mmol) in xylene (15 mL) was stirred and irradiated for 10 min at 140° C. in a microwave. The resulting mixture was quenched with water (30 mL) and extracted with DCM (3×10 mL). The combined organic layers were washed with brine (1×30 mL), dried over anhydrous Na₂SO₄, filtered, and the filtrate was concentrated under reduced pressure. The crude product was purified by reverse phase flash chromatography to afford 4-((2-(3,3-difluorocyclopentyl)-1H-imidazol-4-yl)methyl)-3-fluoropyridine and 2-(3,3-difluorocyclopentyl)-4-((3-fluoropyridin-4-yl)methyl)oxazole. 4-((2-(3,3-Difluorocyclopentyl)-1H-imidazol-4-yl)methyl)-3-fluoropyridine was further purified by Prep-HPLC to afford 4-((2-(3,3-difluorocyclopentyl)-1H-imidazol-4-yl)methyl)-3-fluoropyridine (111 mg, 15%) as a brown solid. ¹H NMR: (400 MHz, DMSO-d₆, ppm): δ 8.41 (d, J=1.6 Hz, 1H), 8.33 (d, J=4.9 Hz, 1H), 7.26-7.20 (m, 1H), 6.70 (s, 1H), 3.99 (s, 2H), 3.53-3.39 (m, 1H), 2.59-2.07 (m, 6H). LCMS ESI-MS m/z:=282 [M+H]⁺. 2-(3,3-Difluorocyclopentyl)-4-((3-fluoropyridin-4-yl)methyl)oxazole was further purified by prep-HPLC to afford 2-(3,3-difluorocyclopentyl)-4-((3-fluoropyridin-4-yl)methyl)oxazole (21 mg, 3%) as a light yellow oil. ¹H NMR: (400 MHz, DMSO-d₆, ppm): 8.45 (d, J=1.6 Hz, 1H), 8.37 (d, J=4.9 Hz, 1H), 7.37 (d, J=1.0 Hz, 1H), 7.28-7.25 (m, 1H), 3.93 (s, 2H), 3.57-3.43 (m, 1H), 2.63-2.45 (m, 2H), 2.38-2.10 (m, 4H). LCMS ESI-MS m/z:=283 [M+H]⁺.

### Example 19A and 19B: 4-((2-(3,3-Dichlorocyclobutyl)-1H-imidazol-4-yl)methyl)pyridine and 2-(3,3-Dichlorocyclobutyl)-4-(pyridine-4-ylmethyl)oxazole

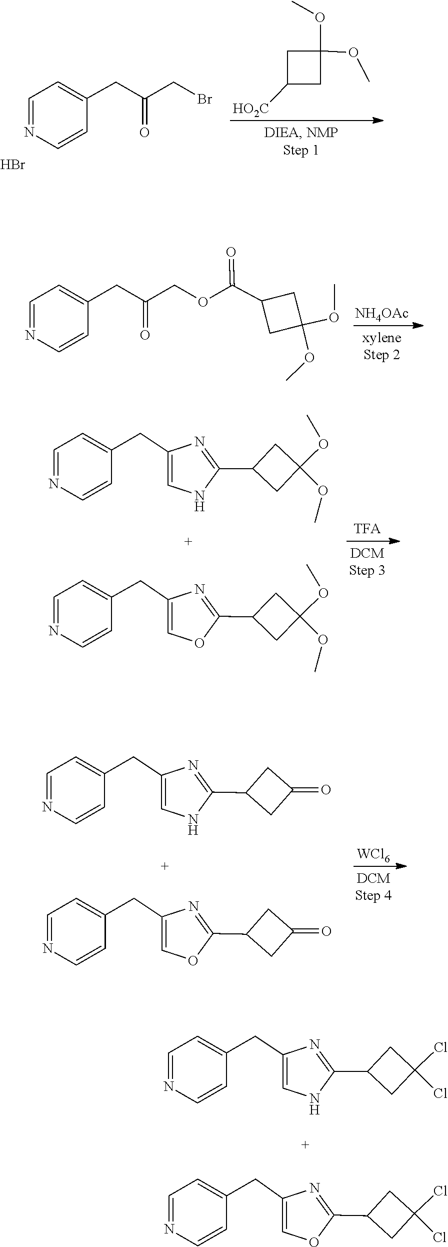

Step 1: 2-Oxo-3-(pyridin-4-yl)propyl-3,3-dimethoxycyclobutane-1-carboxylate. To a stirred solution of 3,3-dimethoxycyclobutane-1-carboxylic acid (3.3 g, 20.3 mmol) in NMP (50 mL) was added DIEA (5.5 g, 42.4 mmol) and 1-bromo-3-(pyridin-4-yl)propan-2-one hydrobromide (5 g, 17 mmol) in portions at rt under a nitrogen atmosphere. The resulting mixture was stirred for 3 h at rt under a nitrogen atmosphere. The resulting mixture was quenched with water (150 mL) and extracted with EtOAc (3×50 mL). The combined organic layers were washed with brine (5×50 mL), dried over anhydrous Na₂SO₄, filtered, and the filtrate was concentrated under reduced pressure to afford 2-oxo-3-(pyridin-4-yl)propyl-3,3-dimethoxycyclobutane-1-carboxylate (2.8 g, 47%) as a brown oil. The product was used directly in the next step without further purification. LCMS ESI-MS m/z:=294 [M+H]⁺.

Step 2: 2-Oxo-3-(pyridin-4-yl)propyl-3,3-dimethoxycyclobutane-1-carboxylate and 4-((2-(3,3-dimethoxycyclobutyl)-1,3-oxazol-4-yl)methyl)pyridine. A mixture of 2-oxo-3-(pyridin-4-yl)propyl-3,3-dimethoxycyclobutane-1-carboxylate (2.8 g, 7.9 mmol) and NH₄OAc (12.2 g, 0.16 mol) in xylene (56 mL) was stirred for 2 h at 140° C. under a nitrogen atmosphere. The resulting mixture was quenched with water (120 mL) and extracted with EtOAc (3×60 mL). The combined organic layers were washed with brine (1×60 mL), dried over anhydrous Na₂SO₄, filtered, and the filtrate was concentrated under reduced pressure. The crude product was purified by reverse phase flash chromatography to afford a mixture of 2-oxo-3-(pyridin-4-yl)propyl-3,3-dimethoxycyclobutane-1-carboxylate and 4-((2-(3,3-dimethoxycyclobutyl)-1,3-oxazol-4-yl)methyl)pyridine (1.2 g) as a brown solid. LCMS ESI-MS m/z:=274 [M+H]⁺ and LCMS (129A-3) ESI-MS m/z:=275 [M+H]⁺.

Step 3: 3-(4-(Pyridin-4-ylmethyl)-1H-imidazol-2-yl)cyclobutan-1-one and 3-(4-(pyridin-4-ylmethyl)-1,3-oxazol-2-yl)cyclobutan-1-one. To a stirred mixture of 2-oxo-3-(pyridin-4-yl)propyl3,3-dimethoxycyclobutane-1-carboxylate and 4-((2-(3,3-dimethoxy cyclobutyl)-1,3-oxazol-4-yl)methyl)pyridine (1.2 g) in DCM (12 mL) was added TFA (12 mL) dropwise at rt under a nitrogen atmosphere. The resulting mixture was stirred overnight at rt under a nitrogen atmosphere. The resulting mixture was concentrated under reduced pressure. The residue was dissolved in DCM (30 mL) and H₂O (30 mL) and basified to pH 9-10 with sat. aq. Na₂CO₃. The resulting mixture was extracted with DCM (3×30 mL) and the combined organic layers were washed with brine (1×100 mL), dried over anhydrous Na₂SO₄, filtered, and the filtrate was concentrated under reduced pressure to afford the mixture of 3-(4-(pyridin-4-ylmethyl)-1H-imidazol-2-yl)cyclobutan-1-one and 3-(4-(pyridin-4-ylmethyl)-1,3-oxazol-2-yl)cyclobutan-1-one (526 mg) as a brown solid. The product was used directly in the next step without further purification. LCMS ESI-MS m/z:=228 [M+H]⁺. LCMS ESI-MS m/z: =229 [M+H]⁺.

Step 4: 4-((2-(3,3-Dichlorocyclobutyl)-1H-imidazol-4-yl)methyl)pyridine and 2-(3,3-Dichlorocyclobutyl)-4-(pyridine-4-ylmethyl)oxazole. To a stirred mixture of 3-(4-(pyridin-4-ylmethyl)-1H-imidazol-2-yl)cyclobutan-1-one and 3-(4-(pyridin-4-ylmethyl)-1,3-oxazol-2-yl)cyclobutan-1-one (526 mg, 1.7 mmol) in DCM (5 mL) was added WCl₆ (1.3 g, 3.3 mmol) in portions at rt under a nitrogen atmosphere. The resulting mixture was stirred for 2 h at rt under a nitrogen atmosphere. The resulting mixture was filtered, and the filter cake was rinsed with DCM (2×10 mL). The filtrate was concentrated under reduced pressure. The crude product was purified by reverse phase flash chromatography to afford 4-((2-(3,3-dichlorocyclobutyl)-1H-imidazol-4-yl)methyl)pyridine and 2-(3,3-dichlorocyclobutyl)-4-(pyridine-4-ylmethyl)oxazole. 4-((2-(3,3-Dichlorocyclobutyl)-1H-imidazol-4-yl)methyl)pyridine was further purified by prep-HPLC to afford 4-((2-(3,3-dichlorocyclobutyl)-1H-imidazol-4-yl)methyl)pyridine (7.5 mg, 1.6%) as a brown solid. ¹H NMR: (300 MHz, DMSO-d₆, ppm): δ 11.78 (d, J=25.0 Hz, 1H), 8.54-8.40 (m, 2H), 7.29-7.17 (m, 2H), 6.84-6.57 (m, 1H), 3.84 (d, J=27.7 Hz, 2H), 3.68 (q, J=8.5 Hz, 1H), 3.21 (d, J=0.9 Hz, 4H). LCMS ESI-MS m/z:=282 [M+H]⁺. 2-(3,3-Dichlorocyclobutyl)-4-(pyridine-4-ylmethyl)oxazole (26.4 mg, 71% purity) was further purified by Prep-HPLC to afford 2-(3,3-dichlorocyclobutyl)-4-(pyridine-4-ylmethyl)oxazole (8.1 mg, 1.7% yield) as a light yellow oil. ¹H NMR: (300 MHz, DMSO-d₆, ppm): δ 8.49-8.45 (m, 2H), 7.90-7.85 (m, 1H), 7.28-7.24 (m, 2H), 3.89 (d, J=8.5 Hz, 1H), 3.84 (t, J=1.2 Hz, 2H), 3.39 (dd, J=11.4, 2.9 Hz, 2H), 3.30-3.21 (m, 2H). LCMS ESI-MS m/z:=283 [M+H]⁺.

### Example 20A and 20B: 4-((2-((2,2-Difluorocyclopropyl)methyl)-1H-imidazol-4-yl)methyl)pyridine and 2-((2,2-Difluorocyclopropyl)methyl)-4-(pyridine-4ylmethyl)oxazole

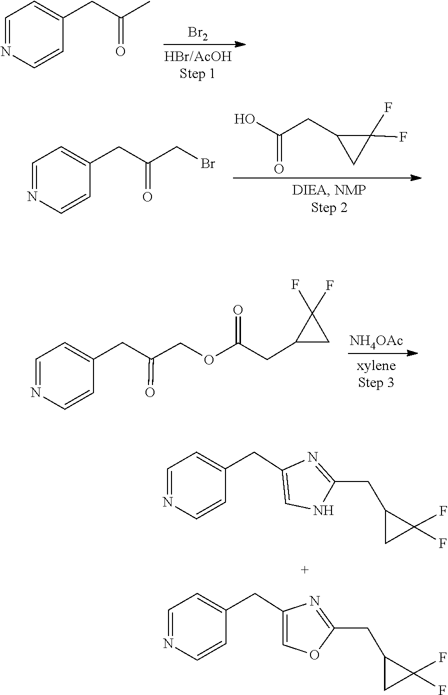

Step 1: 1-Bromo-3-(pyridin-4-yl)propan-2-one hydrobromide. To a stirred solution of 1-(pyridin-4-yl)propan-2-one (50 g, 0.37 mol) in HBr (500 mL, 30% in AcOH) was added Br₂ (59.1 g, 0.37 mol) dropwise at 0° C. under a nitrogen atmosphere. The resulting mixture was stirred for 3 h at rt under a nitrogen atmosphere. The resulting mixture was quenched by the addition of Et₂O (2 L) and stirred for 2 h at rt. The resulting mixture was filtered, and the filter cake was rinsed with Et₂O (3×200 mL). The filter cake was dried under reduced pressure to afford 1-bromo-3-(pyridin-4-yl)propan-2-one hydrobromide (89 g) as a brown solid. The product was used directly in the next step without further purification. LCMS ESI-MS m/z: =214 [M+H−HBr]⁺.

Step 2: 2-Oxo-3-(pyridin-4-yl)propyl-2-(2,2-difluorocyclopropyl) acetate. To a stirred solution of (2,2-difluorocyclopropyl)acetic acid (1.38 g, 10.17 mmol) in NMP (30 mL) was added 1-bromo-3-(pyridin-4-yl)propan-2-one hydrobromide (3 g, 10.17 mmol) and DIEA (2.6 g, 20.3 mmol) in portions at rt under a nitrogen atmosphere. The resulting mixture was stirred for 3 h at rt under a nitrogen atmosphere. The resulting mixture was quenched with water (120 mL) and extracted with EtOAc (3×60 mL). The combined organic layers were washed with brine (5×40 mL), dried over anhydrous Na₂SO₄, filtered, and the filtrate was concentrated under reduced pressure to afford 2-oxo-3-(pyridin-4-yl)propyl 2-(2,2-difluorocyclopropyl) acetate (1.4 g) as a brown oil. The product was used directly in the next step without further purification. LCMS ESI-MS m/z:=270 [M+H]⁺.

Step 3: 4-((2,2-Difluorocyclopropyl)methyl)-1H-imidazol-4-yl)methyl)pyridine and 2-((2-2-Difluorocyclopropyl)methyl)-4-(pyridine-4-ylmethyl)oxazole. A mixture of 2-oxo-3-(pyridin-4-yl)propyl 2-(2,2-difluorocyclopropyl) acetate (1.3 g, 4.8 mmol) and NH₄OAc (7.4 g, 96.6 mmol) in xylene (26 mL) was stirred and irradiated for 10 min at 140° C. in a microwave. The resulting mixture was quenched with water (60 mL) and extracted with EtOAc (3×30 mL). The combined organic layers were washed with brine (1×30 mL), dried over anhydrous Na₂SO₄, filtered, and the filtrate was concentrated under reduced pressure. The crude product was purified by reverse phase flash chromatography to afford 4-((2,2-difluorocyclopropyl)methyl)-1H-imidazol-4-yl)methyl)pyridine and 2-((2-2-difluorocyclopropyl)methyl)-4-(pyridine-4-ylmethyl)oxazole. 4-((2,2-difluorocyclopropyl)methyl)-1H-imidazol-4-yl)methyl)pyridine was further purified by prep-HPLC to afford 4-((2,2-difluorocyclopropyl)methyl)-1H-imidazol-4-yl)methyl)pyridine (46 mg, 4%) as a brown solid. LCMS ESI-MS m/z:=250 [M+H]⁺. 2-((2-2-Difluorocyclopropyl)methyl)-4-(pyridine-4-ylmethyl)oxazole was further purified by prep-HPLC to afford 2-((2-2-difluorocyclopropyl)methyl)-4-(pyridine-4-ylmethyl)oxazole (11 mg, 0.9%) as a light yellow oil. ¹H NMR: (300 MHz, DMSO-d₆, ppm): δ 8.51-8.43 (m, 2H), 7.84 (d, J=1.1 Hz, 1H), 7.30-7.23 (m, 2H), 3.86-3.81 (m, 2H), 2.90 (ddt, J=7.5, 3.1, 1.4 Hz, 2H), 2.04 (ddq, J=13.6, 11.5, 7.5 Hz, 1H), 1.65 (dddd, J=12.8, 11.4, 7.9, 4.9 Hz, 1H), 1.31 (dtd, J=13.6, 7.7, 3.9 Hz, 1H). LCMS ESI-MS m/z:=251 [M+H]⁺.

### Example 21A and 21B: (S)-4-((2-((2,2-Difluorocyclopropyl)methyl)-1H-imidazol-4-yl)methyl)pyridine and (R)-4-((2-((2,2-Difluorocyclopropyl)methyl)-1H-imidazol-4-yl)methyl)pyridine

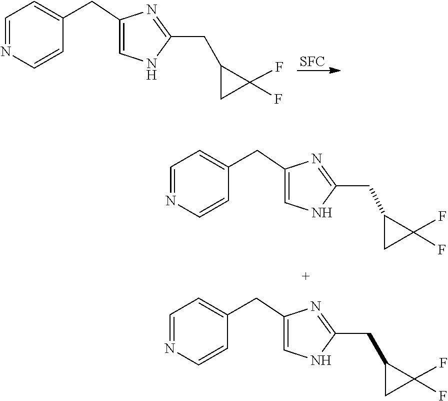

(S)-4-((2-((2,2-Difluorocyclopropyl)methyl)-1H-imidazol-4-yl)methyl)pyridine and (R)-4-((2-((2,2-Difluorocyclopropyl)methyl)-1H-imidazol-4-yl)methyl)pyridine. 4-((2-((2,2-Difluorocyclopropyl)methyl)-1H-imidazol-4-yl)methyl)pyridine (46 mg) was separated by prep-SFC to afford Peak 1 (Example 21A, 8.8 mg) as an off-white solid. ¹H NMR: (300 MHz, DMSO-d₆, ppm): δ 11.60 (s, 1H), 8.44 (d, J=4.9 Hz, 2H), 7.24 (d, J=5.3 Hz, 2H), 6.76 (s, 1H), 3.79 (s, 2H), 2.80 (dd, J=15.7, 7.1 Hz, 1H), 2.63 (dd, J=16.0, 8.0 Hz, 1H), 2.00 (ddd, J=14.3, 11.4, 7.2 Hz, 1H), 1.58 (tq, J=12.2, 7.3, 5.9 Hz, 1H), 1.32-1.19 (m, 1H). LCMS ESI-MS m/z:=250 [M+H]⁺, and (R)-4-((2,2-difluorocyclopropyl)methyl)-1H-imidazol-4-yl)methyl)pyridine (6.1 mg) as an off-white solid. ¹H NMR: (300 MHz, DMSO-d₆, ppm): δ 11.61 (s, 1H), 8.44 (d, J=5.1 Hz, 2H), 7.30-7.20 (m, 2H), 6.76 (s, 1H), 3.80 (s, 2H), 2.80 (dd, J=15.6, 7.1 Hz, 1H), 2.70-2.57 (m, 1H), 2.08-1.91 (m, 1H), 1.57 (tdd, J=12.4, 7.8, 4.7 Hz, 1H), 1.33-1.19 (m, 1H). LCMS ESI-MS m/z:=250 [M+H]⁺.

The absolute stereochemistry was not determined for either isomer.

### Example 22: 2,2,2-Trifluoro-1-(4-(pyridine-4-yl)ethyl)-1H-imidazol-2-yl)ethan-1-one hydrochloride

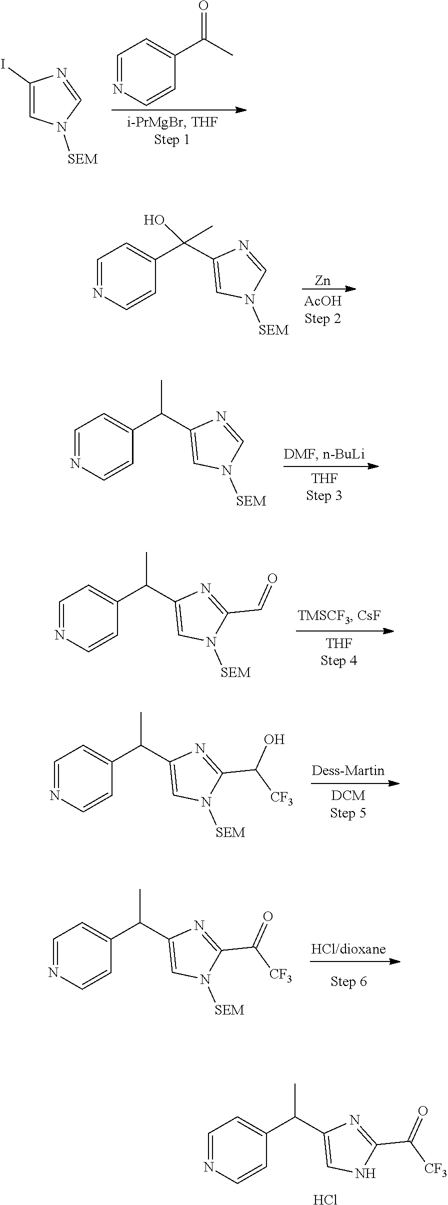

Step 1: 1-(Pyridin-4-yl)-1-(1-((2-(trimethylsilyl)ethoxy)methyl)-1H-imidazol-4-yl)ethan-1-ol. To a solution of 4-iodo-1-((2-(trimethylsilyl)ethoxy)methyl)-1H-imidazole (10 g, 31 mmol) in THF (100 mL) was added, dropwise, i-PrMgBr (33.8 mL, 33.9 mmol, 1.0 M) at 0° C. under a nitrogen atmosphere. The reaction mixture was stirred at 0° C. for 30 min under a nitrogen atmosphere. 4-Acetylpyridine (4.1 g, 33.9 mmol) was added dropwise at 0° C. and the resulting mixture was stirred for 1.5 h at rt under a nitrogen atmosphere. The resulting mixture was quenched with aq. sat. NH₄Cl (200 mL) at 0° C. and extracted with EtOAc (2×100 mL). The combined organic layers were washed with brine (200 mL), dried over anhydrous Na₂SO₄, filtered, and the filtrate was concentrated under reduced pressure. The crude product was purified by reverse phase flash chromatography to afford 1-(pyridine-4-yl)-1-(1-((2-(trimethylsilyl)ethoxy)methyl)-1H-imidazol-4-yl)ethan-1-ol (3.5 g, 36%) as a light yellow oil. LCMS ESI-MS m/z:=320 [M+H]⁺.

Step 2: 4-(1-(1-((2-(Trimethylsilyl)ethoxy)methyl)-1H-imidazol-4-yl)ethyl)pyridine. A mixture of 1-(pyridine-4-yl)-1-(1-((2-(trimethylsilyl)ethoxy)methyl)-1H-imidazol-4-yl)ethan-1-ol (3.5 g, 10.9 mmol) and Zn (8.6 g, 131 mmol) in AcOH (35 mL) was stirred overnight at 80° C. under a nitrogen atmosphere. The resulting mixture was cooled down to rt. The resulting mixture was filtered, the filter cake was washed with MeOH (2×50 mL) and the combined filtrate was concentrated under reduced pressure. The crude product was purified by reverse phase flash chromatography to afford 4-(1-(1-((2-(trimethylsilyl)ethoxy)methyl)-1H-imidazol-4-yl)ethyl)pyridine (2.3 g, 68%) as a yellow oil. LCMS ESI-MS m/z:=304 [M+H]⁺.

Step 3: 4-(1-(Pyridin-4-yl)ethyl)-1-((2-(trimethylsilyl)ethoxy)methyl)-1H-imidazole-2-carbaldehyde. To a solution of 4-(1-(1-((2-(trimethylsilyl)ethoxy)methyl)-1H-imidazol-4-yl)ethyl)pyridine (2.0 g, 6.6 mmol) in THF (40 mL) was added, dropwise, n-BuLi (7.9 mL, 19.8 mmol, 2.5 M) at −78° C. under a nitrogen atmosphere. The reaction mixture was stirred at −78° C. for 30 min under a nitrogen atmosphere. DMF (0.97 g, 13.2 mmol) was added dropwise at −78° C. under a nitrogen atmosphere. The resulting mixture was stirred for 1.5 h at −78° C. under a nitrogen atmosphere. The resulting mixture was quenched with aq. sat. NH₄Cl (50 mL) and extracted with EtOAc (2×30 mL). The combined organic phase was washed with brine (60 mL), dried over anhydrous Na₂SO₄, filtered, and the filtrate was concentrated under reduced pressure. The crude product was purified by reverse phase flash chromatography to afford 4-(1-(pyridin-4-yl)ethyl)-1-((2-(trimethylsilyl)ethoxy)methyl)-1H-imidazole-2-carbaldehyde (0.78 g, 32%) as a light yellow oil. LCMS ESI-MS m/z:=332 [M+H]⁺.

Step 4: 2,2,2-Trifluoro-1-(4-(pyridine-4-yl)ethyl)-1-((2-(trimethylsilyl)ethoxy)methyl)-1H-imidazol-2-yl)ethan-1-ol. To a stirred solution of 4-(1-(pyridin-4-yl)ethyl)-1-((2-(trimethylsilyl)ethoxy)methyl)-1H-imidazole-2-carbaldehyde (0.78 g, 2.4 mmol) and TMSCF₃ (0.5 g, 4.7 mmol) in THF (16 mL) was added CsF (0.54 g, 4.7 mmol) in portions at rt under a nitrogen atmosphere. The resulting mixture was stirred for 2 h at rt under a nitrogen atmosphere. The reaction was quenched with ice water (30 mL) and extracted with EtOAc (3×20 mL). The combined organic layers were washed with brine (50 mL), dried over anhydrous Na₂SO₄, filtered, and the filtrate was concentrated under reduced pressure. The crude product was purified by reverse phase flash chromatography to afford 2,2,2-trifluoro-1-(4-(pyridine-4-yl)ethyl)-1-((2-(trimethylsilyl)ethoxy)methyl)-1H-imidazol-2-yl)ethan-1-ol (0.82 g, 87%) as a light yellow oil. LCMS ESI-MS m/z:=402 [M+H]⁺.

Step 5: 2,2,2-Trifluoro-1-(4-(1-(pyridine-4-yl)ethyl)-1-((2-(trimethylsilyl)ethoxy)methyl)-1H-imidazol-2-yl)ethan-1-one. To a stirred solution of 2,2,2-trifluoro-1-(4-(pyridine-4-yl)ethyl)-1-((2-(trimethylsilyl)ethoxy)methyl)-1H-imidazol-2-yl)ethan-1-ol (720 mg, 1.7 mmol) in DCM (7.2 mL) was added Dess-Martin reagent (1.5 g, 3.5 mmol) in portions at 0° C. under a nitrogen atmosphere. The resulting mixture was stirred overnight at rt under a nitrogen atmosphere. The reaction was quenched with water (30 mL) at rt. The resulting mixture was extracted with DCM (3×10 mL). The combined organic layers were dried over anhydrous Na₂SO₄, filtered, and the filtrate was concentrated under reduced pressure to afford 2,2,2-trifluoro-1-(4-(1-(pyridine-4-yl)ethyl)-1-((2-(trimethylsilyl)ethoxy)methyl)-1H-imidazol-2-yl)ethan-1-one (820 mg) as a yellow oil which was used in the next step directly without further purification. LCMS ESI-MS m/z:=400 [M+H]⁺.

Step 6: 2,2,2-Trifluoro-1-(4-(pyridine-4-yl)ethyl)-1H-imidazol-2-yl)ethan-1-one hydrochloride. To a stirred solution of 2,2,2-trifluoro-1-(4-(1-(pyridine-4-yl)ethyl)-1-((2-(trimethylsilyl)ethoxy)methyl)-1H-imidazol-2-yl)ethan-1-one (820 mg, 2.04 mmol) in dioxane (4 mL) was added HCl (g) in dioxane (4 mL) dropwise at 0° C. under a nitrogen atmosphere. The resulting mixture was stirred for additional 3 h at rt under a nitrogen atmosphere. The resulting mixture was concentrated under vacuum. The crude product was purified by prep-HPLC to afford to afford 2,2,2-trifluoro-1-(4-(pyridine-4-yl)ethyl)-1H-imidazol-2-yl)ethan-1-one hydrochloride (8.5 mg, 1.3%) as a yellow oil. ¹H NMR (400 MHz, Deuterium Oxide, ppm): δ 8.69-8.57 (m, 2H), 8.07-7.78 (m, 2H), 7.59-7.45 (m, 1H), 4.61-4.53 (m, 1H), 1.92-1.57 (m, 3H). LCMS ESI-MS m/z:=270 [M+H-HCl]⁺.

### Example 23A and 23B: (R)-2,2,2-Trifluoro-1-(4-(1-(pyridin-4-yl)ethyl)-1H-imidazol-2-yl)ethan-1-one hydrochloride and (S)-2,2,2-Trifluoro-1-(4-(1-(pyridin-4-yl)ethyl)-1H-imidazol-2-yl)ethan-1-one hydrochloride

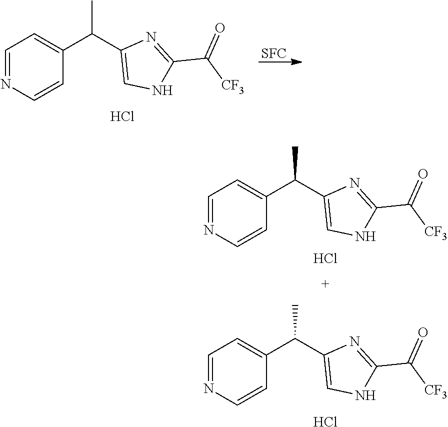

(R)-2,2,2-Trifluoro-1-(4-(1-(pyridin-4-yl)ethyl)-1H-imidazol-2-yl)ethan-1-one hydrochloride and (S)-2,2,2-Trifluoro-1-(4-(1-(pyridin-4-yl)ethyl)-1H-imidazol-2-yl)ethan-1-one hydrochloride. The crude racemic material from Example 22, Step 5, was dissolved in water (3 mL) and basified to pH 8 with TEA and concentrated under reduced pressure. The resulting material was purified by SFC to afford Peak 1 and Peak 2. The crude products were basified to pH 8 with saturated aqueous NaHCO₃ and purified by reverse-phase flash chromatography to afford Peak 1 (20.4 mg, 3%) as a white solid. ¹H NMR (300 MHz, D₂O) δ 8.75-8.63 (m, 2H), 7.97-7.84 (m, 2H), 7.56 (d, J=1.0 Hz, 1H), 4.63 (t, J=7.2 Hz, 1H), 1.69 (d, J=7.2 Hz, 3H). LCMS ESI-MS m/z:=270 [M+H]⁺. Peak 2 (12 mg, 2%) as a white solid. ¹H NMR (300 MHz, D₂O) δ 8.72-8.64 (m, 2H), 7.93-7.85 (m, 2H), 7.56 (d, J=1.0 Hz, 1H), 4.63 (t, J=7.2 Hz, 1H), 1.69 (d, J=7.2 Hz, 3H). LCMS ESI-MS m/z:=270 [M+H]⁺. The absolute stereochemistry was not determined for either isomer.

### Example 24: 4-((2-((3,3-Difluorocyclobutyl)methyl)-1H-imidazol-4-yl)methyl)pyridine

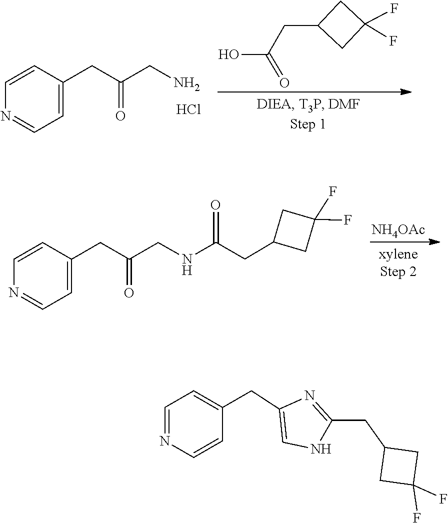 [SMILES: NCC(=O)Cc1ccncc1 ; Cl ; O=C(O)CC1CC(F)(F)C1 ; O=C(CNC(=O)CC1CC(F)(F)C1)Cc1ccncc1 ; FC1(F)CC(Cc2nc(Cc3ccncc3)c[nH]2)C1 ; "NH4OAc"]

Step 1: 2-(3,3-Difluorocyclobutyl)-N-(2-oxo-3-(pyridin-4-yl)propyl)acetamide. To a stirred solution of (3,3-difluorocyclobutyl)acetic acid (338 mg, 2.3 mmol) and DIEA (727 mg, 5.6 mmol) in DMF (3.5 mL) was added T₃P (1.8 g, 2.8 mmol, 50% wt.) and 1-amino-3-(pyridin-4-yl)propan-2-one hydrochloride (350 mg, 1.9 mmol) at 0° C. under a nitrogen atmosphere. The resulting mixture was stirred for 1 h at rt under a nitrogen atmosphere. The resulting mixture was quenched by the addition of water (10 mL) at 0° C. The resulting mixture was extracted with EtOAc (3×5 mL). The combined organic layers were washed with brine (5 mL), dried over anhydrous Na₂SO₄, filtered, and the filtrate was concentrated under reduced pressure. The residue was purified by reverse phase flash chromatography to afford 2-(3,3-difluorocyclobutyl)-N-(2-oxo-3-(pyridin-4-yl)propyl)acetamide (150 mg, 28%) as a light yellow solid. LCMS ESI-MS m/z:=283 [M+H]⁺.

Step 2: 4-((2-((3,3-Difluorocyclobutyl)methyl)-1H-imidazol-4-yl)methyl)pyridine. To a stirred solution of 2-(3,3-difluorocyclobutyl)-N-(2-oxo-3-(pyridin-4-yl)propyl)acetamide (150 mg, 0.53 mmol) and NH₄OAc (819.2 mg, 10.6 mmol) in xylene (3.0 mL) was stirred and irradiated for 1 h at 140° C. in a microwave. The resulting mixture was cooled to rt and quenched with water (10 mL) at rt. The resulting mixture was extracted with EtOAc (2×2 mL). The combined organic layers were washed with brine (1×2 mL), dried over anhydrous Na₂SO₄, filtered, and the filtrate was concentrated under a reduced pressure. The residue was purified by reverse phase flash chromatography to afford 4-((2-((3,3-difluorocyclobutyl)methyl)-1H-imidazol-4-yl)methyl)pyridine (23 mg, 16%) as a yellow solid. ¹H NMR (400 MHz, DMSO-d₆, ppm): δ 11.59 (s, 1H), 8.49-8.38 (m, 2H), 7.25-7.19 (m, 2H), 6.72 (s, 1H), 3.79 (s, 2H), 2.74 (d, J=7.3 Hz, 2H), 2.71-2.55 (m, 2H), 2.49-2.40 (m, 1H), 2.39-2.22 (m, 2H). LCMS ESI-MS m/z:=264 [M+H]⁺.

### Example 25: 4-((2-((1-(Trimethyl)cyclopropyl)methyl)-1H-imidazol-4-yl)methyl)pyridine

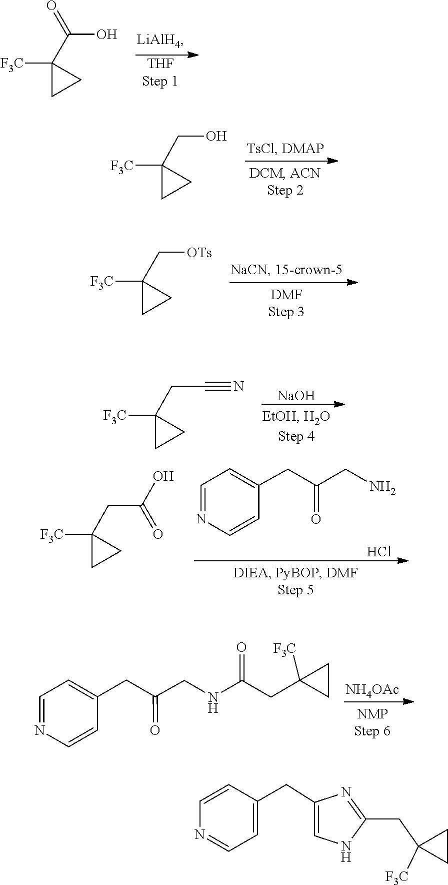

Step 1: (1-(Trifluoromethyl)cyclopropyl)methanol. To a stirred solution of 1-(trifluoromethyl)cyclopropane)-1-carboxylic acid (20 g, 130 mmol) in THF (200 mL) was added in portions LiAlH₄ (4.9 g, 129 mmol) at 0° C. under a nitrogen atmosphere. The resulting mixture was stirred overnight at rt under a nitrogen atmosphere. The reaction was cooled to 0° C., quenched with Na₂SO₄ 10 H₂O (29.4 g) and stirred for 3 h at 0° C. under a nitrogen atmosphere. The resulting mixture was filtered, and the filter cake was rinsed with THF (2×100 mL). The combined filtrate was concentrated under reduced pressure. The crude product, (1-(trifluoromethyl)cyclopropyl)methanol (12.5 g), was used in the next step without further purification.

Step 2: (1-(Trifluoromethyl)cyclopropyl-methyl)-4-methylbenzenesulfonate. To a stirred solution of (1-(trifluoromethyl)cyclopropyl)methanol (12.5 g, 89.2 mmol) andDMAP (2.1 g, 17.8 mmol) in DCM (120 mL) and ACN (120 mL) was added TsCI (25.5 g, 133.8 mmol) dropwise at rt and the resulting mixture was stirred overnight at rt under a nitrogen atmosphere.

The resulting mixture was quenched with water (200 mL) and extracted with EtOAc (2×100 mL). The combined organic phase was washed with brine (100 mL) and dried over anhydrous Na₂SO₄, filtered, and the filtrate was concentrated under reduced pressure. The crude product was purified by silica gel flash chromatography to afford (1-(trifluoromethyl)cyclopropyl-methyl)-4-methylbenzenesulfonate (5.6 g, two steps 15%) as a yellow solid.

Step 3: 2-(1-(Trifluoromethyl)cyclopropyl)acetonitrile. To a stirred solution of (1-(trifluoromethyl)cyclopropyl)methyl 4-methylbenzenesulfonate (5.6 g, 19.0 mmol) and 15-crown-5 (6.3 g, 28.5 mmol) in DMF (56 mL) was added NaCN (1.4 g, 28.5 mmol) in portions at rt and the resulting mixture was stirred for 48 h at 60° C. under a nitrogen atmosphere. The resulting mixture was cooled to rt. The resulting mixture was quenched with ice water (50 mL) and extracted with EtOAc (2×50 mL). The combined organic phase was washed with brine (50 mL), dried over anhydrous Na₂SO₄, filtered, and the filtrate was concentrated under reduced pressure to yield yellow oil. The crude product, 2-(1-(trifluoromethyl)cyclopropyl)acetonitrile (2.1 g), was used in the next step without further purification.

Step 4: (1-(Trifluoromethyl)cyclopropyl)acetic acid. To a stirred solution of 2-(1-(trifluoromethyl)cyclopropyl)acetonitrile (2.1 g, 14 mmol) in EtOH (21 mL) and H₂O (2.1 mL) was added NaOH (2.8 g, 70.4 mmol) in portions at 0° C. under a nitrogen atmosphere. The resulting mixture was stirred overnight at 80° C. under a nitrogen atmosphere. The resulting mixture was cooled to rt. The resulting mixture was concentrated under reduced pressure. The crude product was dissolved with H₂O (20 mL) and extracted with Et₂O (3×10 mL). The aqueous layer was acidified to pH 3 with HCl (aq., 4 M). The resulting mixture was extracted with EtOAc (3×10 mL). The organic layers were concentrated under reduced pressure to yield yellow oil. The crude product, (1-(trifluoromethyl)cyclopropyl)acetic acid (350 mg), was used in the next step without further purification. LCMS ESI-MS m/z:=167 [M−H]⁻.

Step 5: N-(2-Oxo-3-(pyridin-4-yl)propyl)-2-(1 (trifluoromethyl)cyclopropyl)acetamide. To a stirred solution of (1-(trifluoromethyl)cyclopropyl)acetic acid (0.9 g, 5.35 mmol) and PyBOP (4.17 g, 8.02 mmol) in DMF (9 mL) was added DIEA (2.07 g, 16.05 mmol) and 1-amino-3-(pyridin-4-yl)propan-2-one hydrochloride (1.09 g, 5.88 mmol) in portions at rt under a nitrogen atmosphere. The resulting mixture was stirred for 3 h at rt under a nitrogen atmosphere. The resulting mixture was diluted with water (35 mL) and extracted with EtOAc (3×15 mL). The combined organic layers were washed with brine (2×10 mL), dried over anhydrous Na₂SO₄, filtered, and the filtrate was concentrated under reduced pressure to yield a brown solid. The crude product was purified by reverse phase flash chromatography to afford N-(2-oxo-3-(pyridin-4-yl)propyl)-2-(1-(trifluoromethyl)cyclopropyl)acetamide (564 mg, 32%) as a yellow solid. LCMS ESI-MS m/z:=302 [M+H]⁺.

Step 6: 4-((2-((1-(Trimethyl)cyclopropyl)methyl)-1H-imidazol-4-yl)methyl)pyridine. To a stirred mixture of N-(2-oxo-3-(pyridin-4-yl)propyl)-2-(1-(trifluoromethyl)cyclopropyl)acetamide (564 mg, 1.7 mmol) and NH₄OAc (2.62 g, 34.08 mmol) in NMP (11.2 mL) was stirred and irradiated for 5 h at 140° C. in a microwave. The resulting mixture was cooled to rt. The resulting mixture was diluted with water (10 mL) and extracted with EtOAc (3×10 mL). The combined organic layers were washed with brine (10 mL), dried over anhydrous Na2SO4, filtered, and the filtrate was concentrated under reduced pressure. The crude product was purified by reverse phase flash chromatography to afford 4-((2-((1-(trimethyl)cyclopropyl)methyl)-1H-imidazol-4-yl)methyl)pyridine (89 mg, 19%) as a white semi-solid. ¹H NMR (300 MHz, DMSO-d6, ppm): δ 11.69 (s, 1H), 8.43 (d, J=5.6 Hz, 2H), 7.20 (d, J=6.0 Hz, 2H), 6.91-6.52 (m, 1H), 3.79 (s, 2H), 2.97 (s, 2H), 0.91-0.82 (m, 2H), 0.80-0.74 (m, 2H). LCMS ESI-MS m/z:=282 [M+H]⁺.

### Example 26: 4-((2-(2-(Trifluoromethyl)cyclo propyl)-1H-imidazol-4-yl)methyl)pyridine

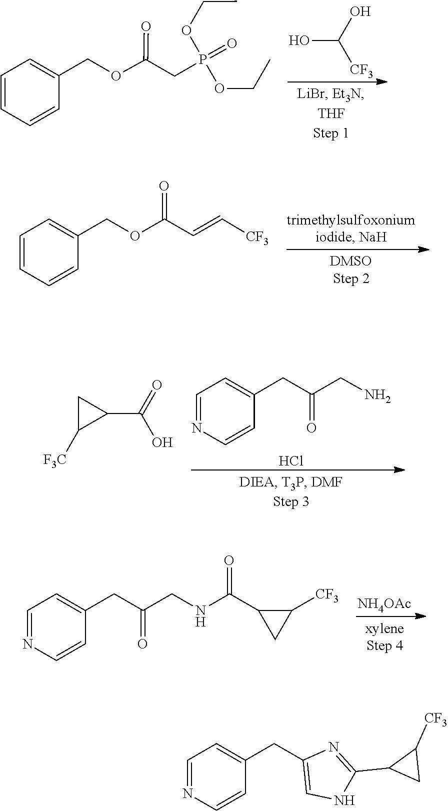

Step 1: 4-((2-(2-(Trifluoromethyl)cyclopropyl)-1H-imidazol-4-yl)methyl)pyridine. To a stirred solution of benzyl 2-(diethoxyphosphoryl)acetate (5.5 g, 19 mmol) and triethylamine (2.3 g, 23 mmol) in THE (55 mL) was added lithium bromide (2.0 g, 23 mmol) and trifluoroacetaldehyde hydrate (2.5 g, 21 mmol) at −10° C. under a nitrogen atmosphere. The mixture was stirred for 3 h at rt under a nitrogen atmosphere. The mixture was concentrated under reduced pressure. The crude product was purified by silica gel column chromatography to afford 4-((2-(2-(trifluoromethyl)cyclopropyl)-1H-imidazol-4-yl)methyl)pyridine (1.45 g, 33%) as a yellow oil.

Step 2: 2-(Trifluoromethyl)cyclopropane-1-carboxylic acid. To a stirred solution of 4-((2-(2-(trifluoromethyl)cyclopropyl)-1H-imidazol-4-yl)methyl)pyridine (1.3 g, 6.3 mmol) and trimethylsulfoxonium iodide (1.4 g, 6.9 mmol) in DMSO (14.5 mL) was added NaH (0.25 g, 6.9 mmol, 60% wt.) in portions at 0° C. under a nitrogen atmosphere. The resulting mixture was stirred for 3 h at rt under a nitrogen atmosphere. The resulting mixture was quenched with ice water (30 mL). The resulting mixture was extracted with EtOAc (1×20 mL). The aqueous phase was acidified to pH 1-2 with HCl (2 M) and extracted with EtOAc (3×15 mL). The combined organic phase was washed with brine (1×10 mL), dried over anhydrous Na₂SO₄, filtered, and the filtrate was concentrated under reduced pressure to afford 2-(trifluoromethyl)cyclopropane-1-carboxylic acid (0.7 g). The crude product was used in the next step directly without further purification. LCMS ESI-MS m/z:=153 [M−H]⁻.

Step 3: N-(2-Oxo-3-(pyridin-4-yl)propyl)-2-(trifluoromethyl)cyclopropane-1-carboxamide. To a stirred solution of 2-(trifluoromethyl)cyclopropane-1-carboxylic acid (700 mg, 4.5 mmol) and DIEA (1.8 g, 13.6 mmol) in DMF (7.0 mL) was added T₃P (4.3 g, 6.8 mmol, 50% wt.) dropwise at 0° C. under nitrogen atmosphere and continued to stir for 10 min. To the mixture was added 1-amino-3-(pyridin-4-yl)propan-2-one hydrochloride (848 mg, 4.5 mmol) in portions at 0° C. The resulting mixture was stirred for 3 h at rt under a nitrogen atmosphere. The reaction was quenched with ice water (10 mL) and the aqueous phase was extracted with EtOAc (3×5 mL). The combined organic layers were washed with brine (10 mL), dried over anhydrous Na₂SO₄, filtered, and concentrated under reduced pressure. The crude product was purified by reverse phase flash chromatography to afford N-(2-oxo-3-(pyridin-4-yl)propyl)-2-(trifluoromethyl)cyclopropane-1-carboxamide (75 mg, 3% over 2 steps) as a yellow oil. LCMS ESI-MS m/z:=287 [M+H]⁺.

Step 4: 4-((2-(2-(Trifluoromethyl)cyclopropyl)-1H-imidazol-4-yl)methyl)pyridine. To a stirred solution of N-[2-oxo-3-(pyridin-4-yl)propyl]-2-(trifluoromethyl)cyclopropane-1-carboxamide (75 mg, 0.16 mmol, 1 eq., purity: 60%) in xylene (1.5 mL) was added NH₄OAc (242 mg, 3.1 mmol) at rt under a nitrogen atmosphere. The resulting mixture was stirred and irradiated for 10 min at 140° C. in a microwave. The resulting mixture was cooled to rt. The reaction was quenched with water (5 mL) at 0° C. and extracted with EtOAc (3×3 mL). The combined organic layers were washed with brine (5 mL), dried over anhydrous Na₂SO₄, filtered, and concentrated under reduced pressure. The crude product was purified by reverse phase flash chromatography to afford 4-((2-(2-(trifluoromethyl)cyclopropyl)-1H-imidazol-4-yl)methyl)pyridine (1.9 mg, 4%) as a yellow oil. ¹H NMR: (300 MHz, DMSO-d₆, ppm): δ 11.99-11.66 (m, 1H), 8.54-8.34 (m, 2H), 7.28-7.19 (m, 2H), 6.81-6.49 (m, 1H), 3.95-3.69 (m, 2H), 2.40-2.14 (m, 2H), 1.36-1.26 (m, 2H). LCMS ESI-MS m/z:=268 [M+H]⁺.

### Example 27: (cis)-4-((2-(3-(trifluoromethyl)tetrahydrofuran-2-yl)-1H-imidazol-4-yl)methyl)pyridine

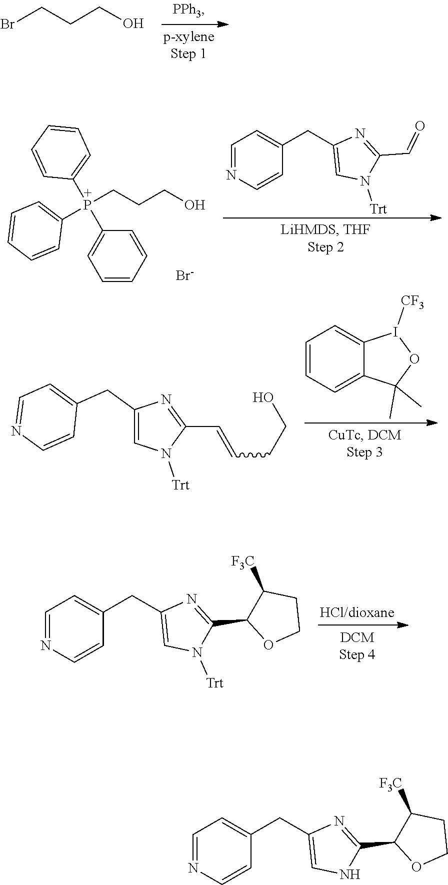

Step 1: (3-Hydroxypropyl) triphenylphosphonium bromide. To a stirred solution of 3-bromopropanol (5 g, 36 mmol) in p-xylene (30 mL) was added PPh₃ (9.5 g, 36.3 mmol) in portions at rt under a nitrogen atmosphere. The resulting mixture was stirred for 6 h at 120° C. under a nitrogen atmosphere. The reaction was cooled to rt. To the resulting mixture was added Et₂O (10 mL) and stirred for 10 min. The precipitated solids were collected by filtration and the filter cake was rinsed with Et₂O (10 mL) to afford (3-hydroxypropyl) triphenylphosphanium bromide (6.8 g) as a white solid. The crude product was used in the next step directly without further purification. LCMS ESI-MS m/z:=321 [M-Br]⁻.

Step 2: 4-(4-(Pyridin-4-ylmethyl)-1-trityl-1-H-imidazol-2-yl)but-3-en-1-ol. To a stirred solution of (3-hydroxypropyl)triphenylphosphanium bromide (6.8 g, 17 mmol) in THF (68 mL) was added LiHMDS (50.8 mL, 50.8 mmol, 1 M in THF) rapidly at −40° C. under a nitrogen atmosphere. The resulting mixture was stirred at −40° C. for 2 h. To the mixture was added 4-(pyridin-4-ylmethyl)-1-(triphenylmethyl) imidazole-2-carbaldehyde (7.3 g, 17 mmol) in portions at −40° C. The resulting mixture was warmed gently to rt and stirred overnight under a nitrogen atmosphere. The reaction was quenched with saturated NH₄Cl (100 mL) at 0° C. The resulting mixture was extracted with EtOAc (3×100 mL). The combined organic phase was washed with brine (50 mL), dried over anhydrous Na₂SO₄, filtered, and concentrated under reduced pressure. The crude product was purified by reverse phase flash chromatography to afford 4-(4-(pyridin-4-ylmethyl)-1-trityl-1H-imidazol-2-yl)but-3-en-1-ol (0.62 g, 4% over 2 steps) as a yellow oil. LCMS ESI-MS m/z:=472 [M+H]⁺.

Step 3: (cis)-4-((2-(3-(trifluoromethyl)tetrahydrofuran-2-yl)-1-trityl-1H-imidazol-4-yl)methyl)pyridine. To a stirred solution of 4-(4-(pyridin-4-ylmethyl)-1-trityl-1H-imidazol-2-yl)but-3-en-1-ol (530 mg, 1.1 mmol) and 3,3-dimethyl-1-(trifluoromethyl)-1,2-benzidoxole (368.2 mg, 1.1 mmol) in DCM (5.3 mL) was added CuTc (2.11 mg, 0.01 mmol) at rt under a nitrogen atmosphere. The resulting mixture was stirred for 4 h at 50° C. under a nitrogen atmosphere. The reaction was cooled to rt. The resulting mixture was quenched with water (5 mL) at rt. The aqueous phase was extracted with DCM (3×5 mL), the extracts were washed with brine (10 mL), dried over anhydrous Na₂SO₄, filtered, and concentrated under reduced pressure. The crude product was purified by reverse phase flash chromatography to afford (cis)-4-((2-(3-(trifluoromethyl)tetrahydrofuran-2-yl)-1-trityl-1H-imidazol-4-yl)methyl)pyridine (110 mg, 18%) as a yellow oil. LCMS ESI-MS m/z:=540 [M+H]⁺.

Step 4: (cis)-4-((2-(3-(trifluoromethyl)tetrahydrofuran-2-yl)-1H-imidazol-4-yl)methyl)pyridine. To a stirred solution of 4-((2-((2R, 3S)-3-(trifluoromethyl)tetrahydrofuran-2-yl)-1-trityl-1H-imidazol-4-yl)methyl)pyridine (110 mg) in DCM (2.2 mL) was added HCl/dioxane (1.1 mL, 4 M) dropwise at 0° C. under a nitrogen atmosphere. The resulting mixture was stirred for 2 h at rt under a nitrogen atmosphere. The resulting mixture was concentrated under reduced pressure. The residue was dissolved in DCM (3 mL) and H₂O (3 mL) and the mixture was basified to pH 9-10 with sat. NaHCO₃. The resulting mixture was extracted with DCM (3×3 mL). The extracts were dried over anhydrous Na₂SO₄, filtered, and concentrated under reduced pressure. The crude product was purified by reverse phase flash chromatography to afford (cis)-4-((2-(3-(trifluoromethyl)tetrahydrofuran-2-yl)-1H-imidazol-4-yl)methyl)pyridine (29 mg, 48%) as a white solid. ¹H NMR: (300 MHz, DMSO-d₆, ppm) δ 12.06 (s, 1H), S 8.47 (d, J=6.0 Hz, 2H), 7.25 (d, J=6.0 Hz, 2H), 6.99-6.10 (m, 1H), 4.96 (d, J=5.3 Hz, 1H), 3.97-3.77 (m, 4H), 3.76-3.61 (m, 1H), 2.42-2.24 (m, 1H), 2.12-1.95 (m, 1H). LCMS ESI-MS m/z:=298 [M+H]⁺.

### Example 28: 4-((2-((Trifluoromethyl)thio)-1H-imidazol-5-yl)methyl)pyridine

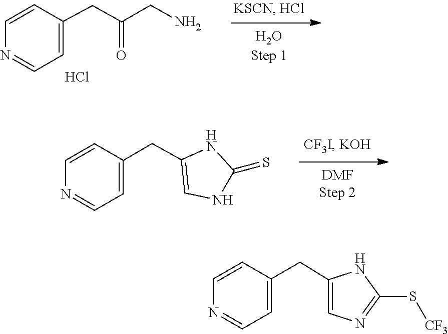 [SMILES: NCC(=O)Cc1ccncc1 ; Cl ; S=c1[nH]cc(Cc2ccncc2)[nH]1 ; FC(F)(F)Sc1ncc(Cc2ccncc2)[nH]1]

Step 1: 4-(Pyridin-4-ylmethyl)-1,3-dihydro-2H-imidazole-2-thione. To a stirred solution of 1-amino-3-(pyridin-4-yl)propan-2-one hydrochloride (3 g, 16 mmol) in H₂O (60 mL) was added potassium thiocyanate (1.5 g, 16 mmol) and HCl (3.2 mL, 19 mmol, 6M) at rt under a nitrogen atmosphere. The resulting mixture was stirred overnight at 60° C. under a nitrogen atmosphere. The resulting mixture was cooled to rt. The resulting mixture was basified to pH 9 with aqueous saturated Na₂CO₃ and extracted with DCM/MeOH (V/V=10:1, 5×50 mL). The combined organic phase was dried over anhydrous Na₂SO₄, filtered, and the filtrate was concentrated under reduced pressure. The crude product was purified by reverse phase flash chromatography to afford 4-(pyridin-4-ylmethyl)-1,3-dihydro-2H-imidazole-2-thione (1.2 g, 34%) as a yellow solid. LCMS ESI-MS m/z:=192 [M+H]⁺.

Step 2: 4-((2-((Trifluoromethyl)thio)-1H-imidazol-5-yl)methyl)pyridine. To a stirred solution of 4-(pyridin-4-ylmethyl)-1,3-dihydro-2H-imidazole-2-thione (1.2 g, 6.3 mmol) in DMF (24 mL) was added KOH (1.0 g, 18.8 mmol) and trifluoroiodomethane (1.4 g, 7.5 mmol) at rt under a nitrogen atmosphere. The resulting mixture was stirred for 2 h at 60° C. under a nitrogen atmosphere. The resulting mixture was cooled to rt. The resulting mixture was quenched with water (60 mL) and extracted with EtOAc (4×20 mL). The combined organic layers were washed with brine (20 mL), dried over anhydrous Na₂SO₄, filtered, and the filtrate was concentrated under reduced pressure. The crude product was purified by reverse phase flash chromatography to afford 4-((2-((trifluoromethyl)thio)-1H-imidazol-5-yl)methyl)pyridine (1.9 mg, 5%) as a white solid. ¹H NMR (400 MHz, DMSO-d₆, ppm): δ 13.23 (s, 1H), 8.46 (d, J=6.0 Hz, 2H), 7.31-7.23 (m, 3H), 3.99-3.89 (m, 2H). LCMS ESI-MS m/z:=260 [M+H]⁺.

### Example 29: (cis)-4-((2-(3-Fluorocyclobutyl)-1H-imidazol-4-yl)methyl)pyridine

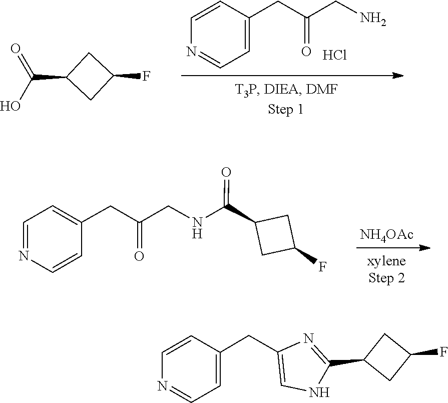 [SMILES: O=C(O)[C@H]1C[C@@H](F)C1 ; NCC(=O)Cc1ccncc1 ; Cl ; O=C(CNC(=O)[C@H]1C[C@@H](F)C1)Cc1ccncc1 ; F[C@H]1C[C@@H](c2nc(Cc3ccncc3)c[nH]2)C1 ; "NH4OAc"]

Step 1: (cis)-3-Fluoro-N-(2-oxo-3-(pyridine-4-yl)propyl)cyclobutene-1-carboxamide. To a solution of (cis)-3-fluorocyclobutane-1-carboxylic acid (0.50 g, 4.2 mmol) and DIEA (1.6 g, 12.7 mmol) in DMF (5 mL) was added with T₃P (3.23 g, 5.08 mmol, 50% wt.) at rt under a nitrogen atmosphere and stirred for 5 min. To the resulting mixture was added 1-amino-3-(pyridin-4-yl) propan-2-one hydrochloride (0.79 g, 4.23 mmol) in portions at rt under a nitrogen atmosphere. The resulting mixture was stirred for 3 h at rt under a nitrogen atmosphere. The resulting mixture was quenched with water (15 mL) and extracted with EtOAc (3×15 mL). The combined organic layers were washed with brine (15 mL), dried over anhydrous Na₂SO₄, filtered, and the filtrate was concentrated under reduced pressure. The crude product was purified by reverse phase flash chromatography to afford (cis)-3-fluoro-N-(2-oxo-3-(pyridine-4-yl)propyl)cyclobutene-1-carboxamide (0.29 g, yield: 27%) as a light yellow oil. LCMS ESI-MS m/z:=251 [M+H]⁺.

Step 2: (cis)-4-((2-(3-Fluorocyclobutyl)-1H-imidazol-4-yl)methyl)pyridine. A mixture of (cis)-3-fluoro-N-(2-oxo-3-(pyridine-4-yl)propyl)cyclobutene-1-carboxamide (290 mg, 1.16 mmol) and NH₄OAc (893 mg, 11.6 mmol) in xylene (5.8 mL) was stirred and irradiated for 10 min at 140° C. in a microwave. The resulting mixture was cooled to rt and was quenched by the addition of water (10 mL) at rt. The resulting mixture was extracted with EtOAc (4×10 mL). The combined organic layers were washed with brine (10 mL), dried over anhydrous Na₂SO₄, filtered, and the filtrate was concentrated under reduced pressure. The crude product was purified by reverse phase flash chromatography to afford (cis)-4-((2-(3-fluorocyclobutyl)-1H-imidazol-4-yl)methyl)pyridine (5.8 mg, 2%) as a light yellow solid. LCMS ESI-MS m/z:=232 [M+H]⁺. ¹H NMR (300 MHz, DMSO-d₆, ppm): δ 11.82-11.67 (m, 1H), 8.54-8.41 (m, 2H), 7.25 (d, J=5.4 Hz, 2H), 6.82-6.51 (m, 1H), 5.12-4.88 (m, 1H), 3.89-3.65 (m, 2H), 2.98-2.82 (m, 1H), 2.69-2.58 (m, 2H), 2.46-2.25 (m, 2H).

### Example 30: (trans)-4-((2-(3-Fluorocyclobutyl)-1H-imidazol-4-yl)methyl)pyridine

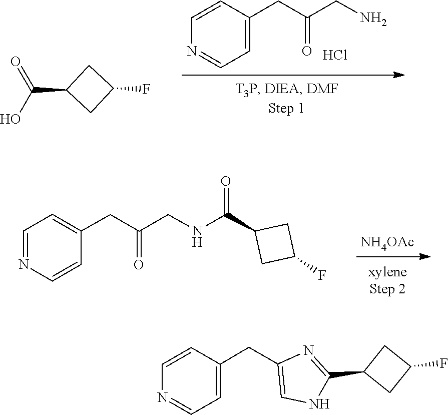 [SMILES: O=C(O)[C@H]1C[C@H](F)C1 ; NCC(=O)Cc1ccncc1 ; Cl ; O=C(CNC(=O)[C@H]1C[C@H](F)C1)Cc1ccncc1 ; F[C@H]1C[C@H](c2nc(Cc3ccncc3)c[nH]2)C1 ; "NH4OAc"]

Step 1: (trans)-3-Fluoro-N-(2-oxo-3-(pyridine-4-yl)propyl)cyclobutene-1-carboxamide. To a stirred solution of (trans)-3-fluorocyclobutane-1-carboxylic acid (500 mg, 4.23 mmol) and DIEA (1.6 g, 12.7 mmol) in DMF (5 mL) was added T₃P (4.04 g, 6.35 mmol, 50% wt.) at 0° C. under a nitrogen atmosphere and stirred for 5 min. The resulting mixture was added 1-amino-3-(pyridin-4-yl)propan-2-one hydrochloride (790.1 mg, 4.2 mmol) in portions at 0° C. and stirred for another 3 h at rt under a nitrogen atmosphere. The reaction was quenched with water (50 mL) at 0° C. and extracted with EtOAc (3×15 mL). The combined organic layers were washed with brine (20 mL), dried over anhydrous Na₂SO₄, filtered, and the filtrate was concentrated under reduced pressure. The crude product was purified by reverse phase flash chromatography to afford (trans)-3-fluoro-N-(2-oxo-3-(pyridine-4-yl)propyl)cyclobutene-1-carboxamide (330 mg, 31%) as a yellow oil. LCMS ESI-MS m/z:=251 [M+H]⁺.

Step 2: (trans)-4-((2-(3-Fluorocyclobutyl)-1H-imidazol-4-yl)methyl)pyridine. A stirred solution of (trans)-3-fluoro-N-(2-oxo-3-(pyridine-4-yl)propyl)cyclobutene-1-carboxamide (330 mg, 1.3 mmol) and NH₄OAc (1.01 g, 13.2 mmol) in xylene (6.6 mL) was irradiated for 10 min at 140° C. in a microwave. The resulting mixture was cooled to rt and was quenched with water (10 mL) at 0° C. The resulting mixture was extracted with EtOAc (4×5 mL). The combined organic phase was washed with brine (10 mL), dried over anhydrous Na₂SO₄, filtered, and the filtrate was concentrated under reduced pressure. The crude product was purified by Prep-HPLC to afford (trans)-4-((2-(3-fluorocyclobutyl)-1H-imidazol-4-yl)methyl)pyridine (30.6 mg, 10%) as a yellow solid. LCMS EI-MS m/z:=232 [M+H]⁺. ¹H NMR (300 MHz, DMSO-d6, ppm) δ 11.60 (s, 1H), 8.45 (d, J=5.1 Hz, 2H), 7.24 (d, J=5.3 Hz, 2H), 6.82-6.50 (m, 1H), 5.40-5.14 (m, 1H), 3.80 (s, 2H), 3.51-3.44 (m, 1H), 2.57-2.51 (t, J=6.6 Hz, 3H), 2.49-2.41 (m, 1H).

### Example 31A and 31B: (cis)-4-((2-(3-(Trifluoromethyl)cyclobutyl)-1H-imidazol-4-yl)methyl)pyridine and (trans)-4-((2-(3-(Trifluoromethyl)cyclobutyl)-1H-imidazol-4-yl)methyl)pyridine

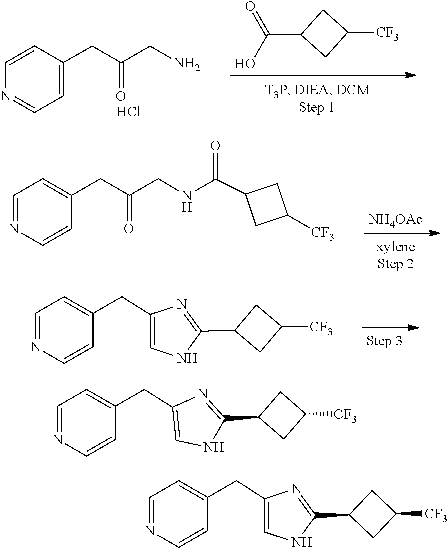

Step 1: N-(2-Oxo-3-(pyridine-4-yl)propyl)-3-(trifluoromethyl)cylcobutane-1-carboxamide. To a stirred solution of 3-(trifluoromethyl)cyclobutane-1-carboxylic acid (432.3 mg, 2.5 mmol) and DIEA (830.9 mg, 6.3 mmol) in DCM (4 mL) was added T₃P (1.6 g, 2.5 mmol, 50 w.t.%) at rt under a nitrogen atmosphere. 1-amino-3-(pyridin-4-yl)propan-2-one hydrochloride (400 mg, 2.1 mmol, 1.0 eq.) was added and the resulting mixture was stirred for 3 h at rt under a nitrogen atmosphere. The reaction was quenched by the addition of water (20 mL) at rt and extracted with DCM (3×20 mL). The combined organic layers were concentrated under reduced pressure. The crude product was purified by reverse phase flash chromatography to afford N-(2-oxo-3-(pyridine-4-yl)propyl)-3-(trifluoromethyl)cylcobutane-1-carboxamide (243 mg, 34%) as a yellow solid. LCMS ESI-MS m/z: 301 [M+H]⁺.

Step 2: 4-((2-(3-(Trifluoromethyl)cyclobutyl)-1H-imidazol-4-yl)methyl)pyridine. A mixture of N-(2-oxo-3-(pyridine-4-yl)propyl)-3-(trifluoromethyl)cylcobutane-1-carboxamide (220 mg, 0.7 mmol) and NH₄OAc (4.1 g, 14 mmol) in xylene (4.5 mL) was stirred and irradiated for 10 min at 140° C. in a microwave. The resulting mixture was cooled to rt and quenched by the addition of water (20 mL) at rt. The resulting mixture was extracted with EtOAc (3×10 mL). The combined organic phase was washed with brine (1×10 mL) and dried over anhydrous Na₂SO₄, filtered, and the filtrate was concentrated under reduced pressure. The crude product was purified by reverse phase flash chromatography to afford 4-((2-(3-(trifluoromethyl)cyclobutyl)-1H-imidazol-4-yl)methyl)pyridine (112 mg, 50%) as a yellow oil. LCMS ESI-MS m/z:=282 [M+H]⁺.

Step 3: (cis)-4-((2-(3-(Trifluoromethyl)cyclobutyl)-1H-imidazol-4-yl)methyl)pyridine and (trans)-4-((2-(3-(Trifluoromethyl)cyclobutyl)-1H-imidazol-4-yl)methyl)pyridine. 4-((2-(3-(Trifluoromethyl)cyclobutyl)-1H-imidazol-4-yl)methyl)pyridine (112 mg) was separated by prep-HPLC with the following conditions to afford Peak 1 (33.3 mg 30%) as a white solid. ¹H NMR: (400 MHz, DMSO-d₆, ppm): δ 11.76-11.59 (m, 1H), 8.51-8.40 (m, 2H), 7.27-7.19 (m, 2H), 6.76-6.54 (m, 1H), 3.90-3.76 (m, 2H), 3.47-3.37 (m, 1H), 3.23-3.07 (m, 1H), 2.44-2.26 (m, 4H); LCMS ESI-MS m/z:=282 [M+H]⁺. Peak 2 (14.4 mg, 13%) as a white solid. ¹H NMR: (400 MHz, DMSO-d₆, ppm): δ 11.73-11.52 (m, 1H), 8.49-8.36 (m, 2H), 7.23-7.11 (m, 2H), 6.66-6.49 (m, 1H), 3.87-3.72 (m, 2H), 3.42-3.32 (m, 1H), 3.21-3.02 (m, 1H), 2.41-2.22 (m, 4H). LCMS ESI-MS m/z:=282 [M+H]⁺.

The absolute stereochemistry was not determined for either isomer.

### Example 32: 4-((2-(4,4-Difluoropyrrolidin-2-yl)-1H-imidazol-4-yl)methyl)pyridine

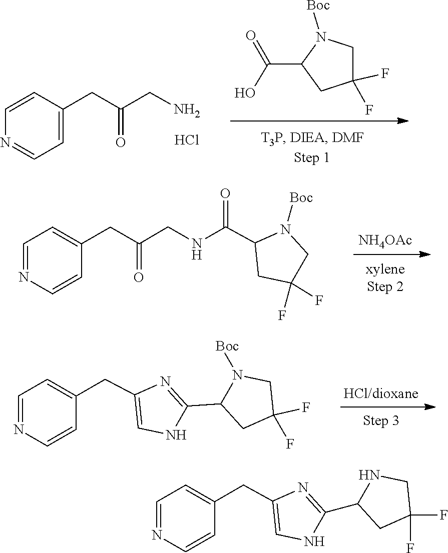

Step 1: tert-Butyl-4,4-difluoro-2-((2-oxo-3-(pyridine-4-yl)propyl)carbamoyl)pyrrolidine-1-carboxylate. To a stirred solution of 1-(tert-butoxycarbonyl)-4,4-difluoropyrrolidine-2-carboxylic acid (1.8 g, 7.07 mmol) and DIEA (2.3 g, 17.7 mmol) in DMF (11 mL) was added T₃P (4.5 g, 7.1 mmol, 50% wt.) at 0° C. under a nitrogen atmosphere. To the resulting mixture was added 1-amino-3-(pyridin-4-yl)propan-2-one hydrochloride (1.1 g, 5.9 mmol) in portions at 0° C. and stirred for 2 h at rt under a nitrogen atmosphere. The reaction was quenched with water (50 mL) at 0° C. and extracted with EtOAc (3×15 mL). The combined organic layers were washed with brine (1×20 mL), dried over anhydrous Na₂SO₄, filtered, and the filtrate was concentrated under reduced pressure. The crude product was purified by reverse phase flash chromatography to afford tert-butyl-4,4-difluoro-2-((2-oxo-3-(pyridine-4-yl)propyl)carbamoyl)pyrrolidine-1-carboxylate (0.4 g, 16%) as a yellow solid. LCMS ESI-MS m/z:=384 [M+H]⁺.

Step 2: tert-Butyl 4,4-Difluoro-2-(4-(pyridin-4-ylmethyl)-1H-imidazol-2-yl)pyrrolidine-1-carboxylate. A stirred solution of tert-butyl-4,4-difluoro-2-((2-oxo-3-(pyridine-4-yl)propyl)carbamoyl)pyrrolidine-1-carboxylate (0.4 g, 1.04 mmol) and NH₄OAc (1.6 g, 20.9 mmol) in xylene (8 mL) was stirred and irradiated for 1 h at 140° C. in a microwave. The resulting mixture was cooled to rt and was quenched with water (10 mL) at 0° C. The resulting mixture was extracted with EtOAc (4×5 mL). The combined organic phase was washed with brine (10 mL), dried over anhydrous Na₂SO₄, filtered, and the filtrate was concentrated under reduced pressure. The crude product was purified by reverse phase flash chromatography to afford tert-butyl 4,4-difluoro-2-(4-(pyridin-4-ylmethyl)-1H-imidazol-2-yl)pyrrolidine-1-carboxylate (0.12 g, 30%) as a yellow oil. LCMS EI-MS m/z:=365 [M+H]⁺.

Step 3: 4-((2-(4,4-Difluoropyrrolidin-2-yl)-1H-imidazol-4-yl)methyl)pyridine. To a stirred solution of tert-butyl 4,4-difluoro-2-(4-(pyridin-4-ylmethyl)-1H-imidazol-2-yl)pyrrolidine-1-carboxylate (120 mg, 0.33 mmol) in DCM (0.6 mL) was added HCl/dioxane (0.6 mL) dropwise at rt under a nitrogen atmosphere. The resulting mixture was stirred 2 h at rt under a nitrogen atmosphere. The resulting mixture was basified to pH 8 with sat. Na₂CO₃ (1 M) at 0° C. The resulting mixture was extracted with DCM (3×5 mL). The combined organic phase was washed with brine (5 mL), dried over anhydrous Na₂SO₄, filtered, and the filtrate was concentrated under reduced pressure. The crude product was purified by reverse phase flash chromatography to afford 4-((2-(4,4-difluoropyrrolidin-2-yl)-1H-imidazol-4-yl)methyl)pyridine (18 mg, 20%) as a yellow oil. ¹H NMR (300 MHz, D₂O, ppm): δ 8.29-8.27 (m, 2H), 7.18-7.16 (m, 2H), 6.80 (s, 1H), 4.42-4.37 (m, 1H), 3.85 (s, 2H), 3.24-3.09 (m, 2H), 2.71-2.32 (m, 2H). LCMS EI-MS m/z:=265 [M+H]⁺.

### Example 33A and 33B: (R)-4-((2-(2,2-Dichlorocyclopropyl)-1H-imidazol-4-yl)methyl)pyridine and (S)-4-((2-(2,2-Dichlorocyclopropyl)-1H-imidazol-4-yl)methyl)pyridine

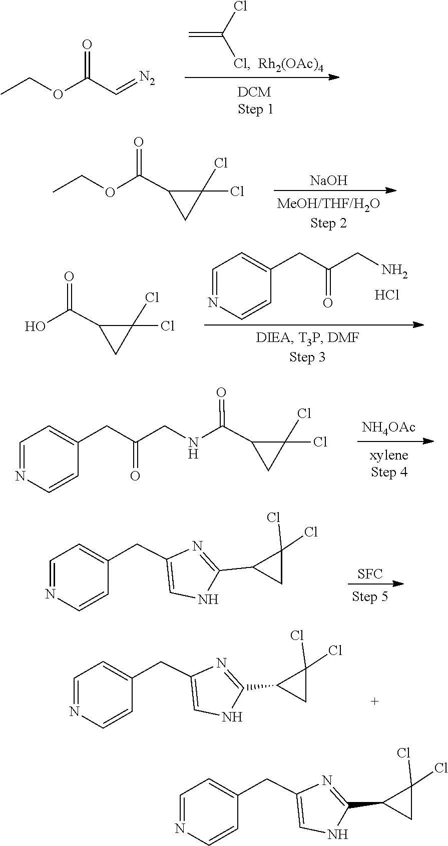

Step 1: Ethyl 2,2-dichlorocyclopropane-1-carboxylate. To a stirred solution of vinylidene chloride (170 g, 1753 mmol) and 1,1,1-tris(acetyloxy) dirhodium-1-yl acetate (775 mg, 1.8 mmol) in DCM (1.4 L) was added ethyl diazoacetate (20.0 g, 175.3 mmol) dropwise at 0-10° C. under a nitrogen atmosphere. The resulting mixture was stirred overnight at rt under a nitrogen atmosphere. The resulting mixture was concentrated under reduced pressure. The crude product was purified by silica gel column flash chromatography to afford ethyl 2,2-dichlorocyclopropane-1-carboxylate (9.5 g, 30%) as a yellow oil. LCMS ESI-MS m/z:=183 [M+H]⁺.

Step 2: 2,2-Dichlorocyclopropane-1-carboxylic acid. To a stirred solution of ethyl 2,2-dichlorocyclopropane-1-carboxylate (9.5 g, 51.9 mmol) in THF (38 mL), water (38 mL) and MeOH (19 mL) was added NaOH (4.2 g, 103.8 mmol) at 0-10° C. under a nitrogen atmosphere. The resulting mixture was stirred for 2 h at rt under a nitrogen atmosphere. The resulting mixture was concentrated to removed MeOH and THF. The resulting mixture was extracted with EtOAc (30 mL). The aqueous phase was acidified to pH 1-2 with HCl (3 M). The resulting mixture was extracted with EtOAc (3×30 mL). The combined organic phase was washed with brine (30 mL), dried over anhydrous Na₂SO₄, filtered, and the filtrate was concentrated under reduced pressure to a yellow oil. The crude product (5.2 g) was used in the next step directly without further purification. LCMS ESI-MS m/z:=155 [M+H]⁺.

Step 3: 2,2-Dichloro-N-(2-oxo-3-(pyridine-4-yl)propyl)cyclopropane-1-carboxamide. To a stirred solution of 2,2-dichlorocyclopropane-1-carboxylic acid (5.2 g, 33.6 mmol) and DIEA (13.0 g, 100.7 mmol) in DMF (52 mL) was added T₃P (32.1 g, 50.3 mmol, 50% wt.) dropwise at rt under a nitrogen atmosphere and stirred for 5 min. Then the resulting mixture was added 1-amino-3-(pyridin-4-yl)propan-2-one hydrochloride (6.3 g, 33.6 mmol) in portions at 0-10° C. under a nitrogen atmosphere. The resulting mixture was stirred for 3 h at rt under a nitrogen atmosphere. The reaction was quenched with water (200 mL) at 0° C. and extracted with EtOAc (3×100 mL). The combined organic layers were washed with brine (100 mL), dried over anhydrous Na₂SO₄, filtered, and the filtrate was concentrated under reduced pressure. The crude product was purified by reverse phase flash chromatography to afford 2,2-dichloro-N-(2-oxo-3-(pyridine-4-yl)propyl)cyclopropane-1-carboxamide (3.1 g, 2 steps, 20%) as a yellow oil. LCMS ESI-MS m/z:=287 [M+H]⁺.

Step 4: 4-((2-(2,2-Dichlorocyclopropyl)-1H-imidazol-4-yl)methyl)pyridine. To a stirred solution of 2,2-dichloro-N-(2-oxo-3-(pyridine-4-yl)propyl)cyclopropane-1-carboxamide (400 mg, 1.4 mmol) in xylene (16 mL) was added NH₄OAc (8.6 g, 111.4 mmol) at rt under a nitrogen atmosphere. The reaction mixture was irradiated with microwave radiation for 30 min at 140° C. under a nitrogen atmosphere. The reaction and five other reactions of 500 mg per batch were worked-up together. The combined resulting mixture was quenched with water (100 mL) at rt and extracted with EtOAc (5×50 mL). The combined organic layers were washed with brine (50 mL), dried over anhydrous Na₂SO₄, filtered, and the filtrate was concentrated under reduced pressure. The crude product was purified by reverse phase flash chromatography to afford 4-((2-(2,2-dichlorocyclopropyl)-1H-imidazol-4-yl)methyl)pyridine (120 mg, 17%) as a yellow oil. LCMS ESI-MS m/z:=268 [M+H]⁺.

Step 5: (R)-4-((2-(2,2-Dichlorocyclopropyl)-1H-imidazol-4-yl)methyl)pyridine and (S)-4-((2-(2,2-Dichlorocyclopropyl)-1H-imidazol-4-yl)methyl)pyridine. 120 mg of 4-((2-(2,2-dichlorocyclopropyl)-1H-imidazol-4-yl)methyl)pyridine was separated by prep-SFC to afford Peak 1 (27.4 mg, 23%) as a colorless oil, ¹H NMR: (400 MHz, DMSO-d₆, ppm): δ 12.04 (s, 1H), 8.47-8.41 (m, 2H), 7.24-7.18 (m, 2H), 6.83 (s, 1H), 3.83 (s, 2H), 2.99 (dd, J=10.9, 8.0 Hz, 1H), 2.22 (t, J=7.7 Hz, 1H), 2.08 (dd, J=10.9, 7.4 Hz, 1H). LCMS ESI-MS m/z: =268 [M+H]⁺. Peak 2 (30.5 mg, 25%) as a colorless oil. ¹H NMR: (400 MHz, DMSO-d₆, ppm): δ 12.28-11.86 (m, 1H), 8.43 (s, 2H), 7.29-7.09 (m, 2H), 7.02-6.57 (m, 1H), 4.01-3.68 (m, 2H), 2.99 (dd, J=10.9, 8.1 Hz, 1H), 2.22 (t, J=7.7 Hz, 1H), 2.08 (dd, J=10.9, 7.4 Hz, 1H). LCMS ESI-MS m/z:=268 [M+H]⁺. The absolute stereochemistry was not determined for either isomer.

### Example 34: 4-((2-(3-Chlorocyclobutyl)-1H-imidazol-4-yl)methyl)pyridine

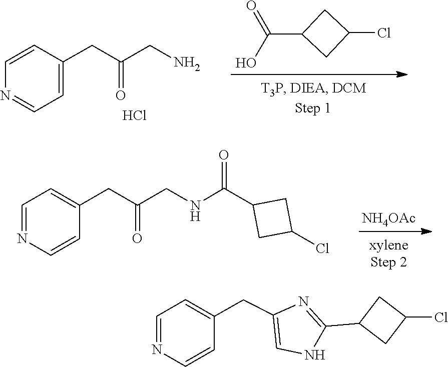 [SMILES: NCC(=O)Cc1ccncc1 ; O=C(O)C1CC(Cl)C1 ; Cl ; O=C(CNC(=O)C1CC(Cl)C1)Cc1ccncc1 ; ClC1CC(c2nc(Cc3ccncc3)c[nH]2)C1 ; "NH4OAc"]

Step 1: 4-((2-(3-Chloro-N-(2-oxo-3-(pyridine-4-yl)propyl)cyclobutene-1-carboxamide. To a solution of 3-chlorocyclobutane-1-carboxylic acid (346.1 mg, 2.6 mmol) and T₃P (1.6 g, 2.6 mmol, 50 w.t.%) in DCM (4 mL) was added DIEA (831 mg, 6.4 mmol) at rt under a nitrogen atmosphere. The resulting mixture was stirred for 5 min and 1-amino-3-(pyridin-4-yl)propan-2-one hydrochloride (400 mg, 2.1 mmol) was added in portions at rt under a nitrogen atmosphere. The resulting mixture was stirred for 3 h at rt and quenched with water (6 mL). The resulting mixture was extracted with DCM (3×5 mL). The combined organic layers were concentrated under reduced pressure. The crude product was purified by reverse phase flash chromatography to afford 4-((2-(3-chloro-N-(2-oxo-3-(pyridine-4-yl)propyl)cyclobutene-1-carboxamide (251 mg, 44%) as light yellow oil. LCMS ESI-MS m/z:=267 [M+H]+

Step 2: 4-((2-(3-Chlorocyclobutyl)-1H-imidazol-4-yl)methyl)pyridine. A mixture of 4-((2-(3-chloro-N-(2-oxo-3-(pyridine-4-yl)propyl)cyclobutene-1-carboxamide (210 mg, 0.79 mmol) and NH₄OAc (1.2 g, 15.7 mmol) in xylene (4 mL) was stirred and irradiated for 10 min at 140° C. in a microwave. The resulting mixture was cooled to rt and quenched by the addition of water (6 mL) at rt. The resulting mixture extracted with EtOAc (3×6 mL). The combined organic layers were washed with brine (6 mL), dried over anhydrous Na₂SO₄, filtered, and the filtrate was concentrated under reduced pressure. The crude product was purified by reverse phase flash chromatography to afford 4-((2-(3-chlorocyclobutyl)-1H-imidazol-4-yl)methyl)pyridine as a light yellow solid. ¹H NMR: (400 MHz, DMSO-d₆, ppm): δ 11.63 (s, 1H), 8.51-8.39 (m, 2H), 7.28-7.19 (m, 2H), 6.73 (s, 1H), 4.72 (ttd, J=7.0, 5.8, 1.0 Hz, 1H), 3.80 (s, 2H), 3.70 (tt, J=9.7, 5.5 Hz, 1H), 2.77 (dddt, J=10.9, 7.6, 5.3, 1.8 Hz, 2H), 2.63-2.53 (m, 2H). LCMS ESI-MS m/z:=248 [M+H]⁺.

### Example 35A and 35B: (cis)-(1R, 3R)-3-(4-Pyridin-4ylmethyl)-1H-imidazol-2-yl)cyclobutane-1-carbonitrile and (trans)-(1S, 3S)-3-(4-Pyridin-4ylmethyl)-1H-imidazol-2-yl)cyclobutane-1-carbonitrile

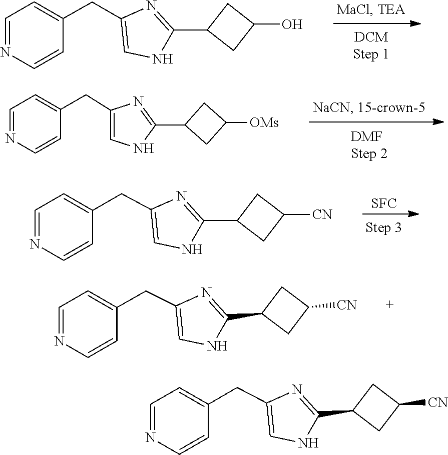

Step 1: 3-(4-Pyridin-4-ylmethyl)-1H-imidazol-2-yl)cyclobutyl methanesulfonate. To a stirred solution of 3-(4-pyridin-4ylmethyl)-1H-imidazol-2-yl)cyclobutan-1-ol (165 mg, 0.72 mmol) and TEA (146 mg, 1.4 mmol) in DCM (1.6 mL) was added MsCl (99 mg, 0.86 mmol) dropwise at 0° C. under a nitrogen atmosphere. The resulting mixture was stirred for 1 h at 0° C. under a nitrogen atmosphere. The resulting mixture was quenched with water (6 mL). The resulting mixture was extracted with DCM (3×6 mL). The combined organic layers were washed with brine (6 mL), dried over anhydrous Na₂SO₄, filtered, and the filtrate was concentrated under reduced pressure to afford 3-(4-pyridin-4-ylmethyl)-1H-imidazol-2-yl)cyclobutyl methanesulfonate (330 mg) as brown oil. The product was used in the next step directly without further purification. LCMS ESI-MS m/z:=308 [M+H]⁺.

Step 2: 3-(4-Pyridin-4ylmethyl)-1H-imidazol-2-yl)cyclobutane-1-carbonitrile. To a stirred solution of 3-(4-pyridin-4-ylmethyl)-1H-imidazol-2-yl)cyclobutyl methanesulfonate (310 mg, 1.0 mmol) and 1,4,7,10,13-pentaoxacyclopentadecane (444.3 mg, 2.0 mmol) in DMF (3.1 mL) was added NaCN (74.1 mg, 1.5 mmol) in portions at rt under a nitrogen atmosphere. The resulting mixture was stirred overnight at 130° C. under a nitrogen atmosphere. The resulting mixture was cooled to rt and was quenched by the addition of water (20 mL) at rt. The resulting mixture was extracted with EtOAc (3×20 mL). The combined organic layers were washed with brine (20 mL), dried over anhydrous Na₂SO₄, filtered, and the filtrate was concentrated under reduced pressure. The crude product was purified by reverse phase flash chromatography to afford 3-(4-pyridin-4ylmethyl)-1H-imidazol-2-yl)cyclobutane-1-carbonitrile (56 mg, 2 steps, 35%) as a light yellow oil. LCMS ESI-MS m/z:=239 [M+H]⁺.

Step 3: (cis)-3-(4-Pyridin-4ylmethyl)-1H-imidazol-2-yl)cyclobutane-1-carbonitrile and (trans)-3-(4-Pyridin-4ylmethyl)-1H-imidazol-2-yl)cyclobutane-1-carbonitrile. 3-(4-Pyridin-4ylmethyl)-1H-imidazol-2-yl)cyclobutane-1-carbonitrile was separated by prep-SFC to afford Peak 1 (2.9 mg, 5%) as a white solid; ¹H NMR: (300 MHz, DMSO-d₆, ppm): δ 11.63 (m, 1H), 8.44 (d, J=5.0 Hz, 2H), 7.23 (d, J=5.0 Hz, 2H), 6.77 (m, 1H), 3.80 (s, 2H), 3.68 (p, J=8.0 Hz, 1H), 3.44-3.37 (m, 1H), 2.58 (t, J=7.6 Hz, 4H). LCMS ESI-MS m/z:=239 [M+H]⁺. Peak 2 (4 mg, 8%) as colorless oil; ¹H NMR (300 MHz, DMSO-d₆, ppm): δ 11.75 (s, 1H), 8.44 (d, J=4.5 Hz, 2H), 7.23 (d, J=5.4 Hz, 2H), 6.71 (s, 1H), 3.81 (s, 2H), 3.49-3.40 (m, 1H), 3.36-3.24 (m, 1H), 2.68-2.52 (m, 4H). LCMS ESI-MS m/z:=239 [M+H]⁺. The absolute stereochemistry was not determined for either isomer.

### Example 36: 3-(4-(Pyridin-4-ylmethyl)-1H-imidazol-2-yl)-1-(trifluoromethyl)cyclobutan-1-ol

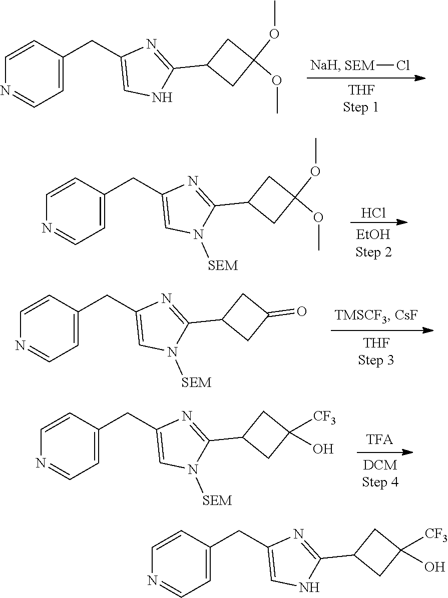

Step 1: 4-((2-(Dimethoxycylcobutyl)-1-((2-(trimethylsilyl)ethoxy)methyl)-1H-imidazol-4-yl)methyl)pyridine. To a stirred solution of 4-((2-(3,3-dimethoxycyclobutyl)-1H-imidazol-4-yl)methyl)pyridine (300 mg, 1.1 mmol) in THF (6 mL) was added NaH (48.3 mg, 1.2 mmol, 60%) in portions at 0° C. and stirred for an additional 0.5 h under a nitrogen atmosphere. SEM-Cl (220.6 mg, 1.3 mmol) was added dropwise at 0° C., the reaction was warmed to rt and stirred for 1.5 h. The resulting mixture was slowly poured into ice water (10 mL) and extracted with EtOAc (2×10 mL). The combined organic phase was washed with brine (15 mL), dried over anhydrous Na₂SO₄, filtered, and the filtrate was concentrated under reduced pressure. The crude product was purified by reverse phase flash chromatography to afford 4-((2-(dimethoxycylcobutyl)-1-((2-(trimethylsilyl)ethoxy)methyl)-1H-imidazol-4-yl)methyl)pyridine (420 mg, 68%) as brown oil. LCMS ESI-MS m/z:=404 [M+H]⁺.

Step 2: 3-(4-Pyridin-4-ylmethyl)-1-((2-(trimethylsilyl)ethoxy)methyl)-1H-imidazol-2-yl)cyclobutan-1-one. To a stirred mixture of 4-((2-(dimethoxycylcobutyl)-1-((2-(trimethylsilyl)ethoxy)methyl)-1H-imidazol-4-yl)methyl)pyridine (420 mg, 0.75 mmol) in EtOH (4.2 mL) was added HCl aq. (0.4 mL, 2.3 mmol, 6 M) dropwise at 0° C. under a nitrogen atmosphere. The resulting mixture was stirred for 4 h at 0° C. under a nitrogen atmosphere. The resulting mixture was basified to pH 9-10 with aq. Na₂CO₃. The resulting mixture was extracted with EtOAc (3×10 mL). The organic layers were combined and dried over anhydrous Na₂SO₄, filtered, and the filtrate was concentrated under reduced pressure. The crude product was purified by reverse phase flash chromatography to afford 3-(4-pyridin-4-ylmethyl)-1-((2-(trimethylsilyl)ethoxy)methyl)-1H-imidazol-2-yl)cyclobutan-1-one (217 mg, 81%) as a brown oil. LCMS ESI-MS m/z:=358 [M+H]⁺.

Step 3: 3-(4-Pyridin-4-ylmethyl)-1-((2-(trimethylsilyl)ethoxy)methyl)-1H-imidazol-2-yl)-1-(trifluoromethyl)cyclobutan-1-ol. To a stirred solution of 3-(4-pyridin-4-ylmethyl)-1-((2-(trimethylsilyl)ethoxy)methyl)-1H-imidazol-2-yl)cyclobutan-1-one (217 mg, 0.61 mmol) in THF (2.2 mL) was added TMSCF₃ (173 mg, 1.2 mmol) over 5 min at rt under a nitrogen atmosphere. CsF (182 mg, 1.2 mmol) was then added in portions at rt under a nitrogen atmosphere. The resulting mixture was stirred for 4 h at rt. The resulting mixture was quenched with water (10 mL) and extracted with EtOAc (3×10 mL). The combined organic layers were washed with brine (10 mL), dried over anhydrous Na₂SO₄, filtered, and the filtrate was concentrated under reduced pressure. The crude product was purified by reverse phase flash chromatography to afford 3-(4-pyridin-4-ylmethyl)-1-((2-(trimethylsilyl)ethoxy)methyl)-1H-imidazol-2-yl)-1-(trifluoromethyl)cyclobutan-1-ol (130 mg, 50%) as a yellow solid. LCMS ESI-MS m/z:=428 [M+H]⁺.

Step 4: 3-(4-(Pyridin-4-ylmethyl)-1H-imidazol-2-yl)-1-(trifluoromethyl)cyclobutan-1-ol. To a stirred solution of 3-(4-pyridin-4-ylmethyl)-1-((2-(trimethylsilyl)ethoxy)methyl)-1H-imidazol-2-yl)-1-(trifluoromethyl)cyclobutan-1-ol (70 mg, 0.16 mmol) in DCM (0.7 mL) was added TFA (0.7 mL) for 4 h at rt under a nitrogen atmosphere. The resulting mixture was concentrated under reduced pressure. The residue was dissolved in DCM (5 mL) and H₂O (5 mL) and the mixture was basified to pH 9-10 with saturated Na₂CO₃. The resulting mixture was extracted with DCM (3×5 mL). The combined organic layers were concentrated under reduced pressure. The crude product was purified by Prep-HPLC to afford 3-(4-(pyridin-4-ylmethyl)-1H-imidazol-2-yl)-1-(trifluoromethyl)cyclobutan-1-ol (12 mg, 25%) as a white solid. ¹H NMR: (400 MHz, DMSO-d₆, ppm): δ 11.66 (d, J=30.9 Hz, 1H), 8.43 (d, J=5.3 Hz, 2H), 7.24 (d, J=4.7 Hz, 2H), 6.67 (t, J=24.6 Hz, 2H), 3.84 (d, J=22.8 Hz, 2H), 3.15 (t, J=8.9 Hz, 1H), 2.70 (t, J=10.3 Hz, 2H), 2.44 (d, J=11.5 Hz, 2H). LCMS ESI-MS m/z: 298 [M+H]⁺.

### Example 37A and 37B: (cis)-4-((2-(3-(Trifluoromethoxy)cyclobutyl)-1H-imidazole-4-yl)methyl)pyridine and (trans)-4-((2-(3-(Trifluoromethoxy)cyclobutyl)-1H-imidazole-4-yl)methyl)pyridine

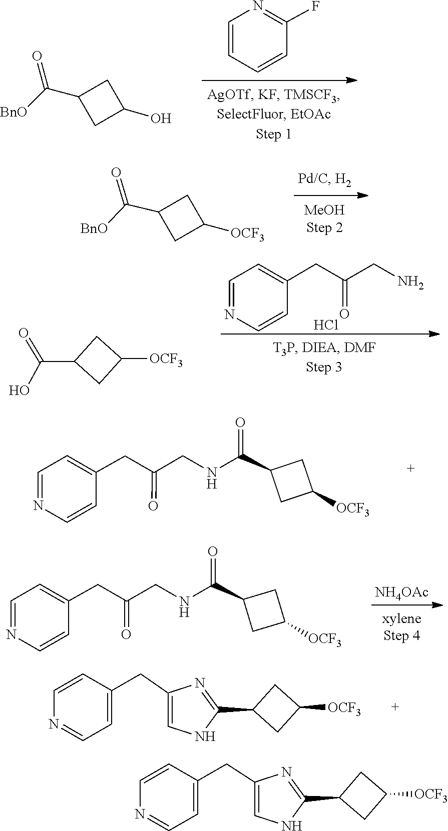

Step 1: Benzyl 3-(trifluoromethoxy)cylcobutane-1-carboxylate. To a stirred solution of benzyl 3-hydroxycyclobutane-1-carboxylate (9.0 g, 43.6 mmol), SelectFluor (5.6 g, 65.5 mmol), KF (10.1 g, 174.5 mmol), AgOTf (33.6 g, 130.9 mmol) in EtOAc (90 mL) was added TMSCF₃ (18.6 g, 130.9 mmol) and 2-fluoropyridine (6.4 g, 65.5 mmol) dropwise at rt under a nitrogen atmosphere. The resulting mixture was stirred overnight at rt under a nitrogen atmosphere. The resulting mixture was quenched with water (100 mL) at rt. The resulting mixture was extracted with EtOAc (3×50 mL). The combined organic layers were washed with brine (100 mL), dried over anhydrous Na₂SO₄, filtered, and concentrated under reduced pressure. The crude product was purified by silica gel column chromatography to afford benzyl 3-(trifluoromethoxy)cylcobutane-1-carboxylate (3.2 g, 27%) as a brown oil.

Step 2: 3-(Trifluoromethoxy)cyclobutene-1-carboxylic acid. A mixture of benzyl 3-(trifluoromethoxy) cyclobutane-1-carboxylate (3.2 g, 0.12 mmol) and Pd/C (0.50 g, 10% wt.) in MeOH (64 mL) was stirred overnight at rt under a hydrogen atmosphere. The resulting mixture was filtered, and the filter cake was rinsed in the funnel with MeOH (2×30 mL). The filtrate was collected and concentrated under reduced pressure to afford 3-(trifluoromethoxy)cyclobutene-1-carboxylic acid (1.6 g) as a brown oil. The crude product was used in the next step directly without further purification. LCMS ESI-MS m/z:=183 [M−H]⁻.

Step 3: (cis)-N-(2-oxo-3-(pyridine-4yl)propyl)-3-(trifluoromethoxy)cyclobutene-1-carboxamide and (trans)-N-(2-oxo-3-(pyridine-4yl)propyl)-3-(trifluoromethoxy)cyclobutene-1-carboxamide. To a stirred solution of 3-(trifluoromethoxy) cyclobutane-1-carboxylic acid (1.6 g, 8.7 mmol) and DIEA (3.4 g, 26.1 mol) in DMF (16 mL) was added T₃P (6.6 g, 10.4 mmol, 50% wt.) at rt under a nitrogen atmosphere and stirred for 5 min. To the resulting mixture was added 1-amino-3-(pyridin-4-yl) propan-2-one hydrochloride (1.6 g, 8.7 mmol) in portions at rt under a nitrogen atmosphere. The resulting mixture was stirred for 2 h at rt under a nitrogen atmosphere. The resulting mixture was quenched with water (20 mL) and extracted with EtOAc (3×20 mL). The combined organic layers were washed with brine (20 mL), dried over anhydrous Na₂SO₄, filtered, and concentrated under reduced pressure. The crude product was purified by prep-HPLC to afford (cis)-N-(2-oxo-3-(pyridine-4yl)propyl)-3-(trifluoromethoxy)cyclobutene-1-carboxamide (0.7 g, 2 steps, 19%) as a light yellow oil and (trans)-N-(2-oxo-3-(pyridine-4yl)propyl)-3-(trifluoromethoxy)cyclobutene-1-carboxamide (0.13 g, 2 steps, 3%) as a light yellow solid. LCMS ESI-MS m/z:=317 [M+H]⁺.

Step 4: (cis)-4-((2-(3-(Trifluoromethoxy)cyclobutyl)-1H-imidazole-4-yl)methyl)pyridine and (trans)-4-((2-(3-(Trifluoromethoxy)cyclobutyl)-1H-imidazole-4-yl)methyl)pyridine. A mixture of (cis)-N-(2-oxo-3-(pyridine-4yl)propyl)-3-(trifluoromethoxy)cyclobutene-1-carboxamide (368 mg, 1.16 mmol) and NH₄OAc (1.8 g, 23.2 mmol) in xylene (1.8 mL) was stirred and irradiated for 10 min at 140° C. in a microwave. The resulting mixture was cooled to rt and quenched by the addition of water (5 mL) at rt and extracted with EtOAc (3×5 mL). The combined organic layers were dried over anhydrous Na₂SO₄, filtered, and concentrated under reduced pressure. The crude product was purified by reverse phase flash chromatography to afford the crude product (230 mg) as a yellow oil. The crude product was purified by prep-HPLC to afford (cis)-4-((2-(3-(trifluoromethoxy)cyclobutyl)-1H-imidazole-4-yl)methyl)pyridine (108 mg, 16%) as a light yellow oil. ¹H NMR: (300 MHz, DMSO-d₆, ppm): δ 11.75 (s, 1H), 8.45 (d, J=5.3 Hz, 2H), 7.24 (d, J=6.4 Hz, 2H), 6.73 (s, 1H), 4.80 (p, J=7.6 Hz, 1H), 3.81 (s, 2H), 3.08-3.02 (m, 1H), 2.70-2.64 (m, 2H), 2.51-2.40 (m, 2H). LCMS ESI-MS m/z:=298 [M+H]⁺.

A mixture of (trans)-N-(2-oxo-3-(pyridine-4yl)propyl)-3-(trifluoromethoxy)cyclobutene-1-carboxamide (130 mg, 0.41 mmol) and NH₄OAc (633.7 mg, 8.2 mmol) in xylene (0.65 mL) was stirred and irradiated for 10 min at 140° C. in a microwave. The resulting mixture was cooled to rt. The resulting mixture was quenched by the addition of water (2 mL) at rt and extracted with EtOAc (3×3 mL). The combined organic layers were dried over anhydrous Na₂SO₄, filtered, and concentrated under reduced pressure. The crude product was purified by reverse phase flash chromatography to afford the crude product. The crude product was purified by prep-HPLC to afford (trans)-4-((2-(3-(trifluoromethoxy)cyclobutyl)-1H-imidazole-4-yl)methyl)pyridine (4.8 mg, 4%) as a white solid. ¹H NMR: (300 MHz, DMSO-d₆, ppm): δ 11.72-11.64 (m, 1H), 8.47-8.43 (m, 2H), 7.26-7.23 (m, 2H), 6.75-6.58 (m, 1H), 5.05 (q, J=6.9 Hz, 1H), 3.88-3.80 (m, 2H), 3.53-3.46 (m, 1H), 2.59-2.57 (m, 4H). LCMS ESI-MS m/z:=298 [M+H]⁺.

### Example 38A and 38B: (cis)-1-Fluoro-3-(4-(pyridine-4-ylmethyl)-1H-imidazol-2-yl)cyclobutene-1-carbonitrile and (trans)-1-Fluoro-3-(4-(pyridine-4-ylmethyl)-1H-imidazol-2-yl)cyclobutene-1-carbonitrile

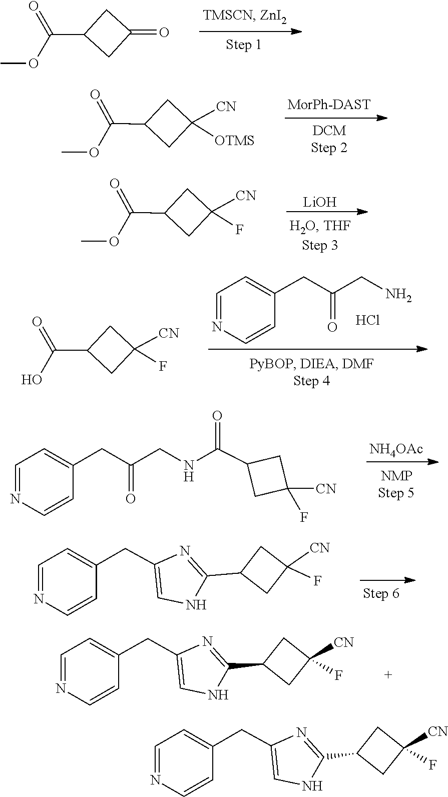

Step 1: Methyl 3-cyano-3-((trimethylsilyl)oxy)cyclobutene-1-carboxylate. To a stirred solution of methyl 3-oxocyclobutane-1-carboxylate (10 g, 78 mmol) and ZnI₂ (0.25 g, 0.8 mmol) was added TMSCN (15.5 g, 156.1 mmol) dropwise at 0-10° C. under a nitrogen atmosphere. The resulting mixture was stirred for 16 h at rt under a nitrogen atmosphere. The reaction mixture was purified by silica gel column chromatography to afford methyl 3-cyano-3-((trimethylsilyl)oxy)cyclobutene-1-carboxylate (13 g, 73%) as a light yellow oil.

Step 2: Methyl 3-cyano-3-fluorocyclobutane-1-carboxylate. To a stirred solution of methyl 3-cyano-3-((trimethylsilyl)oxy)cyclobutene-1-carboxylate (13 g, 57 mmol) in DCM (260 mL) was added 4-morpholinylsulfur trifluoride (1 g, 63 mmol) at 0° C. under a nitrogen atmosphere. The resulting mixture was stirred overnight at rt under a nitrogen atmosphere. The resulting mixture was poured into aqueous NaHCO₃ (100 mL). The resulting mixture was extracted with DCM (3×50 mL). The combined organic phase was washed with brine (1×100 mL), dried over anhydrous Na₂SO₄, filtered and concentrated under reduced pressure. The crude product was purified by silica gel column chromatography to afford methyl 3-cyano-3-fluorocyclobutane-1-carboxylate (5.6 g, yield: 62%) as a yellow oil.

Step 3: 3-Cyano-3-fluorocyclobutane-1-carboxylic acid. To a stirred solution of methyl 3-cyano-3-fluorocyclobutane-1-carboxylate (5.6 g, 35.6 mmol) in THF (28 mL) and H₂O (84 mL) was added LiOH (0.8 g, 35.6 mmol) at 0° C. The resulting mixture was stirred overnight at rt under a nitrogen atmosphere. The reaction was diluted with water (30 mL) at 0° C. and extracted with EtOAc (2×30 mL). The aqueous phase was acidified to pH 1-2 with HCl (3 M) and extracted with EtOAc (4×30 mL). The combined organic layers were dried over anhydrous Na₂SO₄, filtered, and concentrated under reduced pressure. The crude 3-cyano-3-fluorocyclobutane-1-carboxylic acid (3.1 g) was used in the next step directly without further purification. LCMS ESI-MS m/z:=142 [M−H]⁻.

Step 4: 3-Cyano-3-fluoro-N-(2-oxo-3-(pyridine-4-yl)propyl)cyclobutene-1-carboxamide. To a stirred solution of 3-cyano-3-fluorocyclobutane-1-carboxylic acid (3.1 g, 21.7 mmol) and DIEA (8.4 g, 65 mmol) in DMF (31 mL) was added PyBOP (16.9 g, 32.5 mmol) in portions at 0° C. The resulting mixture was stirred for 10 min at 0° C. and then added 1-amino-3-(pyridin-4-yl)propan-2-one hydrochloride (4.1 g, 21.7 mmol) in portions at 0° C. under a nitrogen atmosphere. The resulting mixture was stirred for 4 h at rt under a nitrogen atmosphere. The reaction was monitored by LCMS. The reaction was quenched with ice water (50 mL) and extracted with EtOAc (3×20 mL). The combined organic layers were washed with brine (10 mL), dried over anhydrous Na₂SO₄, filtered, and concentrated under reduced pressure. The crude product was purified by reverse phase flash chromatography and again by prep-HPLC to afford 3-cyano-3-fluoro-N-(2-oxo-3-(pyridine-4-yl)propyl)cyclobutene-1-carboxamide (0.11 g, 2 steps, 1%) as a yellow oil. LCMS ESI-MS m/z:=276 [M+H]⁺.

Step 5: 1-Fluoro-3-(4-(pyridine-4ylmethyl)-1H-imidazol-2-yl)cyclobutene-1-carbonitrile. To a stirred solution of 3-cyano-3-fluoro-N-(2-oxo-3-(pyridine-4-yl)propyl)cyclobutene-1-carboxamide (110 mg, 0.4 mmol) in NMP (2.2 mL) was added NH₄OAc (616 mg, 8 mmol) at rt under a nitrogen atmosphere. The reaction mixture was heated to 140° C. and stirred for 30 min with microwave radiation. The mixture was cooled to rt. The reaction was quenched with water (5 mL) at 0° C. and extracted with EtOAc (3×5 mL). The combined organic layers were washed with brine (5 mL), dried over anhydrous Na₂SO₄, filtered, and concentrated under reduced pressure. The crude product was purified by reverse phase flash chromatography to afford 1-fluoro-3-(4-(pyridine-4ylmethyl)-1H-imidazol-2-yl)cyclobutene-1-carbonitrile (20 mg, 71%) as a yellow oil. LCMS ESI-MS m/z:=257 [M+H]⁺.

Step 6: (cis)-1-Fluoro-3-(4-(pyridine-4-ylmethyl)-1H-imidazol-2-yl)cyclobutene-1-carbonitrile and (trans)-1-Fluoro-3-(4-(pyridine-4-ylmethyl)-1H-imidazol-2-yl)cyclobutene-1-carbonitrile. 1-fluoro-3-(4-(pyridine-4ylmethyl)-1H-imidazol-2-yl)cyclobutene-1-carbonitrile (20 mg) was separated by prep-HPLC to afford Peak 1 (0.8, 82%) as a light yellow oil. ¹H NMR: (400 MHz, DMSO-d₆, ppm): δ 12.01-11.47 (m, 1H), 8.55-8.38 (m, 2H), 7.33-7.17 (m, 2H), 6.92-6.54 (m, 1H), 3.95-3.67 (m, 3H), 3.12-2.88 (m, 4H). LCMS ESI-MS m/z:=257 [M+H]⁻. Peak 2 (0.4 mg) as a light-yellow oil. ¹H NMR: (400 MHz, DMSO-d₆, ppm): δ 11.94-11.73 (m, 1H), 8.52-8.40 (m, 2H), 7.29-7.20 (m, 2H), 6.88-6.56 (m, 1H), 3.93-3.71 (m, 2H), 3.31-3.24 (m, 1H), 3.20-3.07 (m, 2H), 2.93-2.77 (m, 2H). LCMS ESI-MS m/z:=257 [M+H]⁺. The absolute stereochemistry was not determined for either isomer.

### Example 39, 39A, 39B, 39C, and 38D 4-((2-((1R,2R)-2-Chloro-2-fluorocyclopropyl)-1H-imidazol-4-yl)methyl)pyridine, 4-((2-((1R,2S)-2-Chloro-2-fluorocyclopropyl)-1H-imidazol-4-yl)methyl)pyridine, 4-((2-((1S,2R)-2-Chloro-2-fluorocyclopropyl)-1H-imidazol-4-yl)methyl)pyridine, and 4-((2-((1S,2S)-2-Chloro-2-fluorocyclopropyl)-1H-imidazol-4-yl)methyl)pyridine

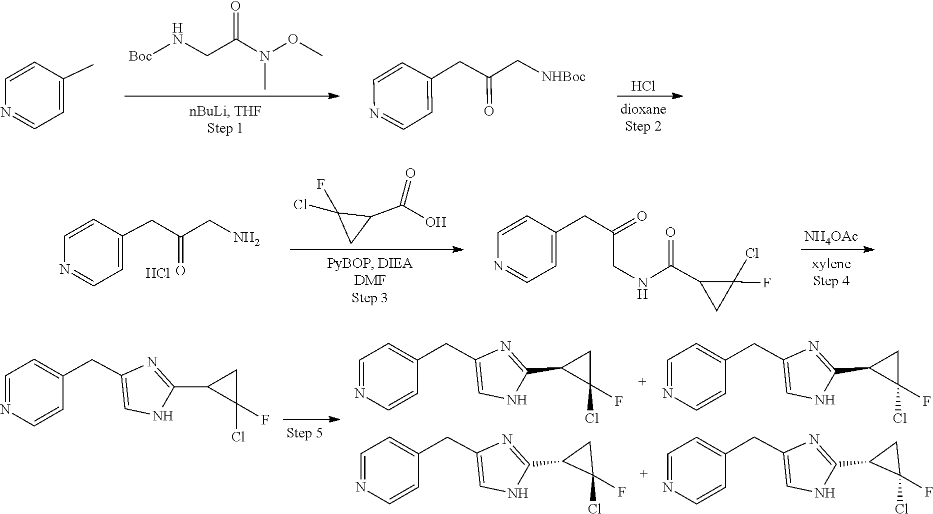

Step 1: tert-Butyl (2-oxo-3-(pyridine-4-yl)propyl)carbamate. To a stirred solution of 4-methylpyridine (10 g, 107 mmol) in THF (200 mL) was added n-BuLi (55.8 mL, 139.6 mmol, 2.5 M) dropwise at −70° C. under a nitrogen atmosphere. The resulting mixture was stirred for 30 min at −70° C. under a nitrogen atmosphere. To the mixture was added tert-butyl (2-(methoxy(methyl)amino)-2-oxoethyl)carbamate (30.5 g, 139.6 mmol) in THF (100 mL) dropwise at −70° C. The resulting mixture was stirred for an additional 1 h at −70° C. under a nitrogen atmosphere. The resulting mixture was quenched by dropping aqueous saturated NH₄Cl (300 mL) at −70° C. The mixture was warmed to rt and extracted with EtOAc (3×300 mL). The combined organic layers were washed with brine (1×300 mL), dried over anhydrous Na₂SO₄, filtered, and concentrated under reduced pressure. The crude product was purified by silica gel column chromatography to afford tert-butyl (2-oxo-3-(pyridine-4-yl)propyl)carbamate (4.5 g, 17%) as a yellow oil. LCMS ESI-MS m/z:=251 [M+H]⁺.

Step 2: 1-Amino-3-(pyridin-4-yl)propan-2-one hydrochloride. To a stirred solution of tert-butyl (2-oxo-3-(pyridine-4-yl)propyl)carbamate (4.5 g, 18 mmol) in dioxane (45 mL) was added HCl/dioxane (22.5 mL, 4 M) dropwise at rt under a nitrogen atmosphere. The resulting mixture was stirred for an additional 3 h at rt under a nitrogen atmosphere. The resulting mixture was filtered, and the filter cake was rinsed in the funnel with EA (50 mL). The filter cake was dried under reduced pressure to afford 1-amino-3-(pyridin-4-yl)propan-2-one hydrochloride (2.1 g) as a yellow solid. The crude product was used in the next step directly without further purification. LCMS EI-MS m/z:=151 [M+H-HCl]⁺.

Step 3: 2-Chloro-2-fluoro-N-(2-oxo-3-(pyridin-4-yl)propyl)cyclopropane-1-carboxamide. To a stirred mixture of 2-chloro-2-fluorocyclopropane-1-carboxylic acid (2.0 g, 14.4 mmol) and DIEA (5.6 g, 43.3 mmol) in DMF (20 mL) was added PyBOP (11.3 g, 21.7 mmol) in portions at 0° C. under a nitrogen atmosphere. The resulting mixture was stirred for 10 min. 1-amino-3-(pyridin-4-yl) propan-2-one hydrochloride (3.2 g, 17.3 mmol) in portions at 0° C. and the resulting mixture was stirred for another 4 h at rt under a nitrogen atmosphere. The reaction was quenched with water (100 mL) at 0° C. and extracted with EtOAc (3×50 mL). The combined organic layers were washed with brine (1×100 mL), dried over anhydrous Na₂SO₄, filtered, and concentrated under reduced pressure. The crude product was purified by reverse phase flash chromatography to afford 2-chloro-2-fluoro-N-(2-oxo-3-(pyridin-4-yl)propyl)cyclopropane-1-carboxamide (1.7 g, 34% (2 steps)) as a brown oil. LCMS EI-MS m/z:=271 [M+H]⁺.

Step 4: 4-((2-(2-Chloro-2-fluorocyclopropyl)-1H-imidazol-4-yl)methyl)pyridine. To a stirred solution of 2-chloro-2-fluoro-N-(2-oxo-3-(pyridin-4-yl)propyl)cyclopropane-1-carboxamide (1.7 g, 6.1 mmol) and NH₄OAc (9.5 g, 122.6 mmol) in xylene (17 mL) was stirred and irradiated for 30 min at 140° C. in a microwave. The resulting mixture was cooled to rt. The resulting mixture was quenched with ice water (10 mL) and extracted with EtOAc (3×10 mL). The combined organic phase was washed with brine (1×10 mL), dried over anhydrous Na₂SO₄, filtered, and concentrated under reduced pressure. The crude product was purified by reverse phase flash chromatography and prep HPLC to afford 4-((2-(2-chloro-2-fluorocyclopropyl)-1H-imidazol-4-yl)methyl)pyridine (0.12 g, 7%) as a yellow oil. ¹H NMR: (400 MHz, DMSO-d₆, ppm) δ 11.95 (s, 1H), 8.53-8.35 (m, 2H), 7.22 (td, J=4.4, 1.6 Hz, 2H), 6.81 (s, 1H), 3.83 (s, 2H), 3.08-2.81 (m, 1H), 2.32-2.07 (m, 1H), 2.04-1.85 (m, 1H). LCMS EI-MS m/z:=252 [M+H]⁺.

Step 5: 4-((2-((1R,2R)-2-chloro-2-fluorocyclopropyl)-1H-imidazol-4-yl)methyl)pyridine, 4-((2-((1R,2S)-2-chloro-2-fluorocyclopropyl)-1H-imidazol-4-yl)methyl)pyridine, 4-((2-((1S,2R)-2-chloro-2-fluorocyclopropyl)-1H-imidazol-4-yl)methyl)pyridine, and -((1S,2S)-2-chloro-2-fluorocyclopropyl)-1H-imidazol-4-yl)methyl)pyridine. 4-((2-(2-chloro-2-fluorocyclopropyl)-1H-imidazol-4-yl)methyl)pyridine (118 mg) was separated by prep-chiral-HPLC to afford Peak 1 (12.5 mg, 11%) as brown solid. ¹H NMR: (400 MHz, DMSO-d₆, ppm) δ 11.96 (s, 1H), 8.44 (d, J=5.5 Hz, 2H), 7.38-7.05 (m, 2H), 6.80 (s, 1H), 3.83 (s, 2H), 3.03 (ddd, J=14.7, 11.8, 8.3 Hz, 1H), 2.14 (ddd, J=16.4, 11.8, 7.7 Hz, 1H), 1.99 (q, J=8.0 Hz, 1H). LCMS EI-MS m/z:=252 [M+H]⁺. Peak 2 (2 mg, 2%) as brown solid. ¹H NMR: (400 MHz, DMSO-d₆, ppm) δ 12.12-11.81 (m, 1H), 8.58-8.3 5 (m, 2H), 7.23 (d, J=5.0 Hz, 2H), 7.69-7.49 (m, 1H), 3.98-3.73 (m, 2H), 2.91-2.78 (m, 1H), 2.27 (dt, J=16.3, 7.9 Hz, 1H), 1.91 (dt, J=11.2, 7.9 Hz, 1H). LCMS EI-MS m/z:=252 [M+H]⁺. Peak 3 (1.2 mg, 1%) as a yellow solid. ¹H NMR: (400 MHz, DMSO-d₆, ppm) δ 12.13-11.73 (m, 1H), 8.64-8.17 (m, 2H), 7.36-6.49 (m, 3H), 4.04-3.70 (m, 2H), 3.13-2.91 (m, 1H), 2.14 (ddd, J=16.4, 11.8, 7.7 Hz, 1H), 2.00 (q, J=8.0 Hz, 1H). LCMS EI-MS m/z:=252 [M+H]⁺. Peak 4 (1.2 mg, 10%) as a yellow solid. ¹H NMR: (400 MHz, DMSO-d₆, ppm) δ 12.12-11.76 (m, 1H), 8.62-8.30 (m, 2H), 7.26-6.59 (m, 3H), 3.99-3.71 (m, 2H), 2.86 (ddd, J=11.3, 8.1, 1.6 Hz, 1H), 2.32-2.21 (m, 1H), 1.91 (dt, J=11.4, 8.0 Hz, 1H). LCMS EI-MS m/z: =252 [M+H]⁺. The absolute stereochemistry was not determined for either isomer.

### Example 40: 4-((2-(1,2,2-Trifluorocyclopropyl)-1H-imidazol-4-yl)methyl)pyridine

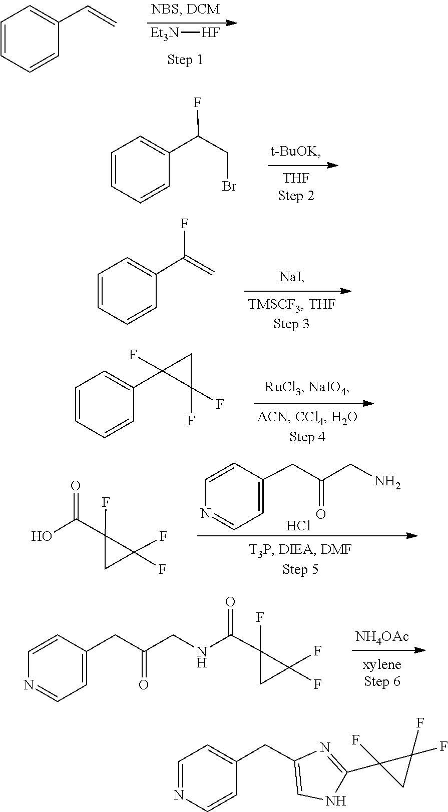

Step 1: (2-Bromo-1-fluoroethyl)benzene. To a stirred solution of styrene (30 g, 288 mmol) and NBS (76.9 g, 432.1 mmol) in DCM (480 mL) was added Et₃N·HF (69.6 g, 432.1 mmol) dropwise at 0° C. under a nitrogen atmosphere. The resulting mixture was stirred for 16 h at rt under a nitrogen atmosphere. The reaction was quenched with water (300 mL) at 0° C. The mixture was basified to pH 8 with aqueous saturated NaHCO₃ and extracted with DCM (3×50 mL). The combined organic layers were washed with brine (200 mL), dried over anhydrous Na₂SO₄, filtered, and concentrated under reduced pressure. The crude product was purified by silica gel column chromatography to afford (2-bromo-1-fluoroethyl)benzene (24 g, 41%) as a yellow oil.

Step 2: (1-Fluorovinyl)benzene. To a stirred solution of (2-bromo-1-fluoroethyl)benzene (24 g, 118 mmol) in THF (240 mL) was added t-BuOK (19.9 g, 177.3 mmol) in portions at 0° C. under a nitrogen atmosphere. The resulting mixture was stirred for 16 h at rt under a nitrogen atmosphere. The resulting mixture was filtered and the filter cake was rinsed in the funnel with THF (3×20 mL). The filtrate was concentrated under reduced pressure. The crude product (8 g) was used in the next step directly without further purification.

Step 3: (1,2,2-Trifluorocyclopropyl)benzene. To a stirred solution of (1-fluorovinyl)benzene (8.0 g, 65.5 mmol) and NaI (24.5 g, 163.7 mmol) in THF (80 mL) was added TMSCF₃ (23.3 g, 163.7 mmol) at rt under a nitrogen atmosphere. The resulting mixture was stirred for 16 h at 80° C. under a nitrogen atmosphere. The mixture was cooled to rt. The reaction was monitored by TLC. The reaction was quenched with water (60 mL) at 0° C. and extracted with EtOAc (3×30 mL). The combined organic layers were washed with brine (50 mL), dried over anhydrous Na₂SO₄, filtered, and concentrated under reduced pressure. The crude product was purified by silica gel column chromatography to afford (1,2,2-trifluorocyclopropyl)benzene (4.5 g, 2 steps 22%) as a brown oil.

Step 4: Trifluorocyclopropane-1-carboxylic acid. To a stirred solution of (1,2,2-trifluorocyclopropyl)benzene (4.5 g, 26.1 mmol) and NaIO₄ (44.2 g, 206.5 mmol) in H₂O (9 mL), ACN (13.5 mL) and CCl₄ (13.5 mL) was added RuCl₃ (0.54 g, 2.6 mmol) at rt under a nitrogen atmosphere. The resulting mixture was stirred for 3 days at 90° C. under a nitrogen atmosphere. The mixture was cooled to rt. The reaction was diluted with water (30 mL). The resulting mixture was extracted with DCM (2×20 mL). The aqueous phase was acidified to pH 1 with HCl (6 M) and extracted with DCM (4×30 mL). The combined organic layers were dried over anhydrous Na₂SO₄, filtered, and concentrated under reduced pressure. The crude product (2.5 g) was used in the next step directly without further purification. LCMS ESI-MS m/z:=139 [M−H]⁻.

Step 5: 1,2,2-Trifluoro-N-(2-oxo-3-(pyridine-4-yl)propyl)cyclopropane-1-carboxamide. To a stirred solution of trifluorocyclopropane-1-carboxylic acid (500 mg, 3.6 mmol) and DIEA (1.4 g, 10.7 mmol) in DMF (5 mL) was added T₃P (2.7 g, 4.3 mmol, 50% wt.) at 0° C. The resulting mixture was stirred for 5 min at 0° C. and added 1-amino-3-(pyridin-4-yl)propan-2-one hydrochloride (799.5 mg, 4.3 mmol) at 0° C. under a nitrogen atmosphere. The resulting mixture was stirred for 2 h at rt. The reaction was quenched with water (20 mL) at 0° C. and extracted with EtOAc (3×10 mL). The combined organic layers were washed with brine (10 mL), dried over anhydrous Na₂SO₄, filtered, and concentrated under reduced pressure. The crude product was purified by reverse phase flash chromatography to afford 1,2,2-trifluoro-N-(2-oxo-3-(pyridine-4-yl)propyl)cyclopropane-1-carboxamide (130 mg, 2 steps, 85%) as a yellow solid. LCMS ESI-MS m/z:=273 [M+H]⁺.

Step 6: 4-((2-(1,2,2-Trifluorocyclopropyl)-1H-imidazol-4-yl)methyl)pyridine. To a stirred solution of 1,2,2-trifluoro-N-(2-oxo-3-(pyridine-4-yl)propyl)cyclopropane-1-carboxamide (130 mg, 0.41 mmol) and NH₄OAc (626 mg, 8.1 mmol) in xylene (2.6 mL) at rt under a nitrogen atmosphere. The resulting mixture was stirred for 3 h at 140° C. under a nitrogen atmosphere. The mixture was cooled to rt. The reaction was diluted with water (5 mL) at 0° C. and extracted with EtOAc (4×5 mL). The combined organic layers were dried over anhydrous Na₂SO₄, filtered, and concentrated under reduced pressure. The crude product was purified by reverse phase flash chromatography to afford the crude product. The crude product was purified by prep-HPLC to afford 4-((2-(1,2,2-trifluorocyclopropyl)-1H-imidazol-4-yl)methyl)pyridine (1.8 mg, 1.7%) as a brown oil. ¹H NMR: (400 MHz, DMSO-d₆, ppm): δ 12.90-12.69 (m, 1H), 8.52-8.41 (m, 2H), 7.28-7.20 (m, 2H), 7.15-6.78 (m, 1H), 4.01-3.81 (m, 2H), 2.62-2.52 (m, 2H). LCMS EI-MS m/z:=254 [M+H]⁺.

### Example A

### Biological Assay

Each reaction was run at a volume of 20 μL containing 50 μM compound (dissolved in DMSO; final concentration of DMSO is 1% v/v), 40 nM human SARM1₍₅₀₋₇₂₄₎, 0.3 mM NMN, 20 μM NAD, 1 mM TCEP, 25 mM HEPES pH 7.4, 10 mM KCl and 10 mM MgCl₂. The reaction was incubated at room temperature for 60 minutes and quenched with 20 μL of 0.4% formic acid. The samples were run on Agilent HPLC 1260 Infinity II with Synergi 2.5 μM Fusion-RP 100 Å (100×3.0 mm) LC column from Phenomenex. Total run time for each sample was 4 minutes. The run was isocratic with 1.5% methanol in 40 mM ammonium acetate pH 6.0. Samples were run at a flow rate of 0.8 mL/min at 55° C. Peak areas of NAD and NAM were determined using OpenLAB CDS (Chem Station edition) software. For dose-response, the compound was diluted serially 1:3 in DMSO and added to the reaction starting at a final compound concentration of 100 μM in 1% DMSO.

IC₅₀ data according to the assay described above is provided in Table 1.

(IC₅₀<1 μM (***); 1 μM≤IC₅₀<10 μM (**); 10 μM≤IC₅₀ 100 μM (*)); and NT=not tested.

TABLE 1

| | Average |
|---|---|
| Ex. No. | IC₅₀ (μM) |
| 1 | ** |
| 2 | ** |
| 3 | ** |
| 4 | *** |
| 4A | * |
| 5A | *** |
| 5B | *** |
| 6 | *** |
| 7 | ** |
| 8 | ** |
| 9 | *** |
| 10A | *** |
| 10B | *** |
| 11A | *** |
| 11B | ** |
| 12 | ** |
| 13 | ** |
| 14 | *** |
| 15A | *** |
| 15B | * |
| 16 | ** |
| 17A | ** |
| 17B | * |
| 18A | ** |
| 18B | * |
| 19A | *** |
| 19B | ** |
| 20A | NT |
| 20B | * |
| 21A | *** |
| 21B | *** |
| 22 | *** |
| 23A | *** |
| 23B | ** |
| 24 | ** |
| 25 | *** |
| 26 | ** |
| 27 | * |
| 28 | *** |
| 29 | *** |
| 30 | ** |
| 31A | ** |
| 31B | ** |
| 32 | ** |
| 33A | *** |
| 33B | *** |
| 34 | *** |
| 35A | ** |
| 35B | *** |
| 36 | ** |
| 37A | *** |
| 37B | ** |
| 38A | ** |
| 38B | ** |
| 39 | *** |
| 39A | *** |
| 39B | *** |
| 39C | *** |
| 39D | *** |
| 40 | *** |

The examples and embodiments described herein are for illustrative purposes only and in some embodiments, various modifications or changes are to be included within the purview of disclosure and scope of the appended claims.

What is claimed is:

1. A compound of Formula (I′): 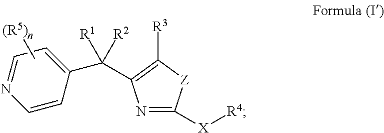
or a pharmaceutically acceptable salt thereof, wherein:
Z is selected from O or N(R⁶);
X is a bond or —C(R⁷)(R⁸)—;
R¹ is H or C₁₋₆alkyl;
R² is H or C₁₋₆alkyl;
R³ is selected from H, halogen, C₁₋₆alkyl, and C₁₋₆haloalkyl;
R⁴ is selected from C₃₋₆cycloalkyl and C₂₋₉heterocycloalkyl, wherein C₃₋₆cycloalkyl and C₂₋₉heterocycloalkyl are substituted with 1, 2, 3, 4, or 5 groups independently selected from halogen, —CN, C₁₋₆haloalkyl, and C₁₋₄haloalkoxy, and wherein C₃₋₆cycloalkyl and C₂₋₉heterocycloalkyl are optionally substituted with 1, 2, 3, 4, or 5 groups independently selected from —OH, —NH₂, —N(H)(C₁₋₄alkyl), —N(C₁₋₄alkyl)₂, C₁₋₄alkyl, and C₁₋₄alkoxy;
each R⁵ is independently selected from halogen, C₁₋₆alkyl, and C₁₋₆haloalkyl;
R⁶ is selected from H and C₁₋₆alkyl;
R⁷ and R⁸ are each independently selected from H, —OR⁹, C₁₋₄alkyl, and C₁₋₄haloalkyl;
each R⁹ is independently selected from H, C₁₋₆alkyl, and C₁₋₆haloalkyl, wherein said C₁₋₆alkyl is optionally substituted with 1, 2, or 3 substituents independently selected from —OH, —CN, —NH₂, —N(H)(C₁₋₄alkyl), —N(C₁₋₄alkyl)₂, halogen, C₁₋₄alkyl, C₁₋₄alkoxy, C₁₋₄haloalkyl, and C₁₋₄haloalkoxy; and
n is 0, 1, 2, 3, or 4.

2. A compound of Formula (I): 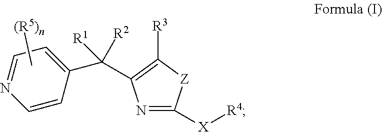
or a pharmaceutically acceptable salt thereof, wherein:
Z is selected from O or N(R⁶);
X is a bond or —C(R⁷)(R⁸)—;
R¹ is H or C₁₋₆alkyl;
R² is H or C₁₋₆alkyl;
R³ is selected from H, halogen, C₁₋₆alkyl, and C₁₋₆haloalkyl;
R⁴ is selected from C₃₋₆cycloalkyl and C₂₋₉heterocycloalkyl, wherein C₃₋₆cycloalkyl and C₂₋₉heterocycloalkyl are substituted with 1, 2, 3, 4, or 5 groups independently selected from halogen and C₁₋₆haloalkyl and wherein C₃₋₆cycloalkyl and C₂₋₉heterocycloalkyl are optionally substituted with 1, 2, 3, 4, or 5 groups independently selected from —OH, —CN, —NH₂, —N(H)(C₁₋₄ alkyl), —N(C₁₋₄alkyl)₂, C₁₋₄alkyl, C₁₋₄alkoxy, and C₁₋₄haloalkoxy;
each R⁵ is independently selected from halogen, C₁₋₆alkyl, and C₁₋₆haloalkyl;
R⁶ is selected from H and C₁₋₆alkyl;
R⁷ and R⁸ are each independently selected from H, —OR⁹, C₁₋₄alkyl, and C₁₋₄haloalkyl;
each R⁹ is independently selected from H, C₁₋₆alkyl, and C₁₋₆haloalkyl, wherein said C₁₋₆alkyl is optionally substituted with 1, 2, or 3 substituents independently selected from —OH, —CN, —NH₂, —N(H)(C₁₋₄alkyl), —N(C₁₋₄alkyl)₂, halogen, C₁₋₄alkyl, C₁₋₄alkoxy, C₁₋₄haloalkyl, and C₁₋₄haloalkoxy; and
n is 0, 1, 2, 3, or 4.

3. The compound of claim 1 or claim 2, or a pharmaceutically acceptable salt thereof, wherein X is a bond.

4. The compound of claim 1 or claim 2, or a pharmaceutically acceptable salt thereof, wherein X is —C(R⁷)(R⁸)—.

5. The compound of claim 4, or a pharmaceutically acceptable salt thereof, wherein X is —CH₂—.

6. The compound of any one of claims 1-5, or a pharmaceutically acceptable salt thereof, wherein Z is N(R⁶).

7. The compound of claim 6, or a pharmaceutically acceptable salt thereof, wherein R⁶ is H.

8. The compound of claim 6, or a pharmaceutically acceptable salt thereof, wherein R⁶ is C₁₋₆alkyl.

9. The compound of any one of claims 1-5, or a pharmaceutically acceptable salt thereof, wherein Z is O.

10. The compound of any one of claims 1-8, or a pharmaceutically acceptable salt thereof, wherein R¹ is H.

11. The compound of any one of claims 1-10, or a pharmaceutically acceptable salt thereof, wherein R² is H.

12. The compound of any one of claims 1-10, or a pharmaceutically acceptable salt thereof, wherein R² is C₁₋₆alkyl.

13. The compound of any one of claims 1-12, or a pharmaceutically acceptable salt thereof, wherein R³ is H.

14. The compound of claim 1, or a pharmaceutically acceptable salt thereof, having Formula (Ia′):

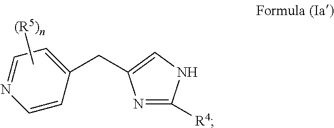

15. The compound of claim 1, or a pharmaceutically acceptable salt thereof, having Formula (Ib′):

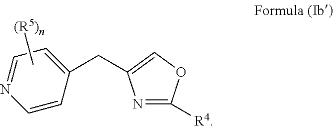

16. The compound of claim 1, or a pharmaceutically acceptable salt thereof, having Formula (Ic′):

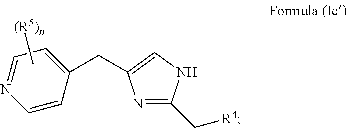

17. The compound of claim 2, or a pharmaceutically acceptable salt thereof, having Formula (Ia):

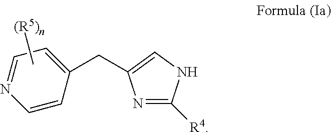

18. The compound of claim 2, or a pharmaceutically acceptable salt thereof, having Formula (Ib):

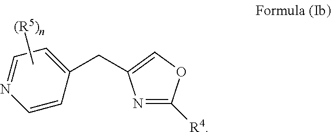

19. The compound of claim 2, or a pharmaceutically acceptable salt thereof, having Formula (Ic):

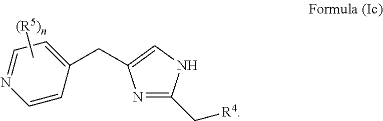

20. The compound of any one of claims 1-16, or a pharmaceutically acceptable salt thereof, wherein R⁴ is C₃₋₆cycloalkyl substituted with 1, 2, 3, 4, or 5 groups independently selected from halogen, —CN, C₁₋₆haloalkyl, and C₁₋₄haloalkoxy, wherein said C₃₋₆cycloalkyl is optionally substituted with 1, 2, 3, 4, or 5 groups independently selected from —OH, —NH₂, —N(H)(C₁₋₄alkyl), —N(C₁₋₄alkyl)₂, C₁₋₄alkyl, and C₁₋₄alkoxy.

21. The compound of claim 20, or a pharmaceutically acceptable salt thereof, wherein R⁴ is C₃₋₆ cycloalkyl substituted with 1, 2, or 3 groups independently selected from halogen, —CN, C₁₋₆ haloalkyl, and C₁₋₄haloalkoxy, wherein said C₃₋₆cycloalkyl is optionally substituted with 1 or 2 groups independently selected from —OH, C₁₋₄alkyl, and C₁₋₄alkoxy.

22. The compound of any one of claims 2-13 and 17-19, or a pharmaceutically acceptable salt thereof, wherein R⁴ is C₃₋₆cycloalkyl substituted with 1, 2, 3, 4, or 5 groups independently selected from halogen and C₁₋₆haloalkyl, and wherein said C₃₋₆cycloalkyl is optionally substituted with 1, 2, 3, 4, or 5 groups independently selected from —OH, —CN, —NH₂, —N(H)(C₁₋₄alkyl), —N(C₁₋₄alkyl)₂, C₁₋₄alkyl, C₁₋₄alkoxy, and C₁₋₄haloalkoxy.

23. The compound of claim 22, or a pharmaceutically acceptable salt thereof, wherein R⁴ is C₃₋₆ cycloalkyl substituted with 1, 2, 3, or 4 groups independently selected from halogen and C₁₋₆haloalkyl, and wherein said C₃₋₆cycloalkyl is optionally substituted with 1, 2, or 3 groups independently selected from —OH, —CN, C₁₋₄alkyl, C₁₋₄alkoxy, and C₁₋₄haloalkoxy.

24. The compound of any one of claims 1-23, or a pharmaceutically acceptable salt thereof, wherein R⁴ is C₃₋₆cycloalkyl substituted only with 1, 2, or 3 groups independently selected from halogen and C₁₋₆haloalkyl.

25. The compound of any one of claims 1-16, or a pharmaceutically acceptable salt thereof, wherein R⁴ is selected from:

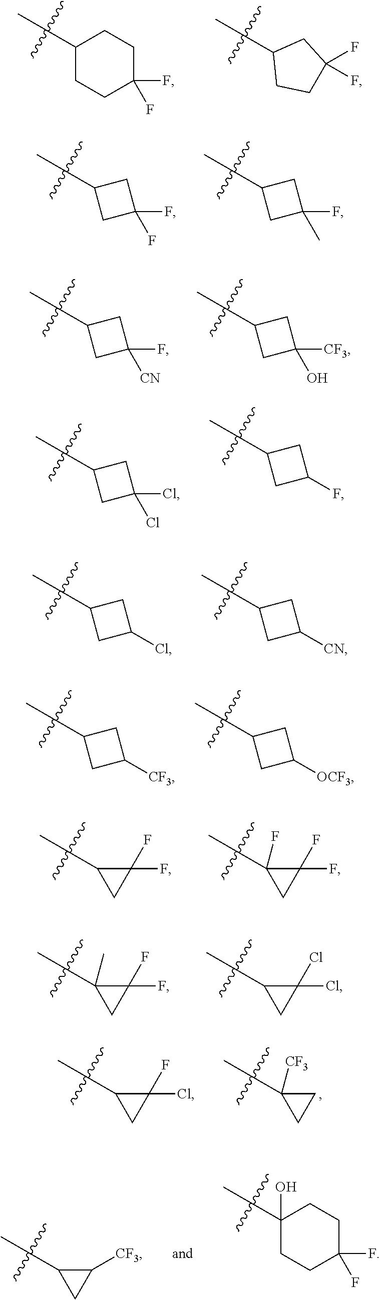

26. The compound of any one of claims 1-19, or a pharmaceutically acceptable salt thereof, wherein R⁴ is selected from:

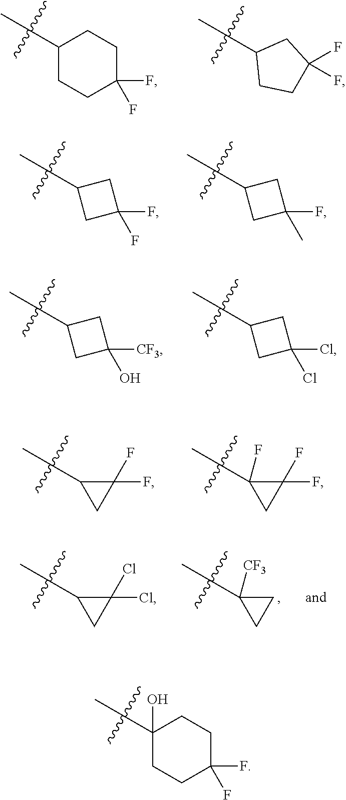

27. The compound of any one of claims 1-16, or a pharmaceutically acceptable salt thereof, wherein R⁴ is C₂₋₉heterocycloalkyl substituted with 1, 2, 3, 4, or 5 groups independently selected from halogen, —CN, C₁₋₆haloalkyl, and C₁₋₄haloalkoxy, wherein said C₂₋₉heterocycloalkyl is optionally substituted with 1, 2, 3, 4, or 5 groups independently selected from —OH, —NH₂, —N(H)(C₁₋₄alkyl), —N(C₁₋₄alkyl)₂, C₁₋₄alkyl, and C₁₋₄alkoxy.

28. The compound of claim 22, or a pharmaceutically acceptable salt thereof, wherein R⁴ is C₂₋₉heterocycloalkyl substituted with 1, 2, or 3 groups independently selected from halogen, —CN, C₁₋₆haloalkyl, and C₁₋₄haloalkoxy, wherein said C₂₋₉heterocycloalkyl is optionally substituted with 1 or 2 groups independently selected from —OH, C₁₋₄alkyl, and C₁₋₄alkoxy.

29. The compound of any one of claims 2-13 and 17-19, or a pharmaceutically acceptable salt thereof, wherein R⁴ is C₂₋₉heterocycloalkyl substituted with 1, 2, 3, 4, or 5 groups independently selected from halogen and C₁₋₆haloalkyl, and wherein said C₂₋₉heterocycloalkyl is optionally substituted with 1, 2, 3, 4, or 5 groups independently selected from —OH, —CN, —NH₂, —N(H)(C₁₋₄alkyl), —N(C₁₋₄alkyl)₂, C₁₋₄alkyl, C₁₋₄alkoxy, and C₁₋₄ haloalkoxy.

30. The compound of claim 28 or claim 29, or a pharmaceutically acceptable salt thereof, wherein R⁴ is C₂₋₉heterocycloalkyl substituted with 1, 2, 3, or 4 groups independently selected from halogen.

31. The compound of any one of claims 27-30, or a pharmaceutically acceptable salt thereof, wherein R⁴ is selected from:

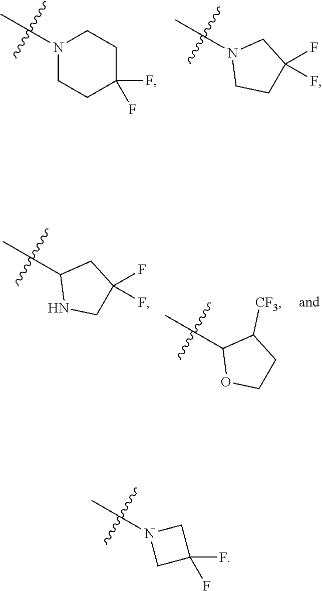 [SMILES: *N1CCC(F)(F)CC1 ; *N1CCC(F)(F)C1 ; *C1CC(F)(F)CN1 ; *C1OCCC1C(F)(F)F ; *N1CC(F)(F)C1]

32. The compound of claim 31, or a pharmaceutically acceptable salt thereof, wherein R⁴ is selected from:

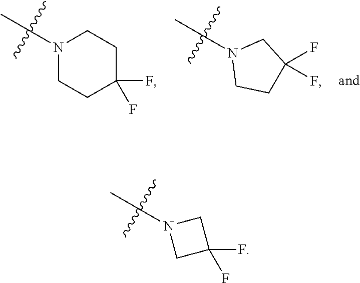 [SMILES: *N1CCC(F)(F)CC1 ; *N1CCC(F)(F)C1 ; *N1CC(F)(F)C1]

33. The compound of any one of claims 1-32, or a pharmaceutically acceptable salt thereof, wherein n is 0.

34. The compound of claim 1 or claim 2, or a pharmaceutically acceptable salt thereof, selected from:

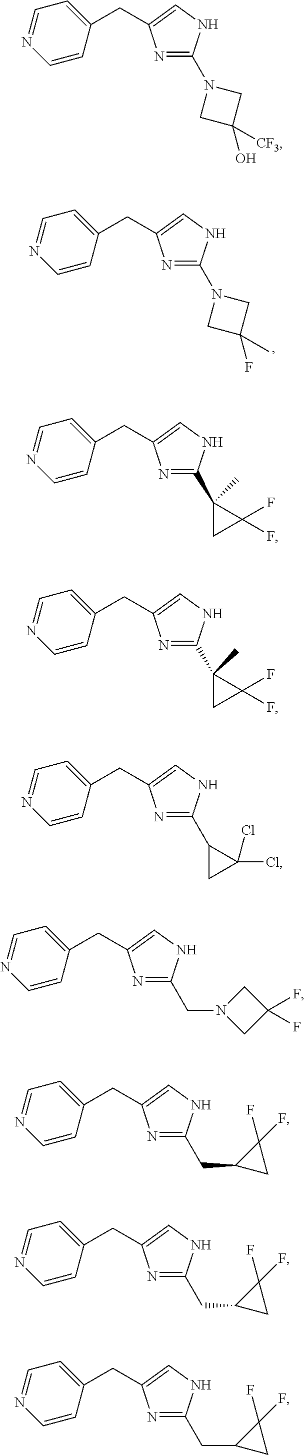

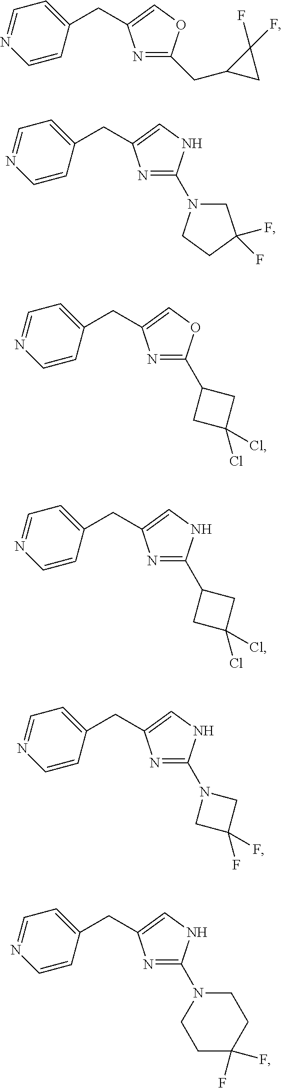 [SMILES: FC1(F)CC1Cc1nc(Cc2ccncc2)co1 ; FC1(F)CCN(c2nc(Cc3ccncc3)c[nH]2)C1 ; ClC1(Cl)CC(c2nc(Cc3ccncc3)co2)C1 ; ClC1(Cl)CC(c2nc(Cc3ccncc3)c[nH]2)C1 ; FC1(F)CN(c2nc(Cc3ccncc3)c[nH]2)C1 ; FC1(F)CCN(c2nc(Cc3ccncc3)c[nH]2)CC1]

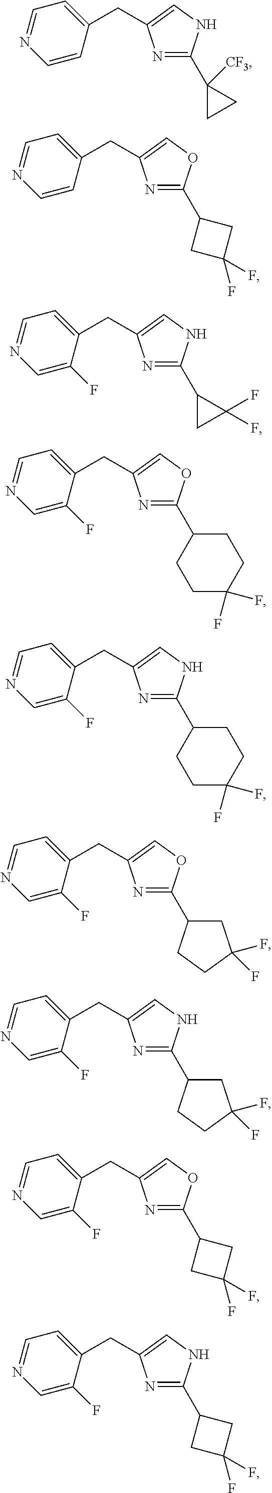

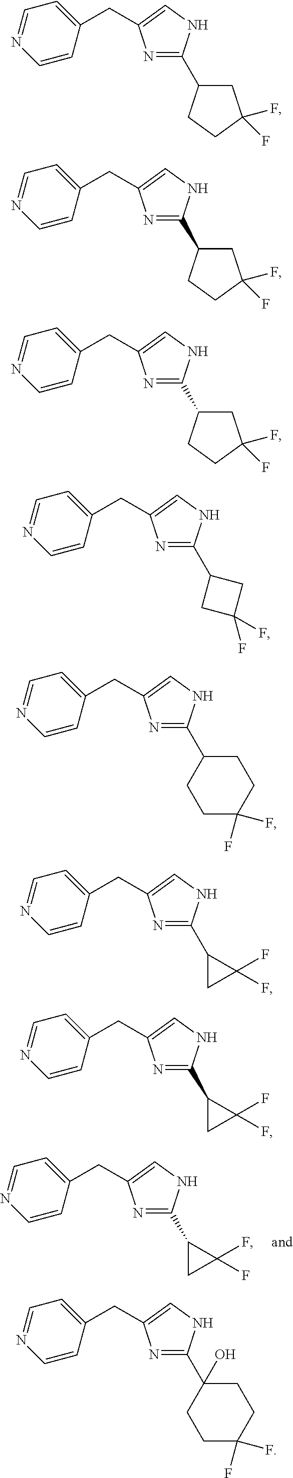

35. The compound of claim 1 or claim 2, or a pharmaceutically acceptable salt thereof, selected from:

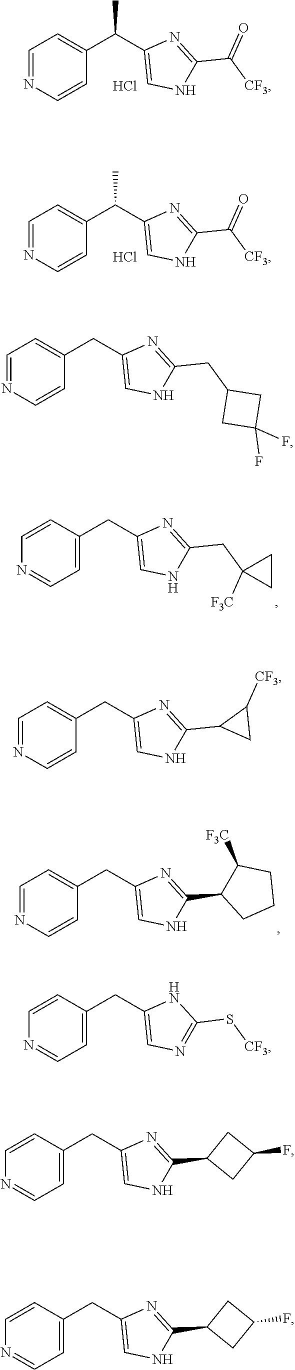

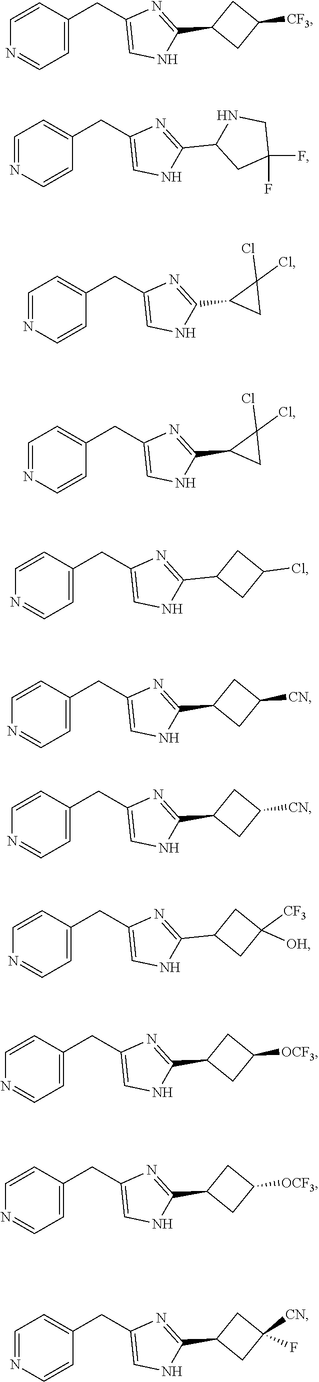

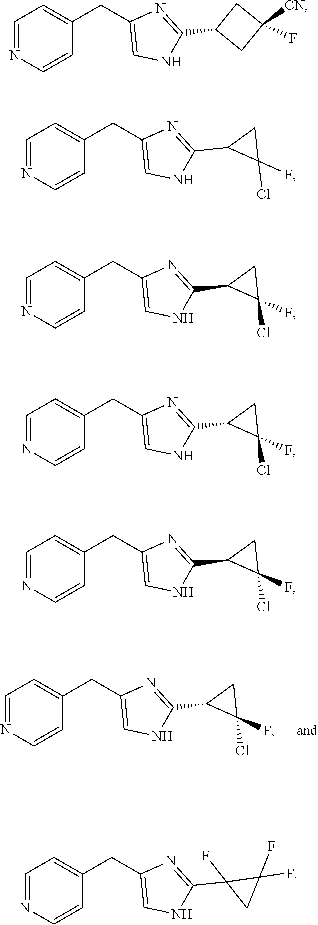

36. A pharmaceutical composition comprising at least one pharmaceutically acceptable carrier and a compound of any one of claims 1-35, or a pharmaceutically acceptable salt or solvate thereof.

37. A method of inhibiting SARM1 comprising contacting the SARM1 with a compound of any one of claims 1-35, or a pharmaceutically acceptable salt or solvate thereof.

38. A method of inhibiting axonal degeneration in a patient in need thereof comprising administering to the patient an inhibiting amount of a compound of any one of claims 1-35, or a pharmaceutically acceptable salt or solvate thereof.

39. The method of claim 38, wherein the axonal degeneration is caused by abnormal reduction or depletion of NAD+ in the axons.

40. A method of treating or preventing a neurological disorder in a patient in need thereof comprising administering to the patient a therapeutically effective amount of a compound of any one of claims 1-35, or a pharmaceutically acceptable salt or solvate thereof.

41. The method of claim 40 wherein the neurological disorder is a neurodegenerative disease.

42. The method of claim 40 or claim 41, further comprising administering to the patient a further pharmaceutically active agent.
